SEQUENCE LISTING (13 sequences)

SEQ ID NO: 1 - protein, 188 aa, Homo sapiens [sequence omitted]

SEQ ID NO: 2 - protein, 188 aa, Homo sapiens [sequence omitted]

SEQ ID NO: 3 - protein, 188 aa, Homo sapiens
MTEYKLVVVGAVGVGKSALTIQLIQNHFVDEYDPTIEDSYRKQVVIDGETCLLDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 4 - protein, 188 aa, Homo sapiens [sequence omitted]

SEQ ID NO: 5 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDDVPMVLVGNKCDLPTRTVDTKQAHELAKSYGIPFIETSAKTRQGVEDAFYTLVREIRQYRMKKLNSSDDGTQGCMGLPCVVM

SEQ ID NO: 6 - protein, 189 aa, Homo sapiens
MTEYKLVVVGADGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 7 - protein, 189 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDDVPMVLVGNKCDLAARTVESRQAQDLARSYGIPYIETSAKTRQGVEDAFYTLVREIRQHKLRKLNPPDESGPGCMSCKCVLS

SEQ ID NO: 8 - protein, 189 aa, Homo sapiens [sequence omitted]

SEQ ID NO: 9 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHHYREQIKRVKDSED

SEQ ID NO: 10 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNTKSFEDIHQYREQIKRVKDSDD

SEQ ID NO: 11 - protein, 108 aa, Homo sapiens
MTEYKLVVVGAGGVGKSALTIQLIQNHFVDEYDPTIEDSYRKQVVIDGETCLLDILDTAGQEEYSAMRDQYMRTGEGFLCVFAINNSKSFADINLYREQIKRVKDSDD

SEQ ID NO: 12 - protein, 119 aa, Homo sapiens
MAANKPKGQNSLALHKVIMVGSGGVGKSALTLQFMYDEFVEDYEPTKADSYRKKVVLDGEEVQIDILDTAGQEDYAAIRDNYFRSGEGFLCVFSITEMESFAATADFREQILRVKEDEN

SEQ ID NO: 13 - protein, 120 aa, Homo sapiens
MAANKSKGQSSLALHKVIMVGSGGVGKSALTLQFMYDEFVEDYEPTKADSYRKKVVLDGEEVQIDILDTAGQEDYAAIRDNYFRSGEGFLLVFSITEHESFTATAEFREQILRVKAEEDK

(19) United States
(12) Patent Application Publication
(10) Pub. No.: US 2024/0270736 A1
(43) Pub. Date: Aug. 15, 2024

(54) MACROCYCLIC HETEROCYCLES AND USES THEREOF

(71) Applicant: Kumquat Biosciences Inc., San Diego, CA (US)

(72) Inventors: Baogen Wu, San Diego, CA (US); Liansheng Li, San Diego, CA (US); Pingda Ren, San Diego, CA (US); Zhiyong Chen, San Diego, CA (US); Siling Zhao, San Diego, CA (US); Xiuwen Zhu, San Diego, CA (US)

(21) Appl. No.: 18/521,254

(22) Filed: Nov. 28, 2023

Related U.S. Application Data
(63) Continuation of application No. PCT/US2023/068245, filed on Jun. 9, 2023.
(60) Provisional application No. 63/351,234, filed on Jun. 10, 2022.

(51) Int. Cl. C07D 417/12; A61K 45/06; C07C 235/88; C07D 207/16; C07D 209/18; C07D 241/44; C07D 401/04; C07D 403/06; C07D 493/06

(52) U.S. Cl. CPC C07D 417/12; A61K 45/06; C07C 235/88; C07D 207/16; C07D 209/18; C07D 241/44; C07D 401/04; C07D 403/06; C07D 493/06

(57) ABSTRACT

The present disclosure provides compounds and pharmaceutically acceptable salts thereof, and methods of using the same. The compounds and methods have a range of utilities as therapeutics, diagnostics, and research tools. In particular, the subject compositions and methods are useful for reducing signaling output of oncogenic proteins.

### CROSS-REFERENCE

This application is a continuation of International Application No. PCT/US2023/068245, filed Jun. 9, 2023, which claims the benefit of U.S. Provisional Application No. 63/351,234, filed Jun. 10, 2022, each of which is incorporated herein by reference in its entirety.

### SEQUENCE LISTING

The instant application contains a Sequence Listing which has been submitted electronically in XML format and is hereby incorporated by reference in its entirety. Said XML copy, created on Nov. 28, 2023, is named 56690_753_301_SL.xml and is 13,993 bytes in size.

### BACKGROUND

Cancer (e.g., tumor, neoplasm, metastases) is the second leading cause of death worldwide estimated to be responsible for about 10 million deaths each year. Many types of cancers are marked with mutations in one or more proteins involved in various signaling pathways leading to unregulated growth of cancerous cells. In some cases, about 25 to 30 percent (%) of tumors are known to harbor Rat sarcoma (Ras) mutations. In particular, mutations in the Kirsten Ras oncogene (K-Ras) are one of the most frequent Ras mutations detected in human cancers, including lung adenocarcinomas (LUADs) and pancreatic ductal adenocarcinoma (PDAC).

Ras proteins have long been considered “undruggable,” due to, in part, high affinity to their substrate guanosine-5′-triphosphate (GTP) and/or their smooth surfaces without any obvious targeting region. The specific G12C Ras gene mutation has been identified as a druggable target to which a number of G12C specific inhibitors have been developed. However, such therapeutics are still of limited application, as the G12C mutation in Ras exhibits a much lower prevalence rate as compared to other known Ras mutations, such as G12D and G12V. Drug resistance and lack of durability impose further limitations to such therapeutics.

### SUMMARY

In view of the foregoing, there remains a considerable need for a new design of therapeutics and diagnostics that can specifically target Ras, Ras mutants and/or associated proteins of Ras to reduce Ras signaling output. Of particular interest are Ras inhibitors, including pan Ras inhibitors capable of inhibiting two or more Ras mutants and/or wildtype Ras, as well as mutant-selective inhibitors targeting mutant Ras proteins such as Ras G12D, G12C, G12S, G13D, and/or G12V, for the treatment of Ras-associated diseases (e.g., cancer). Such compositions and methods can be particularly useful for treating a variety of diseases including, but not limited to, cancers and neoplasia conditions. The present disclosure addresses these needs, and provides additional advantages applicable for diagnosis, prognosis, and/or treatment for a wide diversity of diseases.

In certain aspects, the present disclosure provides a compound of Formula (I):

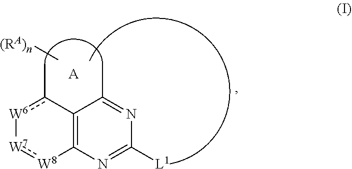

or a pharmaceutically acceptable salt or solvate thereof, wherein:
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R^{6a}) or C(O), W⁷ is N(R^{7b}), W⁸ is C(R⁸)(R^{8a}) or C(O), and each [symbol] indicates a single bond;
R⁶, R^{6a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R⁷ is -L⁷-R¹⁷;
R^{7b} is -L^{7b}-R¹⁷;
L⁷ is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
L^{7b} is selected from a bond, —O—, —C(O)—, —S—, —S(O)₂—, —S(O)—, —P(O)R^{7d}—, —C(R^{7c})₂—, —OC(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
R¹⁷ is selected from C₃₋₁₂ carbocycle and 3- to 12-membered heterocycle, each of which is optionally substituted with one or more R²⁰;
L¹ is -L²-L³-L⁴-L⁵-L⁶-;
L² is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle, wherein —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle are optionally substituted with one, two, or three R^{L};
L³ is selected from C₁₋₆ alkyl, C₂₋₆ alkenyl, C₂₋₆ alkynyl, and 2- to 6-membered heteroalkyl, each of which is optionally substituted with one, two, or three R^{L};
L⁴ and L⁶ are each independently selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₂₋₆ alkenyl, C₂₋₆ alkynyl, 2- to 6-membered heteroalkyl, C₃₋₁₀ carbocycle, and 3- to 10-membered heterocycle, wherein C₁₋₆ alkyl, C₂₋₆ alkenyl, C₂₋₆ alkynyl, 2- to 6-membered heteroalkyl, C₃₋₁₀ carbocycle, and 3- to 10-membered heterocycle are optionally substituted with one, two, or three R^{L};
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 6;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

The compound of Formula (I) may be a compound of Formula (I′):

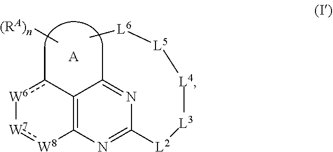

or a pharmaceutically acceptable salt or solvate thereof.

For a compound of Formula (I) or (I′), A may be a 5- to 18-membered heterocycle, such as a 5- to 8-membered monocyclic heterocycloalkenyl. In some embodiments, A is 8- to 12-membered fused bicyclic heterocycloalkenyl.

The compound of Formula (I) may be a compound of Formula (I-A):

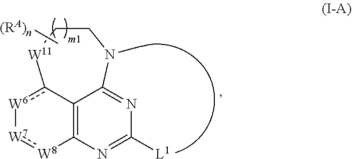

or a pharmaceutically acceptable salt or solvate thereof, wherein:
W¹¹ is selected from —O—, —N(R^{7d})—, —S—, and —C(R^{7c})₂—; and
m1 is 1 or 2,
   such as a compound of Formula (I-A1), (I-A2), or (I-A3):

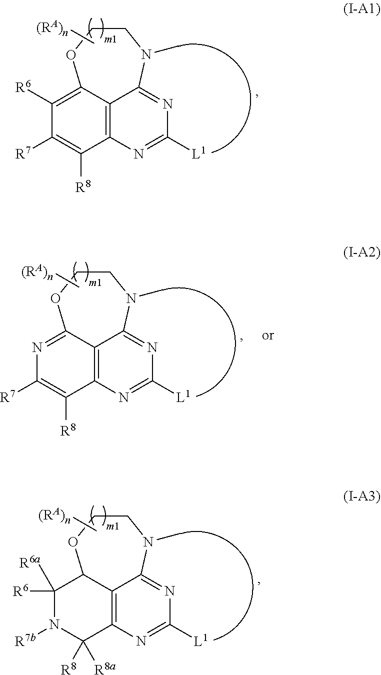

or a pharmaceutically acceptable salt or solvate thereof.

The compound of Formula (I) may be a compound of Formula (I-B):

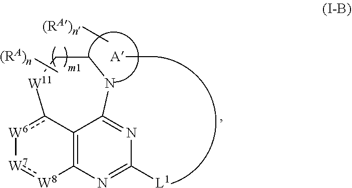

or a pharmaceutically acceptable salt or solvate thereof, wherein:
W¹¹ is selected from —O—, —N(R^{7d})—, —S—, and —C(R^{7c})₂—;
A′ is 5- to 7-membered heterocycle;
R^{A′} is independently selected at each occurrence from R^{A};
n′ is an integer from 0 to (6-n); and
m1 is 1 or 2,
   such as a compound of Formula (I-B1), (I-B2), or (I-B3):

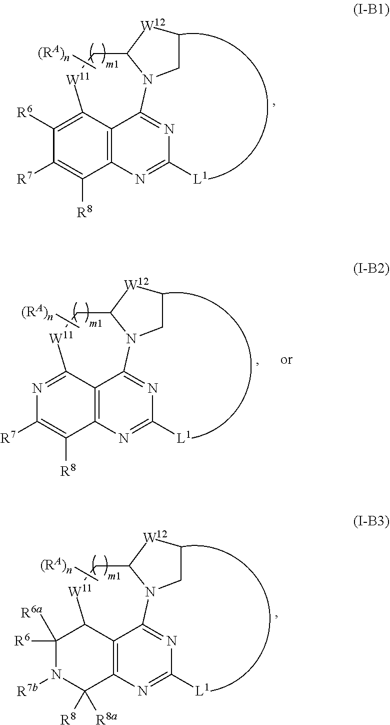

or a pharmaceutically acceptable salt or solvate thereof, wherein W¹² is selected from —O—, —N(R^{7d})—, —C(R^{7c})₂—, —OC(R^{7c})₂—, —N(R^{7d})C(R^{7c})₂—, —C(R^{7c})₂C(R^{7c})₂, —C(R^{7c})₂O—, and —C(R^{7c})₂N(R^{7d})—.

In some embodiments, for a compound of Formula (I-A) or (I-B), W¹¹ is —O— and m1 is 1. In some embodiments, for a compound of Formula (I-B1), (I-B2), or (I-B3), W¹² is selected from —O—, —N(R^{7d})—, —CH₂—, —OCH₂—, —N(R^{7d})CH₂—, —CH₂CH₂, —CH₂O—, and —CH₂N(R^{7d})—.

In some embodiments, for a compound of Formula (I), (I′), (I-A), or (I-B), W⁶ is C(R⁶), W⁷ is C(R⁷), W⁸ is C(R⁸), and each [symbol] indicates a double bond. In some embodiments, W⁶ is N, W⁷ is C(R⁷), W⁸ is C(R⁸), and each [symbol] indicates a double bond. In some embodiments, W⁶ is C(R⁶)(R⁶) or C(O), W⁷ is N(R^{7b}), W⁸ is C(R⁸)(R^{8a}) or C(O), and each [symbol] indicates a single bond.

In some embodiments, for a compound of Formula (I), (I′), (I-A), (I-A1), (I-A3), (I-B1), or (I-B3), R⁶ is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰. In some embodiments, R⁶ is selected from hydrogen, halogen, —CN, C₁₋₆ alkyl, and —OR¹², wherein C₁₋₆ alkyl is optionally substituted with one, two, or three R²⁰, such as R⁶ is halogen.

In some embodiments, for a compound of Formula (I), (I′), (I-A), (I-A1), (I-A2), (I-B1), or (I-B2), L⁷ is selected from a bond, —O—, —N(R^{7d})—, —C(O)—, and —C(R^{7c})₂—. In some embodiments, L⁷ is a bond.

In some embodiments, for a compound of Formula (I), (I′), (I-A), (I-A1), (I-A2), (I-A3), (I-B1), (I-B2), or (I-B3), R⁸ is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰. In some embodiments, R⁸ is selected from hydrogen, halogen, —CN, C₁₋₆ alkyl, and —OR¹², wherein C₁₋₆ alkyl is optionally substituted with one, two, or three R²⁰. In some embodiments, R⁸ is halogen.

In some embodiments, for a compound of Formula (I), (I′), (I-A), (I-A3), or (I-B3), R^{6a} is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰. In some embodiments, R^{6a} is selected from hydrogen, halogen, —CN, C₁₋₆ alkyl, and —OR¹², wherein C₁₋₆ alkyl is optionally substituted with one, two, or three R²⁰. In some embodiments, R⁶ and R^{6a} are each hydrogen. In some embodiments, L^{7b} is selected from a bond, —O—, —C(O)—, and —C(R^{7c})₂—. In some embodiments, L^{7b} is a bond. In some embodiments, R^{8a} is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰. In some embodiments, R^{8a} is selected from hydrogen, halogen, —CN, C₁₋₆ alkyl, and —OR¹², wherein C₁₋₆ alkyl is optionally substituted with one, two, or three R²⁰. In some embodiments, R⁸ and R^{8a} are each hydrogen.

In some embodiments, for a compound of Formula (I), (I′), (I-A), (I-A1), (I-A2), (I-A3), (I-B1), (I-B2), or (I-B3), R¹⁷ is selected from C₆₋₁₂ aryl and 5- to 12-membered heteroaryl, each of which is optionally substituted with one, two, or three R²⁰. In some embodiments, R¹⁷ is selected from C₁₀ aryl and 9-membered heteroaryl, each of which is optionally substituted with one, two, or three R²⁰. In some embodiments, R¹⁷ is selected from naphthalenyl and benzothiophenyl, each of which is optionally substituted with one, two, or three R²⁰. In some embodiments, R¹⁷ is substituted with one, two, or three substituents independently selected from halogen, —CN, C₁₋₃ alkyl, C₂₋₃ alkenyl, C₂₋₃ alkynyl, —OR²², and —N(R²²)(R²³). In some embodiments, R¹⁷ is substituted with one, two, or three substituents independently selected from halogen, —CN, —CH₃, —C═CH, —OH, and —NH₂. In some embodiments, R¹⁷ is substituted with —F, —CN, and —NH₂.

In some embodiments, for a compound of Formula (I, (I) (I-A), (I-A1), (I-A2), (I-A3), (I-B1), (I-B2), or (I-B3), R¹⁷ is selected from

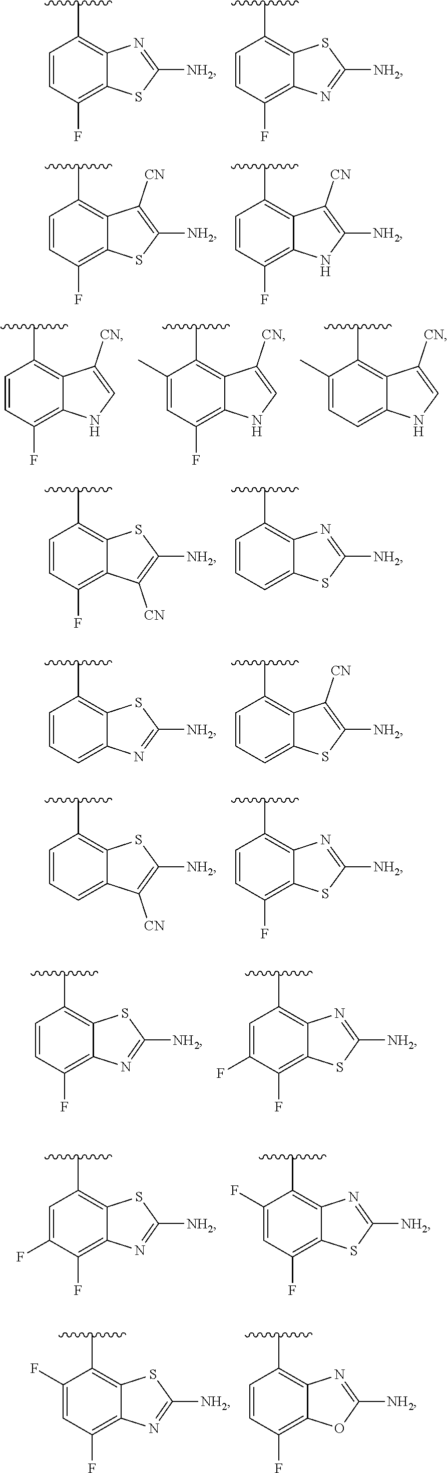

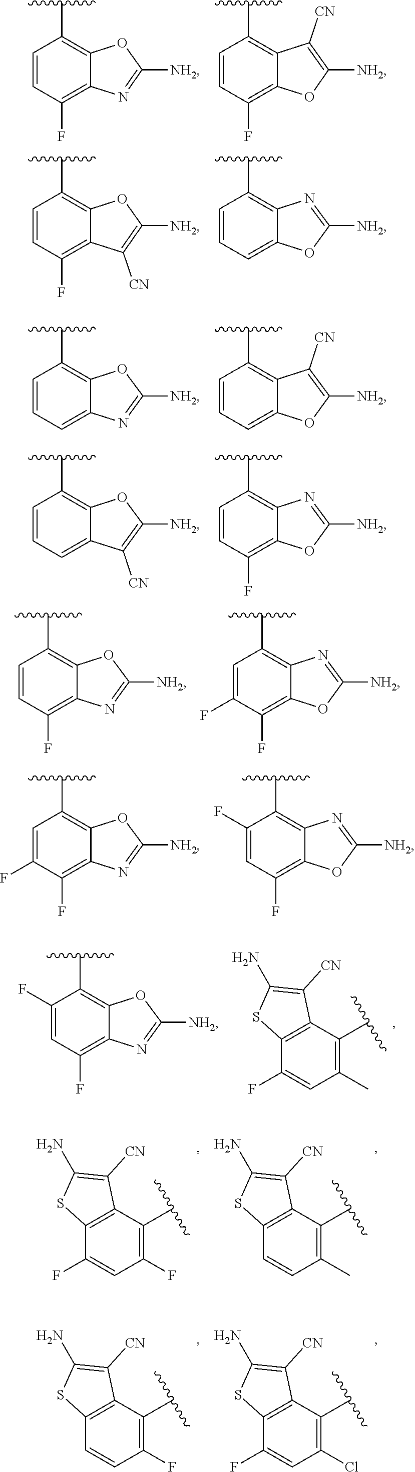

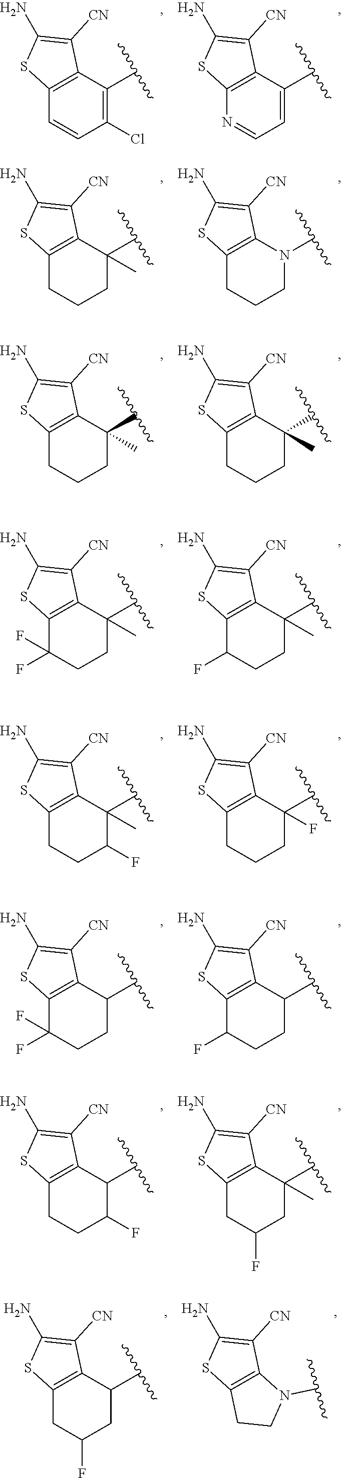

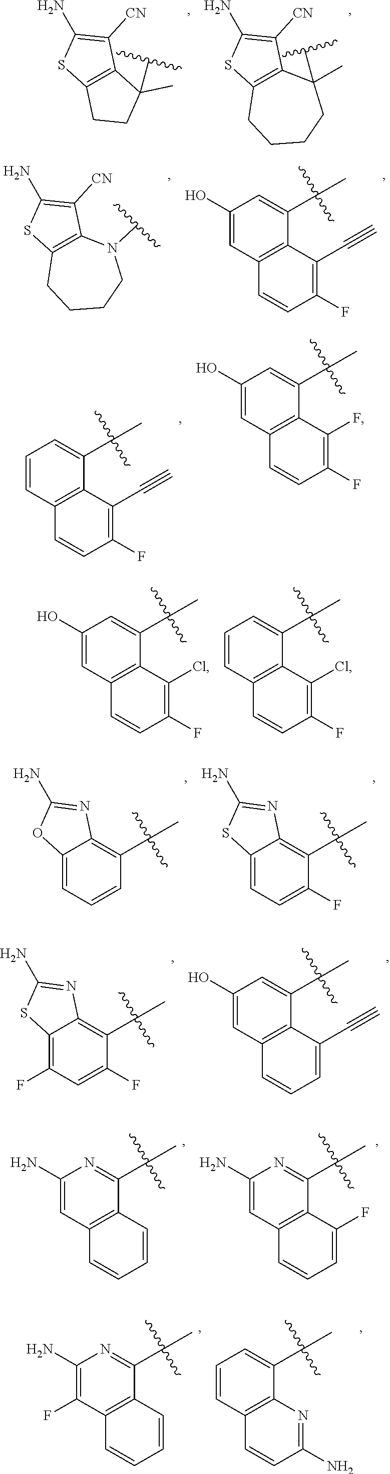

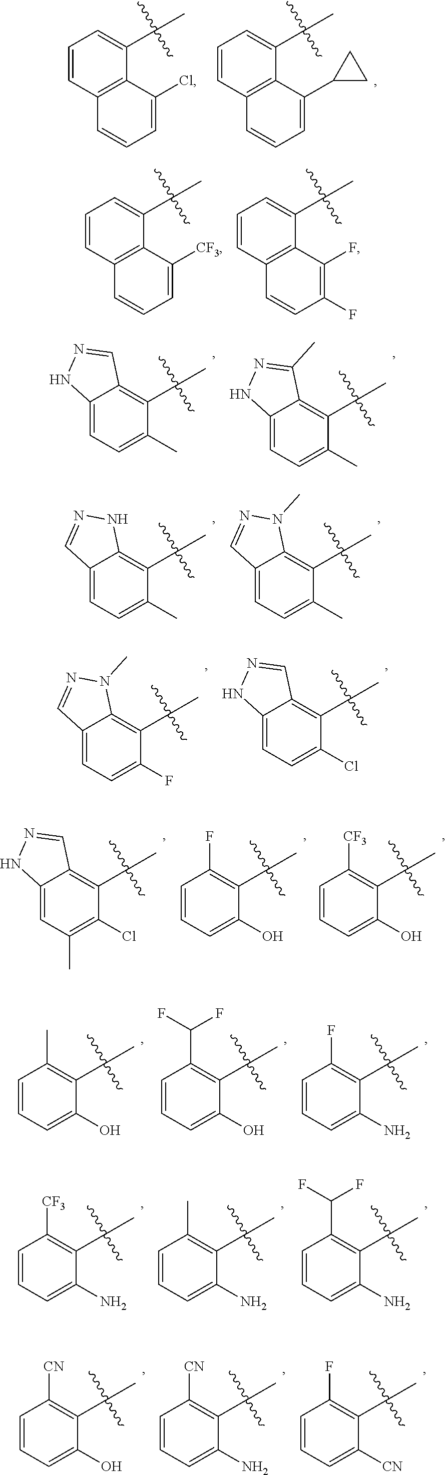

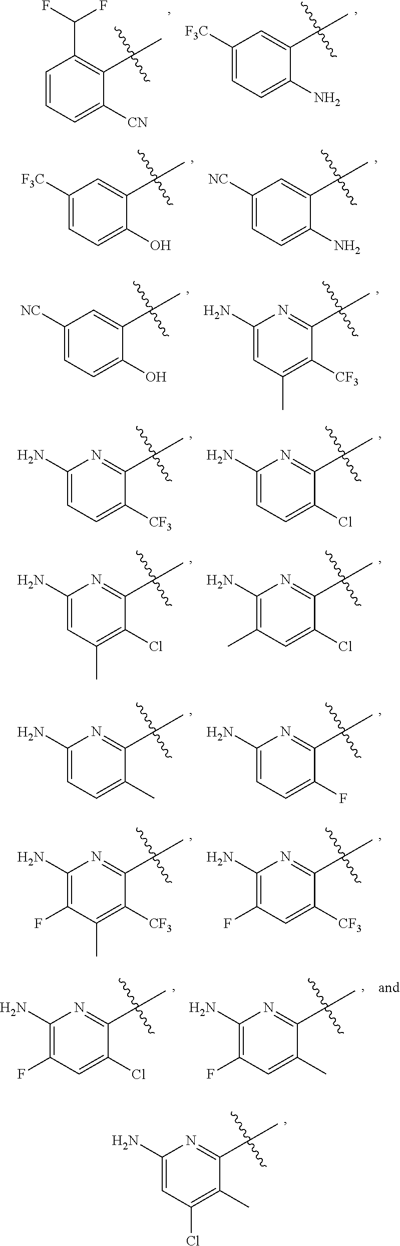

such as R¹⁷ is selected from

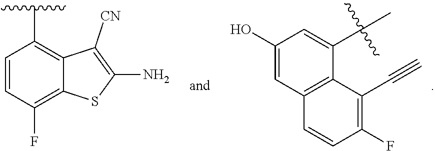 [SMILES: *c1ccc(F)c2sc(N)c(C#N)c12 ; *c1cc(O)cc2ccc(F)c(C#C)c12]

In some embodiments, for a compound of Formula (I), (I′), (I-A), (I-A1), (I-A2), (I-A3), (I-B1), (I-B2), or (I-B3), R² is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 8-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆₆ haloalkyl, C₃₋₈ carbocycle, and 3- to 8-membered heterocycle are optionally substituted with one, two, or three R²⁰. In some embodiments, R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³). In some embodiments, n is an integer from 0 to 3, such as n is 0.

In some embodiments, for a compound of Formula (I), (I′), (I-A), (I-A1), (I-A2), (I-A3), (I-B1), (I-1B2), or (I-1B3), L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—. In some embodiments, L² is selected from —O— and —OC(R^{7c})₂—. In some embodiments, L² is —OCH(3- to 10-membered heterocycle)-, wherein the 3- to 10-membered heterocycle is optionally substituted with one, two, or three substituents selected from halogen and C₁₋₃ alkyl. In some embodiments, L² is selected from

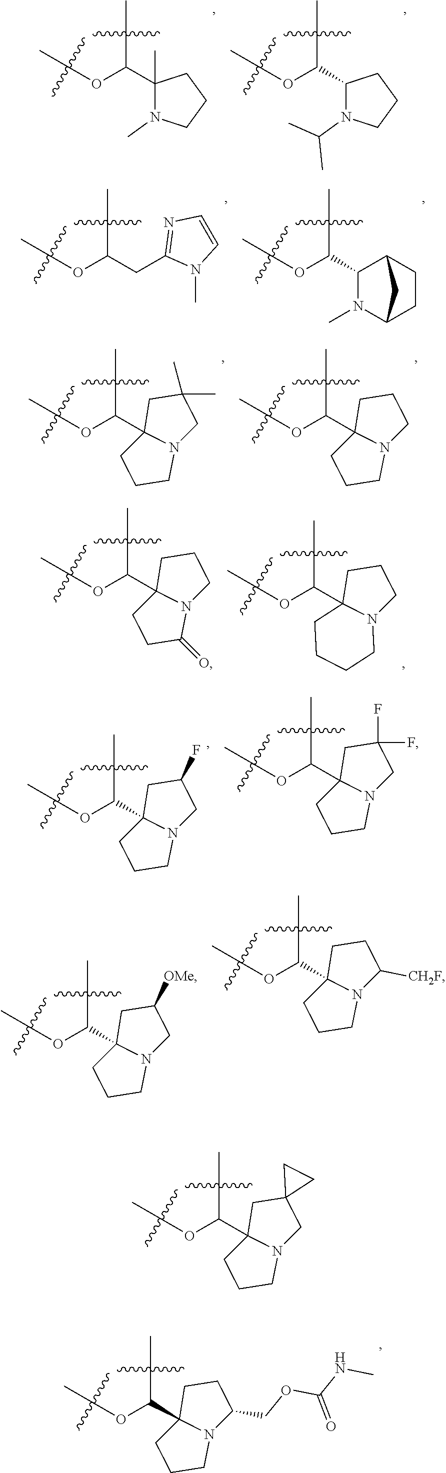

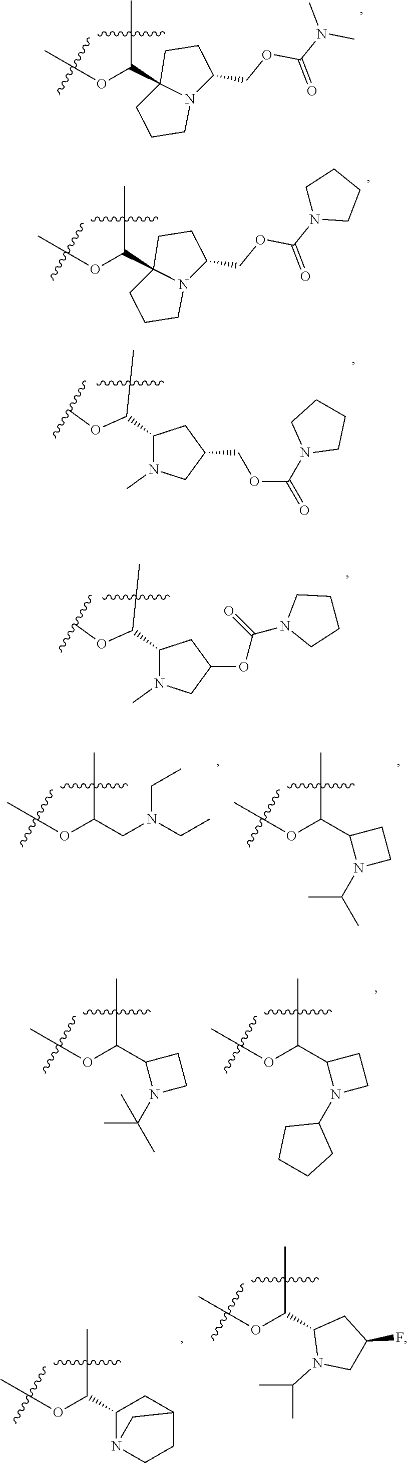

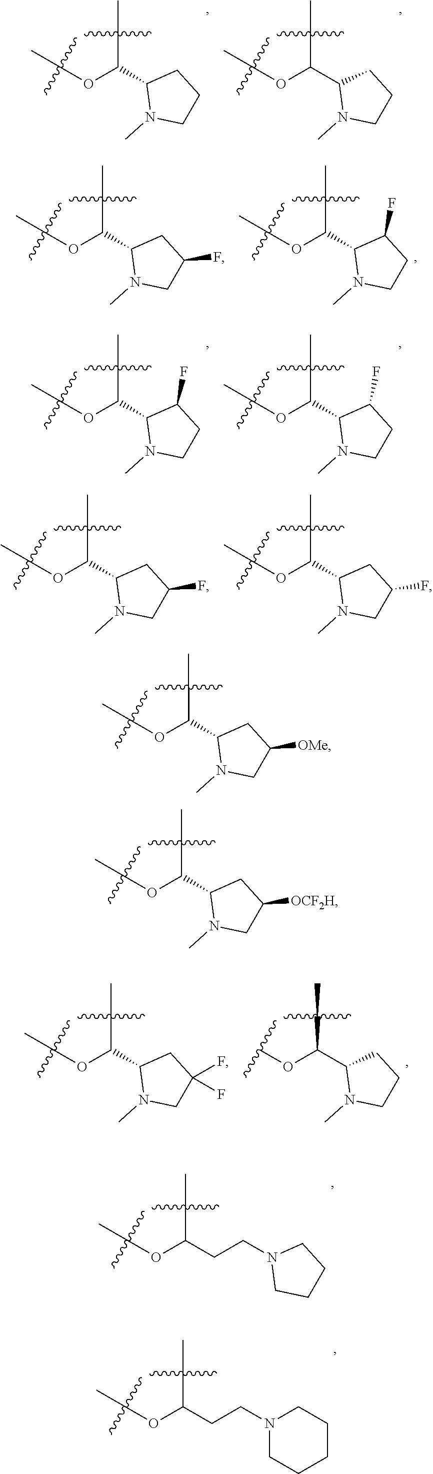

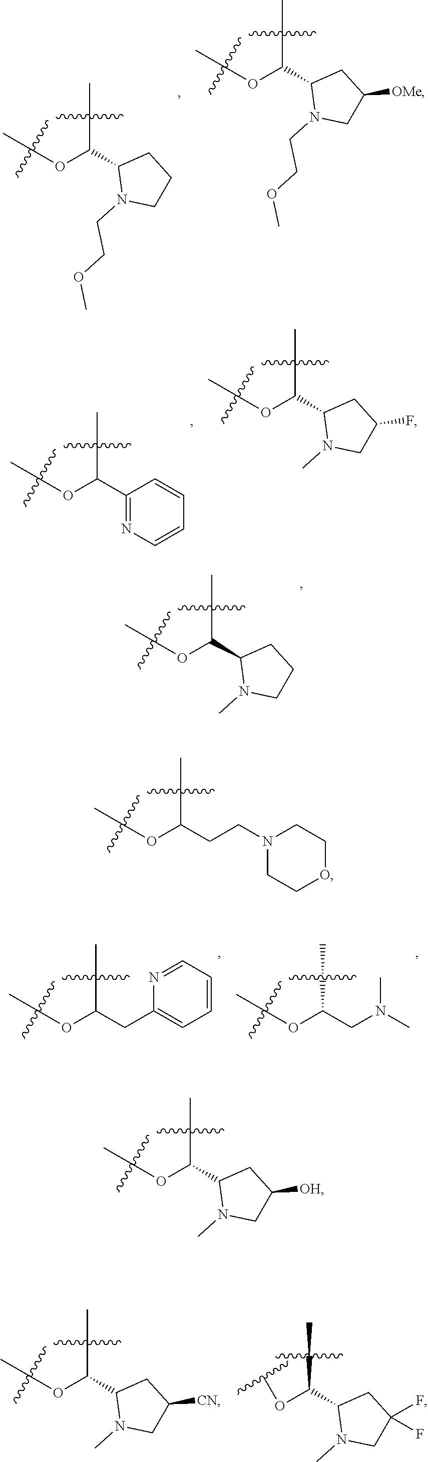

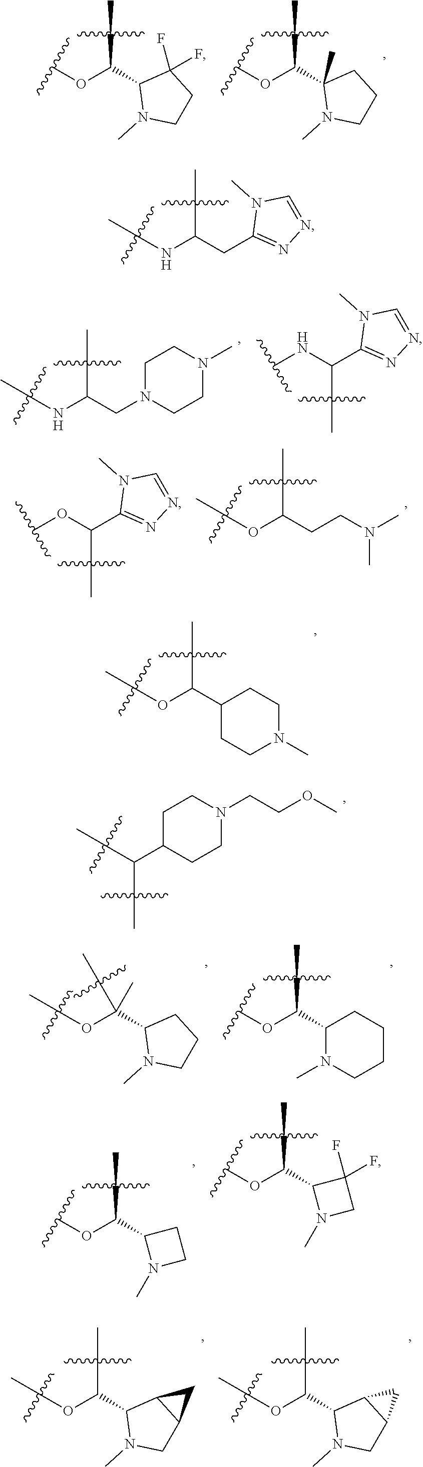

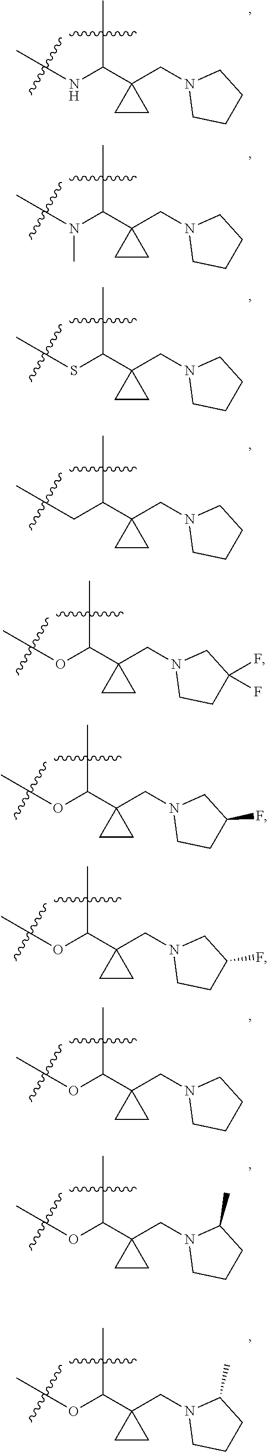

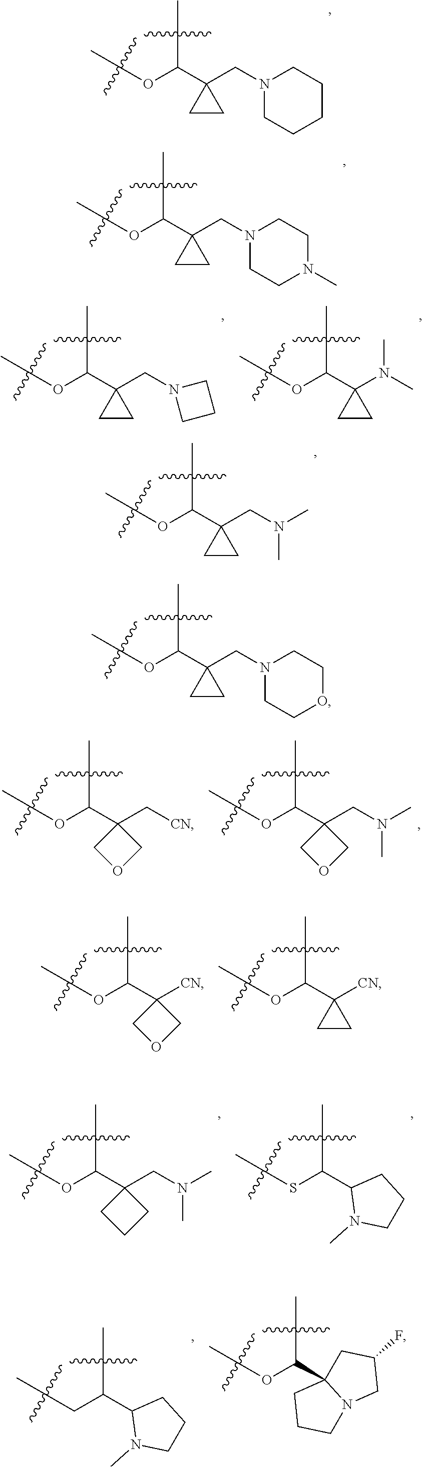

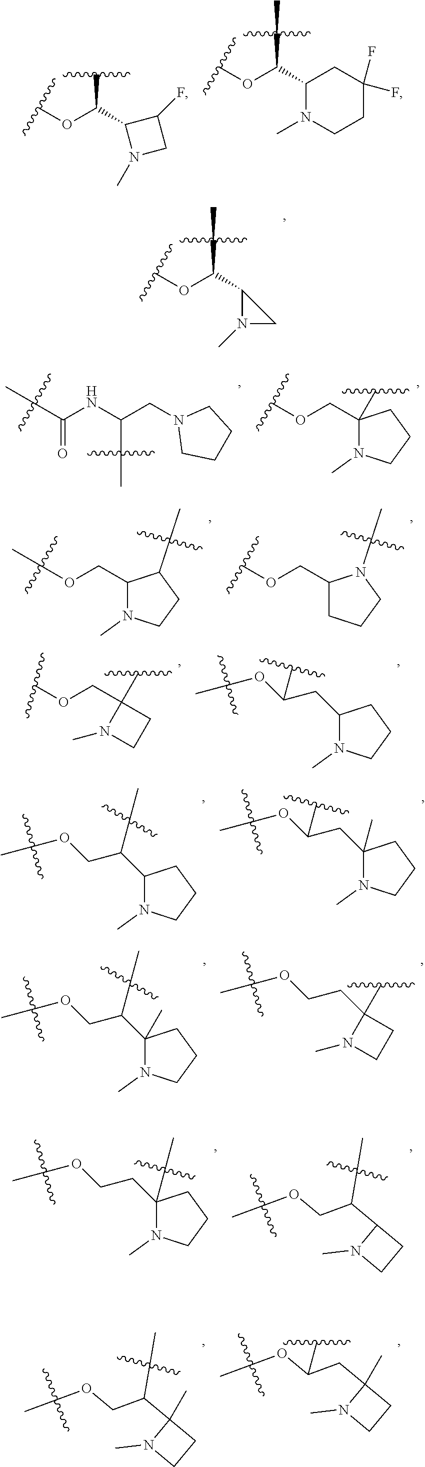

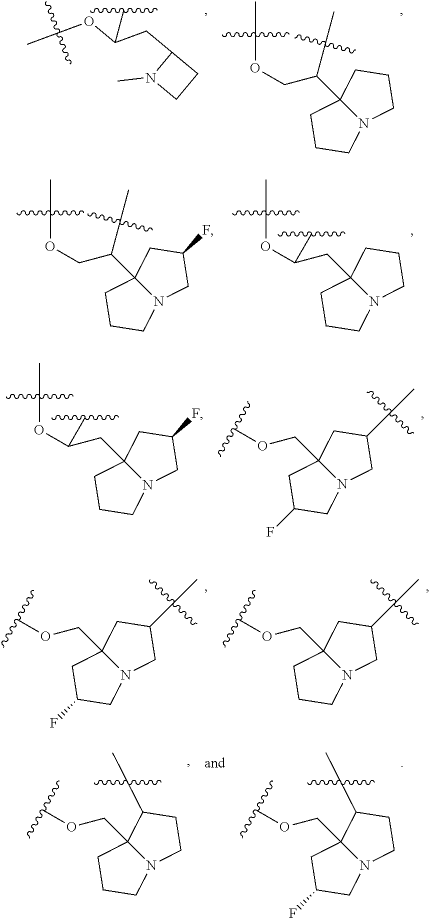

In some embodiments, for a compound of Formula (I), (I′), (I-A), (I-A1), (I-A2), (I-A3), (I-B1), (I-B2), or (I-B3), L³ is selected from C₁₋₆ alkyl, C₂₋₆ alkenyl, and 2- to 6-membered heteroalkyl, each of which is optionally substituted with one, two, or three R^{L}. In some embodiments, L³ is selected from C₁₋₆ alkyl and C₂₋₆ alkenyl. In some embodiments, L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L}. In some embodiments, L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}. In some embodiments, L⁵ is selected from a bond and C₁₋₆ alkyl, wherein C₁₋₆ alkyl is optionally substituted with one, two, or three R^{L}. In some embodiments, L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

In some embodiments, for a compound of Formula (I), (I′), (I-A), (I-A1), (I-A2), (I-A3), (I-B1), (I-B2), or (I-B3), L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—; L³ is selected from C₁₋₆ alkyl, C₂₋₆ alkenyl, and 2- to 6-membered heteroalkyl, each of which is optionally substituted with one, two, or three R^{L}; L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}; L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

In some embodiments, for a compound of Formula (I), (I′), (I-A), (I-A1), (I-A2), (I-A3), (I-B1), (I-B2), or (I-B3), L² is selected from —O— and —OC(R^{7c})₂—; L³ is selected from C₁₋₆ alkyl and C₂₋₆ alkenyl; L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L}; L⁵ is selected from a bond and C₁₋₆ alkyl, wherein C₁₋₆ alkyl is optionally substituted with one, two, or three R^{L}; and L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

In some embodiments, for a compound of Formula (I), (I′), (I-A), (I-A1), (I-A2), (I-A3), (I-B1), (I-B2), or (I-B3), R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰. In some embodiments, R^{L} is independently selected at each occurrence from halogen, oxo, C₁₋₆ alkyl, C₃₋₈ carbocycle, 3- to 10-membered heterocycle, and —C(O)R¹², wherein C₁₋₆ alkyl, C₃₋₈ carbocycle, and 3- to 10-membered heterocycle are optionally substituted with one, two, or three R²⁰.

In some embodiments, for a compound of Formula (I), (I′), (I-A), (I-A1), (I-A2), (I-A3), (I-B1), (I-B2), or (I-B3), R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰. In some embodiments, R^{7c} is independently selected at each occurrence from hydrogen, halogen, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰. In some embodiments, R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰.

In some embodiments, for a compound of Formula (I), (I′), (I-A), (I-A1), (I-A2), (I-A3), (I-B1), (I-B2), or (I-B3), R⁶, if present, is selected from hydrogen and halogen; R^{6a}, if present, is hydrogen; L⁷, if present, is selected from a bond, —O—, —N(R^{7d})—, —C(O)—, and —C(R^{7c})₂—; L^{7b}, if present, is selected from a bond, —O—, —C(O)—, and —C(R^{7c})₂—; R¹⁷ is selected from C₆₋₁₂ aryl and 6- to 12-membered heteroaryl, each of which is optionally substituted with one, two, or three R²⁰; R⁸ is selected from hydrogen, halogen, —CN, C₁₋₆ alkyl, and —OR¹², wherein C₁₋₆ alkyl is optionally substituted with one, two, or three R²⁰; R^{8a}, if present, is hydrogen; R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³); n is an integer from 0 to 3; L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—; L³ is selected from C₁₋₆ alkyl, C₂₋₆ alkenyl, and 2- to 6-membered heteroalkyl, each of which is optionally substituted with one, two, or three R^{L}; L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}; L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

In some embodiments, for a compound of Formula (I), (I′), (I-A), (I-A1), (I-A2), (I-A3), (I-B1), (I-B2), or (I-B3), R⁶, if present, is selected from hydrogen and halogen; R^{6a}, if present, is hydrogen; L⁷, if present, is a bond; L^{7b}, if present, is a bond; R¹⁷ is selected from naphthalenyl and benzothiophenyl, each of which is optionally substituted with one, two, or three R²⁰; R⁸ is halogen; R^{8a}, if present, is hydrogen; n is 0; L² is selected from —O— and —OC(R^{7c})₂—; L³ is selected from C₁₋₆ alkyl and C₂₋₆ alkenyl; L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L}; L⁵ is selected from a bond and C₁₋₆ alkyl, wherein C₁₋₆ alkyl is optionally substituted with one, two, or three R^{L}; and L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

In some embodiments, for a compound of Formula (I), (I′), (I-A), (I-A1), (I-A2), (I-A3), (I-B1), (I-B2), or (I-B3), R¹⁷ is selected from

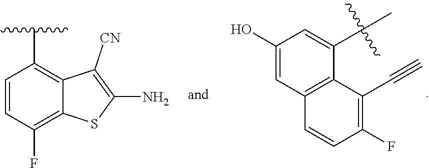 [SMILES: *c1ccc(F)c2sc(N)c(C#N)c12 ; *c1cc(O)cc2ccc(F)c(C#C)c12]

In some embodiments, R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰; R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰; and R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰.

In some embodiments, the present disclosure provides a compound disclosed herein, or a pharmaceutically acceptable salt or solvate thereof.

In certain aspects, the present disclosure provides a compound having the formula B-L^{BE}-E wherein: B is a monovalent form of a compound disclosed herein, such as a compound of Formula (I), (I′), (I-A), (I-A1), (I-A2), (I-A3), (I-B1), (I-B2), or (I-B3); L^{BE} is a covalent linker bonded to B and E; and E is a monovalent form of a degradation enhancer. In some embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885. In some embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.

In some embodiments, L^{BE} is -L^{BE1}-L^{BE2}-L^{BE3}-L^{BE4}-L^{BE5}-; wherein L^{BE1}, L^{BE2}, L^{BE3}, L^{BE4}, and L^{BE5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆ alkylene, (—O—C₁₋₆ alkyl)₂-, (—C₁₋₆ alkyl-O)₂—, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene, wherein C₁₋₆ alkylene, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene are optionally substituted with one, two, or three R²⁰; and wherein each C₁₋₆ alkyl of (—O—C₁₋₆ alkyl)₂- and (—C₁₋₆ alkyl-O)₂— is optionally substituted with one, two, or three R²⁰; and z is independently an integer from 0 to 10.

In some embodiments, L^{BE} is —(O-C₂alkyl)_{z}- and z is an integer from 1 to 10. In some embodiments, L^{BE} is —(C₂alkyl-O—)_{z}— and z is an integer from 1 to 10. In some embodiments, L^{BE} is —(CH₂)_{zz1}L^{BE2}(CH₂O)_{zz2}—, wherein L^{BE2} is a bond, a 5 or 6 membered heterocycloalkylene or heteroarylene, phenylene, —(C₂-C₄)alkynylene, —SO₂— or —NH—; and zz1 and zz2 are independently an integer from 0 to 10. In some embodiments, L^{BE} is —(CH₂)_{zz1}(CH₂O)_{zz2}—, wherein zz1 and zz2 are each independently an integer from 0 to 10. In some embodiments, L^{BE} is a PEG linker. In some embodiments, E is a monovalent form of a compound selected from

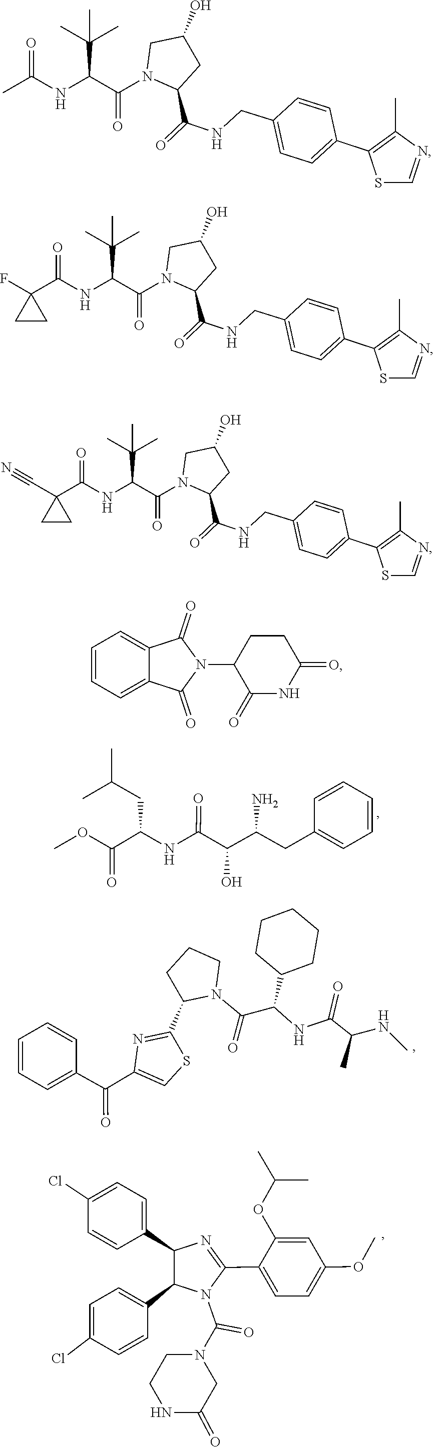

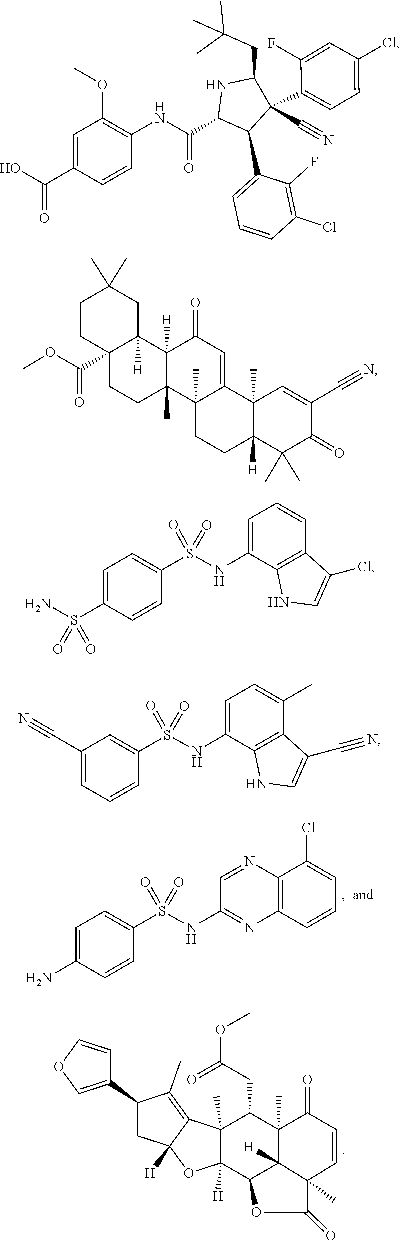

In certain aspects, the present disclosure provides a pharmaceutical composition comprising a compound disclosed herein, such as a compound of Formula (I), (I′), (I-A), (I-A1), (I-A2), (I-A3), (I-B1), (I-B2), or (I-B3), or a pharmaceutically acceptable salt or solvate thereof, and a pharmaceutically acceptable excipient.

In certain aspects, the present disclosure provides a method of treating cancer in a subject in need thereof, comprising administering to the subject a therapeutically effective amount of a compound disclosed herein, such as a compound of Formula (I), (I′), (I-A), (I-A1), (I-A2), (I-A3), (I-B1), (I-B2), or (I-B3), or a pharmaceutically acceptable salt or solvate thereof. In certain aspects, the present disclosure provides a method of treating cancer in a subject comprising a Ras mutant protein, the method comprising: inhibiting the Ras mutant protein of said subject by administering to said subject a compound disclosed herein, such as a compound of Formula (I), (I′), (I-A), (I-A1), (I-A2), (I-A3), (I-B1), (I-B2), or (I-B3), wherein the compound is characterized in that upon contacting the Ras mutant protein, said Ras mutant protein exhibits reduced Ras signaling output. In some embodiments, the cancer is a solid tumor. In some embodiments, the cancer is a hematological cancer. In practicing any of the subject methods, a compound disclosed herein may be provided as a pharmaceutically acceptable salt or solvate.

In certain aspects, the present disclosure provides a method of modulating signaling output of a Ras protein, comprising contacting a Ras protein with an effective amount of a compound disclosed herein, such as a compound of Formula (I), (I′), (I-A), (I-A1), (I-A2), (I-A3), (I-B1), (I-B2), or (I-B3), or a pharmaceutically acceptable salt or solvate thereof, thereby modulating the signaling output of the Ras protein. In certain aspects, the present disclosure provides a method of inhibiting cell growth, comprising administering an effective amount of a compound disclosed herein, such as a compound of Formula (I), (I′), (I-A), (I-A1), (I-A2), (I-A3), (I-B1), (I-B2), or (I-B3), or a pharmaceutically acceptable salt or solvate thereof, to a cell expressing a Ras protein, thereby inhibiting growth of said cells.

Any of the above methods may further comprise administering an additional agent. In some embodiments, the additional agent comprises (1) an inhibitor of MEK; (2) an inhibitor of epidermal growth factor receptor (EGFR) and/or of mutants thereof; (3) an immunotherapeutic agent; (4) a taxane; (5) an anti-metabolite; (6) an inhibitor of FGFR1 and/or FGFR2 and/or FGFR3 and/or of mutants thereof; (7) a mitotic kinase inhibitor; (8) an anti-angiogenic drug; (9) a topoisomerase inhibitor; (10) a platinum-containing compound; (12) an inhibitor of c-MET and/or of mutants thereof; (13) an inhibitor of BCR-ABL and/or of mutants thereof; (14) an inhibitor of ErbB2 (Her2) and/or of mutants thereof; (15) an inhibitor of AXL and/or of mutants thereof; (16) an inhibitor of NTRK1 and/or of mutants thereof; (17) an inhibitor of RET and/or of mutants thereof; (18) an inhibitor of A-Raf and/or B-Raf and/or C-Raf and/or of mutants thereof; (19) an inhibitor of ERK and/or of mutants thereof; (20) an MDM2 inhibitor; (21) an inhibitor of mTOR; (23) an inhibitor of IGF1/2 and/or of IGF1-R; (24) an inhibitor of CDK9; (25) an inhibitor of farnesyl transferase; (26) an inhibitor of SHIP pathway; (27) an inhibitor of SRC; (28) an inhibitor of JAK; (29) a PARP inhibitor, (31) a ROS1 inhibitor; (32) an inhibitor of SHP pathway, or (33) an inhibitor of Src, FLT3, HDAC, VEGFR, PDGFR, LCK, Bcr-Abl or AKT; (34) an inhibitor of KrasG12C mutant; (35) a SHC inhibitor (e.g., PP2, AID371185); (36) a GAB inhibitor; (38) a PI-3 kinase inhibitor; (39) a MARPK inhibitor; (40) a CDK4/6 inhibitor; (41) a MAPK inhibitor; (42) a SHP2 inhibitor; (43) a checkpoint immune blockade agent; (44) a SOS1 inhibitor; or (45) a SOS2 inhibitor. In some embodiments, the additional agent comprises an inhibitor of SHP2 selected from RMC-4630, TNO155, JAB-3068, IACS-13909/BBP-398, SHP099, ERAS-601, and RMC-4550. In some embodiments, the additional agent comprises an inhibitor of SOS selected from RMC-5845, BI-3406, BI-1701963, MRTX0902, and BAY 293. In some embodiments, the additional agent comprises an inhibitor of EGFR selected from afatinib, erlotinib, gefitinib, lapatinib, cetuximab panitumumab, osimertinib, olnutinib, and EGF-816. In some embodiments, the additional agent comprises an inhibitor of MEK selected from trametinib, cobimetinib, binimetinib, selumetinib, refametinib, and AZD6244. In some embodiments, the additional agent comprises an inhibitor of ERK selected from ulixertinib, MK-8353, LTT462, AZD0364, SCH772984, BIX02189, LY3214996, and ravoxertinib. In some embodiments, the additional agent comprises an inhibitor of CDK4/6 selected from palbociclib, ribociclib, and abemaciclib. In some embodiments, the additional agent comprises an inhibitor of BRAF selected from sorafenib, vemurafenib, dabrafenib, encorafenib, regorafenib, and GDC-879.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 depicts a sequence alignment of various wild type Ras proteins including K-Ras, H-Ras, N-Ras, RalA, and RalB, from top to bottom.

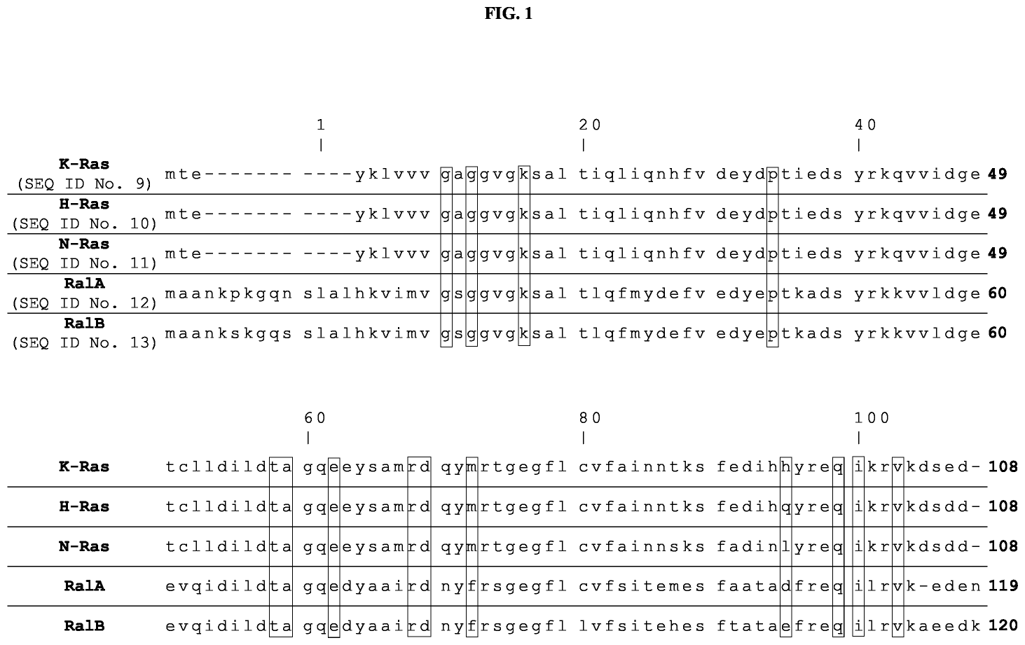

### DETAILED DESCRIPTION

Unless defined otherwise, all technical and scientific terms used herein have the same meaning as is commonly understood by one of skill in the art to which this disclosure belongs. In the event that there are a plurality of definitions for terms herein, those in this section prevail. All patents, patent applications, publications and published nucleotide and amino acid sequences (e.g., sequences available in GenBank or other databases) referred to herein are incorporated by reference. Chemical structures are named herein according to IUPAC conventions as implemented in ChemDraw® software (Perkin Elmer, Inc., Cambridge, MA). The section headings used herein are for organizational purposes only and are not to be construed as limiting the subject matter described. As used in the specification and claims, the singular forms “a”, “an” and “the” include plural references unless the context clearly dictates otherwise. Furthermore, use of the term “including” as well as other forms, such as “include”, “includes”, and “included”, is not limiting. The section headings used herein are for organizational purposes only and are not to be construed as limiting the subject matter described.

The term “C_{x-y}” or “Cₓ-C_{y}” when used in conjunction with a chemical moiety, such as alkyl, alkenyl, or alkynyl, is meant to include groups that contain from x to y carbons in the chain. For example, the term “C_{x-y} alkyl” refers to substituted or unsubstituted saturated hydrocarbon groups, including straight-chain alkyl and branched-chain alkyl groups, that contain from x to y carbons in the chain.

“Alkyl” refers to substituted or unsubstituted saturated hydrocarbon groups, including linear and branched alkyl groups. An alkyl group may contain from one to twelve carbon atoms (e.g., C₁₋₁₂ alkyl), such as one to eight carbon atoms (C₁₋₈ alkyl) or one to six carbon atoms (C₁₋₆ alkyl). Exemplary alkyl groups include methyl, ethyl, n-propyl, isopropyl, n-butyl, isobutyl, sec-butyl, tert-butyl, pentyl, isopentyl, neopentyl, hexyl, septyl, octyl, nonyl, and decyl. An alkyl group is attached to the rest of the molecule by a single bond. Unless stated otherwise specifically in the specification, an alkyl group is optionally substituted by one or more substituents such as those substituents described herein.

“Haloalkyl” refers to an alkyl group that is substituted by one or more halogens. Exemplary haloalkyl groups include trifluoromethyl, difluoromethyl, trichloromethyl, 2,2,2-trifluoroethyl, 1,2-difluoroethyl, 3-bromo-2-fluoropropyl, and 1,2-dibromoethyl.

“Alkenyl” refers to substituted or unsubstituted hydrocarbon groups, including linear and branched alkenyl groups, containing at least one double bond. An alkenyl group may contain from two to twelve carbon atoms (e.g., C₂₋₁₂ alkenyl), such as two to eight carbon atoms (C₂₋₈ alkenyl) or two to six carbon atoms (C₂₋₆ alkenyl). Exemplary alkenyl groups include ethenyl (i.e., vinyl), prop-1-enyl, but-1-enyl, pent-1-enyl, penta-1,4-dienyl, and the like. Unless stated otherwise specifically in the specification, an alkenyl group is optionally substituted by one or more substituents such as those substituents described herein.

“Alkynyl” refers to substituted or unsubstituted hydrocarbon groups, including linear and branched alkynyl groups, containing at least one triple bond. An alkynyl group may contain from two to twelve carbon atoms (e.g., C₂₋₁₂ alkynyl), such as two to eight carbon atoms (C₂₋₈ alkynyl) or two to six carbon atoms (C₂₋₆ alkynyl). Exemplary alkynyl groups include ethynyl, propynyl, butynyl, pentynyl, hexynyl, and the like. Unless stated otherwise specifically in the specification, an alkynyl group is optionally substituted by one or more substituents such as those substituents described herein.

“Alkylene” or “alkylene chain” refers to substituted or unsubstituted divalent saturated hydrocarbon groups, including linear alkylene and branched alkylene groups, that contain from one to twelve carbon atoms (e.g., C₁₋₁₂ alkylene), such as one to eight carbon atoms (C₁₋₈ alkylene) or one to six carbon atoms (C₁₋₆ alkylene). Exemplary alkylene groups include methylene, ethylene, propylene, and n-butylene. Similarly, “alkenylene” and “alkynylene” refer to alkylene groups, as defined above, which comprise one or more carbon-carbon double or triple bonds, respectively. The points of attachment of the alkylene, alkenylene or alkynylene chain to the rest of the molecule can be through one carbon or any two carbons of the chain. Unless stated otherwise specifically in the specification, an alkylene, alkenylene, or alkynylene group is optionally substituted by one or more substituents such as those substituents described herein.

“Heteroalkyl”, “heteroalkenyl” and “heteroalkynyl” refer to substituted or unsubstituted alkyl, alkenyl and alkynyl groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkyl group has a chain length of 3 to 8 atoms. Connection to the rest of the molecule may be through either a heteroatom or a carbon in the heteroalkyl, heteroalkenyl, or heteroalkynyl chain. Unless stated otherwise specifically in the specification, a heteroalkyl, heteroalkenyl, or heteroalkynyl group is optionally substituted by one or more substituents such as those substituents described herein.

“Heteroalkylene”, “heteroalkenylene” and “heteroalkynylene” refer to substituted or unsubstituted alkylene, alkenylene and alkynylene groups, respectively, in which one or more, such as 1, 2 or 3, of the carbon atoms are replaced with a heteroatom, such as O, N, P, Si, S, or combinations thereof. Any nitrogen, phosphorus, and sulfur heteroatoms present in the chain may optionally be oxidized, and any nitrogen heteroatoms may optionally be quaternized. If given, a numerical range refers to the chain length in total. For example, a 3- to 8-membered heteroalkylene group has a chain length of 3 to 8 atoms. The points of attachment of the heteroalkylene, heteroalkenylene or heteroalkynylene chain to the rest of the molecule can be through either one heteroatom or one carbon, or any two heteroatoms, any two carbons, or any one heteroatom and any one carbon in the heteroalkylene, heteroalkenylene or heteroalkynylene chain. Unless stated otherwise specifically in the specification, a heteroalkylene, heteroalkenylene, or heteroalkynylene group is optionally substituted by one or more substituents such as those substituents described herein.

“Carbocycle” refers to a saturated, unsaturated or aromatic ring in which each atom of the ring is a carbon atom. Carbocycle may include C₃₋₁₀ monocyclic rings, C₆₋₁₂ bicyclic rings, C₇₋₁₈ polycyclic rings, C₅₋₁₂ spirocyclic rings, and C₆₋₁₂ bridged rings. Each ring of a bicyclic or polycyclic carbocycle may be selected from saturated, unsaturated, and aromatic rings. In some embodiments, the carbocycle is a C₆₋₁₂ aryl group, such as C₆₋₁₀ aryl. In some embodiments, the carbocycle is a C₃₋₁₂ cycloalkyl group. In some embodiments, the carbocycle is a C₅₋₁₂ cycloalkenyl group. In an exemplary embodiment, an aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocycle. A carbocycle may comprise a fused ring, a bridged ring, a spirocyclic ring, a saturated ring, an unsaturated ring, an aromatic ring, or any combination thereof. Exemplary carbocycles include cyclopentyl, cyclohexyl, cyclohexenyl, adamantly, phenyl, indanyl, and naphthyl. Unless state otherwise specifically in the specification, a carbocycle is optionally substituted by one or more substituents such as those substituents described herein.

“Heterocycle” refers to a saturated, unsaturated or aromatic ring comprising one or more heteroatoms, for example 1, 2 or 3 heteroatoms selected from O, S and N. Heterocycle may include 3- to 10-membered monocyclic rings, 6- to 12-membered bicyclic rings, 7- to 18-membered polycyclic rings, 5- to 12-membered spirocyclic rings, and 6- to 12-membered bridged rings. Each ring of a bicyclic or polycyclic heterocycle may be selected from saturated, unsaturated, and aromatic rings. The heterocycle may be attached to the rest of the molecule through any atom of the heterocycle, valence permitting, such as a carbon or nitrogen atom of the heterocycle. In some embodiments, the heterocycle is a 5- to 10-membered heteroaryl group, such as 5- or 6-membered heteroaryl. In some embodiments, the heterocycle is a 3- to 12-membered heterocycloalkyl group. A heterocycle may comprise a fused ring, a bridged ring, a spirocyclic ring, a saturated ring, an unsaturated ring, an aromatic ring, or any combination thereof. In an exemplary embodiment, a heterocycle, e.g., pyridyl, may be fused to a saturated or unsaturated ring, e.g., cyclohexane, cyclopentane, or cyclohexene. Exemplary heterocycles include pyrrolidinyl, pyrrolyl, imidazolyl, pyrazolyl, triazolyl, piperidinyl, pyridinyl, pyrimidinyl, pyridazinyl, pyrazinyl, thiophenyl, oxazolyl, thiazolyl, morpholinyl, indazolyl, indolyl, and quinolinyl. Unless stated otherwise specifically in the specification, a heterocycle is optionally substituted by one or more substituents such as those substituents described herein.

“Heteroaryl” refers to a 5- to 12-membered aromatic ring that comprises at least one heteroatom, such as 1, 2 or 3 heteroatoms, selected from O, S and N. As used herein, the heteroaryl ring may be selected from monocyclic or bicyclic-including fused, spirocyclic and bridged ring systems—wherein at least one of the rings in the ring system is aromatic. The heteroatom(s) in the heteroaryl may optionally be oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryl groups include, but are not limited to, azepinyl, benzimidazolyl, benzisothiazolyl, benzisoxazolyl, benzofuranyl, benzothiazolyl, benzothiophenyl, benzoxazolyl, furanyl, imidazolyl, indazolyl, indolyl, isoquinolinyl, isothiazolyl, isoxazolyl, oxadiazolyl, oxazolyl, purinyl, pyrazinyl, pyrazolidinyl, pyrazolyl, pyridazinyl, pyridazolyl, pyridyl, pyrimidinyl, pyrrolyl, quinazolinyl, quinolinyl, quinoxalinyl, tetrahydroquinolinyl, thiadiazolyl, thiazolyl, and thienyl groups. Unless stated otherwise specifically in the specification, a heteroaryl is optionally substituted by one or more substituents such as those substituents described herein.

Unless stated otherwise, hydrogen atoms are implied in structures depicted herein as necessary to satisfy the valence requirement.

A waved line [symbol] drawn across a bond or a dashed bond [symbol] are used interchangeably herein to denote where a bond disconnection or attachment occurs. For example, in the structure

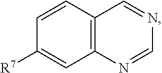

if R⁷ is 2-fluoro-6-hydroxyphenyl as in

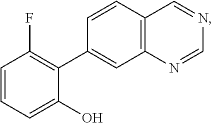 [SMILES: Oc1cccc(F)c1-c1ccc2cncnc2c1]

then R⁷ may be depicted as

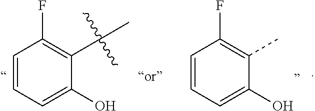

The term “substituted” refers to moieties having substituents replacing a hydrogen on one or more carbons or heteroatoms of the structure. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, heteroatoms such as nitrogen may have any permissible substituents of organic compounds described herein which satisfy the valences of the heteroatoms.

A compound disclosed herein, such as a compound of Formula (I), is optionally substituted by one or more, such as 1, 2 or 3 substituents selected from:
halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from:
halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —OC(O)R²⁵, —C(O)N(R²²)(R²³), —C(O)C(O)N(R²²)(R²³), —N(R²⁴)C(O)R²⁵, —S(O)₂R²⁵, and —S(O)₂N(R²²)(R²³)—, wherein C₁₋₆ alkyl, C₂₋₆ alkenyl, C₂₋₆ alkynyl, C₃₋₁₀ carbocycle, —CH₂—(C_{3- 10} carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², and —N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle, wherein C₃₋₁₀ carbocycle and 3- to 10-membered heterocycle are optionally substituted with one, two, or three groups independently selected from halogen and C₁₋₆ alkyl;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three groups independently selected from halogen, C₁₋₆ alkyl, C₁₋₆ haloalkyl, C₁₋₆ alkoxy, C₃₋₁₀ carbocycle, and 3- to 10-membered heterocycle.

In some embodiments, a compound disclosed herein, such as a compound of Formula (I), is optionally substituted by one or more, such as 1, 2 or 3 substituents selected from halogen, oxo, ═NH, —CN, —NO₂, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OH, —OCH₃, —OCH₂CH₃, —NH₂, —NHCH₃, and —NHCH₂CH₃, wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three groups independently selected from halogen, oxo, ═NH, —CN, —NO₂, —CH₃, —CH₂CH₃, —CH(CH₃)₂, —C(CH₃)₃, —OH, —OCH₃, —OCH₂CH₃, —NH₂, —NHCH₃, and —NHCH₂CH₃.

It will be understood by those skilled in the art that substituents can themselves be substituted, if appropriate. Unless specifically stated as “unsubstituted”, references to chemical moieties herein are understood to include substituted variants. For example, reference to a “heteroaryl” group or moiety implicitly includes both substituted and unsubstituted variants.

Where bivalent substituent groups are specified herein by their conventional chemical formulae, written from left to right, they are intended to encompass the isomer that would result from writing the structure from right to left, e.g., —CH₂O— is also intended to encompass —OCH₂—.

“Optional” or “optionally” means that the subsequently described event or circumstances may or may not occur, and that the description includes instances where the event or circumstance occurs and instances in which it does not. For example, an “optionally substituted” group may be either unsubstituted or substituted.

Compounds of the present disclosure also include crystalline and amorphous forms of those compounds, pharmaceutically acceptable salts, and active metabolites having the same type of activity, including, for example, polymorphs, pseudopolymorphs, solvates, hydrates, unsolvated polymorphs (including anhydrates), conformational polymorphs, amorphous forms of the compounds, and mixtures thereof.

The compounds described herein may exhibit their natural isotopic abundance, or one or more of the atoms may be artificially enriched in a particular isotope having the same atomic number, but an atomic mass or mass number different from the atomic mass or mass number predominantly found in nature. All isotopic variations of the compounds of the present disclosure, whether radioactive or not, are encompassed within the scope of the present disclosure. For example, hydrogen has three naturally occurring isotopes, denoted ¹H (protium), ²H (deuterium), and ³H (tritium). Protium is the most abundant isotope of hydrogen in nature. Enriching for deuterium may afford certain therapeutic advantages, such as increased in vivo half-life and/or exposure, or may provide a compound useful for investigating in vivo routes of drug elimination and metabolism. Examples of isotopes that may be incorporated into compounds of the present disclosure include, but are not limited to, ²H, ³H, ¹³C, ¹⁴C, ¹⁵N, ¹⁸O, ¹⁷O, ³⁵S, ³⁶Cl, and ¹⁸F. Of particular interest are compounds of Formula (I) enriched in tritium or carbon-14, which can be used, for example, in tissue distribution studies; compounds of the disclosure enriched in deuterium-especially at a site of metabolism-resulting, for example, in compounds having greater metabolic stability; and compounds of Formula (I) enriched in a positron emitting isotope, such as ¹¹C, ¹⁸F, ¹⁵O and ¹³N, which can be used, for example, in Positron Emission Topography (PET) studies. Isotopically-enriched compounds may be prepared by conventional techniques well known to those skilled in the art.

As used herein, the phrase “of the formula”, “having the formula” or “having the structure” is not intended to be limiting and is used in the same way that the term “comprising” is commonly used. For example, if one structure is depicted, it is understood that all stereoisomer and tautomer forms are encompassed, unless stated otherwise.

Certain compounds described herein contain one or more asymmetric centers and can thus give rise to enantiomers, diastereomers, and other stereoisomeric forms, the asymmetric centers of which can be defined, in terms of absolute stereochemistry, as (R)— or (S)—. In some embodiments, in order to optimize the therapeutic activity of the compounds of the disclosure, e.g., to treat cancer, it may be desirable that the carbon atoms have a particular configuration (e.g., (R,R), (S,S), (S,R), or (R,S)) or are enriched in a stereoisomeric form having such configuration. The compounds of the disclosure may be provided as racemic mixtures. Accordingly, the disclosure relates to racemic mixtures, pure stereoisomers (e.g., enantiomers and diastereomers), stereoisomer-enriched mixtures, and the like, unless otherwise indicated. When a chemical structure is depicted herein without any stereochemistry, it is understood that all possible stereoisomers are encompassed by such structure. Similarly, when a particular stereoisomer is shown or named herein, it will be understood by those skilled in the art that minor amounts of other stereoisomers may be present in the compositions of the disclosure unless otherwise indicated, provided that the utility of the composition as a whole is not eliminated by the presence of such other isomers. Individual stereoisomers may be obtained by numerous methods that are known in the art, including preparation using chiral synthons or chiral reagents, resolution using chiral chromatography using a suitable chiral stationary phase or support, or by chemically converting them into diastereomers, separating the diastereoisomers by conventional means such as chromatography or recrystallization, then regenerating the original stereoisomer.

Additionally, where applicable, all cis-trans or E/Z isomers (geometric isomers), tautomeric forms and topoisomeric forms of the compounds described herein are included with the scope of the disclosure unless otherwise specified.

The term “pharmaceutically acceptable” refers to a material that is not biologically or otherwise unacceptable when used in the subject compositions and methods. For example, the term “pharmaceutically acceptable carrier” refers to a material—such as an adjuvant, excipient, glidant, sweetening agent, diluent, preservative, dye, colorant, flavor enhancer, surfactant, wetting agent, dispersing agent, suspending agent, stabilizer, isotonic agent, solvent or emulsifier—that can be incorporated into a composition and administered to a patient without causing unacceptable biological effects or interacting in an unacceptable manner with other components of the composition. Such pharmaceutically acceptable materials typically have met the required standards of toxicological and manufacturing testing, and include those materials identified as suitable inactive ingredients by the U.S. Food and Drug Administration.

The terms “salt” and “pharmaceutically acceptable salt” refer to a salt prepared from a base or an acid. Pharmaceutically acceptable salts are suitable for administration to a patient, such as a mammal (for example, salts having acceptable mammalian safety for a given dosage regime). Salts can be formed from inorganic bases, organic bases, inorganic acids and organic acids. In addition, when a compound contains both a basic moiety, such as an amine, pyridine or imidazole, and an acidic moiety, such as a carboxylic acid or tetrazole, zwitterions may be formed and are included within the term “salt” as used herein. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc., and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. An effective amount of an active agent may be administered in a single dose or in multiple doses. A component may be described herein as having at least an effective amount, or at least an amount effective, such as that associated with a particular goal or purpose, such as any described herein. The term “effective amount” also applies to a dose that will provide an image for detection by an appropriate imaging method. The specific dose may vary depending on one or more of: the particular agent chosen, the dosing regimen to be followed, whether it is administered in combination with other compounds, timing of administration, the tissue to be imaged, and the physical delivery system in which it is carried.

As used herein, “treating” or “treatment” refers to an approach for obtaining beneficial or desired results with respect to a disease, disorder, or medical condition (such as cancer) in a subject, including but not limited to the following: (a) preventing the disease or medical condition from occurring, e.g., preventing the reoccurrence of the disease or medical condition or prophylactic treatment of a subject that is pre-disposed to the disease or medical condition; (b) ameliorating the disease or medical condition, e.g., eliminating or causing regression of the disease or medical condition in a subject; (c) suppressing the disease or medical condition, e.g., slowing or arresting the development of the disease or medical condition in a subject; or (d) alleviating symptoms of the disease or medical condition in a subject. For example, “treating cancer” would include preventing cancer from occurring, ameliorating cancer, suppressing cancer, and alleviating the symptoms of cancer. Also, a therapeutic benefit is achieved with the eradication or amelioration of one or more of the physiological symptoms associated with the underlying disorder such that an improvement is observed in the subject, notwithstanding that the subject may still be afflicted with the underlying disorder.

A “therapeutic effect”, as that term is used herein, encompasses a therapeutic benefit and/or prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “antagonist” and “inhibitor” are used interchangeably, and they refer to a compound having the ability to inhibit a biological function (e.g., activity, expression, binding, protein-protein interaction) of a target protein (e.g., K-Ras). Accordingly, the terms “antagonist” and “inhibitor” are defined in the context of the biological role of the target protein. While preferred antagonists herein specifically interact with (e.g., bind to) the target, compounds that inhibit a biological activity of the target protein by interacting with other members of the signal transduction pathway of which the target protein is a member are also specifically included within this definition.

The term “selective inhibition” or “selectively inhibit” refers to the ability of a biologically active agent to preferentially reduce the target signaling activity as compared to off-target signaling activity, via direct or indirect interaction with the target.

The terms “subject” and “patient” refer to an animal, such as a mammal, for example a human. The methods described herein can be useful in both human therapeutics and veterinary applications. In some embodiments, the subject is a mammal, such as a human. “Mammal” includes humans and both domestic animals such as laboratory animals and household pets (e.g., cats, dogs, swine, cattle, sheep, goats, horses, rabbits), and non-domestic animals such as wildlife and the like.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” refers to either natural and/or unnatural or synthetic amino acids, including glycine and both the D or L optical isomers, and amino acid analogs and peptidomimetics.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. A polynucleotide may comprise one or more modified nucleotides, such as methylated nucleotides and nucleotide analogs, such as peptide nucleic acid (PNA), morpholino and locked nucleic acid (LNA), glycol nucleic acid (GNA), threose nucleic acid (TNA), 2′-fluoro, 2′-OMe, and phosphorothiolated DNA. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component or other conjugation target.

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

An “antigen” is a moiety or molecule that contains an epitope, and, as such, also specifically binds to an antibody. An “antigen binding unit” may be whole or a fragment (or fragments) of a full-length antibody, a structural variant thereof, a functional variant thereof, or a combination thereof. A full-length antibody may be, for example, a monoclonal, recombinant, chimeric, deimmunized, humanized and human antibody. Examples of a fragment of a full-length antibody may include, but are not limited to, variable heavy (VH), variable light (VL), a heavy chain found in camelids, such as camels, llamas, and alpacas (VHH or V_{H}H), a heavy chain found in sharks (V-NAR domain), a single domain antibody (sdAb, e.g., “nanobody”) that comprises a single antigen-binding domain, Fv, Fd, Fab, Fab′, F(ab′)2, and “r IgG” (or half antibody). Examples of modified fragments of antibodies may include, but are not limited to scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, minibodies (e.g., (VH-VL-CH3)2, (scFv-CH3)2, ((scFv)2-CH3+CH3), ((scFv)2-CH3) or (scFv-CH3-scFv)2), and multibodies (e.g., triabodies or tetrabodies).

The term “antibody” and “antibodies” encompass any antigen binding units, including without limitation: monoclonal antibodies, human antibodies, humanized antibodies, camelised antibodies, chimeric antibodies, and any other epitope-binding fragments.

“Prodrug” is meant to indicate a compound that may be converted under physiological conditions or by solvolysis to a biologically active compound described herein (e.g., a compound of Formula (I)). Thus, the term “prodrug” refers to a precursor of a biologically active compound that is pharmaceutically acceptable. In some aspects, a prodrug is inactive when administered to a subject but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam); Higuchi, T., et al., “Pro-drugs as Novel Delivery Systems,” (1987) A.C.S. Symposium Series, Vol. 14; and Bioreversible Carriers in Drug Design, ed. Edward B. Roche, American Pharmaceutical Association and Pergamon Press each of which is incorporated in full by reference herein). The term “prodrug” is also meant to include any covalently bonded carriers, which release the active compound in vivo when such prodrug is administered to a mammalian subject. Prodrugs of an active compound, as described herein, are typically prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of a hydroxy functional group, or acetamide, formamide and benzamide derivatives of an amine functional group in the active compound, and the like.

The term “in vivo” refers to an event that takes place in a subject's body. The term “ex vivo” refers to an event that first takes place outside of the subject's body for a subsequent in vivo application into a subject's body. For example, an ex vivo preparation may involve preparation of cells outside of a subject's body for the purpose of introduction of the prepared cells into the same or a different subject's body. The term “in vitro” refers to an event that takes place outside of a subject's body. For example, an in vitro assay encompasses any assay run outside of a subject's body. In vitro assays encompass cell-based assays in which cells alive or dead are employed. In vitro assays also encompass a cell-free assay in which no intact cells are employed.

The disclosure is also meant to encompass the in vivo metabolic products of the disclosed compounds. Such products may result from, for example, the oxidation, reduction, hydrolysis, amidation, esterification, and the like of the administered compound, primarily due to enzymatic processes. Accordingly, the disclosure includes compounds produced by a process comprising administering a compound disclosed herein to a mammal for a period of time sufficient to yield a metabolic product thereof. Such products are typically identified by administering a radiolabeled compound of the disclosure in a detectable dose to an animal, such as rat, mouse, guinea pig, monkey, or to a human, allowing sufficient time for metabolism to occur, and isolating its conversion products from the urine, blood or other biological samples.

The term “Ras” or “RAS” refers to a protein in the Rat sarcoma (Ras) superfamily of small GTPases, such as in the Ras subfamily. The Ras superfamily includes, but is not limited to, the Ras subfamily, Rho subfamily, Rab subfamily, Rap subfamily, Arf subfamily, Ran subfamily, Rheb subfamily, RGK subfamily, Rit subfamily, Miro subfamily, and Unclassified subfamily. In some embodiments, a Ras protein is selected from the group consisting of KRAS (also used interchangeably herein as K-Ras, K-ras, or Kras), HRAS (or H-Ras), NRAS (or N-Ras), MRAS (or M-Ras), ERAS (or E-Ras), RRAS2 (or R-Ras2), RALA (or RalA), RALB (or RalB), RIT1, and any combination thereof, such as from KRAS, HRAS, NRAS, RALA, RALB, and any combination thereof.

The terms “mutant Ras” and “Ras mutant”, as used interchangeably herein, refer to a Ras protein with one or more amino acid mutations, such as with respect to a common reference sequence such as a wild-type (WT) sequence. In some embodiments, a mutant Ras is selected from a mutant KRAS, mutant HRAS, mutant NRAS, mutant MRAS, mutant ERAS, mutant RRAS2, mutant RALA, mutant RALB, mutant RIT1, and any combination thereof, such as from a mutant KRAS, mutant HRAS, mutant NRAS, mutant RALA, mutant RALB, and any combination thereof. In some embodiments, a mutation can be an introduced mutation, a naturally occurring mutation, or a non-naturally occurring mutation. In some embodiments, a mutation can be a substitution (e.g., a substituted amino acid), insertion (e.g., addition of one or more amino acids), or deletion (e.g., removal of one or more amino acids). In some embodiments, two or more mutations can be consecutive, non-consecutive, or a combination thereof. In some embodiments, a mutation can be present at any position of Ras. In some embodiments, a mutation can be present at position 12, 13, 62, 92, 95, or any combination thereof of Ras relative to SEQ ID No. 1 when optimally aligned. In some embodiments, a mutant Ras may comprise about or at least about 1, 2, 3, 4, 5, 6, 7, 8, 9, 10, 11, 12, 13, 14, 15, 16, 17, 18, 19, 20, 25, 30, 35, 40, 45, 50, or more than 50 mutations. In some embodiments, a mutant Ras may comprise up to about 1, 2, 3, 4, 5, 6, 7, 8, 9, 10, 11, 12, 13, 14, 15, 16, 17, 18, 19, 20, 25, 30, 35, 40, 45, or 50 mutations. In some embodiments, the mutant Ras is about or up to about 500, 400, 300, 250, 240, 233, 230, 220, 219, 210, 208, 206, 204, 200, 195, 190, 189, 188, 187, 186, 185, 180, 175, 174, 173, 172, 171, 170, 169, 168, 167, 166, 165, 160, 155, 150, 125, 100, 90, 80, 70, 60, 50, or fewer than 50 amino acids in length. In some embodiments, an amino acid of a mutation is a proteinogenic, natural, standard, non-standard, non-canonical, essential, non-essential, or non-natural amino acid. In some embodiments, an amino acid of a mutation has a positively charged side chain, a negatively charged side chain, a polar uncharged side chain, a non-polar side chain, a hydrophobic side chain, a hydrophilic side chain, an aliphatic side chain, an aromatic side chain, a cyclic side chain, an acyclic side chain, a basic side chain, or an acidic side chain. In some embodiments, a mutation comprises a reactive moiety. In some embodiments, a substituted amino acid comprises a reactive moiety. In some embodiments, a mutant Ras can be further modified, such as by conjugation with a detectable label. In some embodiments, a mutant Ras is a full-length or truncated polypeptide. For example, a mutant Ras can be a truncated polypeptide comprising residues 1-169 or residues 11-183 (e.g., residues 11-183 of a mutant RALA or mutant RALB).

As used herein, the term “corresponding to” or “corresponds to” as applied to an amino acid residue in a polypeptide sequence refers to the correspondence of such amino acid relative to a reference sequence when optimally aligned (e.g., taking into consideration of gaps, insertions and mismatches; wherein alignment may be primary sequence alignment or three-dimensional structural alignment of the folded proteins). For instance, the serine residue in a Ras G12S mutant refers to the serine corresponding to residue 12 of SEQ ID No. 4, which can serve as a reference sequence. For instance, the aspartate residue in a Ras G12D mutant refers to the aspartate corresponding to residue 12 of SEQ ID No. 2, which can serve as a reference sequence. When an amino acid of a mutant Ras protein corresponds to an amino acid position in the WT Ras protein, it will be understood that although the mutant Ras protein amino acid may be a different amino acid (e.g., G12D wherein the wildtype G at position 12 is replaced by an aspartate at position 12 of SEQ ID. No. 1), the mutant amino acid is at the position corresponding to the wildtype amino acid (e.g., of SEQ ID No. 1). In embodiments, a modified Ras mutant protein disclosed herein may comprise truncations at C-terminus, or truncations at the N-terminal end preceding the serine residue. The serine residue in such N-terminal truncated modified mutant is still considered corresponding to position 12 of SEQ ID No. 1. In addition, a serine residue at position 12 of SEQ ID No. 4 finds a corresponding residue in SEQ ID Nos. 6 and 8.

The term “leaving group” is used herein in accordance with their well understood meanings in Chemistry and refers to an atom or group of atoms which breaks away from the rest of the molecule, taking with it the electron pair which used to be the bond between the leaving group and the rest of the molecule.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In embodiments, the degradation enhancer is capable of binding to an E3 ubiquitin ligase protein or a protein complex comprising an E3 ubiquitin ligase protein. In embodiments, the degradation enhancer is capable of binding to an E2 ubiquitin-conjugating enzyme. In embodiments, the degradation enhancer is capable of binding to a protein complex comprising an E2 ubiquitin-conjugating enzyme and an E3 ubiquitin ligase protein.

### Compounds

The compounds of Formula (I) disclosed herein-including the compounds of Formula (I′), (I-A), (I-A1), (I-A2), (I-A3), (I-B), (I-B1), (I-B2), and (I-B3)—or a pharmaceutically acceptable salt or solvate thereof, are K-Ras inhibitors and have a wide range of applications in therapeutics, diagnostics, and other biomedical research.

In certain aspects, the present disclosure provides a compound of Formula (I):

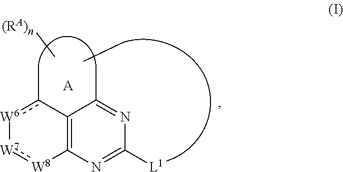

or a pharmaceutically acceptable salt or solvate thereof, wherein:
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R⁶) or C(O), W⁷ is N(R^{7b}), W⁸ is C(R⁸)(R^{8a}) or C(O), and each [symbol] indicates a single
R⁶, R^{6a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R⁷ is -L⁷-R¹⁷;
R^{7b} is -L^{7b}-R¹⁷;
L⁷ is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
L^{7b} is selected from a bond, —O—, —C(O)—, —S—, —S(O)₂—, —S(O)—, —P(O)R^{7d}—, —C(R^{7c})₂—, —OC(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
R¹⁷ is selected from C₃₋₁₂ carbocycle and 3- to 12-membered heterocycle, each of which is optionally substituted with one or more R²⁰;
L¹ comprises one or more groups independently selected from C₁₋₁₅ alkylene, C₂₋₁₅ alkenylene, C₂₋₁₅ alkynylene, C₃₋₁₈ carbocycle, 3- to 18-membered heterocycle, and combinations thereof, each of which is optionally substituted with one or more R^{L}, wherein one or more of the carbon atoms of the C₁₋₁₅ alkylene, C₂₋₁₅ alkenylene, and C₂₋₁₅ alkynylene are optionally replaced with a heteroatom independently selected at each replacement from 0, N, P, Si, and S;
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 6;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

In certain aspects, the present disclosure provides a compound of Formula (I):

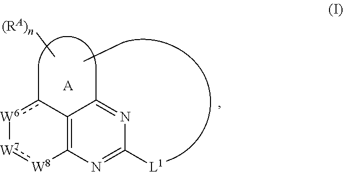

or a pharmaceutically acceptable salt or solvate thereof, wherein:
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R^{6a}) or C(O), W⁷ is N(R^{7b}), W⁸ is C(R⁸)(R^{8a}) or C(O), and each [symbol] indicates a single bond;
R⁶, R^{6a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R⁷ is -L⁷-R¹⁷;
R^{7b} is -L^{7b}-R¹⁷;
L⁷ is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
L^{7b} is selected from a bond, —O—, —C(O)—, —S—, —S(O)₂—, —S(O)—, —P(O)R^{7d}—, —C(R^{7c})₂—, —OC(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
R¹⁷ is selected from C₃₋₁₂ carbocycle and 3- to 12-membered heterocycle, each of which is optionally substituted with one or more R²⁰;
L¹ is -L²-L³-L⁴-L⁵-L⁶-;
L² is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle, wherein —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle are optionally substituted with one, two, or three R^{L};
L³ is selected from C₁₋₆ alkyl, C₂₋₆ alkenyl, C₂₋₆ alkynyl, and 2- to 6-membered heteroalkyl, each of which is optionally substituted with one, two, or three R^{L};
L⁴ and L⁶ are each independently selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₂₋₆ alkenyl, C₂₋₆ alkynyl, 2- to 6-membered heteroalkyl, C₃₋₁₀ carbocycle, and 3- to 10-membered heterocycle, wherein C₁₋₆ alkyl, C₂₋₆ alkenyl, C₂₋₆ alkynyl, 2- to 6-membered heteroalkyl, C₃₋₁₀ carbocycle, and 3- to 10-membered heterocycle are optionally substituted with one, two, or three R^{L};
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 6;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

In some embodiments, the compound of Formula (I) is a compound of Formula (I′):

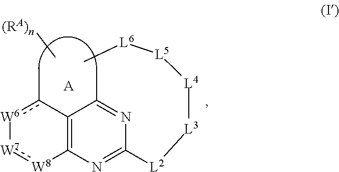

or a pharmaceutically acceptable salt or solvate thereof.

In some embodiments, for a compound of Formula (I) or (I), A is C₅₋₁₈ carbocycle, such as C₅₋₁₂ carbocycle. In some embodiments, A is C₅₋₈ carbocycle, such as C₅₋₈ cycloalkenyl. In some embodiments, A is C₈₋₁₂ membered carbocycle, such as C₈-12 fused bicyclic cycloalkenyl. In some embodiments, A is 5- to 18-membered heterocycle, such as 5- to 12-membered heterocycle. In some embodiments, A is 5- to 8-membered heterocycle, such as 5- to 8-membered monocyclic heterocycloalkenyl. In some embodiments, A is 8- to 12-membered heterocycle, such as 8- to 12-membered fused bicyclic heterocycloalkenyl. In some embodiments, A is selected from C₅₋₁₂ carbocycle and 5- to 12-membered heterocycle, such as A is selected from C₅₋₈ monocyclic cycloalkenyl, C₈₋₁₂ fused bicyclic cycloalkenyl, 5- to 8-membered monocyclic heterocycloalkenyl, and 8- to 12-membered fused bicyclic heterocycloalkenyl. In some embodiments, A is selected from C₁₀₋₁₈ spiro-fused tricyclic carbocycle and 10- to 18-membered spiro-fused tricyclic heterocycle. In some embodiments, A is 7- to 8-membered heterocycle, such as 7-membered heterocycle. In some embodiments, A is 10- to 12-membered fused bicyclic heterocycle, such as 10-membered fused bicyclic heterocycle. In some embodiments, A is 5- to 18-membered heterocycle, wherein the heterocycle comprises at least one nitrogen atom. In some embodiments, A is selected from C₅₋₈ monocyclic carbocycle, C₈₋₁₂ bicyclic fused carbocycle, C₇₋₁₈ bicyclic spirocyclic carbocycle, C₁₂₋₁₈ fused tricyclic carbocycle, C₁₀₋₁₈ spiro-fused tricyclic carbocycle, 5- to 8-membered monocyclic heterocycle, 8- to 12-membered bicyclic fused heterocycle, 7- to 18-membered bicyclic spirocyclic heterocycle, 12- to 18-membered fused tricyclic heterocycle, and 10- to 18-membered spiro-fused tricyclic heterocycle.

For the avoidance of any doubt, references herein to the number of ring atoms contained in A, such as C₅₋₁₈ carbocycle and 5- to 18-membered heterocycle, refer to the ring or ring system depicted in Formula (I) or (I′) as A, inclusive of 3 carbon atoms that are explicitly depicted in Formula (I) and (I′). For example, for a compound of Formula (I-A), A is a 7-membered ring when m1 is 1. In the example below, A is a 12-membered ring:

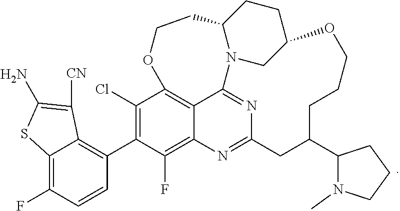 [SMILES: CN1CCCC1C1CCCO[C@H]2CC[C@H]3CCOc4c(Cl)c(-c5ccc(F)c6sc(N)c(C#N)c56)c(F)c5nc(nc(c45)N3C2)C1]

In some embodiments, the compound of Formula (I) is a compound of Formula (I-A):

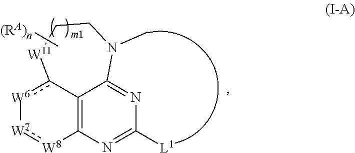

or a pharmaceutically acceptable salt or solvate thereof, wherein:
W¹¹ is selected from —O—, —N(R^{7d})—, —S—, and —C(R^{7c})₂—; and
m1 is 1 or 2.

In some embodiments, the compound of Formula (I) is a compound of Formula (I-B):

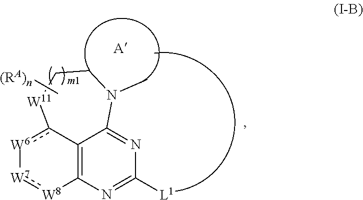

or a pharmaceutically acceptable salt or solvate thereof, wherein:
W¹¹ is selected from —O—, —N(R^{7d})—, —S—, and —C(R^{7c})₂—;
A′ is 5- to 7-membered heterocycle;
R^{A′} is selected from R^{A};
n′ is an integer from 0 to (6-n); and
m1 is 1 or 2.

In some embodiments, for a compound of Formula (I-A) or (I-B), W¹¹ is selected from —O— and —N(R^{7d}), such as W¹¹ is selected from —O— and —NH—. In some embodiments, W¹¹ is selected from —O— and —C(R^{7c})₂—, such as W¹¹ is selected from —O— and —CH₂—. In some embodiments, W¹¹ is —O—. In some embodiments, m1 is 1. In some embodiments, m1 is 2. In some embodiments, W¹¹ is —O— and m1 is 1.

In some embodiments, for a compound of Formula (I-B), A′ is 5- to 7-membered heterocycle, such as 5-membered heterocycle. In some embodiments, A′ is 6-membered heterocycle. In some embodiments, A′ is 7-membered heterocycle. In some embodiments, A′ contains one ring nitrogen atom.

In some embodiments, for a compound of Formula (I-B), R^{A′} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 8-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 8-membered heterocycle are optionally substituted with one, two, or three R²⁰. In some embodiments, R^{A′} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³). In some embodiments, n′ is 0 or 1, such as n′ is 0. In some embodiments, n′ is 1 and n is an integer from 0 to 5.

In some embodiments, for a compound of Formula (I-B), W¹¹ is —O—; R^{A′} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 8-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 8-membered heterocycle are optionally substituted with one, two, or three R²⁰; n′ is 0 or 1; and n is an integer from 0 to 5.

In some embodiments, for a compound of Formula (I), (I′), (I-A), or (I-B), W⁶ is C(R⁶), W⁷ is C(R⁷), W⁸ is C(R⁸), and each [symbol] indicates a double bond. In some embodiments, W⁶ is N, W⁷ is C(R⁷), W⁸ is C(R⁸), and each [symbol] indicates a double bond. In some embodiments, W⁶ is C(R⁶)(R^{6a}) or C(O), W⁷ is N(R^{7b}), W⁸ is C(R⁸)(R^{8a}) or C(O), and each [symbol] indicates a single bond.

In some embodiments, the compound of Formula (I-A) is a compound of Formula (I-A1), (I-A2), or (I-A3):

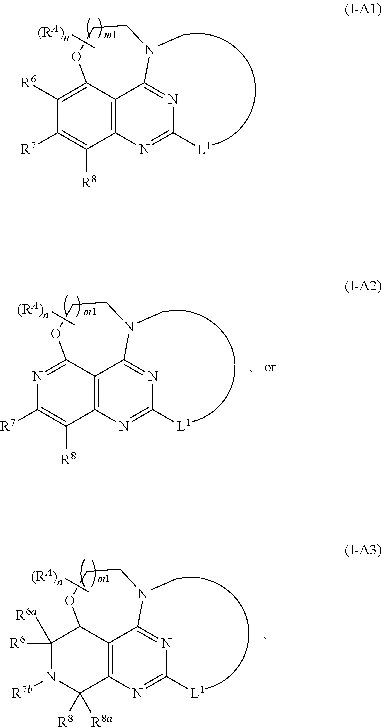

or a pharmaceutically acceptable salt or solvate thereof.

In some embodiments, the compound of Formula (I-B) is a compound of Formula (I-B1), (I-B2), or (I-B3):

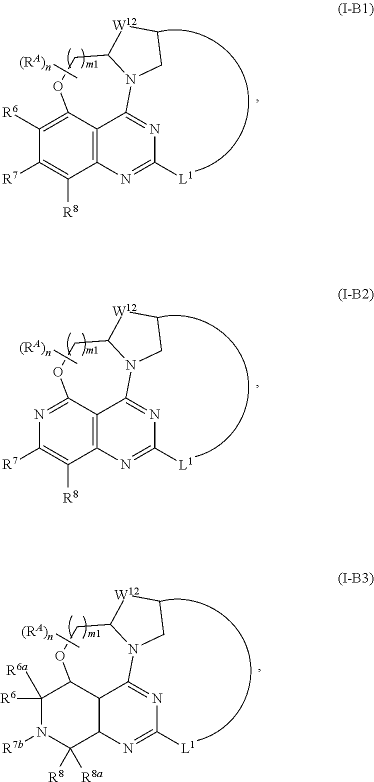

or a pharmaceutically acceptable salt or solvate thereof, wherein W¹² is selected from —O—, —N(R^{7d})—, —C(R^{7d})₂—, —OC(R^{7c})₂—, —N(R^{7d})C(R^{7c})₂—, —C(R^{7c})₂C(R^{7c})₂, —C(R^{7c})₂O—, and —C(R^{7c})₂N(R^{7d})—.

In some embodiments, for a compound of Formula (I-B), (I-B1), (I-B2), or (I-B3), W¹² is selected from —O—, —N(R^{7d})—, —CH₂—, —OCH₂—, —N(R^{7d})CH₂—, —CH₂CH₂, —CH₂O—, and —CH₂N(R^{7d})—. In some embodiments, W¹² is selected from —CH₂—, —CH₂CH₂, —CH₂O—, and —CH₂N(R^{7d})—. In some embodiments, W¹² is —CH₂—. In some embodiments, W¹² is —CH₂CH₂. In some embodiments, W¹² is —CH₂O—. In some embodiments, W¹² is —CH₂N(R^{7d})—.

In some embodiments, for a compound of Formula (I), (I′), (I-A), (I-A1), (I-A3), (I-B), (I-B1), or (I-B3), R⁶ is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰. In some embodiments, R⁶ is selected from hydrogen, halogen, —CN, C₁₋₆ alkyl, and —OR¹², wherein C₁₋₆ alkyl is optionally substituted with one, two, or three R²⁰, such as R⁶ is halogen. In some embodiments, R⁶ is selected from hydrogen, halogen, —CN, C₁₋₆ alkyl, —OH, and —OCH₃. In some embodiments, R⁶ is F or Cl, such as R⁶ is Cl.

In some embodiments, for a compound of Formula (I), (I′), (I-A), (I-A3), (I-B), or (I-B3), R⁶ is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰. In some embodiments, R^{6a} is selected from hydrogen, halogen, —CN, C₁₋₆ alkyl, and —OR¹², wherein C₁₋₆ alkyl is optionally substituted with one, two, or three R²⁰. In some embodiments, R^{6a} is hydrogen. In some embodiments, R⁶ and R^{6a} are each hydrogen.

In some embodiments, for a compound of Formula (I), (I′), (I-A), (I-A1), (I-A2), (I-B), (I-B1), or (I-B2), L⁷ is selected from a bond, —O—, —N(R^{7d})—, —C(O)—, and —C(R^{7c})₂—. In some embodiments, L⁷ is selected from a bond, —O—, —NH—, —C(O)—, and —CH₂—. In some embodiments, L⁷ is a bond.

In some embodiments, for a compound of Formula (I), (I′), (I-A), (I-A3), (I-B), or (I-B3), L^{7b} is selected from a bond, —O—, —C(O)—, and —C(R^{7c})₂—. In some embodiments, L^{7b} is selected from a bond, —O—, —C(O)—, and —CH₂—. In some embodiments, L^{7b} is a bond.

In some embodiments, for a compound of Formula (I), (I′), (I-A), (I-A1), (I-A2), (I-A3), (I-B), (I-B1), (I-B2), or (I-B3), R¹⁷ is selected from C₆₋₁₂ aryl and 5- to 12-membered heteroaryl, each of which is optionally substituted with one, two, or three R²⁰. In some embodiments, R¹⁷ is selected from C₁₀ aryl and 9-membered heteroaryl, each of which is optionally substituted with one, two, or three R²⁰. In some embodiments, R¹⁷ is selected from naphthalenyl and benzothiophenyl, each of which is optionally substituted with one, two, or three R²⁰. In some embodiments, R¹⁷ is benzothiophenyl optionally substituted with one, two, or three R²⁰. In some embodiments, R¹⁷ is selected from C₃₋₁₀ cycloalkyl, 3- to 10-membered heterocycloalkyl, C₆₋₁₀ aryl, and 5- to 10-membered heteroaryl, each of which is optionally substituted with one, two, or three R²⁰. In some embodiments, R¹⁷ is selected from bicyclic C₄₋₁₀ cycloalkyl, bicyclic 4- to 10-membered heterocycloalkyl, bicyclic C₇₋₁₀ aryl, and bicyclic 7- to 10-membered heteroaryl, each of which is optionally substituted with one, two, or three R²⁰. In some embodiments, R¹⁷ is selected from bridged bicyclic C₄₋₁₀ cycloalkyl, bridged bicyclic 4- to 10-membered heterocycloalkyl, bridged bicyclic C₇₋₁₀ aryl, and bridged bicyclic 7- to 10-membered heteroaryl, each of which is optionally substituted with one, two, or three R²⁰. In some embodiments, R¹⁷ is selected from fused bicyclic C₄₋₁₀ cycloalkyl, fused bicyclic 4- to 10-membered heterocycloalkyl, fused bicyclic C₇₋₁₀ aryl, and fused bicyclic 7- to 10-membered heteroaryl, each of which is optionally substituted with one, two, or three R²⁰. In some embodiments, R¹⁷ is optionally substituted with one or more R²⁰, such as one, two, three, four, five, six, or seven R²⁰.

In some embodiments, for a compound of Formula (I), (I′), (I-A), (I-A1), (I-A2), (I-A3), (I-B), (I-B1), (I-B2), or (I-B3), R¹⁷ is selected from

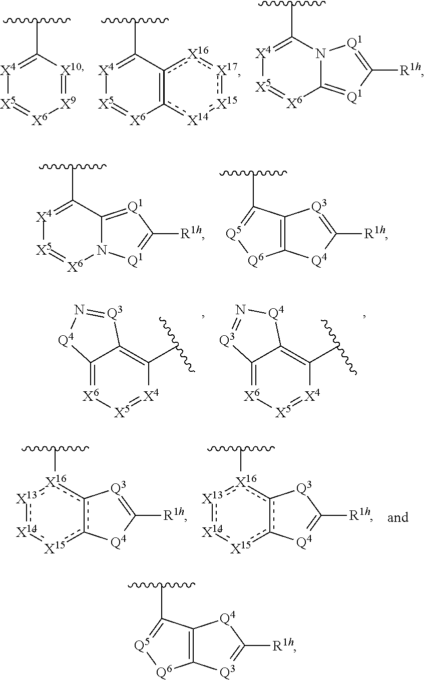

wherein:
Q¹, Q³, and Q⁵ are independently selected from N and C(R^{1d});
Q⁴ and Q⁶ are independently selected from O, S, C(R^{1a})(R^{1b}), and N(R^{1c});
X⁴, X⁵, X⁶, X⁹, and X¹⁰ are independently selected from C(R^{1a}) and N;
X¹³ is selected from a bond, C(R^{1a}), N, C(O), C(R^{1a})(R^{1b}), C(O)C(R^{1a})(R^{1b}), C(R^{1a})(R^{1b})C(R^{1a})(R^{1b}), C(R^{1a})(R^{1b})N(R^{1c}), and N(R^{1c});
X¹⁴, X¹⁵, X¹⁷, and X¹⁸ are independently selected from C(O), C(R^{1a}), N, C(R^{1a})(R^{1b}), and N(R^{1c});
X¹⁶ is selected from C, N, and C(R^{1a});
each R^{1a}, R^{1b}, R^{1d}, and R^{1h} is independently selected from hydrogen, halogen, —CN, C₁₋₆ alkyl, C₁₋₆ haloalkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or R^{1a} and R^{1b} bonded to the same carbon are joined to form 3- to 10-membered heterocycle or C₃₋₁₀ carbocycle, wherein 3- to 10-membered heterocycle and C₃₋₁₀ carbocycle are optionally substituted with one, two, or three R²⁰; or two R^{1a} bonded to adjacent atoms are joined to form 3- to 10-membered heterocycle or C₃₋₁₀ carbocycle, wherein 3- to 10-membered heterocycle and C₃₋₁₀ carbocycle are optionally substituted with one, two, or three R²⁰; or R^{1h} and one of R^{1a}, R^{1b}, R¹¹, and R^{1d} bonded to adjacent atoms are joined to form 3- to 10-membered heterocycle or C₃₋₁₀ carbocycle, wherein 3- to 10-membered heterocycle and C₃₋₁₀ carbocycle are optionally substituted with one, two, or three R²⁰; and
each R^{1c} is independently selected from hydrogen, C₁₋₆ alkyl, C₂₋₆ alkenyl, C₂₋₆ alkynyl, 3- to 10-membered heterocycle, and C₃₋₁₀ carbocycle, wherein C₁₋₆ alkyl, C₂₋₆ alkenyl, C₂₋₆ alkynyl, 3- to 10-membered heterocycle, and C₃₋₁₀ carbocycle are optionally substituted with one, two, or three R²⁰.

In some embodiments, R¹⁷ is selected from

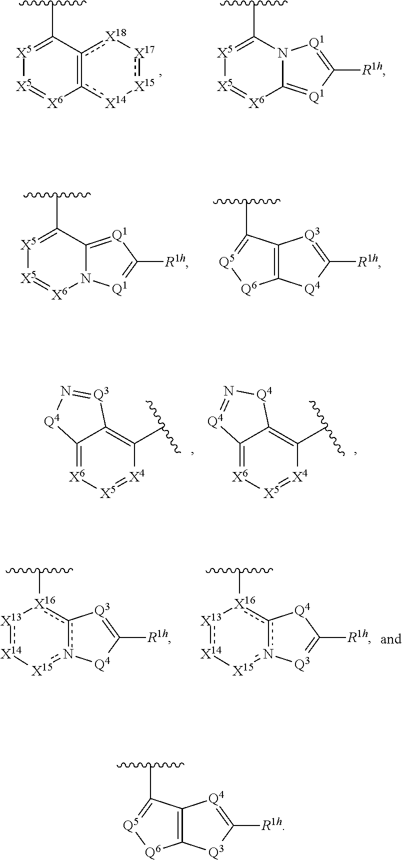

In some embodiments, R¹⁷ is selected from

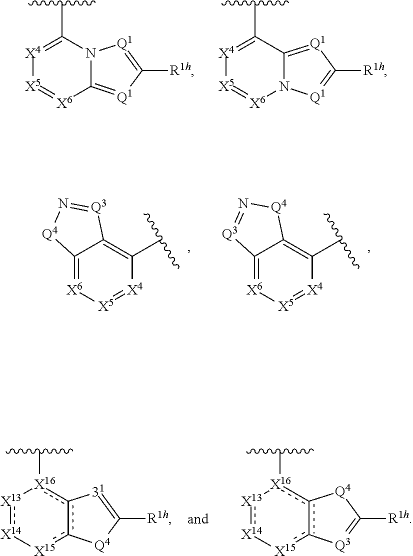

In some embodiments, for a compound of Formula (I), (I′), (I-A), (I-A1), (I-A2)(I-A3) (I-B), (I-B1), (I-B2), or (I-B3), R¹⁷ is selected from

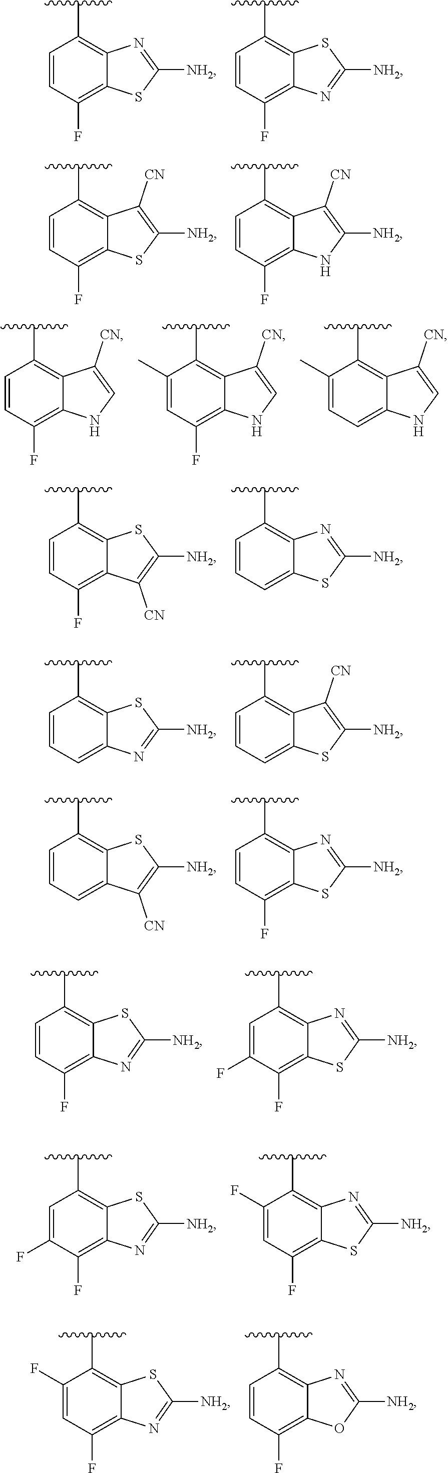

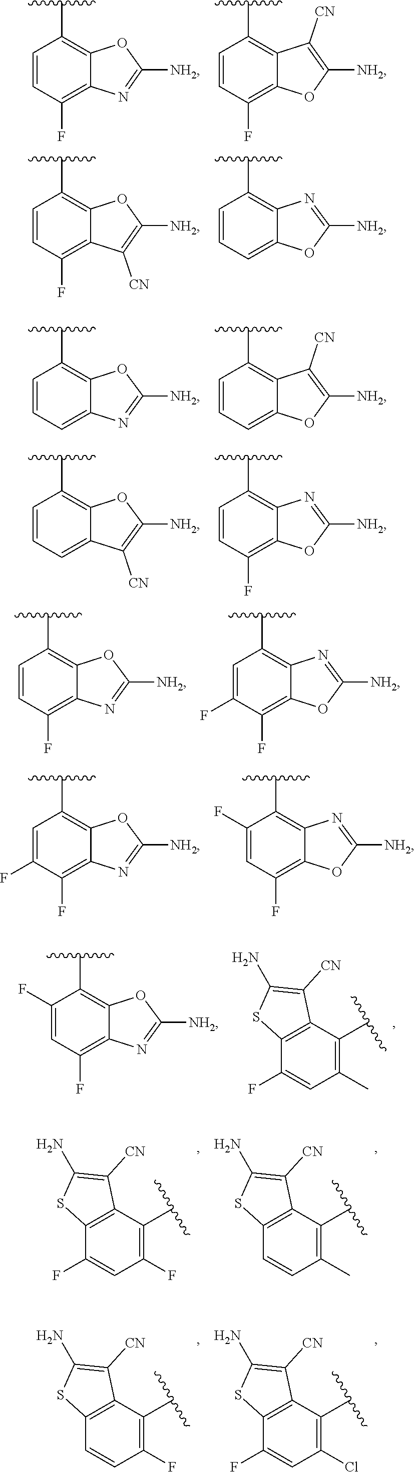

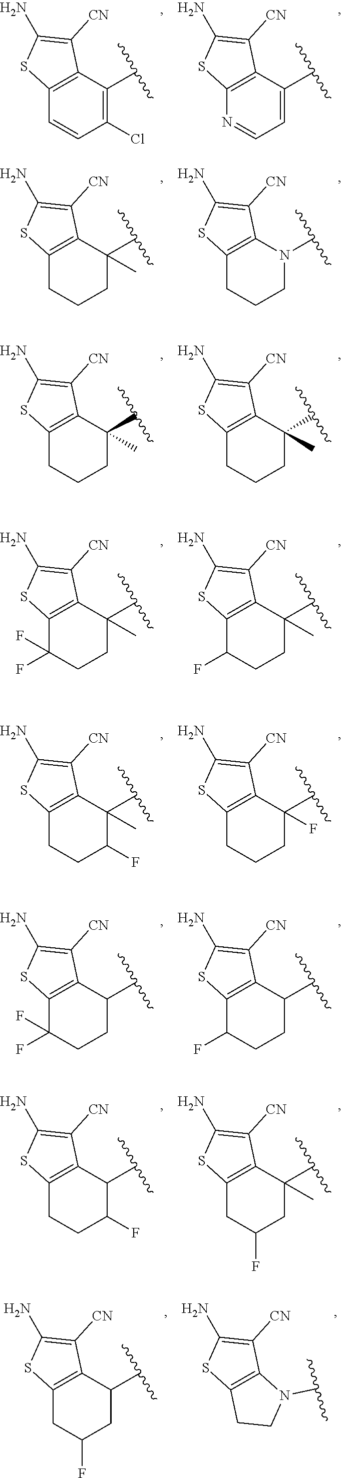

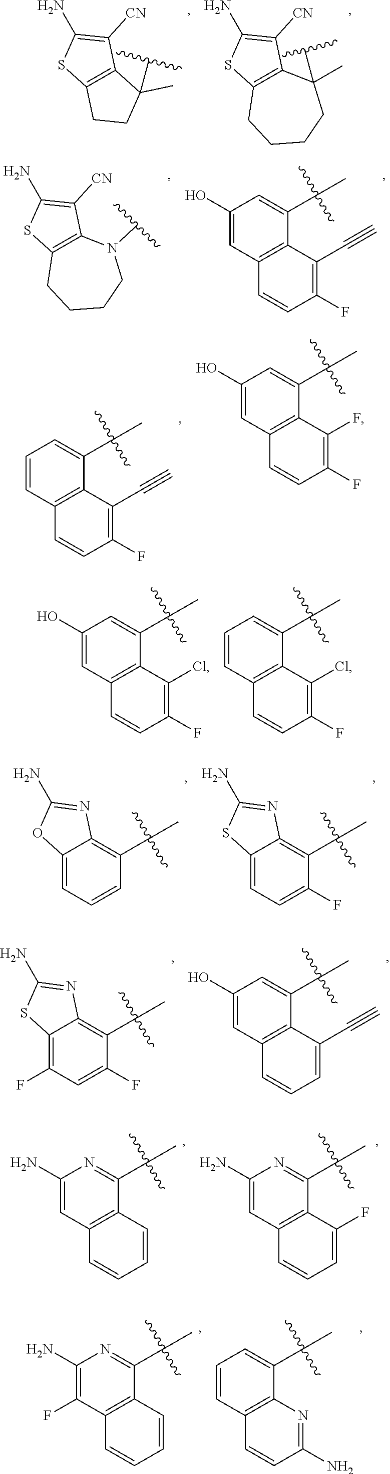

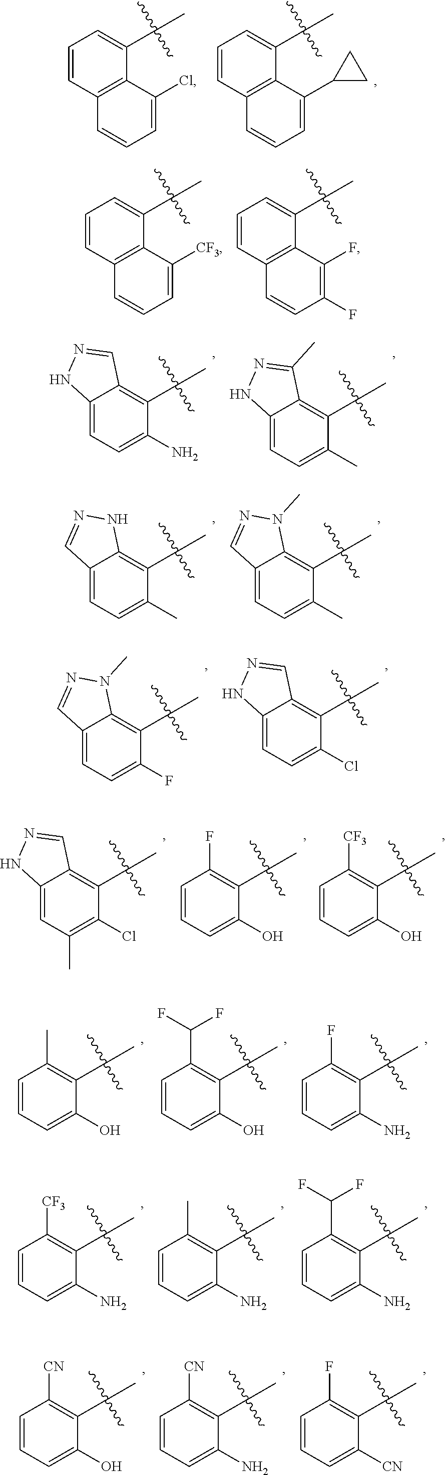

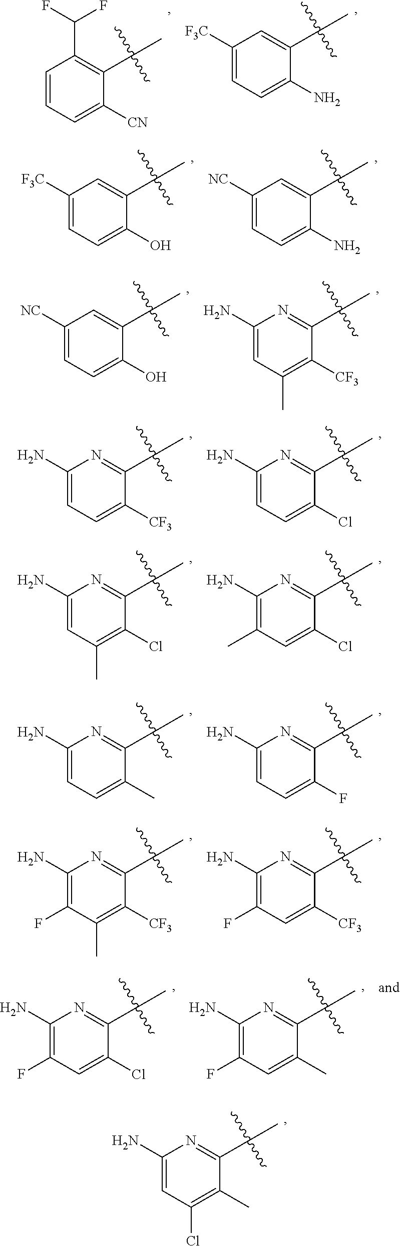

In some embodiments, R¹⁷ is selected from

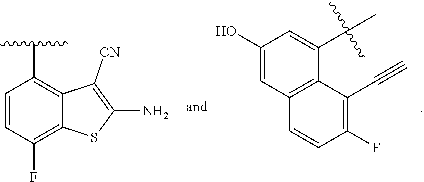 [SMILES: *c1ccc(F)c2sc(N)c(C#N)c12 ; *c1cc(O)cc2ccc(F)c(C#C)c12]

In some embodiments, R¹⁷

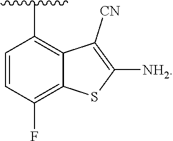 [SMILES: *c1ccc(F)c2sc(N)c(C#N)c12]

In some embodiments, R¹⁷ is

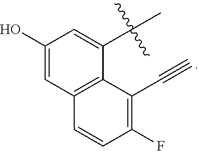 [SMILES: *c1cc(O)cc2ccc(F)c(C#C)c12]

In some embodiments, for a compound of Formula (I), (I′), (I-A), (I-A1), (I-A2), (I-A3), (I-B), (I-B1), (I-B2), or (I-B3), R¹⁷ is selected from

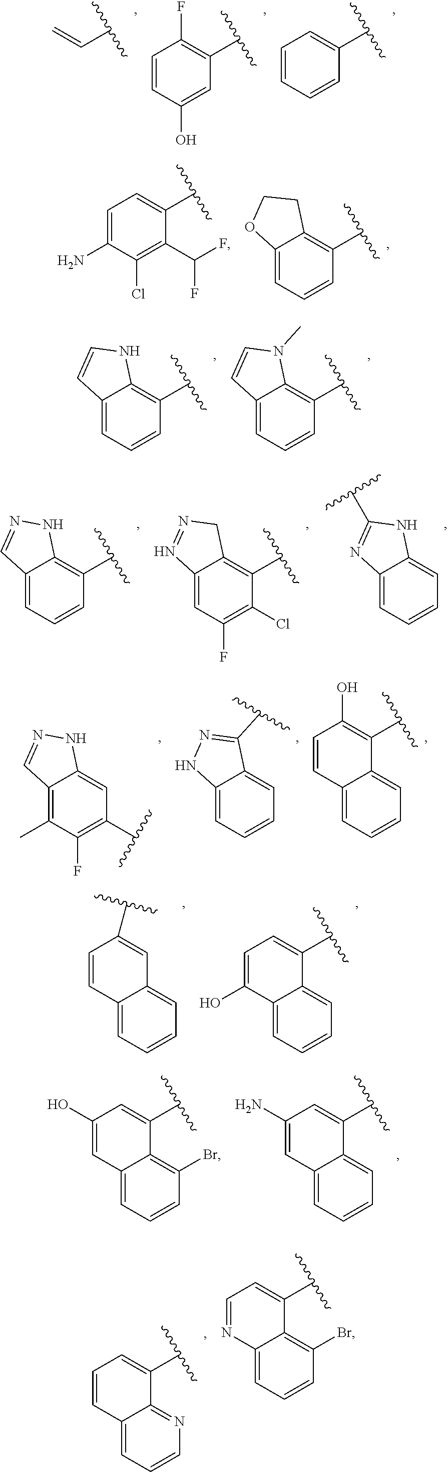

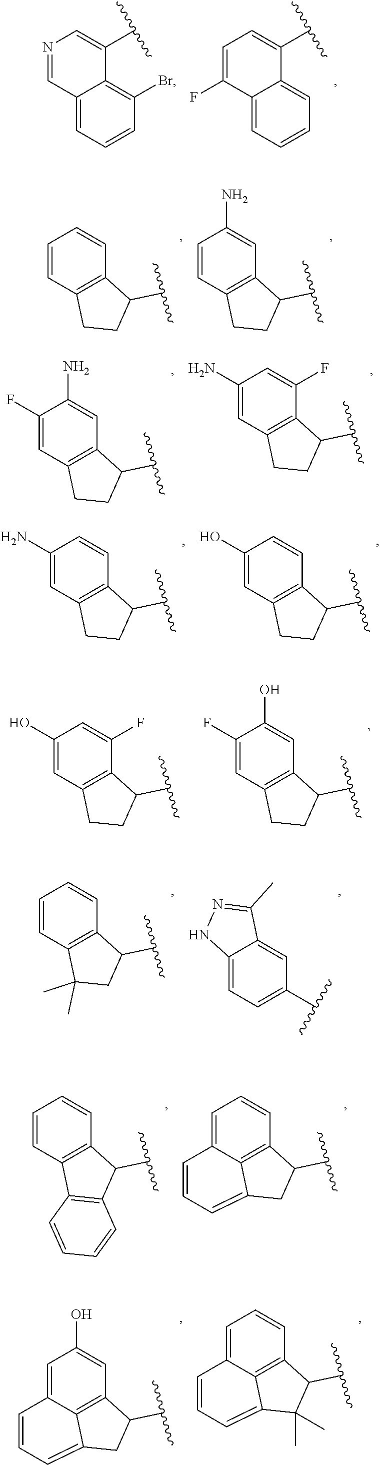

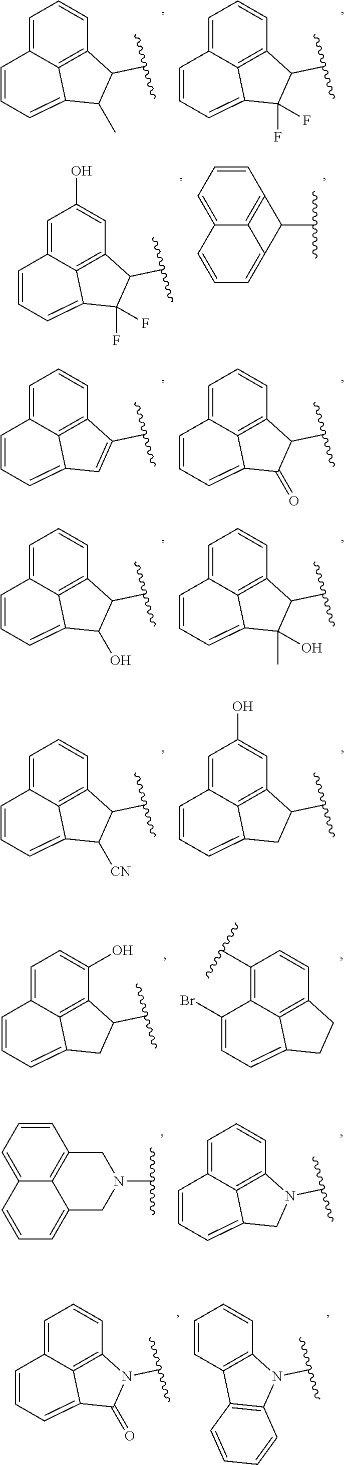

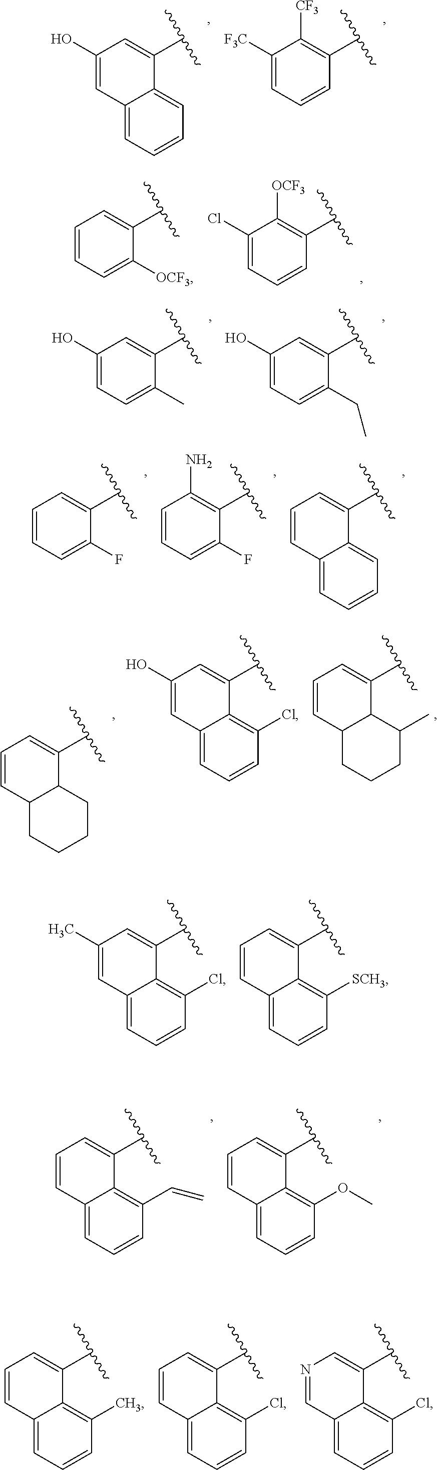

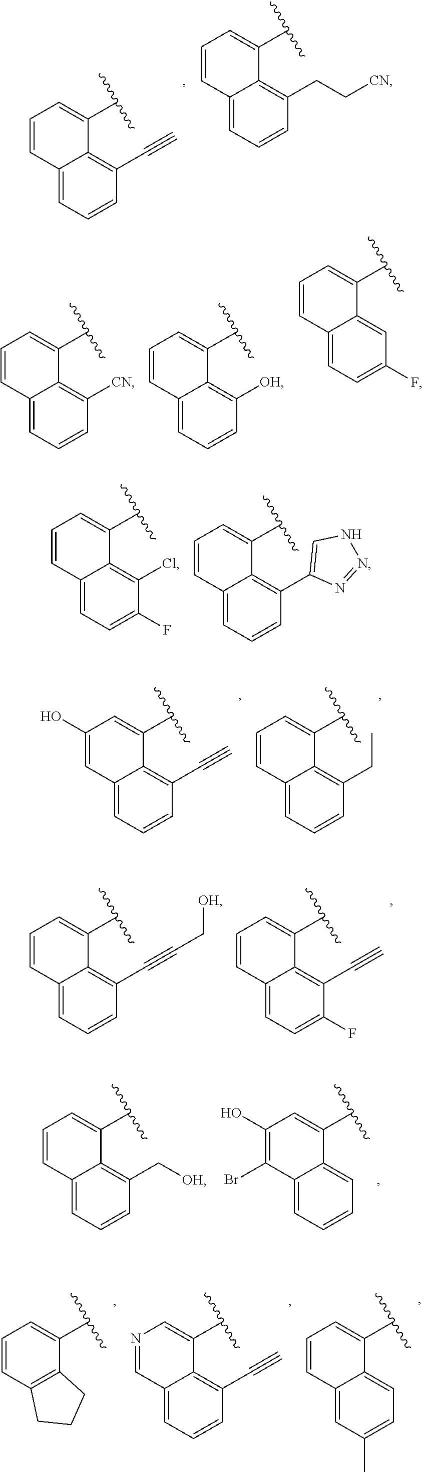

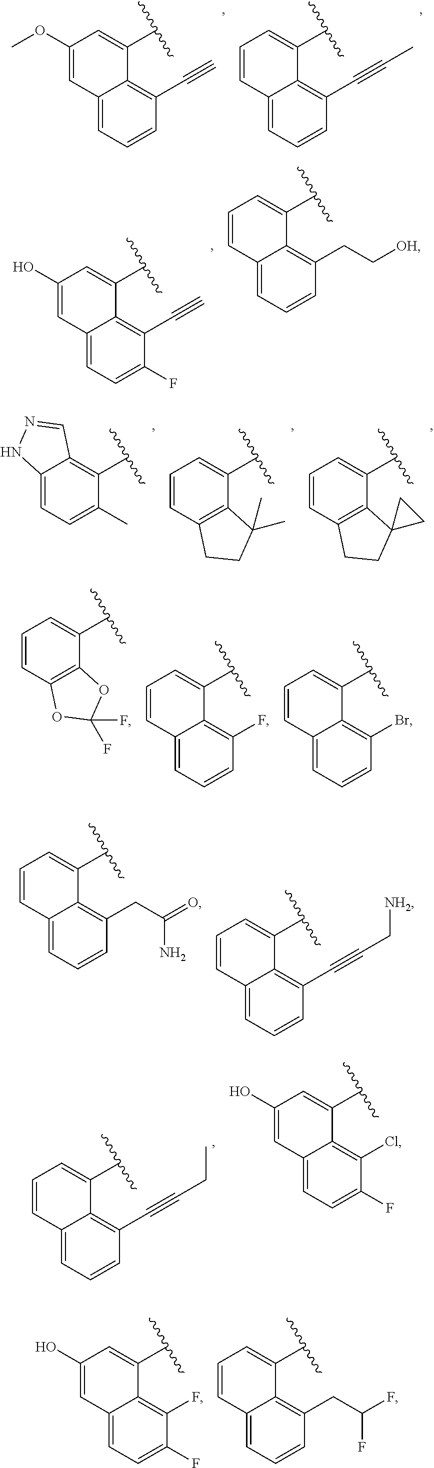

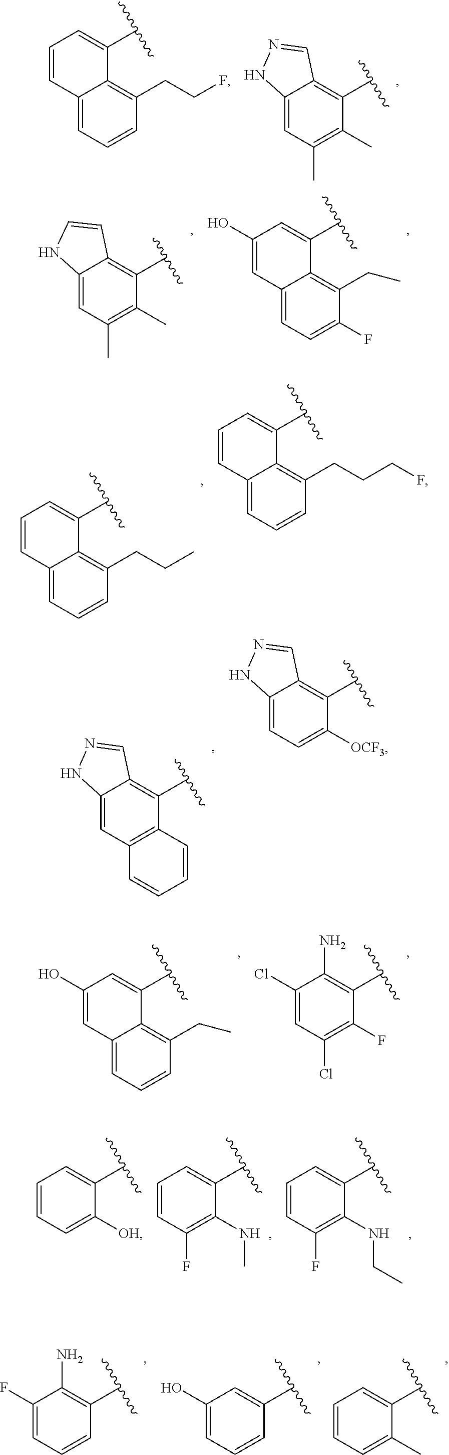

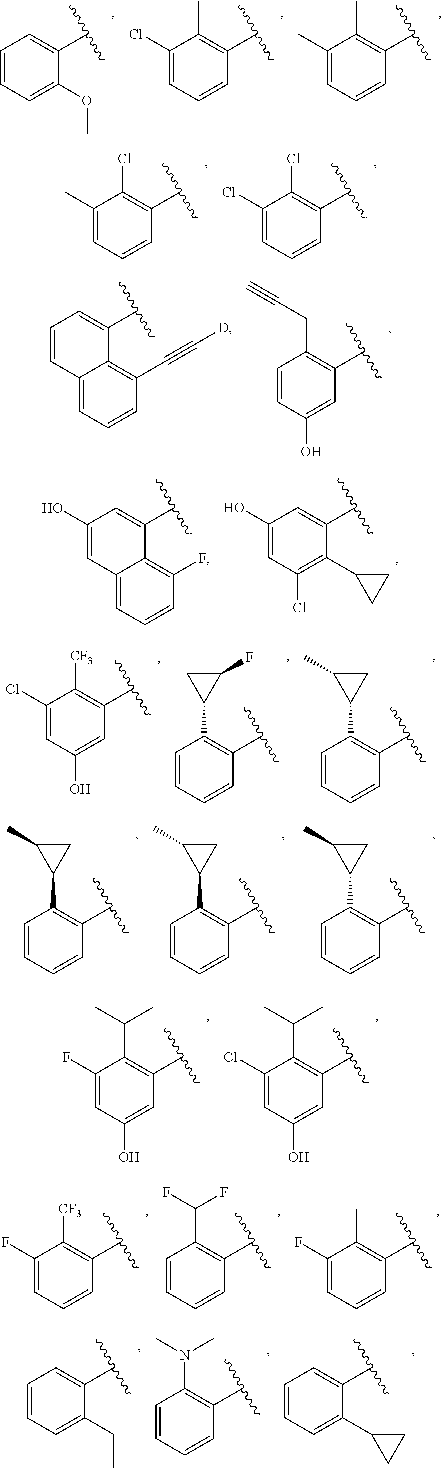

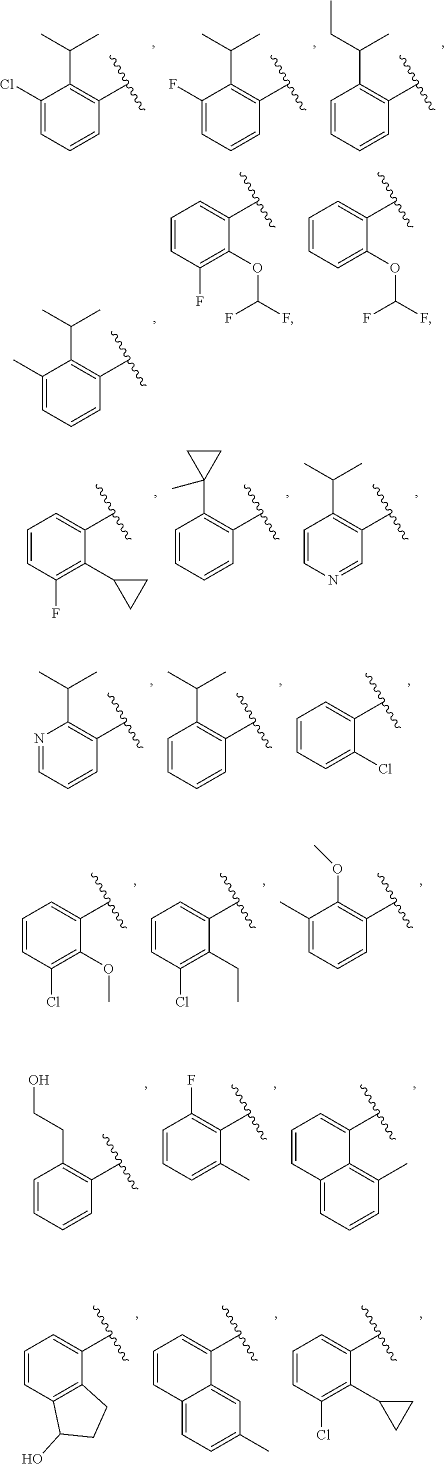

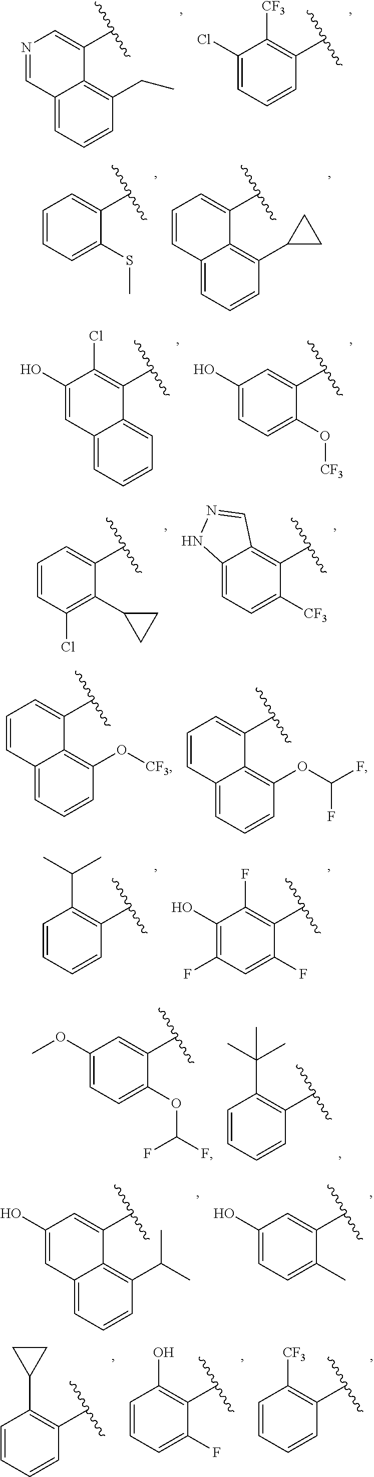

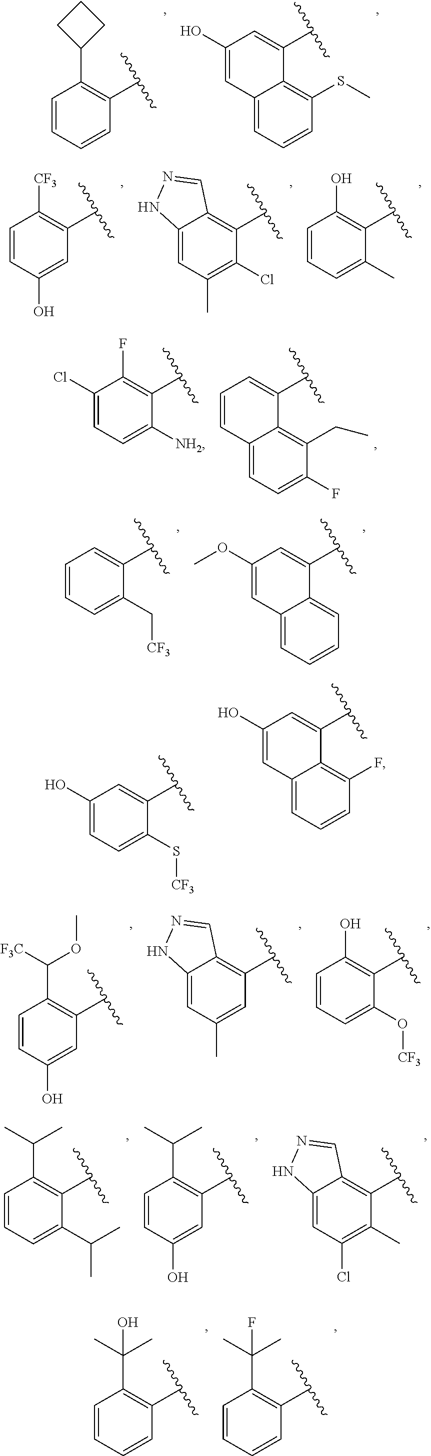

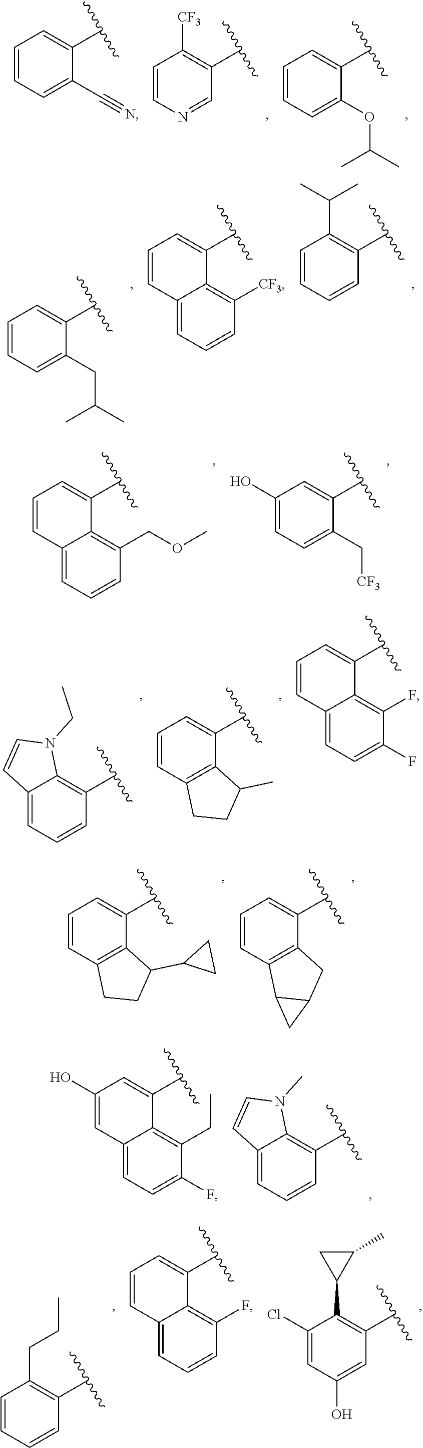

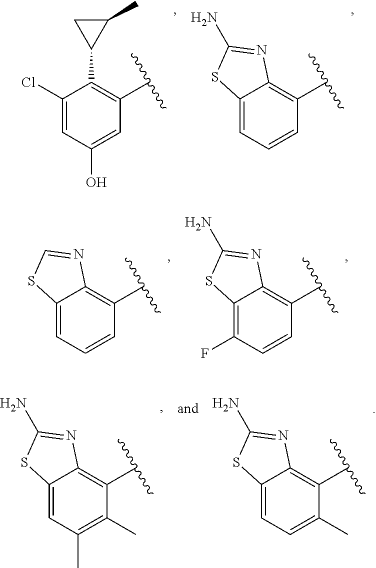 [SMILES: *c1cc(O)cc(Cl)c1[C@@H]1C[C@H]1C ; *c1cccc2sc(N)nc12 ; *c1cccc2scnc12 ; *c1ccc(F)c2sc(N)nc12 ; *c1c(C)c(C)cc2sc(N)nc12 ; *c1c(C)ccc2sc(N)nc12]

In some embodiments R¹⁷ is selected from

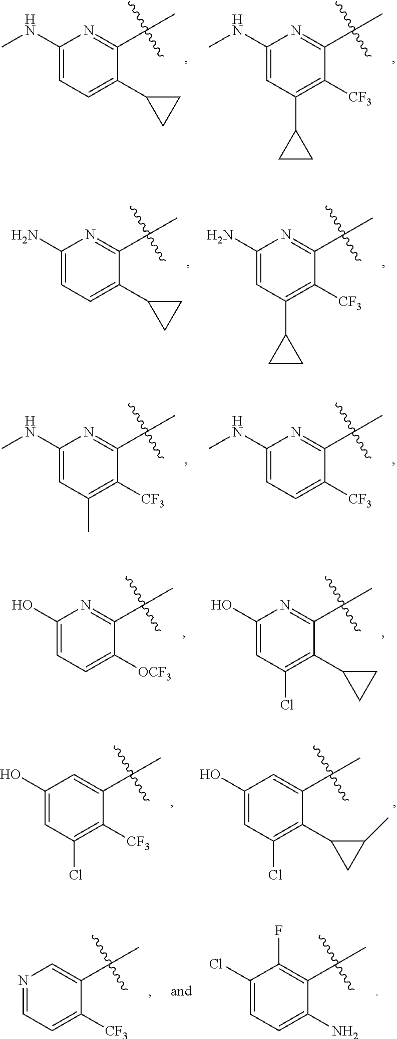

In some embodiments, for a compound of Formula (I), (I′), (I-A), (I-A1), (I-A2), (I-A3), (I-B), (I-B1), (I-B2), or (I-B3), R¹⁷ is substituted with one, two, or three substituents independently selected from halogen, —CN, C₁₋₆ alkyl, C₂₋₆ alkenyl, C₂₋₆ alkynyl, C₃₋₆ cycloalkyl, —OR²², —SR²², and —N(R²²)(R²³), wherein C₁₋₆ alkyl, C₂₋₆ alkenyl, C₂₋₆ alkynyl, and C₃₋₆ cycloalkyl are optionally substituted with one, two, or three substituents independently selected from halogen, C₁₋₆ alkyl, C₁₋₆ haloalkyl, and —OR²². In some embodiments, R¹⁷ is substituted with one, two, or three substituents independently selected from halogen, —CN, C₁₋₃ alkyl, C₂₋₃ alkenyl, C₂₋₃ alkynyl, —OR²², and —N(R²²)(R²³). In some embodiments, R¹⁷ is substituted with one, two, or three substituents independently selected from halogen, —CN, —CH₃, —C═CH, —OH, and —NH₂. In some embodiments, R¹⁷ is substituted with —F, —CN, and —NH₂. In some embodiments, R¹⁷ is substituted with —F, —C═CH, and —OH.

In some embodiments, for a compound of Formula (I), (I′), (I-A), (I-A1), (I-A2), (I-A3), (I-B), (I-B1), (I-B2), or (I-B3), R⁸ is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰. In some embodiments, R⁸ is selected from hydrogen, halogen, —CN, C₁₋₆ alkyl, and —OR¹², wherein C₁₋₆ alkyl is optionally substituted with one, two, or three R²⁰. In some embodiments, R⁸ is halogen, such as R⁸ is —F.

In some embodiments, for a compound of Formula (I), (I′), (I-A), (I-A3), (I-B), or (I-B3), R^{8a} is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰. In some embodiments, R^{8a} is selected from hydrogen, halogen, —CN, C₁₋₆ alkyl, and —OR¹², wherein C₁₋₆ alkyl is optionally substituted with one, two, or three R²⁰. In some embodiments, R^{8a} is hydrogen. In some embodiments, R⁸ and R^{8a} are each hydrogen.

In some embodiments, for a compound of Formula (I), (I′), (I-A), (I-A1), (I-A2), (I-A3), (I-B), (I-B1), (I-B2), or (I-B3), R^{A} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 8-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 8-membered heterocycle are optionally substituted with one, two, or three R²⁰. In some embodiments, R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³). In some embodiments, R^{A} is independently selected at each occurrence from halogen, oxo, —CN, C₁₋₃ alkyl, C₁₋₃ haloalkyl, —OH, —OCH₃, —NH₂, —NHMe, —C(O)H, —C(O)CH₃, —C(O)NHMe, and —C(O)NH₂. In some embodiments, R^{A} is independently selected at each occurrence from —Cl, —F, oxo, —CN, —CH₃, —CF₃, —OH, —OCH₃, —NH₂, and —NHMe. In some embodiments, R^{A} is independently selected at each occurrence from C₁₋₆ alkyl optionally substituted with one, two, or three R²⁰. In some embodiments, R^{A} is independently selected at each occurrence from C₁₋₆ alkyl and C₁₋₆ haloalkyl. In some embodiments, R^{A} is independently selected at each occurrence from —CH₃, —CH₂F, —CHF₂, and —CF₃. In some embodiments, n is an integer from 0 to 3. In some embodiments, n is 0. In some embodiments, n is 1. In some embodiments, n is 2. In some embodiments, n is 3.

In some embodiments, for a compound of Formula (I), (I′), (I-A), (I-A1), (I-A2), (I-A3), (I-B), (I-B1), (I-B2), or (I-B3), L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—. In some embodiments, L² is selected from —(C₁₋₆ alkyl)-(C₃₋₁₀ carbocycle)-, —(C₁₋₆ alkyl)-(3- to 10-membered heterocycle)-, —O—(C₁₋₆ alkyl)-(C₃₋₁₀ carbocycle)-, —O—(C₁₋₆ alkyl)-(3- to 10-membered heterocycle)-, —N(R^{7d})—(C₁₋₆ alkyl)-(C₃₋₁₀ carbocycle)-, —N(R^{7d})—(C₁₋₆ alkyl)-(3- to 10-membered heterocycle)-, —S—(C₁₋₆ alkyl)-(C₃₋₁₀ carbocycle)-, and —S—(C₁₋₆ alkyl)-(3- to 10-membered heterocycle)-, each of which is optionally substituted with one, two, or three R^{L}. In some embodiments, L² is selected from —O—, —OC(R^{7c})₂—, and —O—(C₁₋₆ alkyl)-(3- to 10-membered heterocycle)-, wherein —O—(C₁₋₆ alkyl)-(3- to 10-membered heterocycle)- is optionally substituted with one, two, or three R^{L}. In some embodiments, L² is selected from —O— and —OC(R^{7c})₂—. In some embodiments, L² is —OCH(3- to 10-membered heterocycle)-, wherein the 3- to 10-membered heterocycle is optionally substituted with one, two, or three substituents selected from halogen and C₁₋₃ alkyl. In some embodiments, L² is selected from

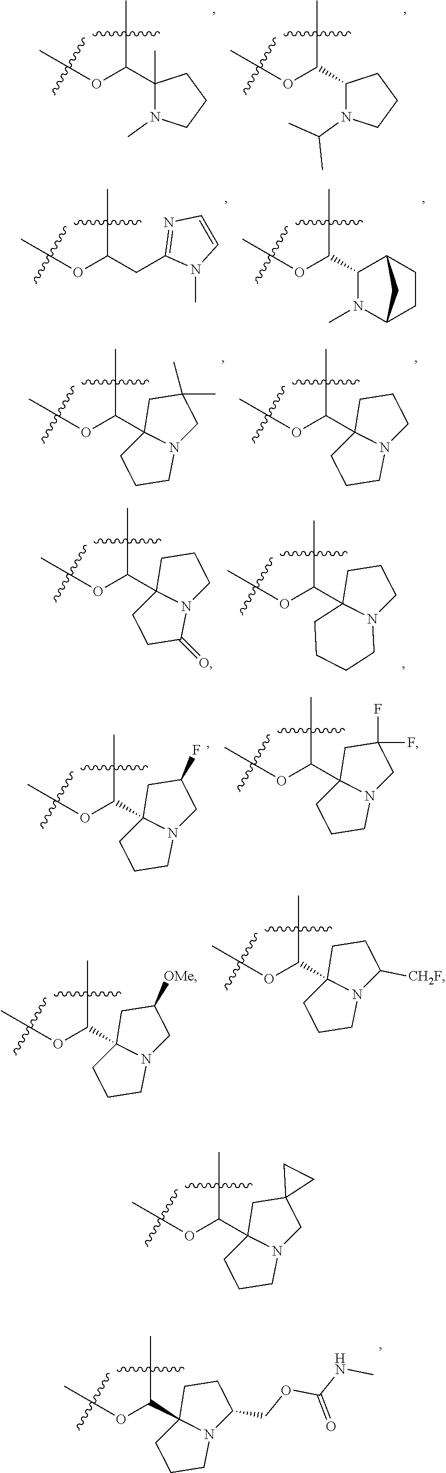

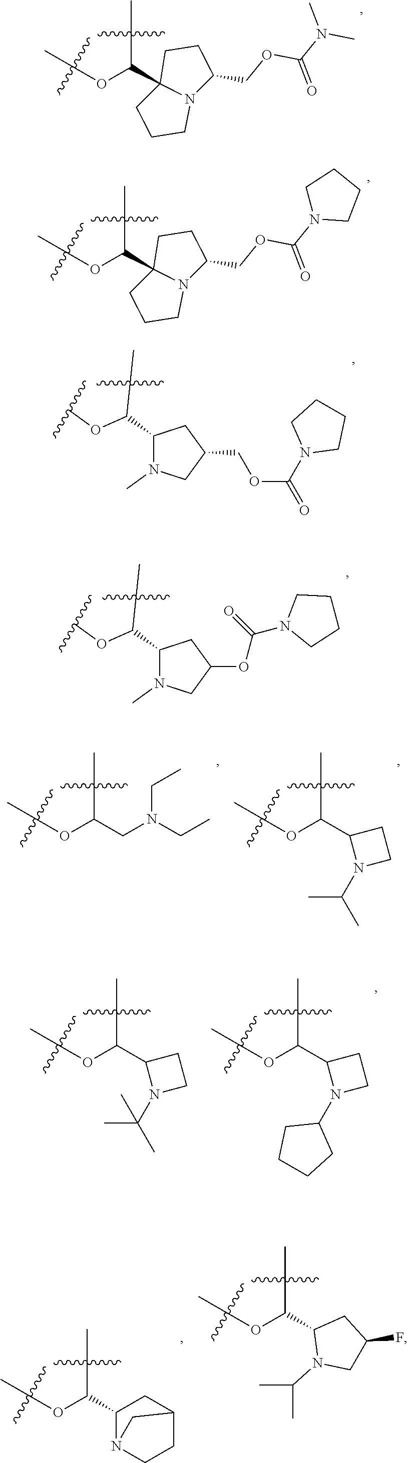

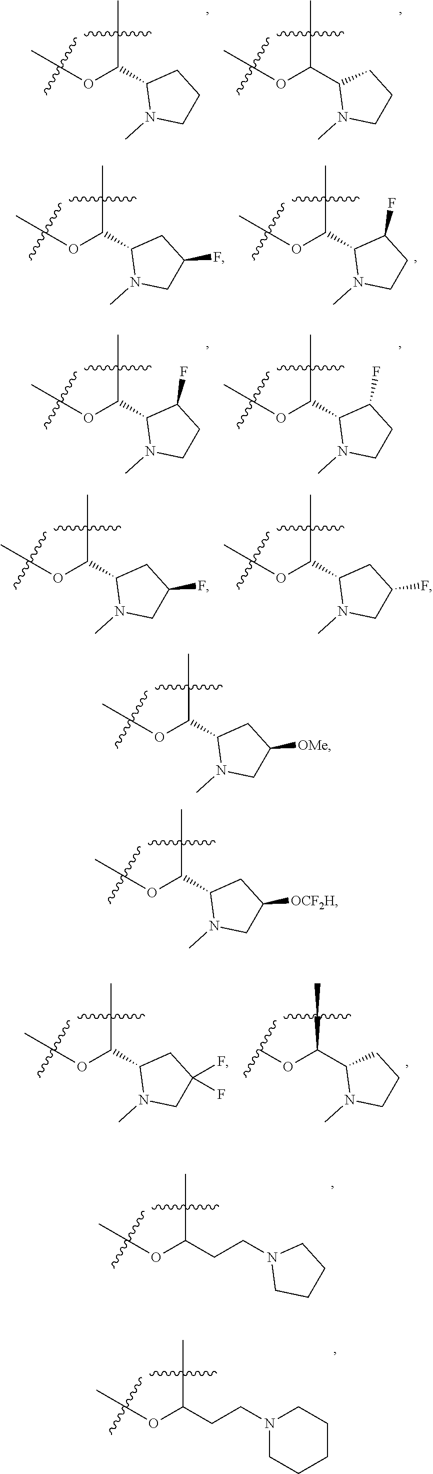

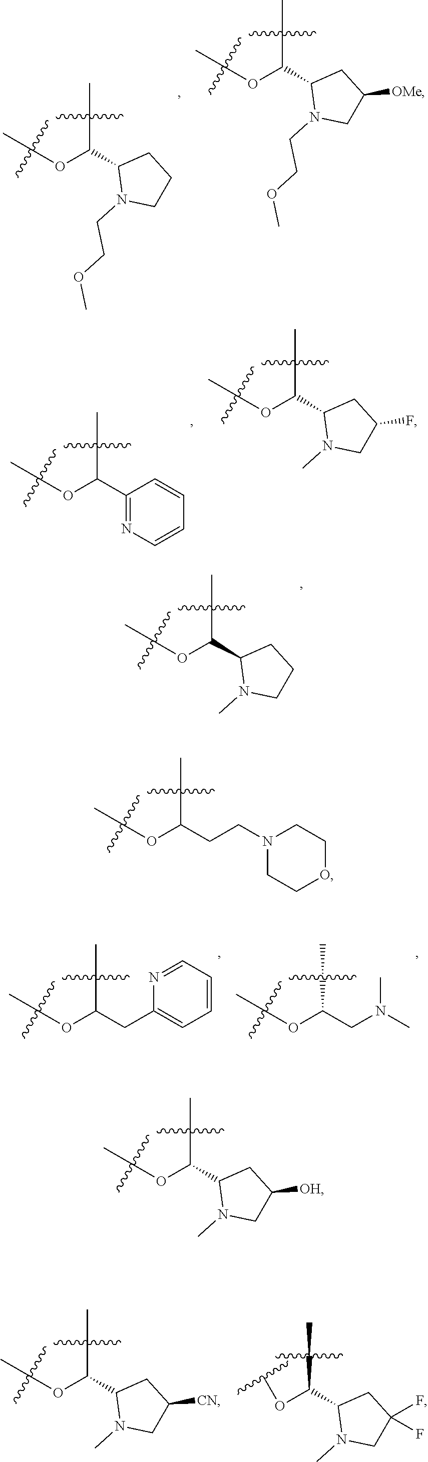

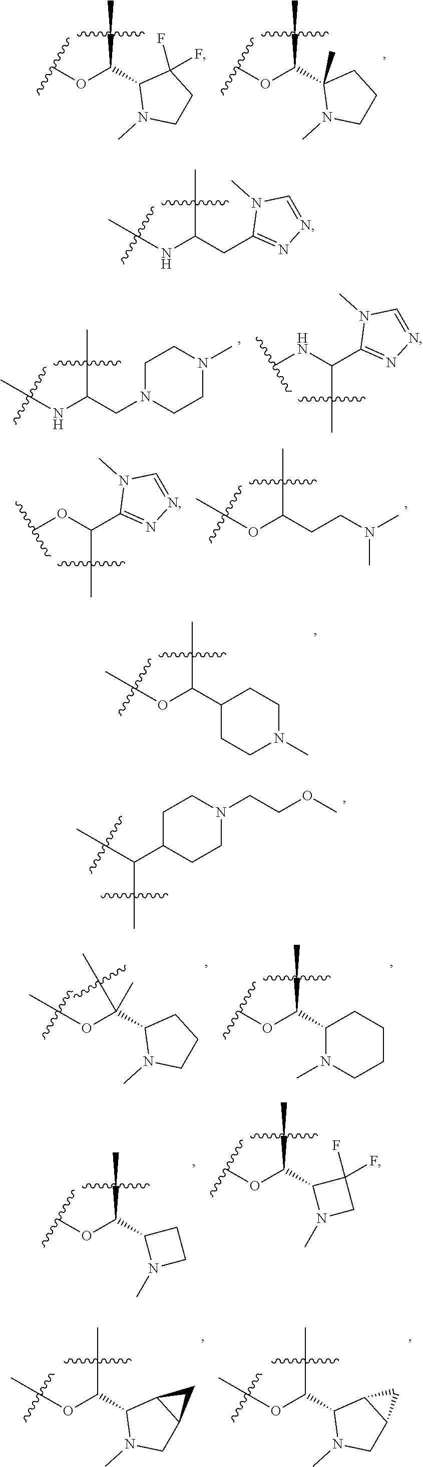

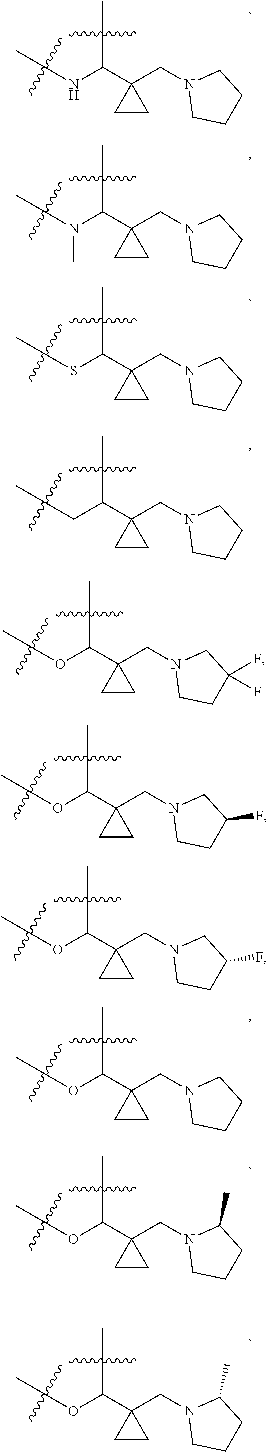

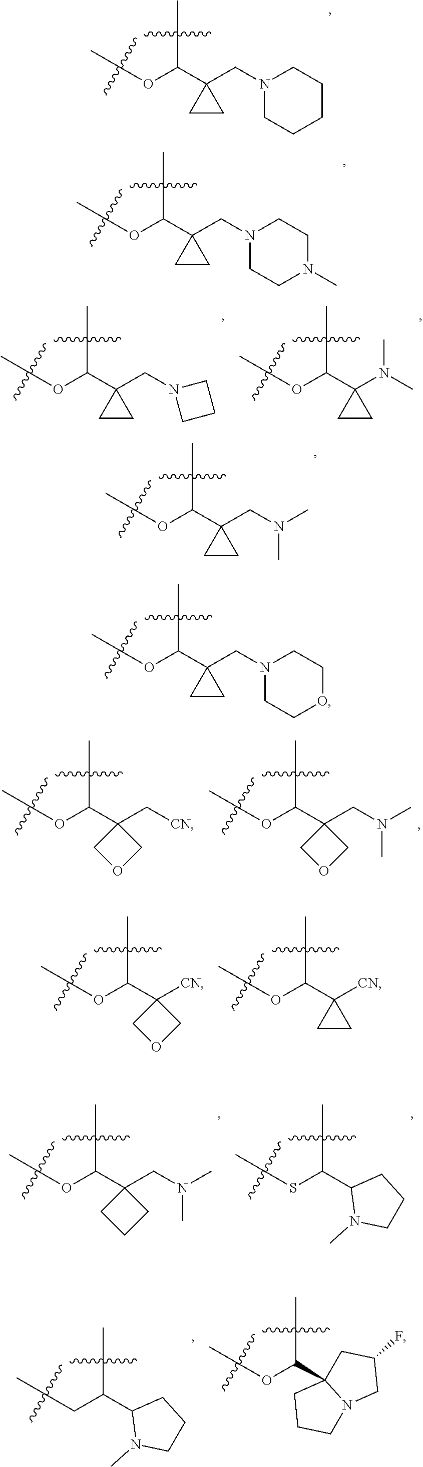

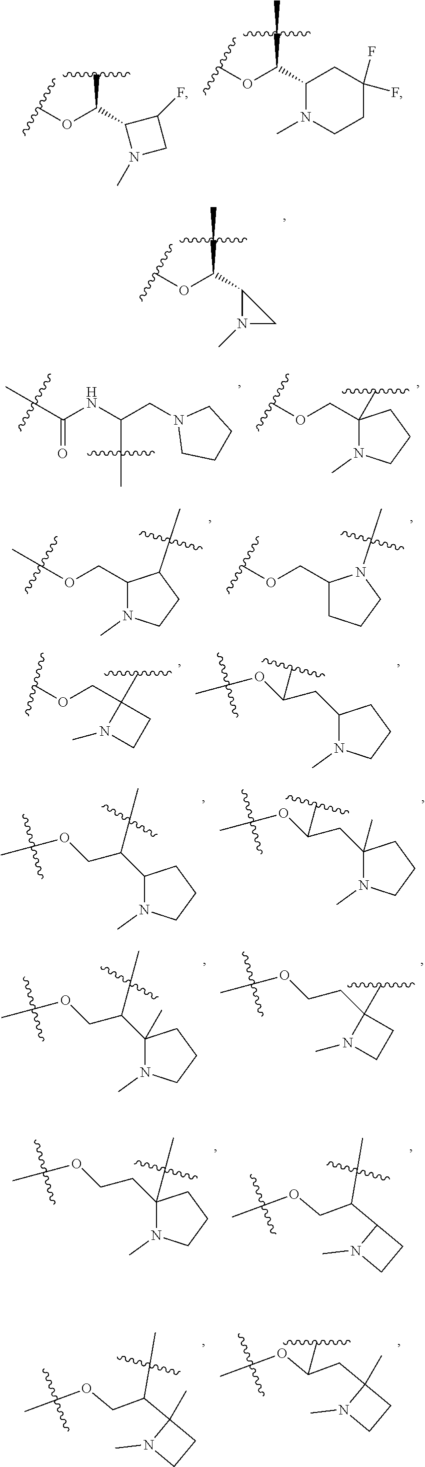

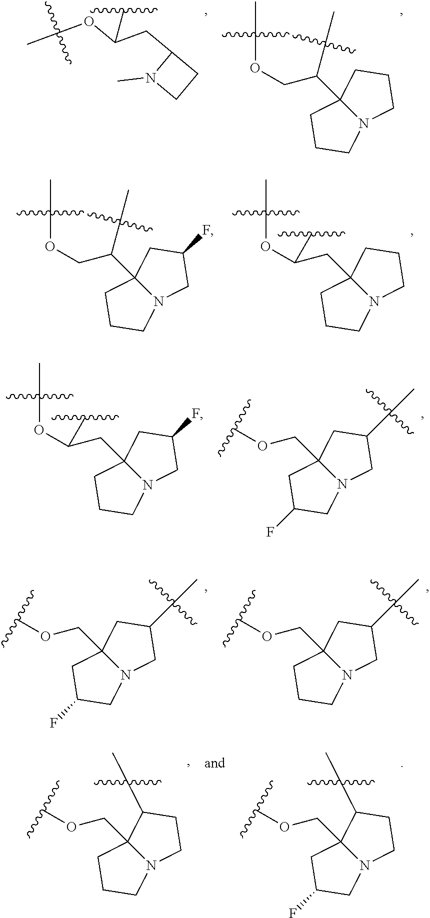

such as L² is selected from

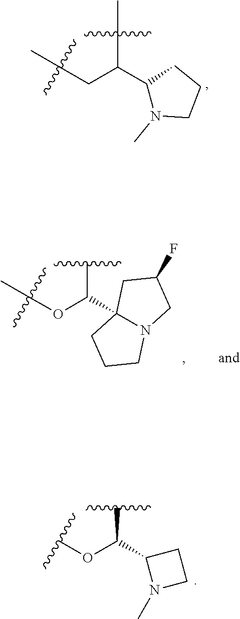

In some embodiments, for a compound of Formula (I), (I′), (I-A), (I-A1), (I-A2) (I-A3, (I-B) (I-B1), (I-B2), or (I-B3), L³ is selected from C₁₋₆ alkyl, C₂₋₆ alkenyl, and 2- to 6-membered heteroalkyl, each of which is optionally substituted with one, two, or three R^{L}. In some embodiments, L³ is selected from C₁₋₆ alkyl and C₂₋₆ alkenyl, each of which is optionally substituted with one, two, or three R^{L}. In some embodiments, L³ is selected from C₁₋₆ alkyl and C₂₋₆ alkenyl. In some embodiments, L³ is selected from C₂₋₄ alkyl and C₂₋₄ alkenyl, each of which is optionally substituted with oxo.

In some embodiments, for a compound of Formula (I), (I′), (I-A), (I-A1), (I-A2), (I-A3), (I-B), (I-B1), (I-B2), or (I-B3), L⁴ is selected from a bond, —O—, —N(R^{7d})—, —C(O)—, —S(O)₂—, —S(O)—, —C(R^{7c})₂—, —OC(R^{7c})₂—, —N(R^{7d})C(R^{7c})₂—, —C(O)C(R^{7c})₂—, —S(O)₂C(R^{7c})₂—, —S(O)C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S(O)₂—, —C(R^{7c})₂S(O)—, —N(R^{7d})C(O)—, —N(R^{7d})S(O)₂—, —N(R^{7d})S(O)—, —C(O)N(R^{7d})—, —S(O)₂N(R^{7d})—, —S(O)N(R^{7d})—, —OC(O)—, —OS(O)₂—, —OS(O)—, —C(O)O—, —S(O)₂O—, —S(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L4 is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L}. In some embodiments, L⁴ is selected from —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}. In some embodiments, L⁴ is a bond.

In some embodiments, for a compound of Formula (I), (I′), (I-A), (I-A1), (I-A2), (I-A3), (I-B), (I-B1), (I-B2), or (I-B3), -L²-L³-L⁴- is selected from

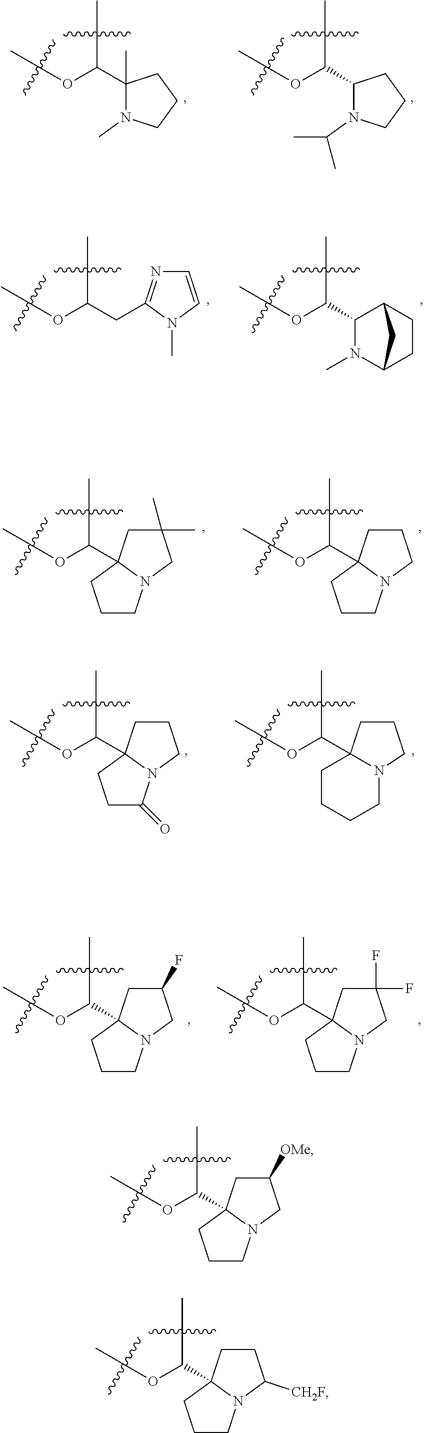

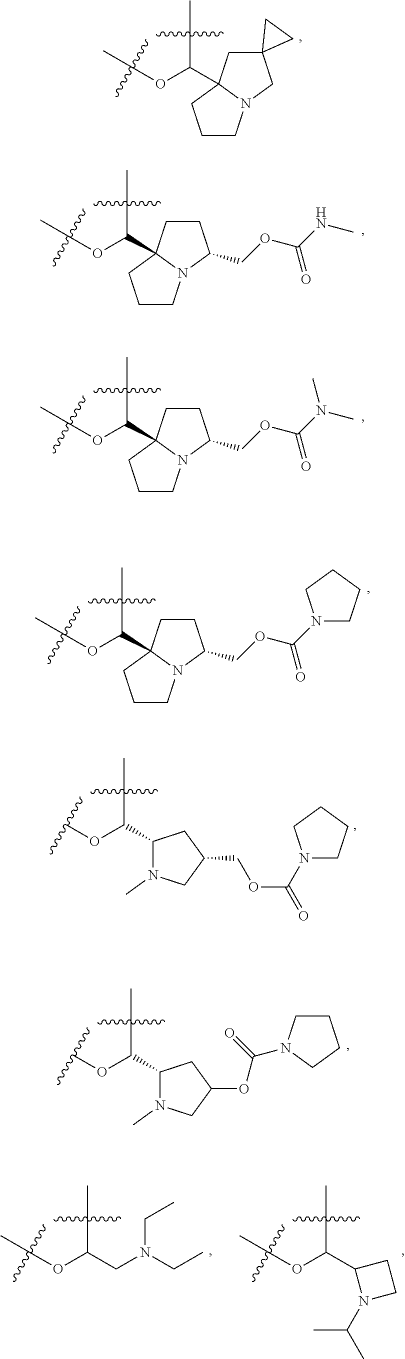

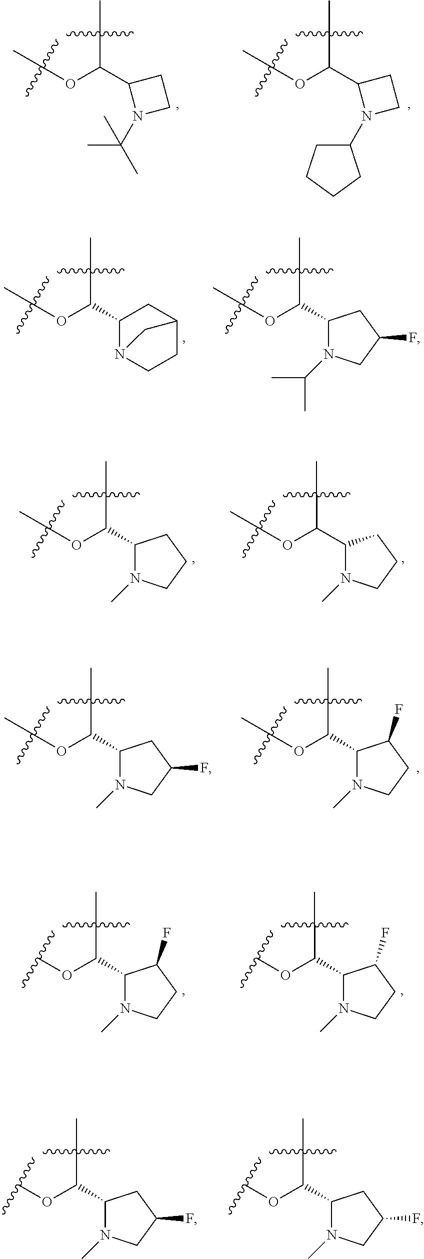

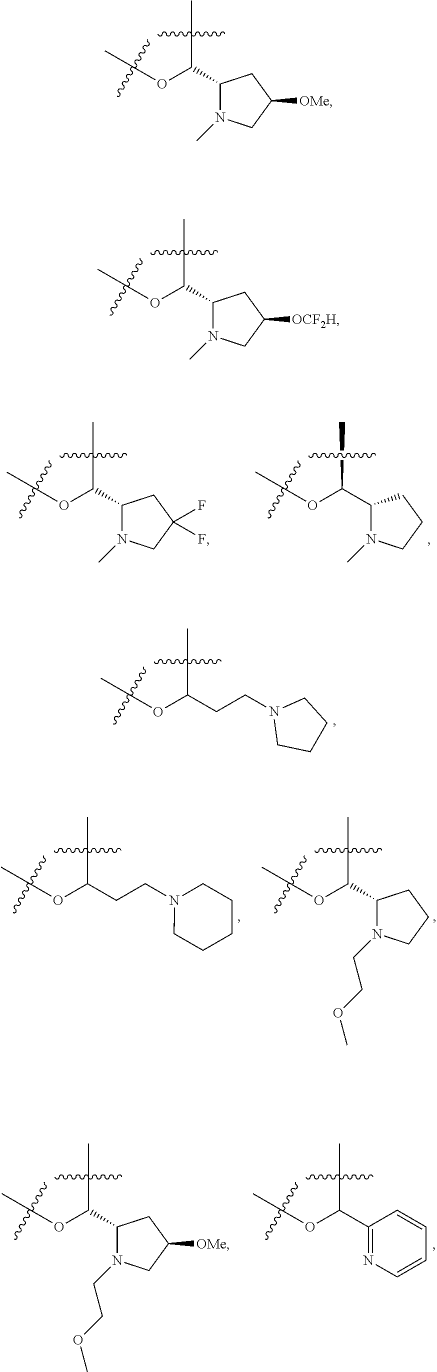

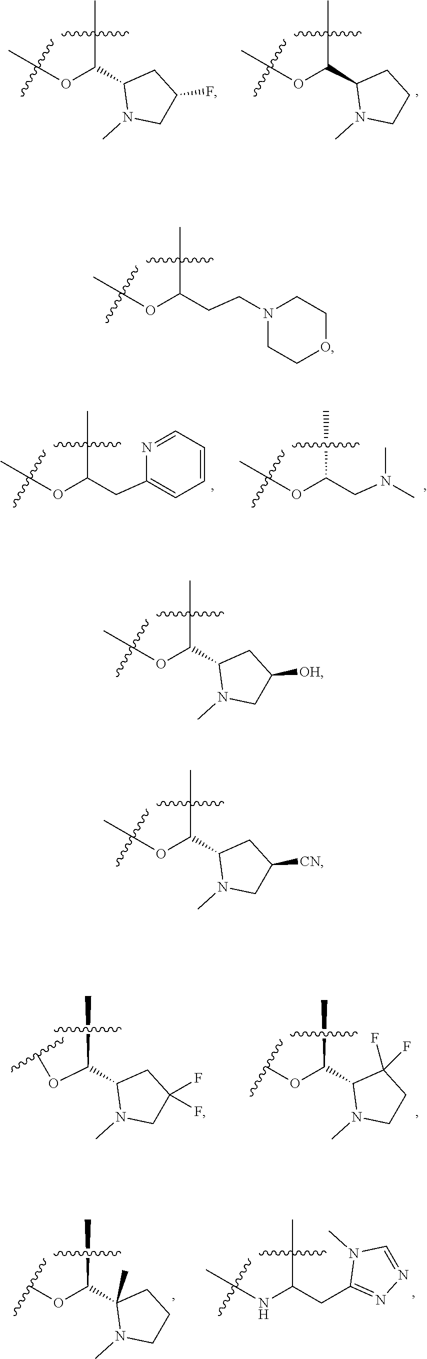

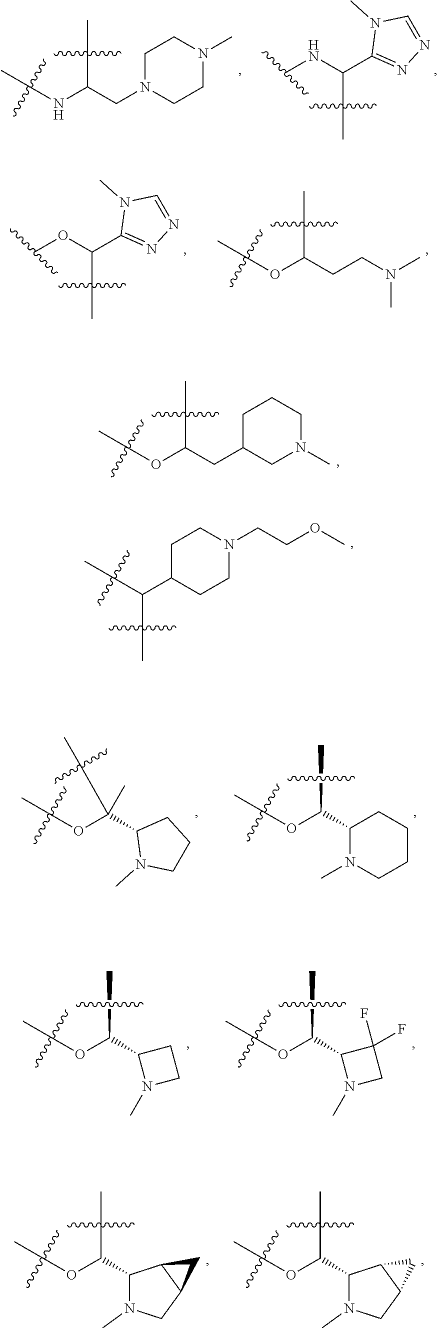

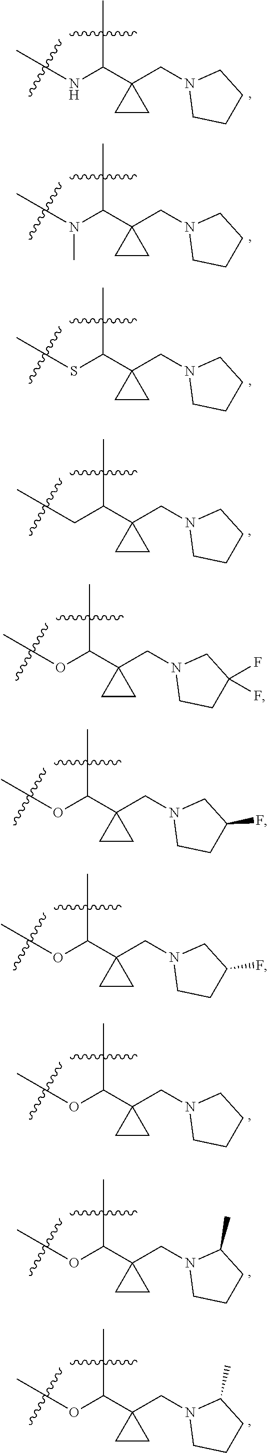

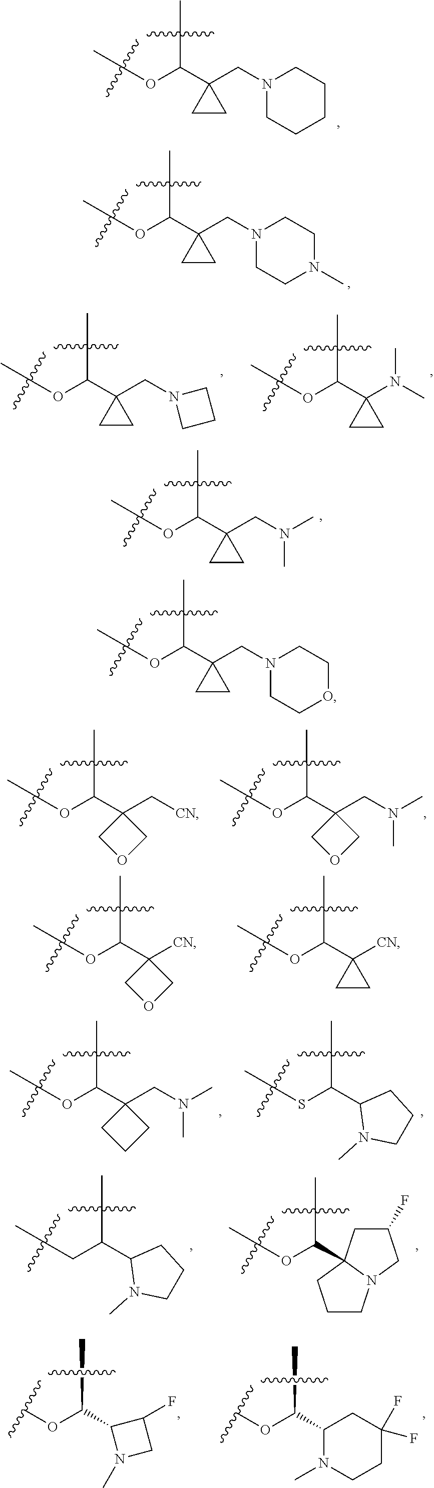

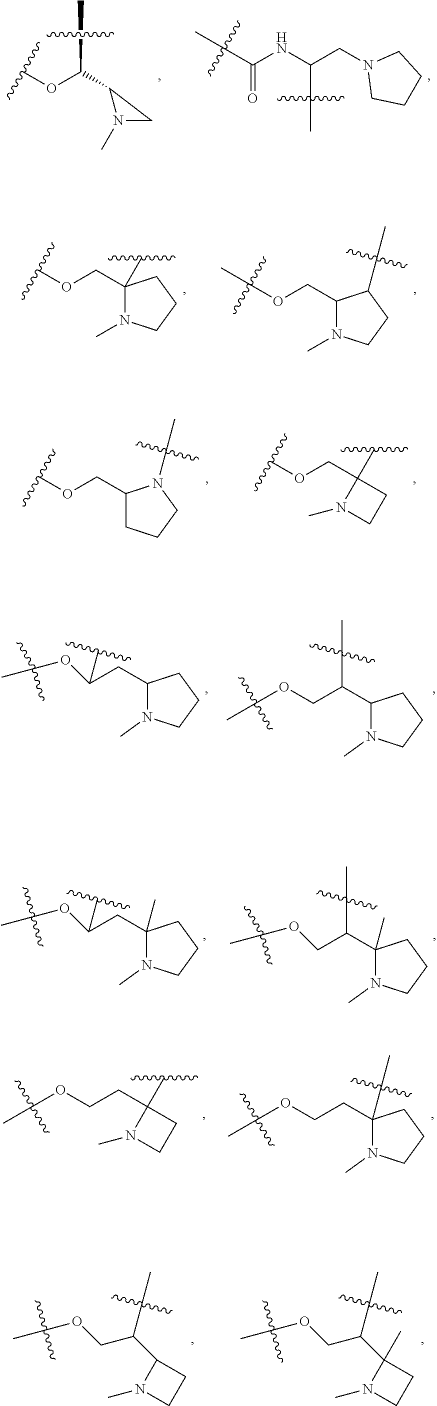

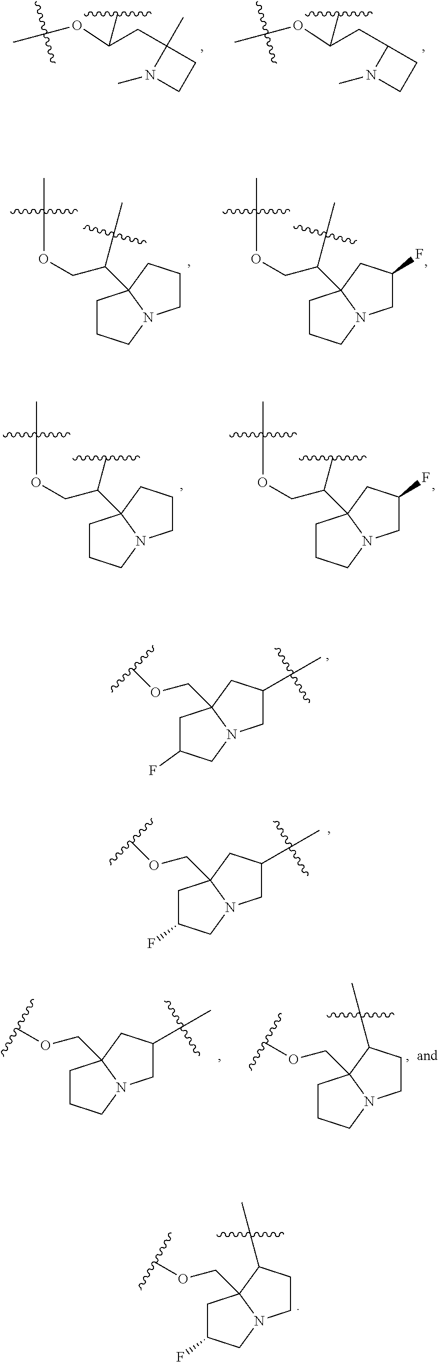

In some embodiments, for a compound of Formula (I), (I′), (I-A), (I-A1), (I-A2), (I-A3), (I-B), (I-B1), (I-B2), or (I-B3), L⁵ is selected from a bond, C₁₋₆ alkyl, C₂₋₆ alkenyl, C₃₋₁₀ carbocycle, and 3- to 10-membered heterocycle, wherein C₁₋₆ alkyl, C₂₋₆ alkenyl, C₃₋₁₀ carbocycle, and 3- to 10-membered heterocycle are optionally substituted with one, two, or three R^{L}. In some embodiments, L⁵ is selected from a bond, C₁₋₆ alkyl, C₃₋₆ carbocycle, and 3- to 6-membered heterocycle, wherein C₁₋₆ alkyl, C₃₋₆ carbocycle, and 3- to 6-membered heterocycle are optionally substituted with one, two, or three R^{L}. In some embodiments, L⁵ is selected from a bond and C₁₋₆ alkyl, wherein C₁₋₆ alkyl is optionally substituted with one, two, or three R^{L}. In some embodiments, L⁵ is selected from C₁₋₆ alkyl, C₃₋₆ carbocycle, and 3- to 6-membered heterocycle, wherein C₁₋₆ alkyl, C₃₋₆ carbocycle, and 3- to 6-membered heterocycle are optionally substituted with one, two, or three R^{L}. In some embodiments, L⁵ is a bond.

In some embodiments, for a compound of Formula (I), (I′), (I-A), (I-A1), (I-A2), (I-A3), (I-B), (I-B1), (I-B2), or (I-B3), L⁶ is selected from a bond, —O—, —N(R^{7d})—, —C(O)—, —S(O)₂—, —S(O)—, —C(R^{7c})₂—, —OC(R^{7c})₂—, —N(R^{7d})C(R^{7c})₂—, —C(O)C(R^{7c})₂—, —S(O)₂C(R^{7c})₂—, —S(O)C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S(O)₂—, —C(R^{7c})₂S(O)—, —N(R^{7d})C(O)—, —N(R^{7d})S(O)₂—, —N(R^{7d})S(O)—, —C(O)N(R^{7d})—, —S(O)₂N(R^{7d})—, —S(O)N(R^{7d})—, —OC(O)—, —OS(O)₂—, —OS(O)—, —C(O)O—, —S(O)₂O—, —S(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}. In some embodiments, L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}. In some embodiments, L⁶ is selected from —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}. In some embodiments, L⁶ is a bond.

In some embodiments, for a compound of Formula (I), (I′), (I-A), (I-A1), (I-A2), (I-A3), (I-B), (I-B1), (I-B2), or (I-B3):
L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—;
L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

In some embodiments, for a compound of Formula (I), (I′), (I-A), (I-A1), (I-A2), (I-A3), (I-B), (I-B1), (I-B2), or (I-B3):
L² is selected from —O— and —OC(R^{7c})₂—;
L³ is selected from C₁₋₆ alkyl and C₂₋₆ alkenyl;
L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L};
L⁵ is selected from a bond and C₁₋₆ alkyl, wherein C₁₋₆ alkyl is optionally substituted with one, two, or three R^{L}; and
L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

In some embodiments, for a compound of Formula (I), (I′), (I-A), (I-A1), (I-A2), (I-A3), (I-B), (I-B1), (I-B2), or (I-B3), R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —N(R¹²)(R¹³), —C(O)OR¹², —N(R¹⁴)C(O)N(R¹²)(R¹³), —N(R¹⁴)S(O)₂R¹⁵, —C(O)R¹², —S(O)R¹⁵, —OC(O)R¹⁵, —C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰. In some embodiments, R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰. In some embodiments, R^{L} is independently selected at each occurrence from halogen, oxo, C₁₋₆ alkyl, C₃₋₈ carbocycle, 3- to 10-membered heterocycle, and —C(O)R¹², wherein C₁₋₆ alkyl, C₃₋₈ carbocycle, and 3- to 10-membered heterocycle are optionally substituted with one, two, or three R²⁰. In some embodiments, R^{L} is independently selected at each occurrence from halogen, oxo, C₁₋₆ alkyl, and —C(O)R¹², wherein C₁₋₆ alkyl is optionally substituted with one, two, or three R²⁰.

In some embodiments, for a compound of Formula (I), (I′), (I-A), (I-A1), (I-A2), (I-A3), (I-B), (I-B1), (I-B2), or (I-B3), R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —C(O)OR¹², —N(R¹⁴)C(O)N(R¹²)(R¹³), —N(R¹⁴)S(O)₂R¹⁵, —C(O)R¹², —S(O)R¹⁵, —OC(O)R¹⁵, —C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰. In some embodiments, R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰. In some embodiments, R^{7c} is independently selected at each occurrence from hydrogen, halogen, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰. In some embodiments, R^{7c} is independently selected at each occurrence from hydrogen and C₁₋₃ alkyl.

In some embodiments, for a compound of Formula (I), (I′), (I-A), (I-A1), (I-A2), (I-A3), (I-B), (I-B1), (I-B2), or (I-B3), R^{7d} is independently selected at each occurrence from hydrogen, C₁₋₆ alkyl, C₂₋₆ alkenyl, C₂₋₆ alkynyl, C₃₋₁₀ carbocycle, 3- to 10-membered heterocycle, —C(O)R¹², —S(O)R¹⁵, —C(O)N(R¹²)(R¹³), and —S(O)₂R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰. In some embodiments, R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰. In some embodiments, R^{7d} is independently selected at each occurrence from hydrogen, C₁₋₃ alkyl, and —C(O)R¹².

In some embodiments, for a compound of Formula (I), (I′), (I-A), (I-A1), (I-A2), (I-A3), (I-B), (I-B1), (I-B2), or (I-B3):
R⁶ is selected from hydrogen and halogen;
R^{6a} is hydrogen;
L⁷ is selected from a bond, —O—, —N(R^{7d})—, —C(O)—, and —C(R^{7c})₂—;
L^{7b} is selected from a bond, —O—, —C(O)—, and —C(R^{7c})₂—;
R¹⁷ is selected from C₆₋₁₂ aryl and 6- to 12-membered heteroaryl, each of which is optionally substituted with one, two, or three R²⁰;
R⁸ is selected from hydrogen, halogen, —CN, C₁₋₆ alkyl, and —OR¹², wherein C₁₋₆ alkyl is optionally substituted with one, two, or three R²⁰;
R^{8a} is hydrogen;
R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³);
n is an integer from 0 to 3;
L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—;
L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and
L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
and further optionally wherein:
R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰; and
R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰;
and further optionally wherein:
R¹⁷ is selected from

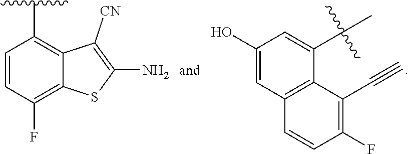 [SMILES: *c1ccc(F)c2sc(N)c(C#N)c12 ; "and" ; *c1cc(O)cc2ccc(F)c(C#C)c12]

In some embodiments, for a compound of Formula (I), (I′), (I-A), (I-A1), (I-A2), (I-A3), (I-B), (I-B1), (I-B2), or (I-B3):
R⁶ is selected from hydrogen and halogen;
R^{6a} is hydrogen;
L⁷ is a bond;
L^{7b} is a bond;
R¹⁷ is selected from naphthalenyl and benzothiophenyl, each of which is optionally substituted with one, two, or three R²⁰;
R⁸ is halogen;
R^{8a} is hydrogen;
n is 0;
L² is selected from —O— and —OC(R^{7c})₂—;
L³ is selected from C₁₋₆ alkyl and C₂₋₆ alkenyl;
L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L};
L⁵ is selected from a bond and C₁₋₆ alkyl, wherein C₁₋₆ alkyl is optionally substituted with one, two, or three R^{L}; and
L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L};
and further optionally wherein:
R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰; and
R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰;
and further optionally wherein:
R¹⁷ is selected from

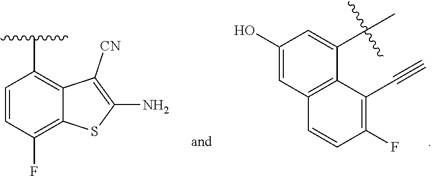 [SMILES: *c1ccc(F)c2sc(N)c(C#N)c12 ; *c1cc(O)cc2ccc(F)c(C#C)c12]

In some embodiments, for a compound of Formula (I) or (I):
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R⁶) or C(O), W⁷ is N(R^{7b}), W⁸ is C(R⁸)(R^{8a}) or C(O), and each [symbol] indicates a single bond;
R⁶, R^{6a}, R⁸, and R^{8a} are each independently selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰;
R⁷ is -L⁷-R¹⁷;
R^{7b} is -L^{7b}-R¹⁷;
L⁷ is selected from a bond, —O—, —N(R^{7d})—, —C(O)—, and —C(R^{7c})₂—;
L^{7b} is selected from is selected from a bond, —O—, —C(O)—, and —C(R^{7c})₂—;
R¹⁷ is selected from C₆₋₁₂ aryl and 5- to 12-membered heteroaryl, each of which is optionally substituted with one, two, or three R²⁰;
L¹ is -L²-L³-L⁴-L⁵-L⁶-;
L² is selected from

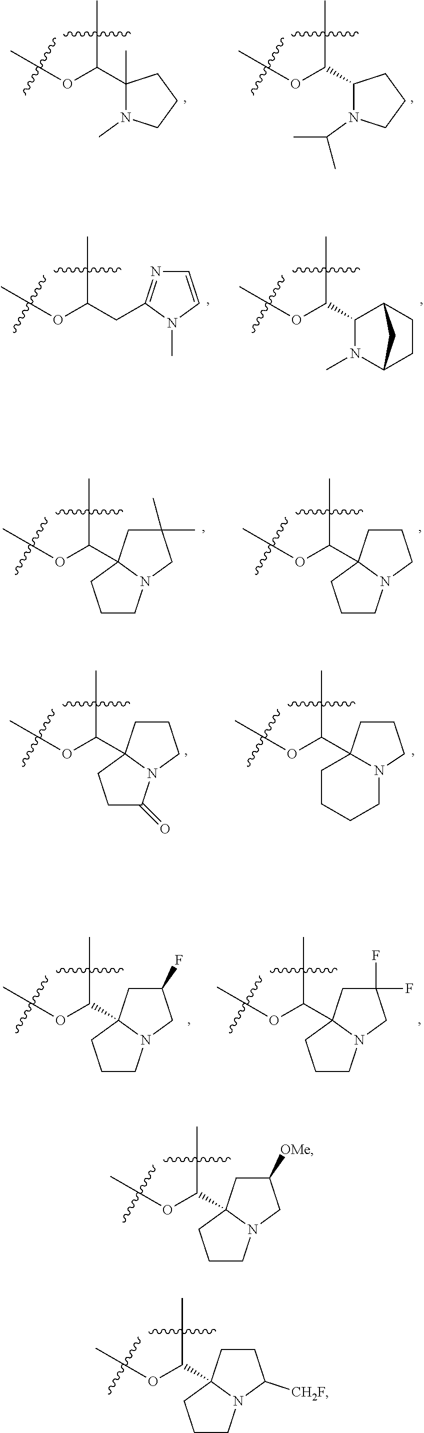

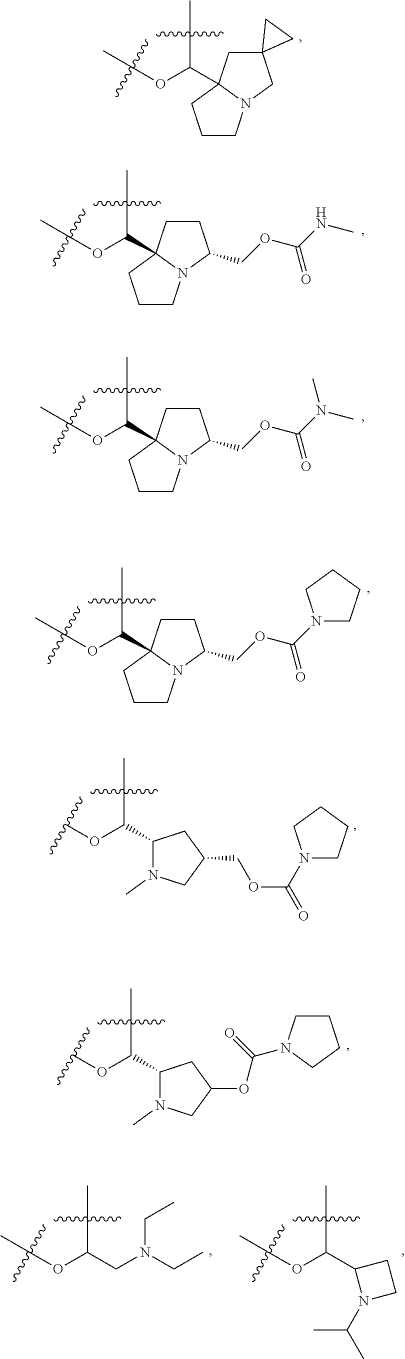

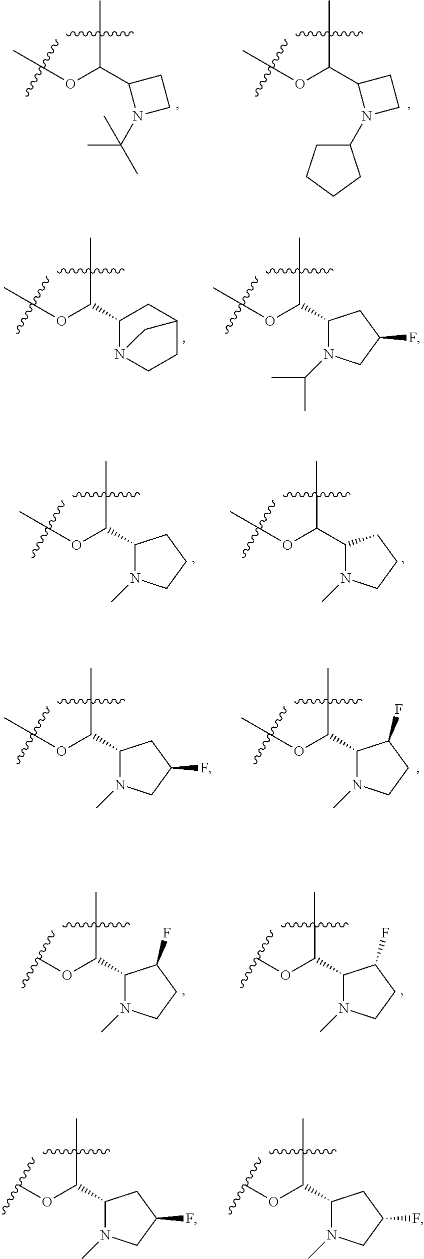

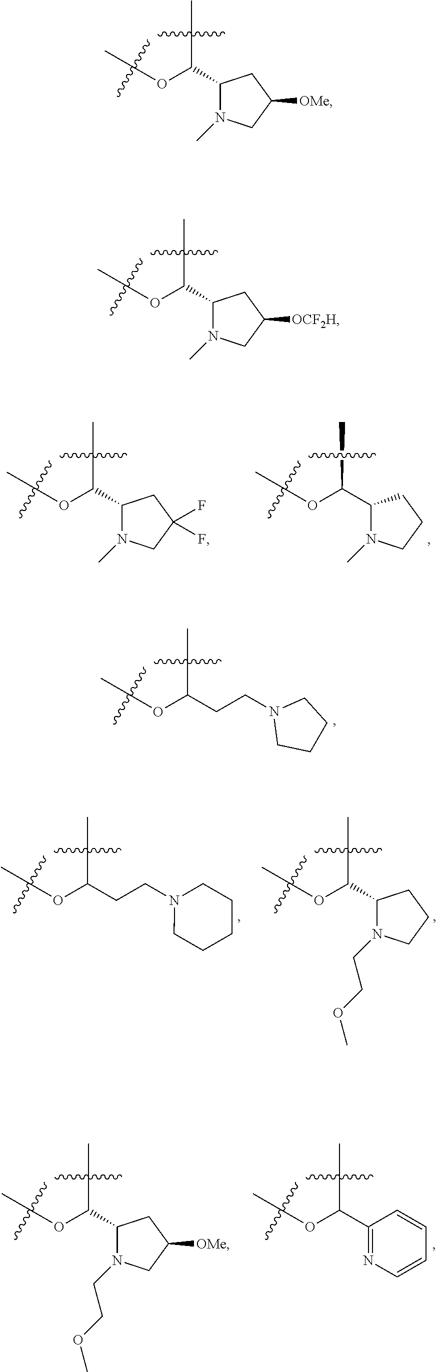

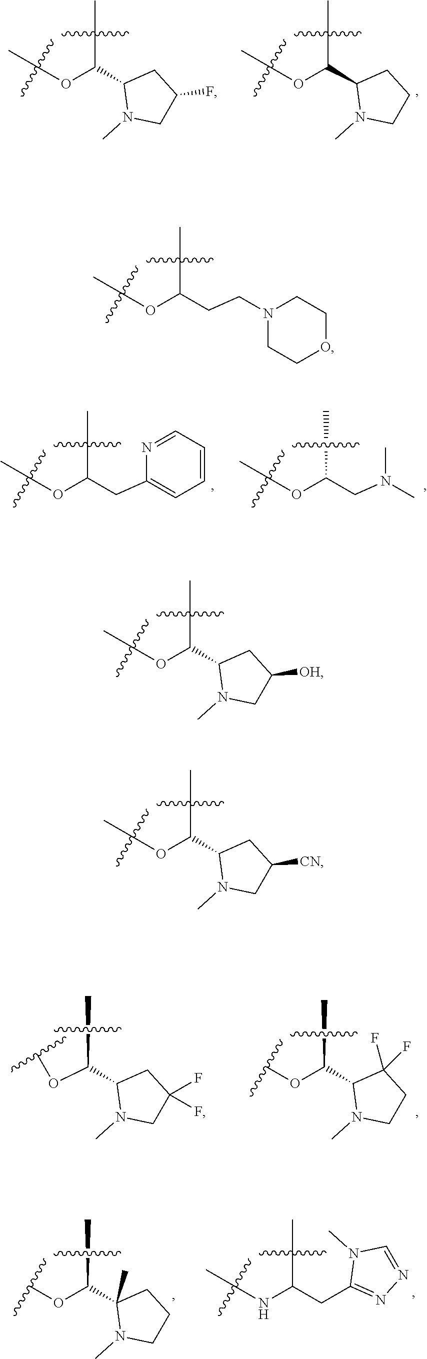

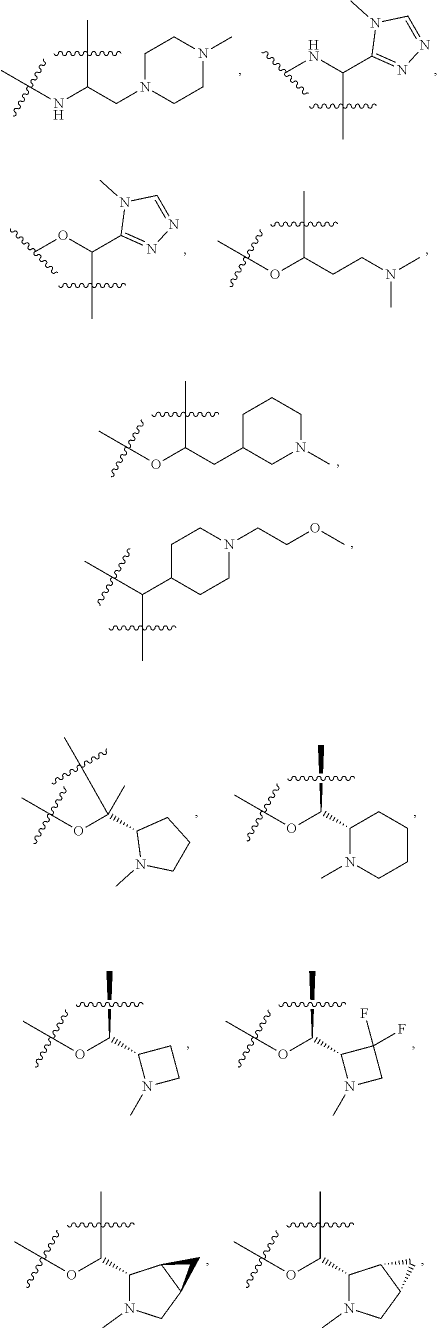

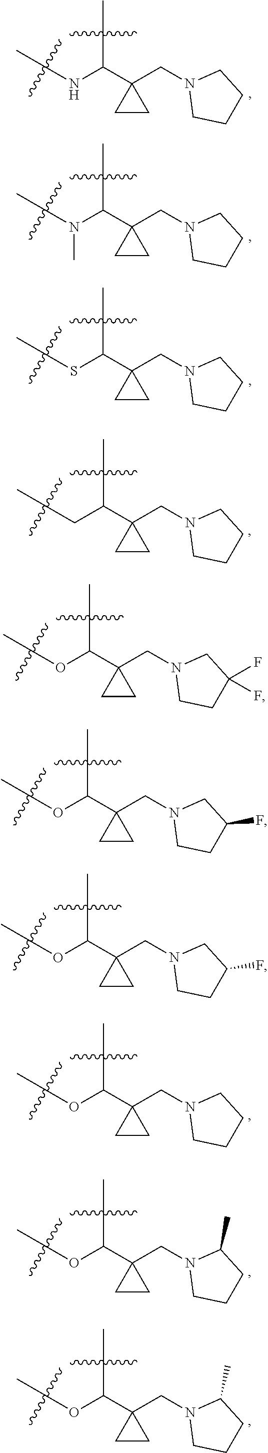

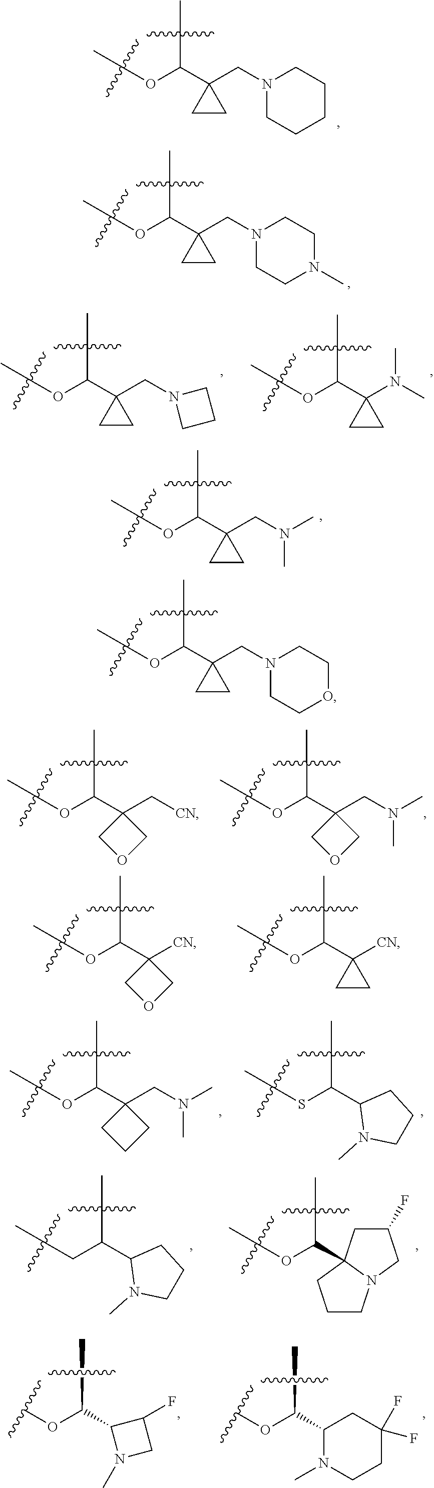

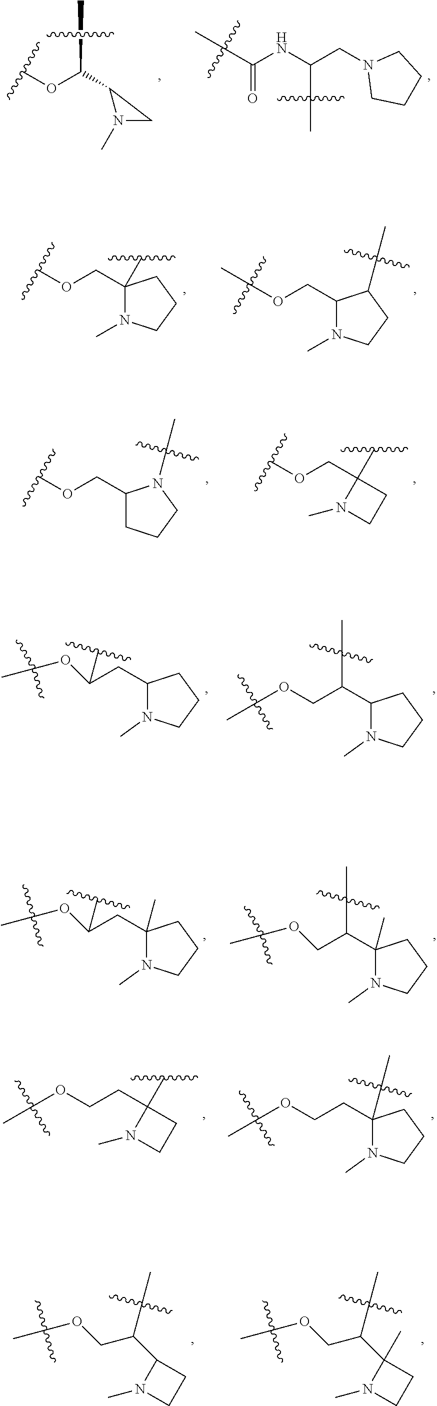

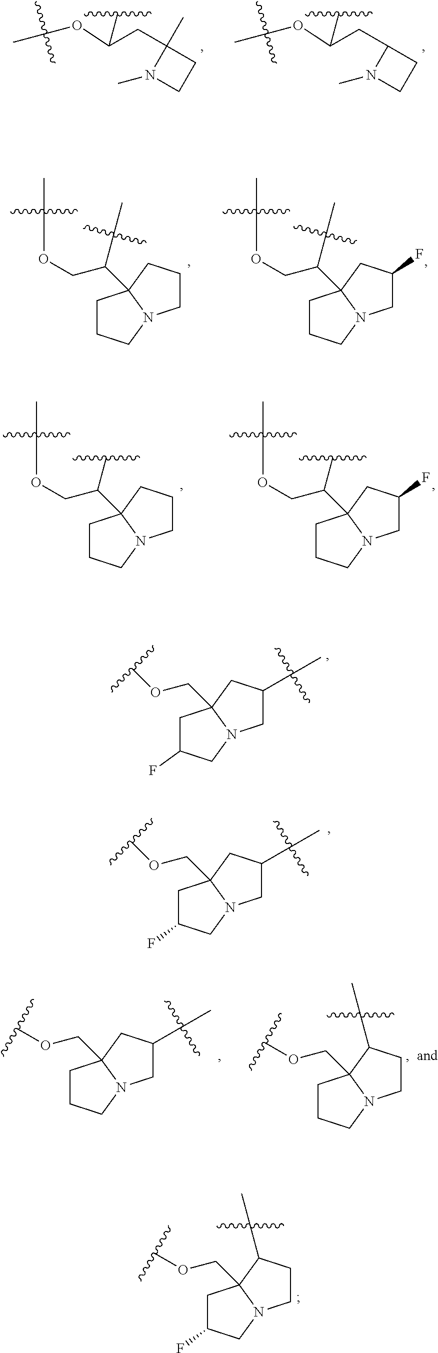

L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{1d})—, —C(O)—, —OC(R^{1c})₂—, —N(R^{1d})C(R^{1c})₂—, —C(O)C(R^{1c})₂—, —N(R^{1d})C(O)—, —C(O)N(R^{1d})—, —OC(O)—, —C(O)O—, C₃₋₁₀ carbocycle, and 3- to 6-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₂₋₆ alkenyl, 2- to 6-membered heteroalkyl, C₃₋₁₀ carbocycle, and 3- to 10-membered heterocycle, wherein C₁₋₆ alkyl, C₂₋₆ alkenyl, 2- to 6-membered heteroalkyl, C₃₋₁₀ carbocycle, and 3- to 10-membered heterocycle are optionally substituted with one, two, or three R^{L};
L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 6;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

In some embodiments, for a compound of Formula (I) or (I):
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R^{6a}) or C(O), W⁷ is N(R^{7b}), W⁸ is C(R⁸)(R^{8a}) or C(O), and each [symbol] indicates a single
R⁶, R^{6a}, R⁸, and R^{8a} are each independently selected from hydrogen and halogen;
R⁷ is R¹⁷;
R^{7b} is R¹⁷;
R¹⁷ is selected from C₃₋₁₂ carbocycle and 3- to 12-membered heterocycle, each of which is optionally substituted with one or more R²;
L¹ is -L²-L³-L⁴-L⁵-L⁶-;
L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—;
L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L};
L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 3;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

In some embodiments, a compound of Formula (I), (I′), (I-A), (I-A1), (I-A2), (I-A3), (I-B), (I-B1), (I-B2), or (I-B3) is substituted with one R^{A}, R^{L}, R^{7c}, or R^{7d} that is capable of forming a covalent bond with a Ras amino acid, such as the 12ₜₕ or 13^{th} amino acid of a mutant Ras or amino acid corresponding to the 12^{th} or 13^{th} amino acid of a mutant KRas protein. In some embodiments, the one R^{A}, R^{L}, R^{7c}, or R^{7d} is capable of forming a covalent bond with a Ras amino acid sidechain. In some embodiments, the one R^{A}, R^{L}, R^{7c}, or R^{7d} is capable of forming a covalent bond with a KRas amino acid, such as the 12^{th} amino acid of a human KRas protein. In some embodiments, the one R^{A}, R^{L}, R^{7c}, or R^{7d} is capable of forming a covalent bond with the 12 amino acid of a mutant KRas protein selected from KRas G12D and KRas G12C. In some embodiments, the one R^{A}, R^{L}, R^{7c}, or R^{7d} is capable of forming a covalent bond with the 13′ amino acid of a human KRas protein. In some embodiments, the one R^{A}, R^{L}, R^{7c}, or R^{7d} is capable of forming a covalent bond with the 13′ amino acid of a mutant KRas protein selected from KRas G13D and KRas G13C.

In some embodiments, for a compound of Formula (I), (I′), (I-A), (I-A1), (I-A2), (I-A3), (I-B), (I-B1), (I-B2), or (I-B3), R^{A}, R^{L}, R^{7c}, and R^{7d} are incapable of forming a covalent bond with a Ras amino acid sidechain. In some embodiments, R^{A}, R^{L}, R^{7c}, and R^{7d} are incapable of forming a covalent bond with a KRas amino acid, such as the 12^{th} amino acid of a human KRas protein. In some embodiments, R^{A}, R^{L}, R^{7c}, and R^{7d} are incapable of forming a covalent bond with the 12^{th} amino acid of a mutant KRas protein selected from KRas G12D and KRas G12C. In some embodiments, R^{A}, R^{L}, R^{7c}, and R^{7d} are incapable of forming a covalent bond with the 13′ amino acid of a human KRas protein. In some embodiments, R^{A}, R^{L}, R^{7c}, and R^{7d} are incapable of forming a covalent bond with the 13′ amino acid of a mutant KRas protein selected from KRas G13D and KRas G13C. In some embodiments, R^{A}, R^{L}, R^{7c}, and R^{7d} are incapable of forming a covalent bond with the 12′ amino acid of a human KRas protein selected from KRas wildtype, KRas G12D, KRas G12C, KRas G12V, KRas G13D, KRas G13C, and KRas G13V. In some embodiments, R^{A}, R^{L}, R^{7c}, and R^{7d} are incapable of forming a covalent bond with the 13¹ amino acid of human KRas protein selected from KRas wildtype, KRas G12D, KRas G12C, KRas G12V, KRas G13D, KRas G13C, and KRas G13V.

In certain aspects, the present disclosure provides a compound selected from

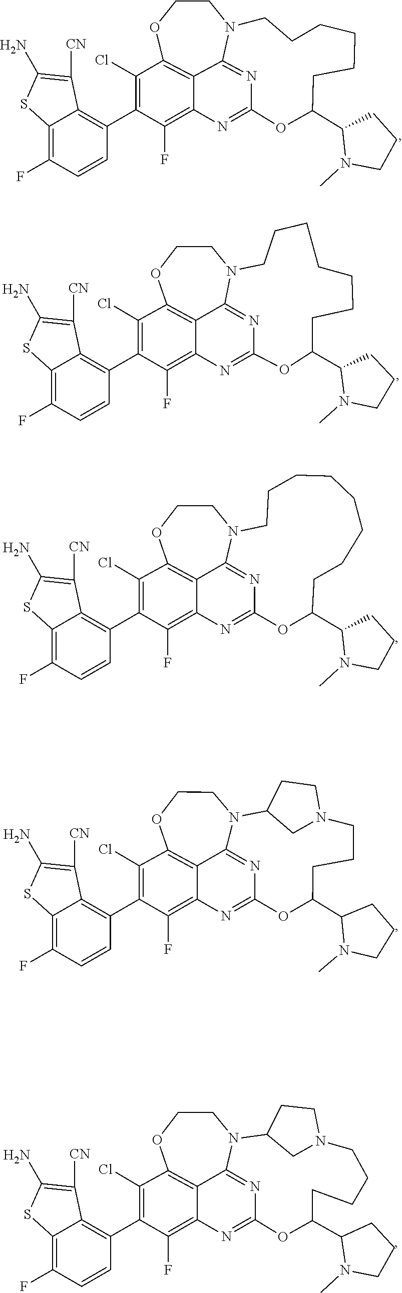

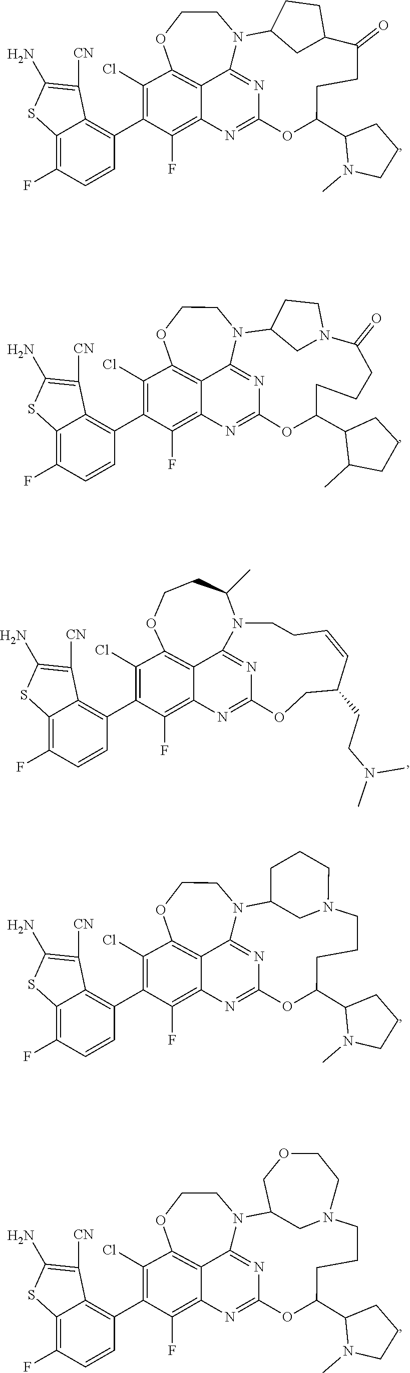 [SMILES: CN1CCCC1C1CCC(=O)C2CCC(C2)N2CCOc3c(Cl)c(-c4ccc(F)c5sc(N)c(C#N)c45)c(F)c4nc(nc2c34)O1 ; CC1CCCC1C1CCCC(=O)N2CCC(C2)N2CCOc3c(Cl)c(-c4ccc(F)c5sc(N)c(C#N)c45)c(F)c4nc(nc2c34)O1 ; C[C@@H]1CCOc2c(Cl)c(-c3ccc(F)c4sc(N)c(C#N)c34)c(F)c3nc4nc(c23)N1CC/C=C\[C@H](CCN(C)C)CO4 ; CN1CCCC1C1CCCN2CCCC(C2)N2CCOc3c(Cl)c(-c4ccc(F)c5sc(N)c(C#N)c45)c(F)c4nc(nc2c34)O1 ; CN1CCCC1C1CCCN2CCOCC(C2)N2CCOc3c(Cl)c(-c4ccc(F)c5sc(N)c(C#N)c45)c(F)c4nc(nc2c34)O1]

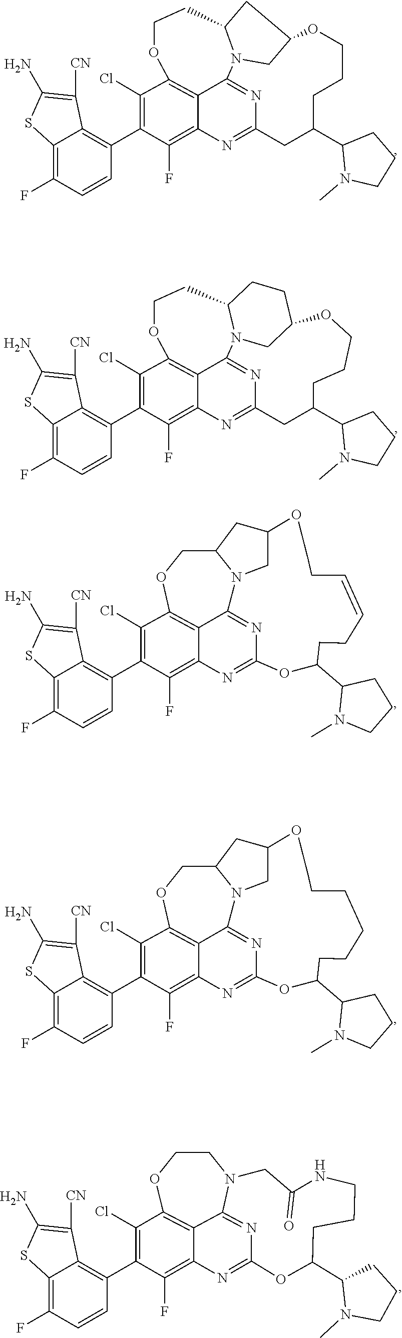

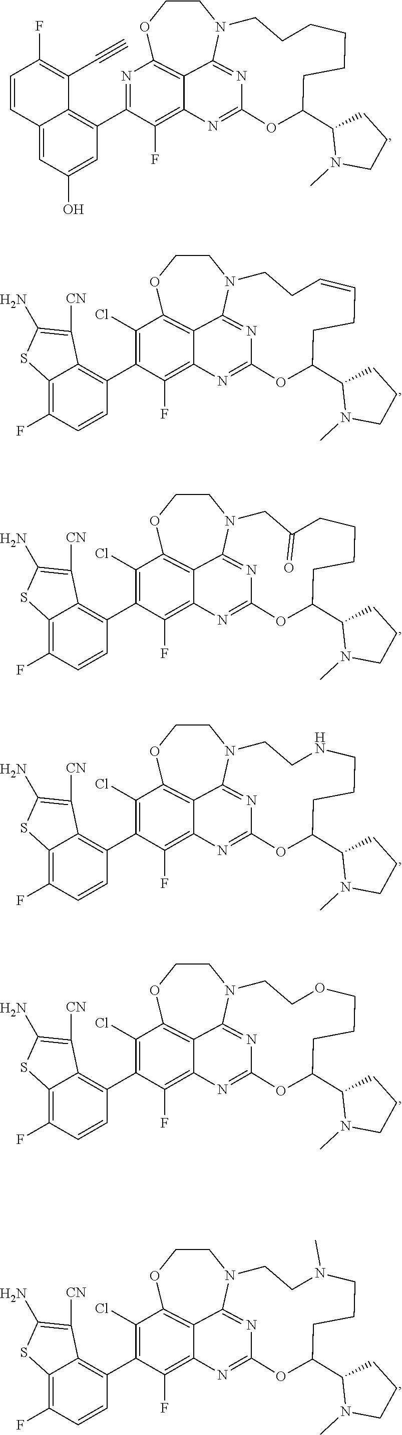 [SMILES: C#Cc1c(F)ccc2cc(O)cc(-c3nc4c5c6nc(nc5c3F)OC([C@@H]3CCCN3C)CCCCCCN6CCO4)c12 ; CN1CCC[C@H]1C1CC/C=C\CCN2CCOc3c(Cl)c(-c4ccc(F)c5sc(N)c(C#N)c45)c(F)c4nc(nc2c34)O1 ; CN1CCC[C@H]1C1CCCCC(=O)CN2CCOc3c(Cl)c(-c4ccc(F)c5sc(N)c(C#N)c45)c(F)c4nc(nc2c34)O1 ; CN1CCC[C@H]1C1CCCNCCN2CCOc3c(Cl)c(-c4ccc(F)c5sc(N)c(C#N)c45)c(F)c4nc(nc2c34)O1 ; CN1CCC[C@H]1C1CCCOCCN2CCOc3c(Cl)c(-c4ccc(F)c5sc(N)c(C#N)c45)c(F)c4nc(nc2c34)O1 ; CN1CCCC([C@@H]2CCCN2C)Oc2nc3c4c(c(Cl)c(-c5ccc(F)c6sc(N)c(C#N)c56)c(F)c4n2)OCCN3CC1]

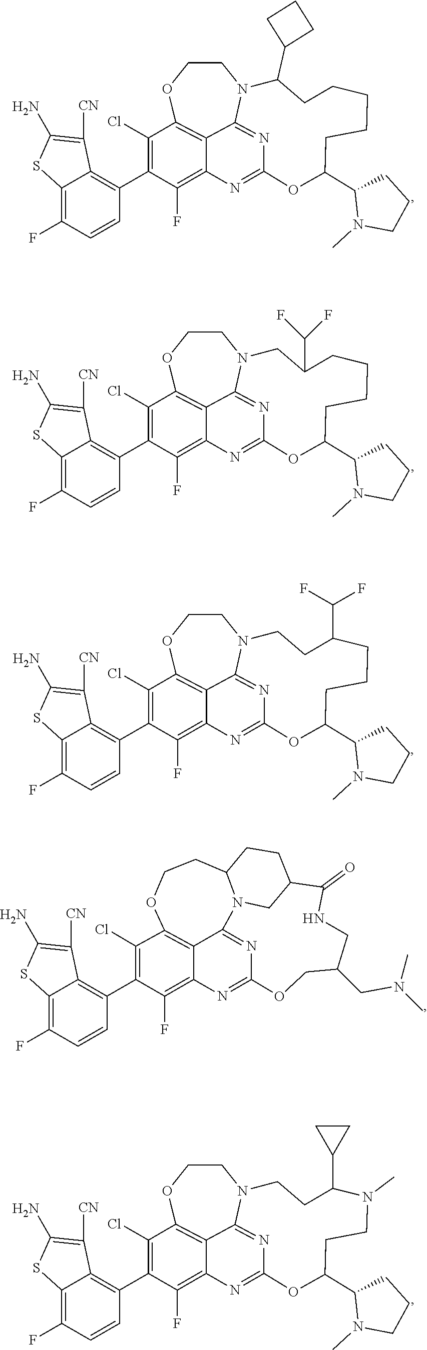 [SMILES: CN1CCC[C@H]1C1CCCCCC(C2CCC2)N2CCOc3c(Cl)c(-c4ccc(F)c5sc(N)c(C#N)c45)c(F)c4nc(nc2c34)O1 ; CN1CCC[C@H]1C1CCCCC(C(F)F)CN2CCOc3c(Cl)c(-c4ccc(F)c5sc(N)c(C#N)c45)c(F)c4nc(nc2c34)O1 ; CN1CCC[C@H]1C1CCCC(C(F)F)CCN2CCOc3c(Cl)c(-c4ccc(F)c5sc(N)c(C#N)c45)c(F)c4nc(nc2c34)O1 ; CN(C)CC1CNC(=O)C2CCC3CCOc4c(Cl)c(-c5ccc(F)c6sc(N)c(C#N)c56)c(F)c5nc(nc(c45)N3C2)OC1 ; CN1CCC([C@@H]2CCCN2C)Oc2nc3c4c(c(Cl)c(-c5ccc(F)c6sc(N)c(C#N)c56)c(F)c4n2)OCCN3CCC1C1CC1]

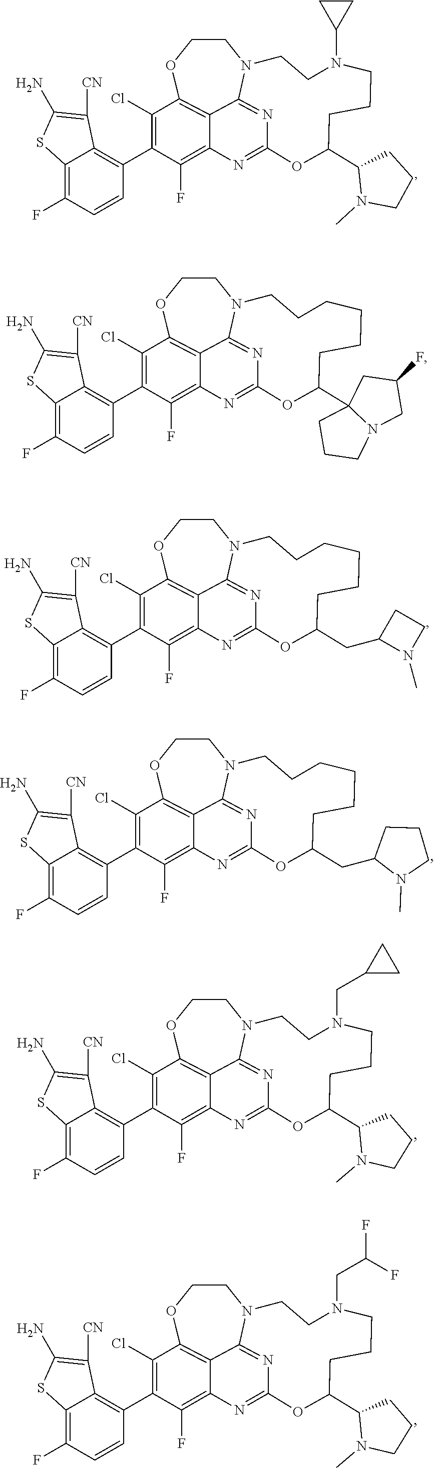

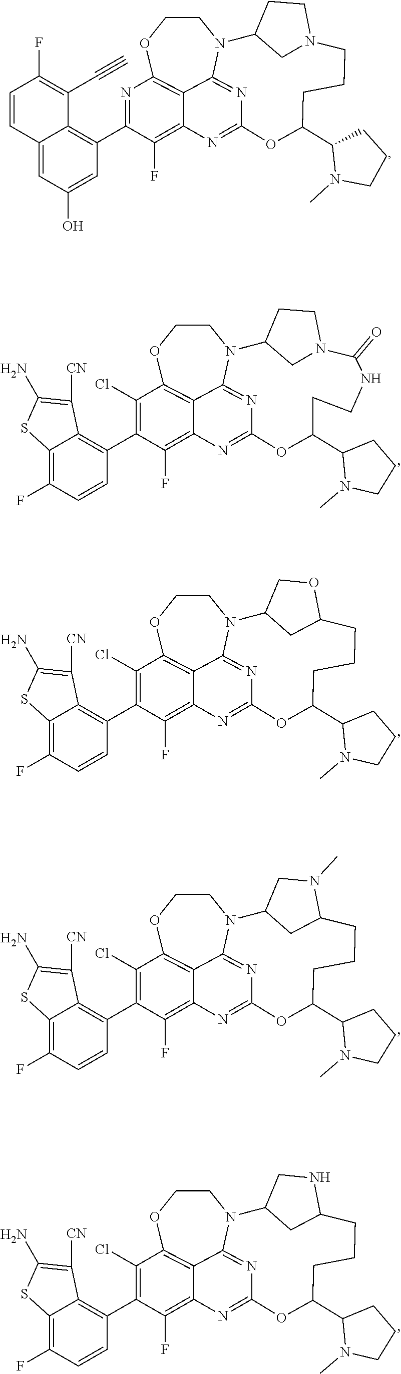 [SMILES: C#Cc1c(F)ccc2cc(O)cc(-c3nc4c5c6nc(nc5c3F)OC([C@@H]3CCCN3C)CCCN3CCC(C3)N6CCO4)c12 ; CN1CCCC1C1CCNC(=O)N2CCC(C2)N2CCOc3c(Cl)c(-c4ccc(F)c5sc(N)c(C#N)c45)c(F)c4nc(nc2c34)O1 ; CN1CCCC1C1CCCC2CC(CO2)N2CCOc3c(Cl)c(-c4ccc(F)c5sc(N)c(C#N)c45)c(F)c4nc(nc2c34)O1 ; CN1CC2CC1CCCC(C1CCCN1C)Oc1nc3c4c(c(Cl)c(-c5ccc(F)c6sc(N)c(C#N)c56)c(F)c4n1)OCCN32 ; CN1CCCC1C1CCCC2CC(CN2)N2CCOc3c(Cl)c(-c4ccc(F)c5sc(N)c(C#N)c45)c(F)c4nc(nc2c34)O1]

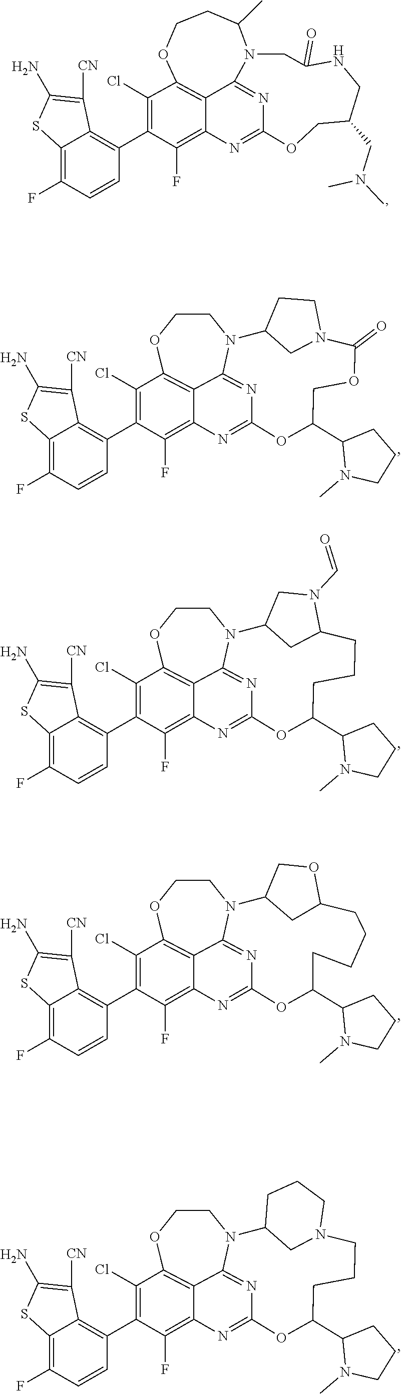

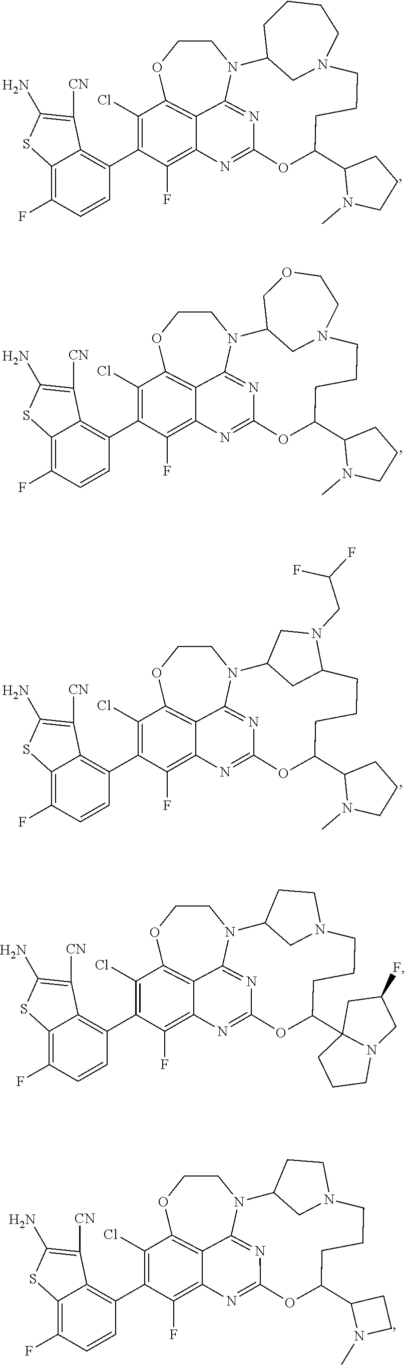 [SMILES: CN1CCCC1C1CCCN2CCCCC(C2)N2CCOc3c(Cl)c(-c4ccc(F)c5sc(N)c(C#N)c45)c(F)c4nc(nc2c34)O1 ; CN1CCCC1C1CCCN2CCOCC(C2)N2CCOc3c(Cl)c(-c4ccc(F)c5sc(N)c(C#N)c45)c(F)c4nc(nc2c34)O1 ; CN1CCCC1C1CCCC2CC(CN2CC(F)F)N2CCOc3c(Cl)c(-c4ccc(F)c5sc(N)c(C#N)c45)c(F)c4nc(nc2c34)O1 ; N#Cc1c(N)sc2c(F)ccc(-c3c(Cl)c4c5c6nc(nc5c3F)OC(C35CCCN3C[C@H](F)C5)CCCN3CCC(C3)N6CCO4)c12 ; CN1CCC1C1CCCN2CCC(C2)N2CCOc3c(Cl)c(-c4ccc(F)c5sc(N)c(C#N)c45)c(F)c4nc(nc2c34)O1]

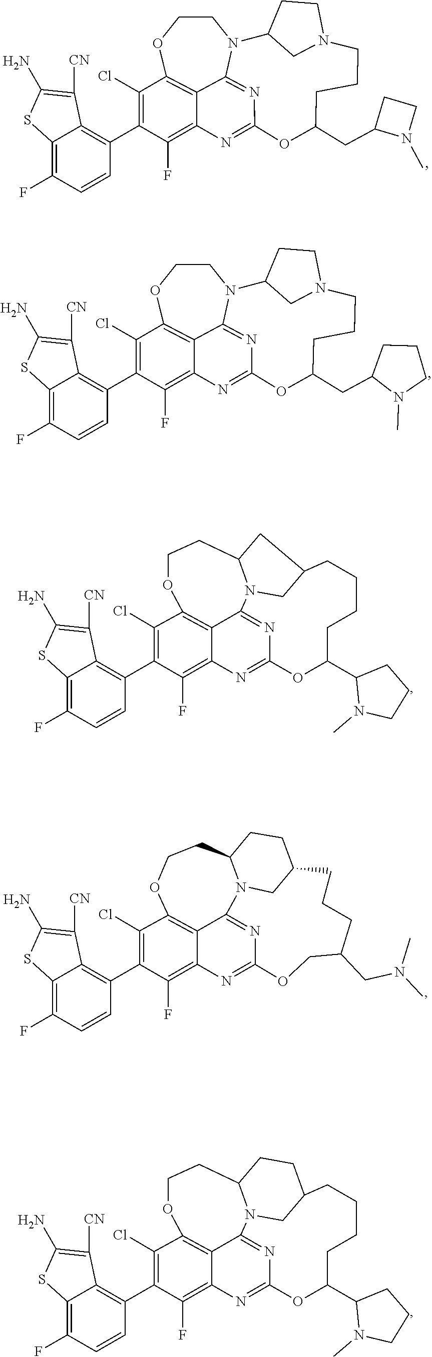 [SMILES: CN1CCC1CC1CCCN2CCC(C2)N2CCOc3c(Cl)c(-c4ccc(F)c5sc(N)c(C#N)c45)c(F)c4nc(nc2c34)O1 ; CN1CCCC1CC1CCCN2CCC(C2)N2CCOc3c(Cl)c(-c4ccc(F)c5sc(N)c(C#N)c45)c(F)c4nc(nc2c34)O1 ; CN1CCCC1C1CCCCC2CC3CCOc4c(Cl)c(-c5ccc(F)c6sc(N)c(C#N)c56)c(F)c5nc(nc(c45)N3C2)O1 ; CN(C)CC1CCC[C@H]2CC[C@@H]3CCOc4c(Cl)c(-c5ccc(F)c6sc(N)c(C#N)c56)c(F)c5nc(nc(c45)N3C2)OC1 ; CN1CCCC1C1CCCCC2CCC3CCOc4c(Cl)c(-c5ccc(F)c6sc(N)c(C#N)c56)c(F)c5nc(nc(c45)N3C2)O1]

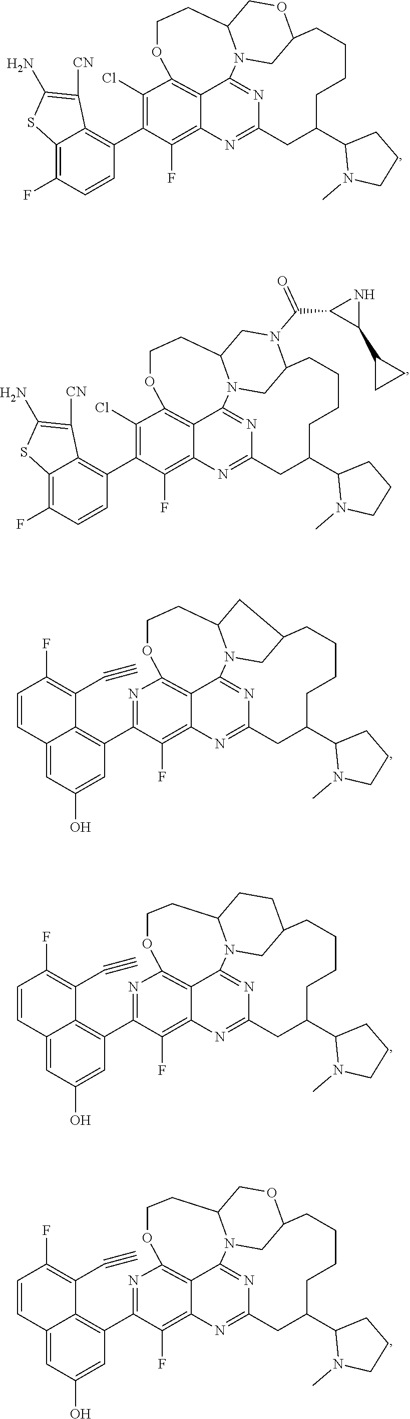 [SMILES: CN1CCCC1C1CCCCC2CN3c4nc(nc5c(F)c(-c6ccc(F)c7sc(N)c(C#N)c67)c(Cl)c(c45)OCCC3CO2)C1 ; CN1CCCC1C1CCCCC2CN3c4nc(nc5c(F)c(-c6ccc(F)c7sc(N)c(C#N)c67)c(Cl)c(c45)OCCC3CN2C(=O)[C@@H]2N[C@H]2C2CC2)C1 ; C#Cc1c(F)ccc2cc(O)cc(-c3nc4c5c6nc(nc5c3F)CC(C3CCCN3C)CCCCC3CC(CCO4)N6C3)c12 ; C#Cc1c(F)ccc2cc(O)cc(-c3nc4c5c6nc(nc5c3F)CC(C3CCCN3C)CCCCC3CCC(CCO4)N6C3)c12 ; C#Cc1c(F)ccc2cc(O)cc(-c3nc4c5c6nc(nc5c3F)CC(C3CCCN3C)CCCCC3CN6C(CCO4)CO3)c12]

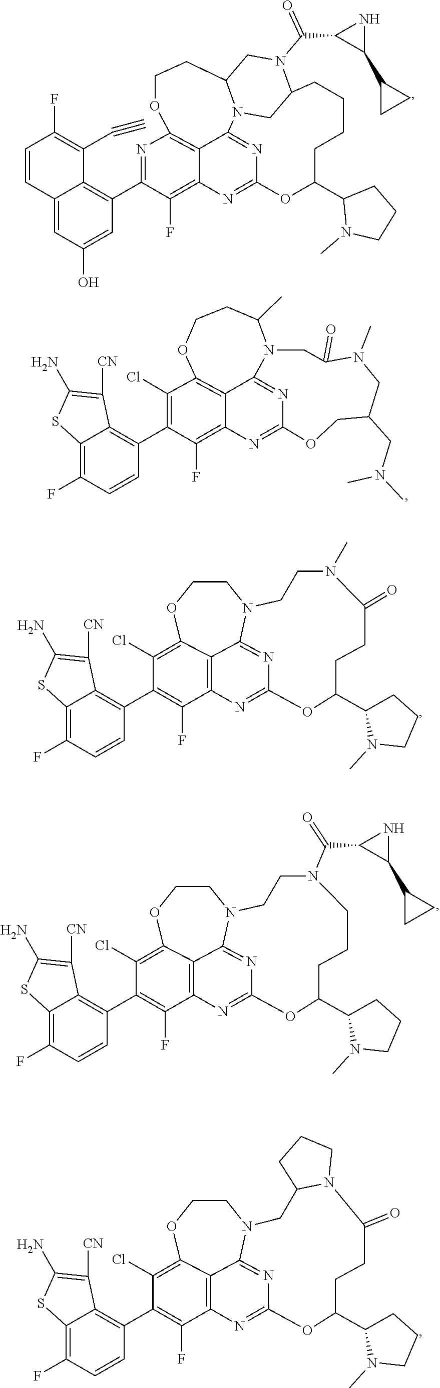 [SMILES: C#Cc1c(F)ccc2cc(O)cc(-c3nc4c5c6nc(nc5c3F)OC(C3CCCN3C)CCCCC3CN6C(CCO4)CN3C(=O)[C@@H]3N[C@H]3C3CC3)c12 ; CC1CCOc2c(Cl)c(-c3ccc(F)c4sc(N)c(C#N)c34)c(F)c3nc4nc(c23)N1CC(=O)N(C)CC(CN(C)C)CO4 ; CN1CCN2CCOc3c(Cl)c(-c4ccc(F)c5sc(N)c(C#N)c45)c(F)c4nc(nc2c34)OC([C@@H]2CCCN2C)CCC1=O ; CN1CCC[C@H]1C1CCCN(C(=O)[C@@H]2N[C@H]2C2CC2)CCN2CCOc3c(Cl)c(-c4ccc(F)c5sc(N)c(C#N)c45)c(F)c4nc(nc2c34)O1 ; CN1CCC[C@H]1C1CCC(=O)N2CCCC2CN2CCOc3c(Cl)c(-c4ccc(F)c5sc(N)c(C#N)c45)c(F)c4nc(nc2c34)O1]

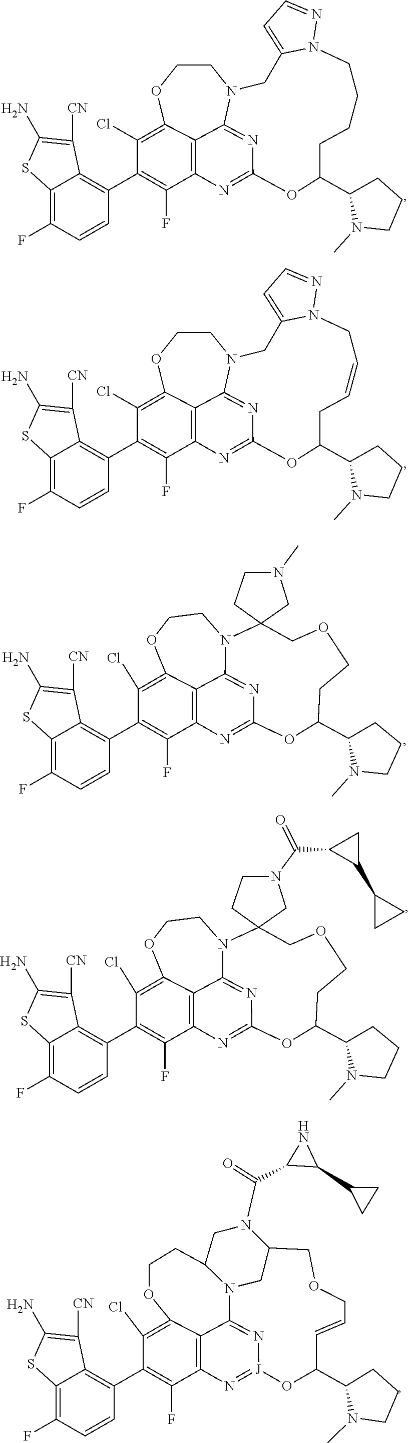

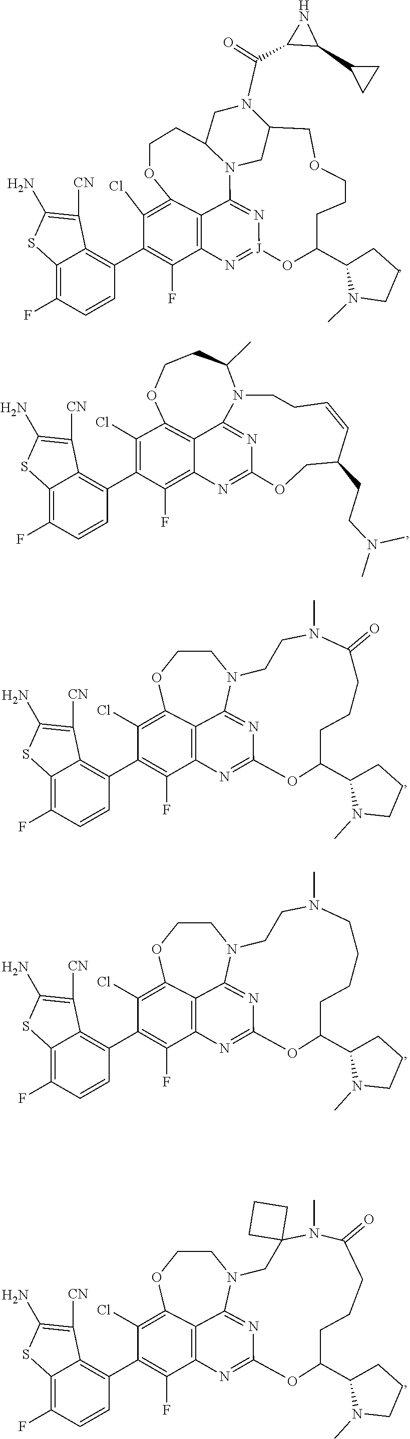

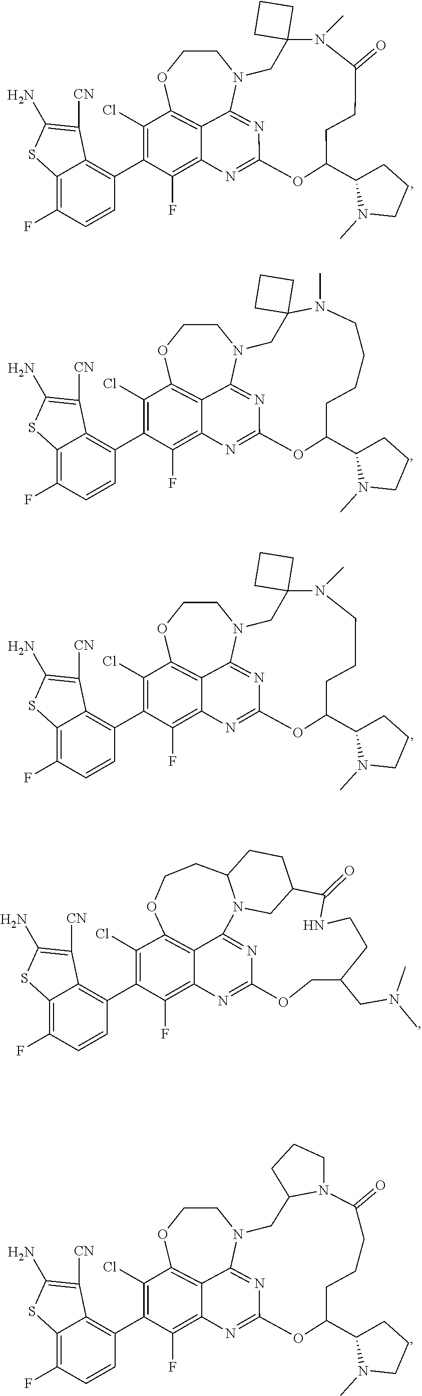

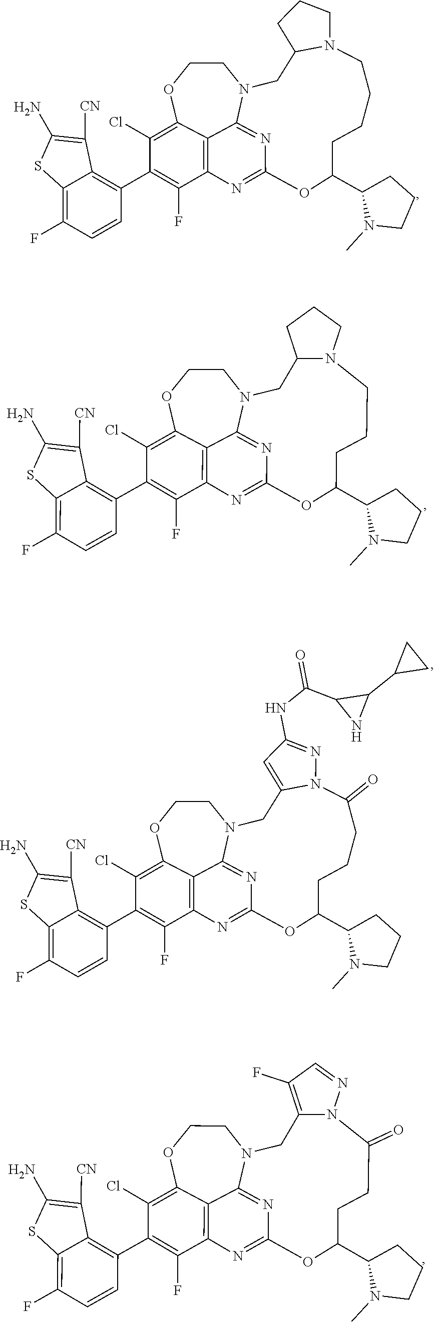 [SMILES: CN1CCC[C@H]1C1CCCCN2CCCC2CN2CCOc3c(Cl)c(-c4ccc(F)c5sc(N)c(C#N)c45)c(F)c4nc(nc2c34)O1 ; CN1CCC[C@H]1C1CCCN2CCCC2CN2CCOc3c(Cl)c(-c4ccc(F)c5sc(N)c(C#N)c45)c(F)c4nc(nc2c34)O1 ; CN1CCC[C@H]1C1CCCC(=O)n2nc(NC(=O)C3NC3C3CC3)cc2CN2CCOc3c(Cl)c(-c4ccc(F)c5sc(N)c(C#N)c45)c(F)c4nc(nc2c34)O1 ; CN1CCC[C@H]1C1CCC(=O)n2ncc(F)c2CN2CCOc3c(Cl)c(-c4ccc(F)c5sc(N)c(C#N)c45)c(F)c4nc(nc2c34)O1]

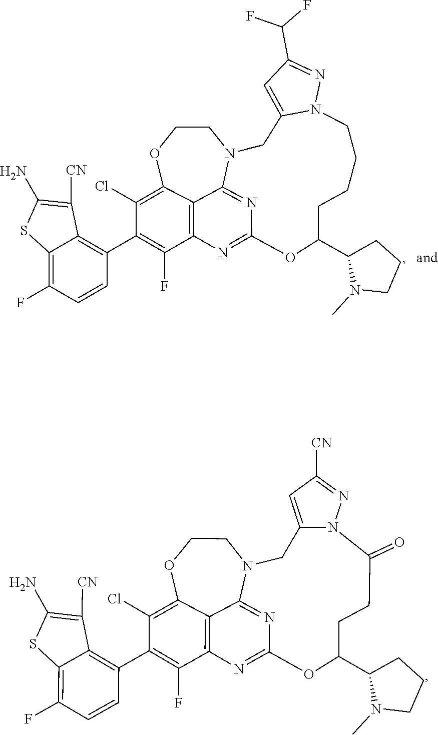 [SMILES: CN1CCC[C@H]1C1CCCCn2nc(C(F)F)cc2CN2CCOc3c(Cl)c(-c4ccc(F)c5sc(N)c(C#N)c45)c(F)c4nc(nc2c34)O1 ; CN1CCC[C@H]1C1CCC(=O)n2nc(C#N)cc2CN2CCOc3c(Cl)c(-c4ccc(F)c5sc(N)c(C#N)c45)c(F)c4nc(nc2c34)O1]

or a pharmaceutically acceptable salt or solvate thereof.

In some embodiments, a compound of Formula (I), such as a compound of Formula (I′), (I-A), (I-A1), (I-A2), (I-A3), (I-B), (I-B1), (I-B2), or (I-B3), is provided as a substantially pure stereoisomer. In some embodiments, the stereoisomer is provided in at least 80% enantiomeric excess, such as at least 85%, at least 90%, at least 95%, at least 96%, at least 97%, at least 98%, at least 99%, or at least 99.9% enantiomeric excess.

In some embodiments, the present disclosure provides an atropisomer of a compound described herein, such as a compound of Formula (I). In some embodiments, the atropisomer is provided in enantiomeric excess. In some embodiments, the atropisomer is provided in at least 80% enantiomeric excess, such as at least 85%, at least 90%, at least 95%, at least 96%, at least 97%, at least 98%, at least 99%, or at least 99.9% enantiomeric excess. In some embodiments, the compound or modified protein of Formula (I) is preferably used as a non-racemic mixture, wherein one atropisomer is present in excess of its corresponding enantiomer or epimer. Typically, such mixture contains a mixture of the two isomers in a ratio of at least 9:1, preferably at least 19:1. In some embodiments, the atropisomer is provided in at least 96% enantiomeric excess, meaning the compound has less than 2% of the corresponding enantiomer. In some embodiments, the atropisomer is provided in at least 96% diastereomeric excess, meaning the compound has less than 2% of the corresponding diastereomer.

The term “atropisomers” refers to conformational stereoisomers which occur when rotation about a single bond in the molecule is prevented, restricted, or greatly slowed as a result of steric interactions with other parts of the molecule and wherein the substituents at both ends of the single bond are asymmetrical (i.e., optical activity arises without requiring an asymmetric carbon center or stereocenter). Where the rotational barrier about the single bond is high enough, and interconversion between conformations is slow enough, separation and isolation of the isomeric species may be permitted. Atropisomers are enantiomers (or epimers) without a single asymmetric atom. Atropisomers are typically considered stable if the barrier to interconversion is high enough to permit the atropisomers to undergo little or no interconversion at room temperature for a least a week, preferably at least a year. In some embodiments, an atropisomeric compound of the disclosure does not undergo more than about 5% interconversion to its opposite atropisomer at room temperature during one week when the atropisomeric compound is in substantially pure form, which is generally a solid state. In some embodiments, an atropisomeric compound of the disclosure does not undergo more than about 5% interconversion to its opposite atropisomer at room temperature (approximately 25° C.) during one year. The present chemical entities, pharmaceutical compositions, and methods are meant to include all such possible atropisomers, including racemic mixtures, diastereomeric mixtures, epimeric mixtures, optically pure forms of single atropisomers, and intermediate mixtures.

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases or inorganic or organic acids to form a pharmaceutically acceptable salt. In some embodiments, such salts are prepared in situ during the final isolation and purification of the compounds described herein, or by separately reacting a purified compound in its free form with a suitable acid or base, and isolating the salt thus formed.

In some embodiments, the compounds described herein exist as solvates. In some embodiments are methods of treating diseases by administering such solvates. Further described herein are methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein are conveniently prepared or formed during the processes described herein. By way of example only, hydrates of the compounds described herein are conveniently prepared by recrystallization from an aqueous/organic solvent mixture, using organic solvents including, but not limited to, dioxane, tetrahydrofuran, or MeOH. In addition, the compounds provided herein exist in unsolvated as well as solvated forms. In general, the solvated forms are considered equivalent to the unsolvated forms for the purposes of the compounds and methods provided herein.

In certain aspects, the present disclosure provides a compound of the formula B-L^{BE}-E wherein:
B is a monovalent form of a compound described herein;
L^{BE} is a covalent linker bonded to B and E; and
E is a monovalent form of a degradation enhancer.

A “degradation enhancer” is a compound capable of binding a ubiquitin ligase protein (e.g., E3 ubiquitin ligase protein) or a compound capable of binding a protein that is capable of binding to a ubiquitin ligase protein to form a protein complex capable of conjugating a ubiquitin protein to a target protein. In some embodiments, the degradation enhancer is capable of binding to an E3 ubiquitin ligase protein or a protein complex comprising an E3 ubiquitin ligase protein. In some embodiments, the degradation enhancer is capable of binding to an E2 ubiquitin-conjugating enzyme. In some embodiments, the degradation enhancer is capable of binding to a protein complex comprising an E2 ubiquitin-conjugating enzyme and an E3 ubiquitin ligase protein.

In some embodiments, the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HERS, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885. In some embodiments, the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1. In some embodiments, the degradation enhancer is a compound described in Ishida and Ciulli, SLAS Discovery 2021, Vol. 25(4) 484-502, which is incorporated by reference in its entirety for any purpose, for example VH032, VH101, VH298, thalidomide, bestatin, methyl bestatin, nutlin, idasanutlin, bardoxolone, bardoxolone methyl, indisulam (E7070), E7820, chloroquinoxaline sulfonamide (CQS), nimbolide, KB02, ASTX660, lenalidomide, or pomalidomide.

In some embodiments, the degradation enhancer is a compound described in US20180050021, WO2016146985, WO2018189554, WO2018119441, WO2018140809, WO2018119448, WO2018119357, WO2018118598, WO2018102067, WO201898280, WO201889736, WO201881530, WO201871606, WO201864589, WO201852949, WO2017223452, WO2017204445, WO2017197055, WO2017197046, WO2017180417, WO2017176958, WO201711371, WO2018226542, WO2018223909, WO2018189554, WO2016169989, WO2016146985, CN105085620B, CN106543185B, U.S. Ser. No. 10/040,804, U.S. Pat. No. 9,938,302, U.S. Ser. No. 10/144,745, U.S. Ser. No. 10/145,848, U.S. Pat. Nos. 9,938,264, 9,632,089, 9,821,068, 9,758,522, 9,500,653, 9,765,019, 8,507,488, 8,299,057, US20180298027, US20180215731, US20170065719, US20170037004, US20160272639, US20150291562, or US20140356322, each of which is incorporated by reference in its entirety for any purpose.

In some embodiments, L^{B}E is -L^{BE1}-L^{BE2}-L^{BE3}-L^{BE4}-L^{BE5}-;
L^{BE1}, L^{BE2}, L^{BE3}, L^{BE4}, and L^{BE5} are independently a bond, —O—, —N(R¹²)—, —C(O)—, —N(R¹²)C(O)—, —C(O)N(R¹²)—, —S—, —S(O)₂—, —S(O)—, —S(O)₂N(R¹²)—, —S(O)N(R¹²)—, —N(R¹²)S(O)—, —N(R¹²)S(O)₂—, C₁₋₆ alkylene, (—O—C₁₋₆ alkyl)₂-, (—C₁₋₆ alkyl-O)₂—, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene, wherein C₁₋₆ alkylene, C₂₋₆ alkenylene, C₂₋₆ alkynylene, C₁₋₆ haloalkylene, C₃₋₁₂ cycloalkylene, C₁₋ᵣ heterocycloalkylene, C₆₋₁₂ arylene, or C₁₋₁₁ heteroarylene are optionally substituted with one, two, or three R²⁰; and wherein each C₁₋₆ alkyl of (—O—C₁₋₆ alkyl)₂- and (—C₁₋₆ alkyl-O)₂— is optionally substituted with one, two, or three R²⁰; and
z is independently an integer from 0 to 10.

In some embodiments, L^{BE} is —(O—C₂ alkyl)_{z}- and z is an integer from 1 to 10. In some embodiments, L^{BE} is —(C₂ alkyl-O—)₂— and z is an integer from 1 to 10. In some embodiments, L^{BE} is —(CH₂)_{zz1}L^{BE2}(CH₂O)_{zz2}—, wherein L^{BE2} is a bond, a 5- or 6-membered heterocyclene, phenylene, —C₂₋₄ alkynylene, —SO₂— or —NH—; and zz1 and zz2 are independently an integer from 0 to 10. In some embodiments, L^{BE} is —(CH₂)_{zz1}(CH₂O)_{zz2}—, wherein zz1 and zz2 are each independently an integer from 0 to 10. In some embodiments, L^{BE} is a PEG linker (e.g., divalent linker of 1 to 10 ethylene glycol subunits). In some embodiments, E is a monovalent form of a compound selected from

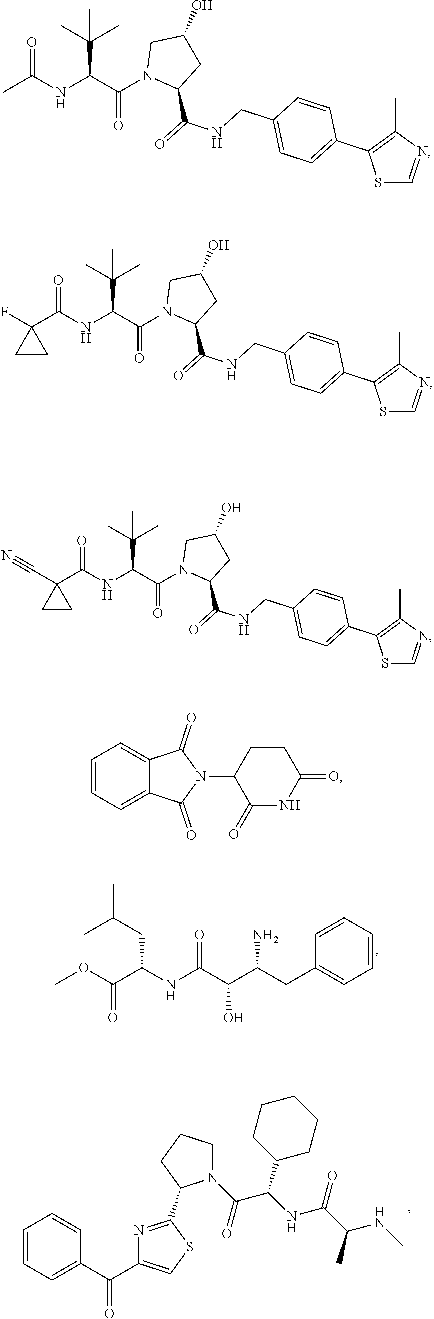 [SMILES: CC(=O)N[C@H](C(=O)N1C[C@H](O)C[C@H]1C(=O)NCc1ccc(-c2scnc2C)cc1)C(C)(C)C ; Cc1ncsc1-c1ccc(CNC(=O)[C@@H]2C[C@@H](O)CN2C(=O)[C@@H](NC(=O)C2(F)CC2)C(C)(C)C)cc1 ; Cc1ncsc1-c1ccc(CNC(=O)[C@@H]2C[C@@H](O)CN2C(=O)[C@@H](NC(=O)C2(C#N)CC2)C(C)(C)C)cc1 ; O=C1CCC(N2C(=O)c3ccccc3C2=O)C(=O)N1 ; COC(=O)[C@H](CC(C)C)NC(=O)[C@@H](O)[C@H](N)Cc1ccccc1 ; CN[C@@H](C)C(=O)N[C@H](C(=O)N1CCC[C@H]1c1nc(C(=O)c2ccccc2)cs1)C1CCCCC1]

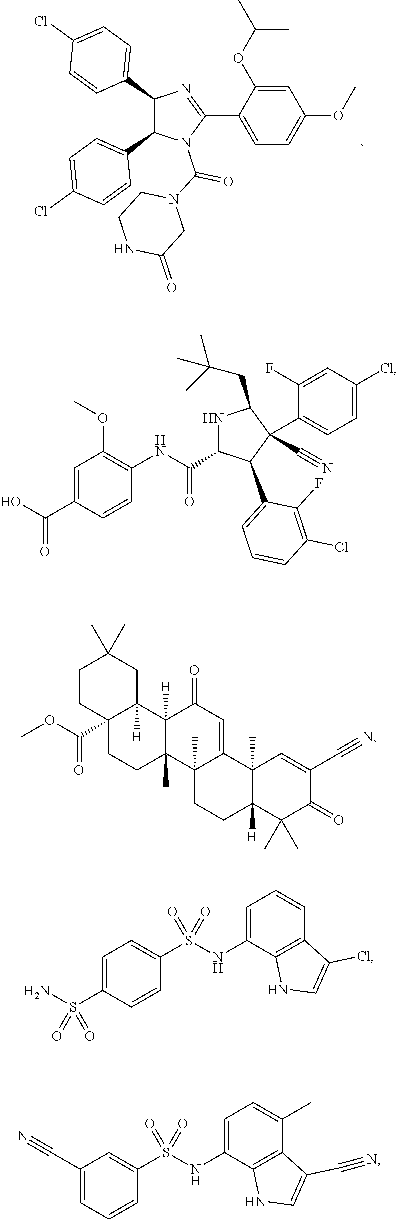

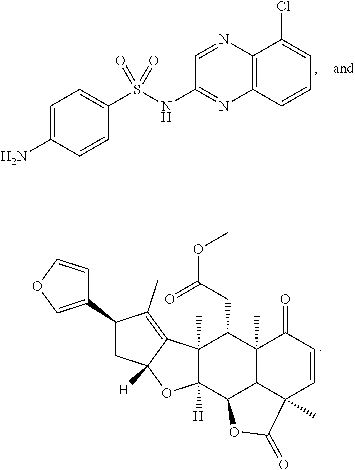

In some embodiments, E is a monovalent form of a compound selected from

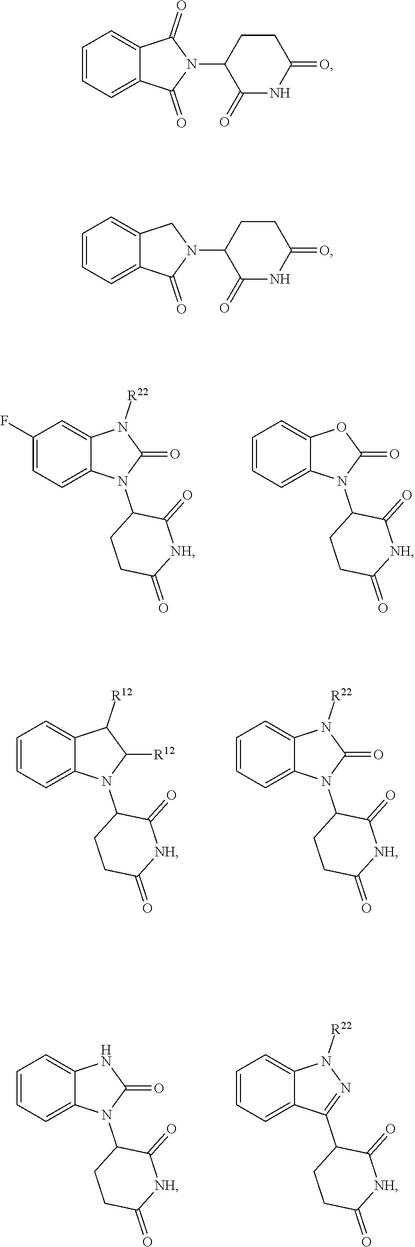

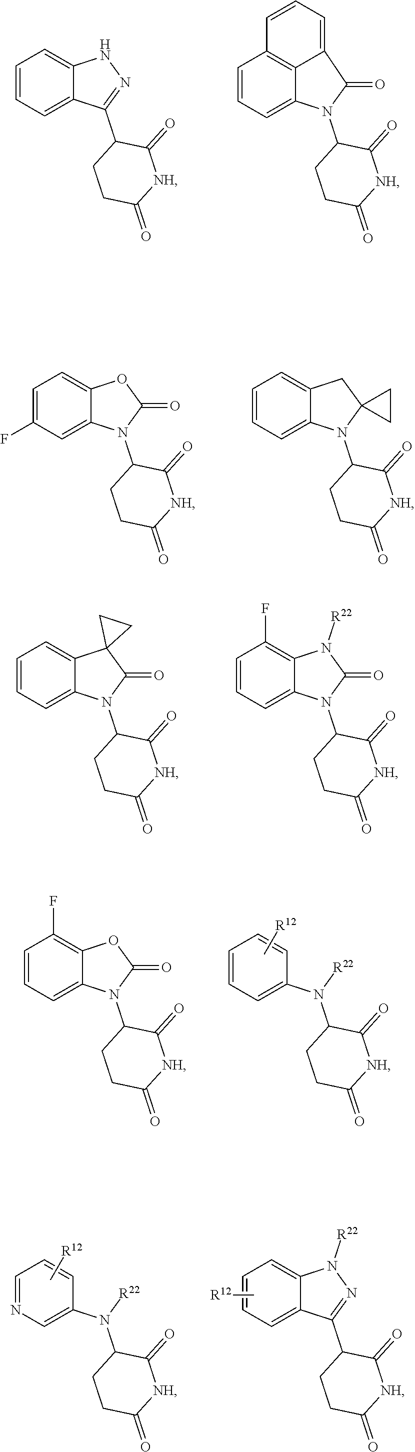

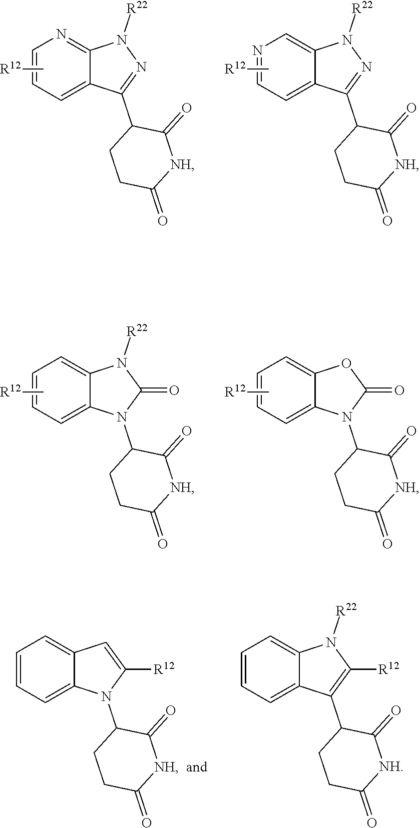

The chemical entities described herein can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in Schemes 1-7, the steps in some cases may be performed in a different order than the order shown in Schemes 1-7. Various modifications to these synthetic reaction schemes may be made and will be suggested to one skilled in the art having referred to the present disclosure. Numberings or R groups in each scheme typically have the same meanings as those defined elsewhere herein unless otherwise indicated.

Unless specified to the contrary, the reactions described herein take place at atmospheric pressure, generally within a temperature range from −10° C. to 200° C. Further, except as otherwise specified, reaction times and conditions are intended to be approximate, e.g., taking place at about atmospheric pressure within a temperature range of about −10° C. to about 110° C. over a period of about 1 to about 24 hours; reactions left to run overnight average a period of about 16 hours.

In general, compounds of the disclosure may be prepared by the following reaction schemes:

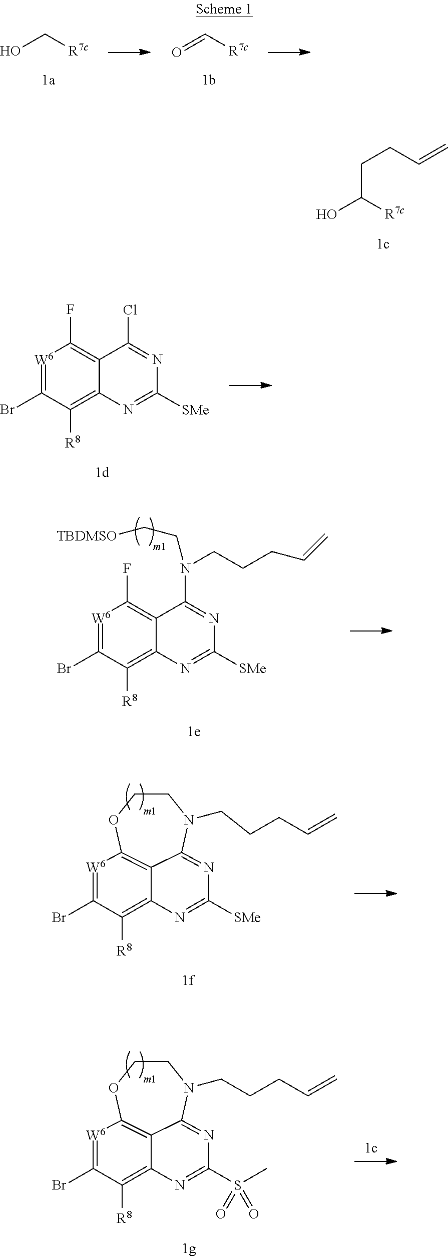

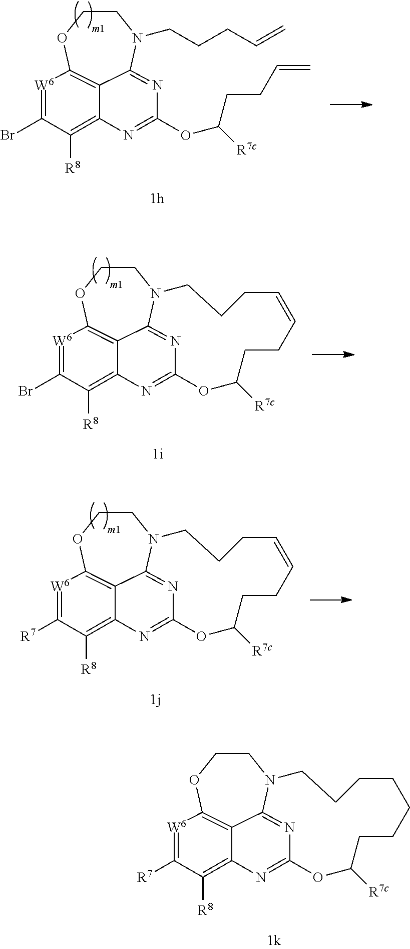

In some embodiments, a compound of Formula 1k may be prepared according to Scheme 1. For example, heteroaryl amine 1e can be formed from chloride 1d via a nucleophilic aromatic substitution reaction. Ring closure to 1f can be followed by an oxidation reaction to provide sulfone 1g. Addition of secondary alcohol 1c (prepared in two steps from primary alcohol 1a) can afford 1h, which can undergo a ring-closing metathesis reaction-such as a Grubbs metathesis reaction—to provide macrocyclic alkene 1i. Substitution of the aryl bromide with a suitable boronic ester can provide R⁷-substituted macrocycle 1j, which may optionally be subjected to one or more subsequent reactions, such as a hydrogenation reaction, to provide a compound of Formula 1k.

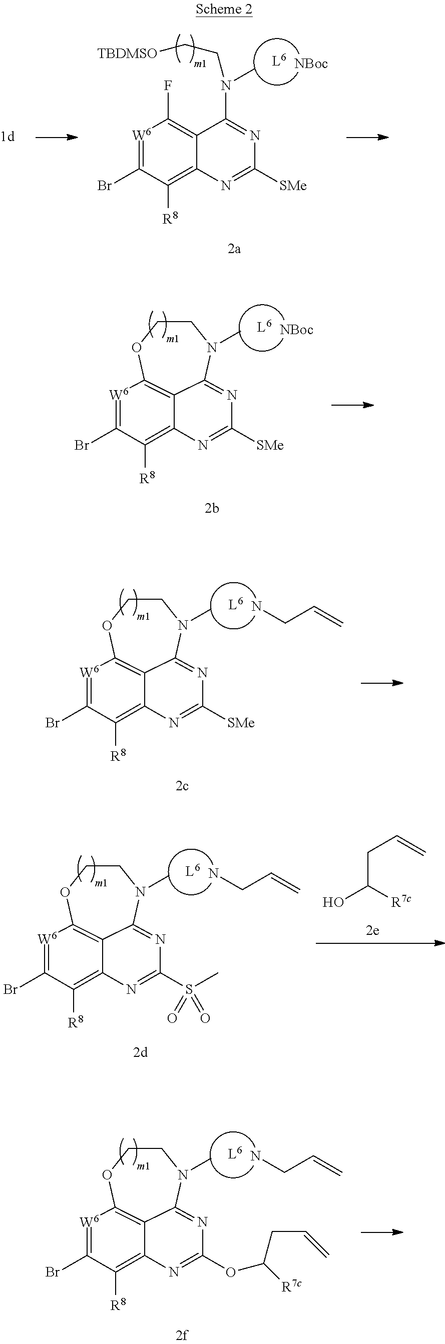

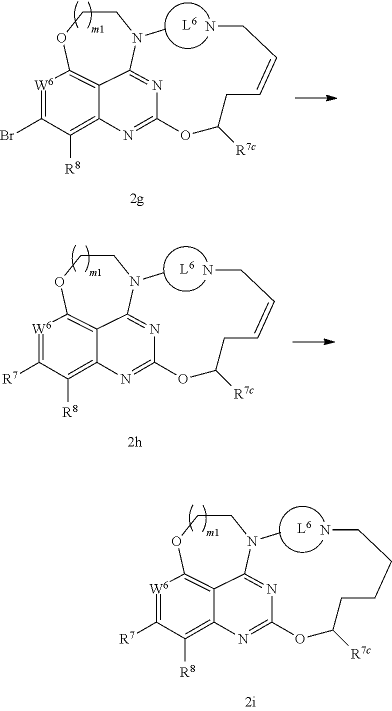

In some embodiments, a compound of Formula 2i may be prepared according to Scheme 2. For example, heteroaryl amine 2a can be formed from heteroaryl chloride 1d via a nucleophilic aromatic substitution reaction. Ring closure to 2b can be followed by Boc deprotection and allylation to provide allyl amine 2c. Oxidation can provide sulfone 2d, which can be substituted with secondary alcohol 2e (prepared in two steps analogously to 1c) to afford 2f. A ring-closing metathesis reaction-such as a Grubbs metathesis reaction—can provide macrocyclic alkene 2g. Substitution of the aryl bromide can proceed via a cross-coupling reaction-such as a Suzuki reaction with a suitable organoboron reagent—to provide R⁷-substituted macrocycle 2h. Optionally, 2h may be subjected to one or more subsequent reactions, such as a hydrogenation reaction, to provide a compound of Formula 2i.

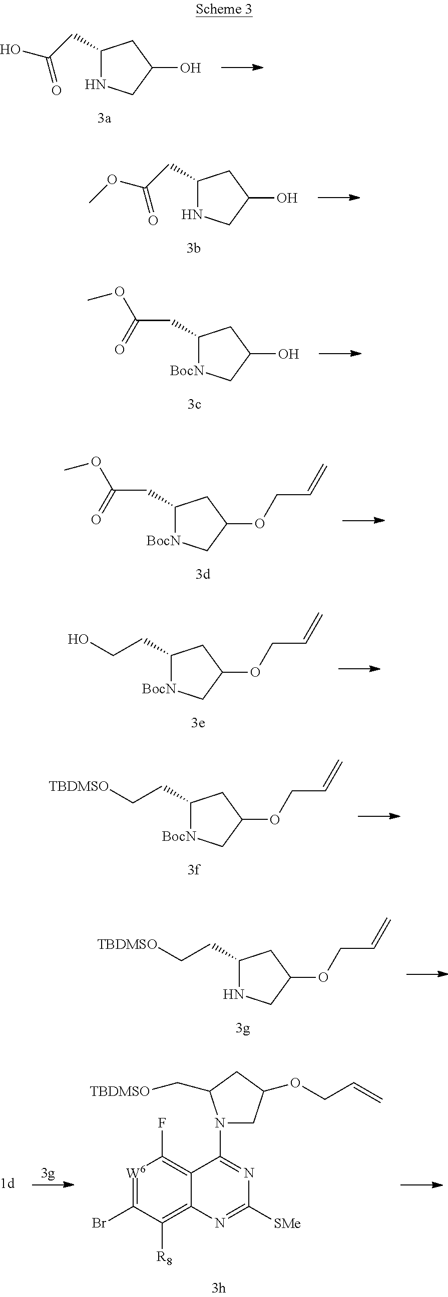

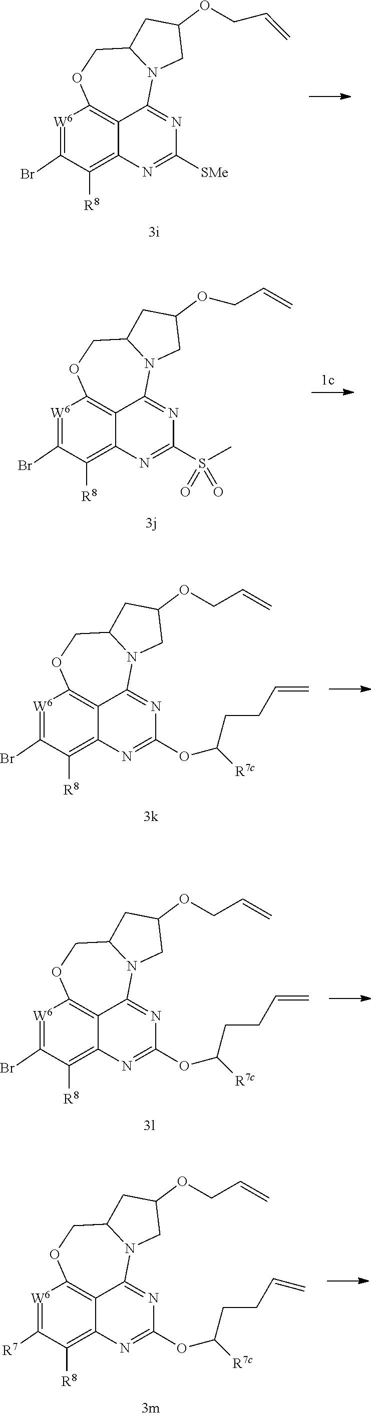

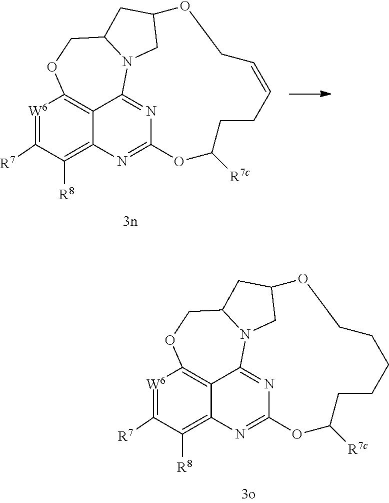

In some embodiments, a compound of Formula 3o may be prepared according to Scheme 3. For example, esterification of carboxylic acid 3a can provide methyl ester 3b, which can be Boc-protected to provide 3c. Allylation to 3d can be followed by a reduction (3e), installation of a TBDMS protecting group (3f) and Boc-deprotection to afford 3g. Heteroaryl amine 3h can then be formed from heteroaryl chloride 1d via a nucleophilic aromatic substitution reaction with 3g. Ring closure to tricycle 3i can be followed by oxidation to sulfone 3j, which can be substituted with secondary alcohol 1c to afford 3k. Substitution of the aryl bromide can proceed via a cross-coupling reaction-such as a Suzuki reaction with a suitable organoborono reagent—to provide R⁷-substituted tricycle 3m. A ring-closing metathesis reaction-such as a Grubbs metathesis reaction—can provide macrocyclic alkene 3n, which is optionally hydrogenated to the alkane of Formula 3o.

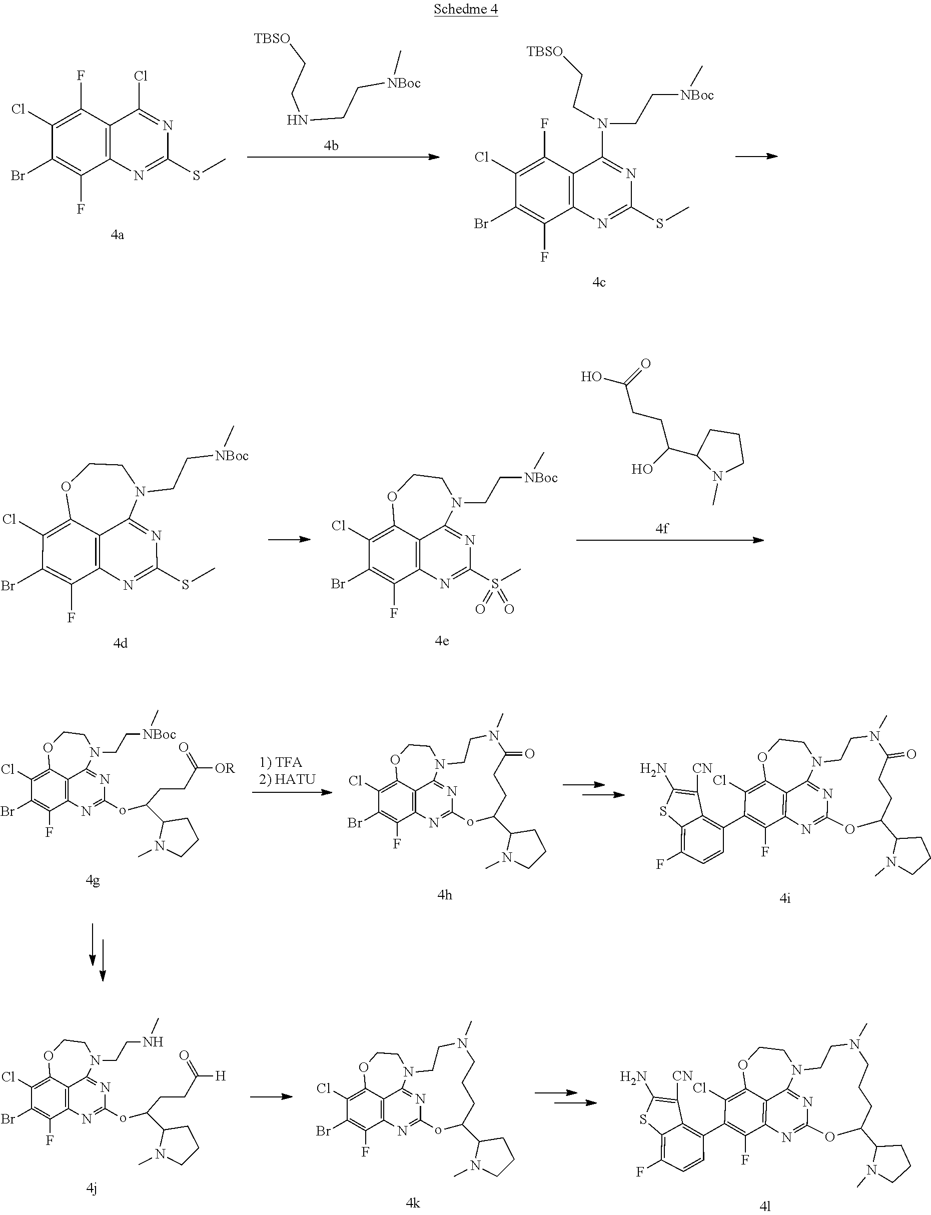

In some embodiments, a compound of Formula 4i or 4l may be prepared according to Scheme 4. For example, heteroaryl amine 4c can be formed from heteroaryl chloride 4a via a nucleophilic aromatic substitution reaction with 4b. Ring closure to tricycle 4d can be followed by oxidation to sulfone 4e, which can be substituted with secondary alcohol 4f to afford 4g. Boc-deprotection and peptide coupling can provide 4h, which can undergo a cross-coupling reaction-such as a Stille reaction with a suitable organostannane—and optionally one or more subsequent manipulations (e.g., a deprotection) to provide an amide of Formula 4i. Alternatively, 4g can undergo Boc-deprotection and reduction to aldehyde 4j. Reductive amination to tertiary amine 4k can be followed by one or more manipulations, such as a cross-coupling reaction and deprotection, to afford a compound of Formula 41.

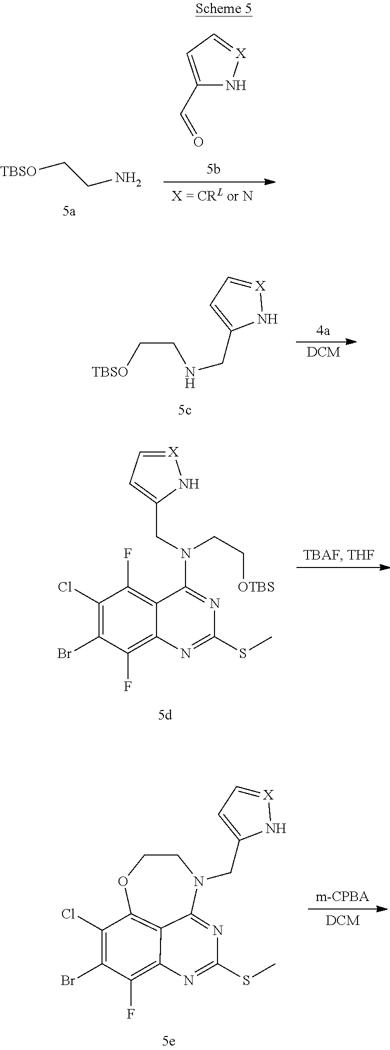

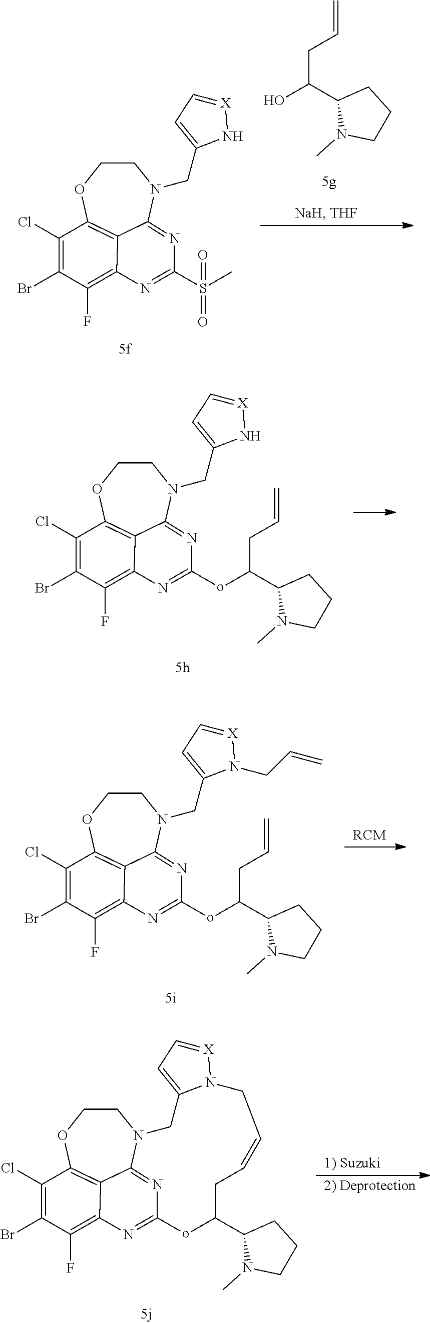

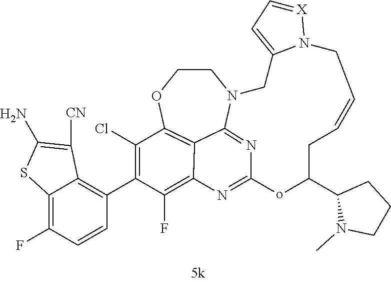

In some embodiments, a compound of Formula 5k may be prepared according to Scheme 5. For example, secondary amine 5c can be formed by reductive amination with aldehyde 5b. Substitution of heteroaryl chloride 4a with 5c can provide 5d, which can readily be cyclized to tricycle 5e. Oxidation to sulfone 5f and substitution with alcohol 5g to provide 5h can be followed with allylation to provide diene 51. A ring-closing metathesis reaction-such as a Grubbs metathesis reaction—can provide macrocyclic alkene 5j, which is optionally substituted with R⁷ via a cross-coupling reaction-such as a Suzuki reaction—to provide a compound of Formula 5k.

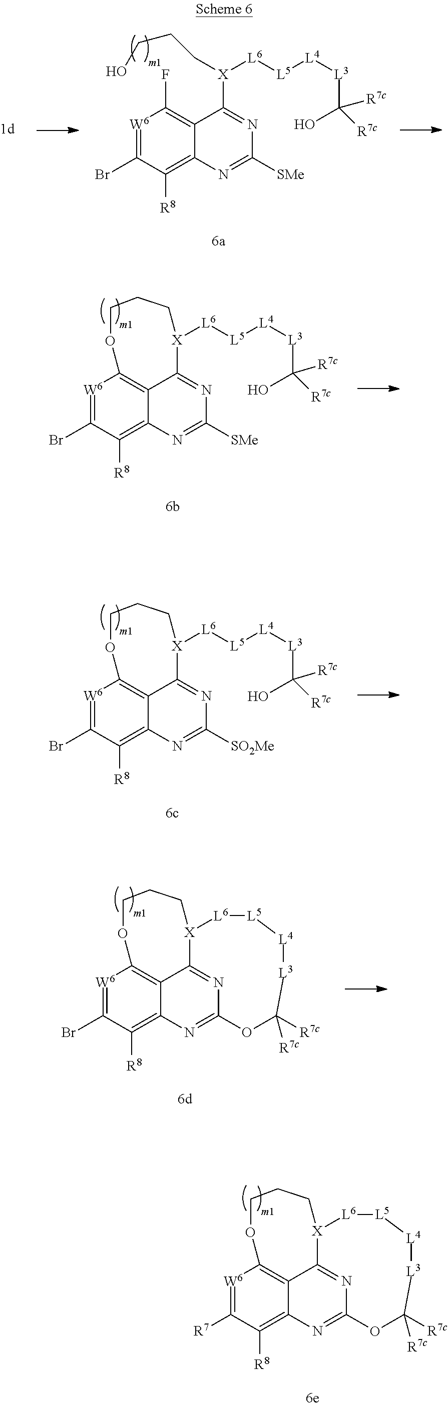

In some embodiments, a compound of Formula 6e may be prepared according to Scheme 6. For example, substitution of heteroaryl chloride 1d with a suitable nucleophile (e.g., HO(CH₂)ₘ₁CH₂CH₂XL⁶L⁵L⁴L³C(R^{7c})₂OH) can provide 6a, which can readily be cyclized to tricycle 6b. Oxidation to sulfone 6c and cyclization with the alcohol can provide macrocycle 6d. Substitution of the aryl bromide can proceed via a cross-coupling reaction-such as a Suzuki reaction with a suitable organoborono reagent—to provide an R⁷-substituted compound of Formula 6e.

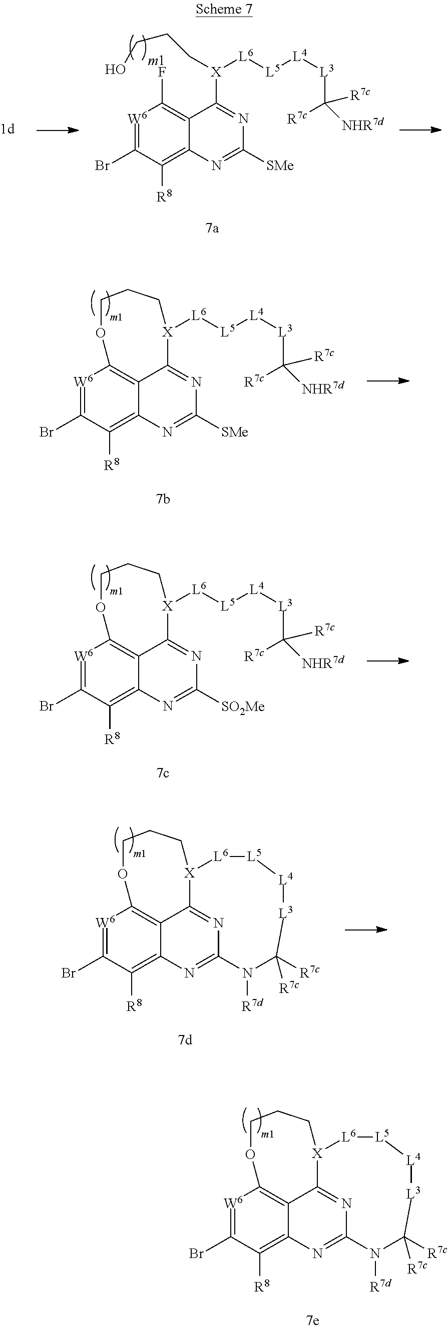

In some embodiments, a compound of Formula 7e may be prepared according to Scheme 7. For example, substitution of heteroaryl chloride 1d with a suitable nucleophile (e.g., HO(CH₂)ₘ₁CH₂CH₂XL⁶L⁵L⁴L³-C(R^{7c})₂NHR^{7d}) can provide 7a, which can readily be cyclized to tricycle 7b. Oxidation to sulfone 7c and cyclization with the alcohol can provide macrocycle 7d. Substitution of the aryl bromide can proceed via a cross-coupling reaction—such as a Suzuki reaction with a suitable organoborono reagent—to provide an R⁷-substituted compound of Formula 7e.

In some embodiments, a compound of the (present disclosure, for example, a compound of a formula given in Table 1, was synthesized according to one of the general routes outlined in Schemes 1-7, Example 1, or by methods generally known in the art. In some embodiments, exemplary compounds may include, but are not limited to, a compound selected from Table 1, or a salt or solvate thereof.

TABLE 1

| No. | Structure | Chemical Name | [M + H ]⁺ |
|---|---|---|---|
| 101 | 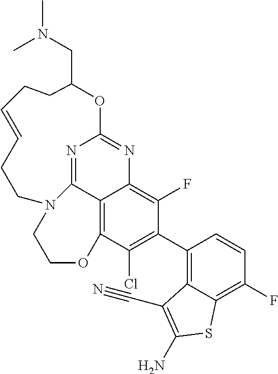 [SMILES: CN(C)CC1CC/C=C/CCN2CCOc3c(Cl)c(-c4ccc(F)c5sc(N)c(C#N)c45)c(F)c4nc(nc2c34)O1] | (E)-2-amino-4-(15-chloro-4- ((dimethylamino)methyl)-17-fluoro- 5,6,9,10,12,13-hexahydro-4H-2,18- (azeno)benzo[6,7][1,4]oxazepino[5,4- d][1]oxa[3,5]diazacyclododecin-16-yl)-7- fluorobenzo[b]thiophene-3-carbonitrile (E double bond geometry tentatively assigned) | 597.1 |
| 102 | 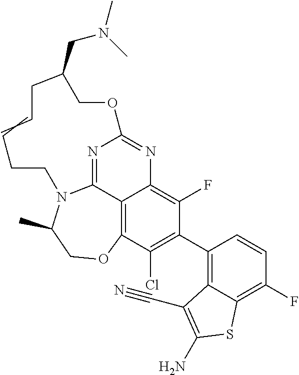 [SMILES: C[C@@H]1COc2c(Cl)c(-c3ccc(F)c4sc(N)c(C#N)c34)c(F)c3nc4nc(c23)N1CCC=CC[C@@H](CN(C)C)CO4] | 2-amino-4-((5S,12R)-16-chloro-5- ((dimethylamino)methyl)-18-fluoro-12-methyl- 5,6,9,10,13,14-hexahydro-4H,12H-2,19- (azeno)benzo[2,3][1,5]oxazocino[4,5- d][1]oxa[3,5]diazacyclododecin-17-yl)-7- fluorobenzo[b]thiophene-3-carbonitrile (diastereomer mixture 1) | 624.8 |
| 103 | 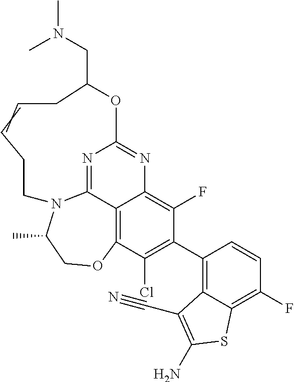 [SMILES: C[C@H]1COc2c(Cl)c(-c3ccc(F)c4sc(N)c(C#N)c34)c(F)c3nc4nc(c23)N1CCC=CCC(CN(C)C)O4] | 2-amino-4-((11S)-14-chloro-4- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 596.9 |
| 104 | 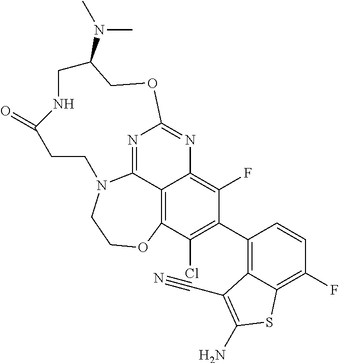 [SMILES: CN(C)[C@@H]1CNC(=O)CCN2CCOc3c(Cl)c(-c4ccc(F)c5sc(N)c(C#N)c45)c(F)c4nc(nc2c34)OC1] | 2-amino-4-((5R)-15-chloro-5-(dimethylamino)-17- fluoro-8-oxo-5,6,7,8,9,10,12,13-octahydro-4H- 2,18-(azeno)benzo[6,7][1,4]oxazepino[5,4- d][1]oxa[3,5,9]triazacyclododecin-16-yl)-7- fluorobenzo[b]thiophene-3-carbonitrile (diastereomer mixture 1) | 599.8 |
| 105 | 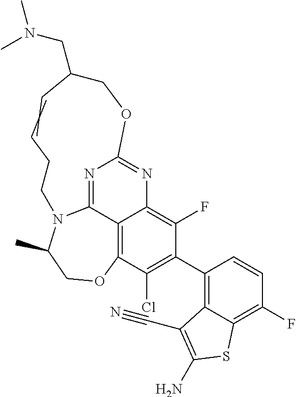 [SMILES: C[C@@H]1COc2c(Cl)c(-c3ccc(F)c4sc(N)c(C#N)c34)c(F)c3nc4nc(c23)N1CCC=CC(CN(C)C)CO4] | 2-amino-4-((11R)-15-chloro-5- ((dimethylamino)methyl)-17-fluoro-11-methyl- 4,5,8,9,12,13-hexahydro-11H-2,18- (azeno)benzo[2,3][1,5]oxazocino[4,5- d][1]oxa[3,5]diazacycloundecin-16-yl)-7- fluorobenzo[b]thiophene-3-carbonitrile (diastereomer mixture 3) | 611.1 |
| 106 | 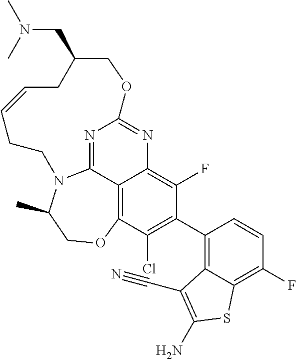 [SMILES: C[C@@H]1COc2c(Cl)c(-c3ccc(F)c4sc(N)c(C#N)c34)c(F)c3nc4nc(c23)N1CC/C=C\C[C@@H](CN(C)C)CO4] | 2-amino-4-((5S,12R,Z)-16-chloro-5- ((dimethylamino)methyl)-18-fluoro-12-methyl- 5,6,9,10,13,14-hexahydro-4H,12H-2,19- (azeno)benzo[2,3][1,5]oxazocino[4,5- d][1]oxa[3,5]diazacyclododecin-17-yl)-7- fluorobenzo[b]thiophene-3-carbonitrile (Z double bond geometry tentatively assigned) (diastereomer mixture 4) | 624.8 |
| 107 | 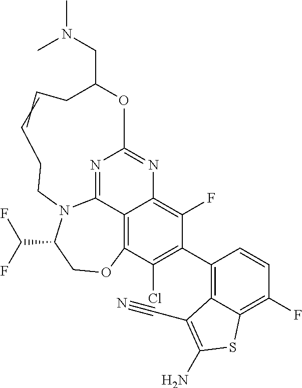 [SMILES: CN(C)CC1CC=CCCN2c3nc(nc4c(F)c(-c5ccc(F)c6sc(N)c(C#N)c56)c(Cl)c(c34)OC[C@@H]2C(F)F)O1] | 2-amino-4-((11R)-14-chloro-11-(difluoromethyl)- 4-((dimethylamino)methyl)-16-fluoro- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 632.9 |
| 108 | 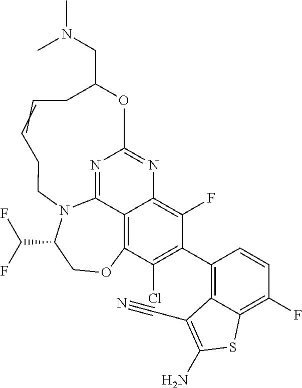 [SMILES: CN(C)CC1CC=CCCN2c3nc(nc4c(F)c(-c5ccc(F)c6sc(N)c(C#N)c56)c(Cl)c(c34)OC[C@@H]2C(F)F)O1] | 2-amino-4-((11R)-14-chloro-11-(difluoromethyl)- 4-((dimethylamino)methyl)-16-fluoro- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 3) | 632.9 |
| 109 | 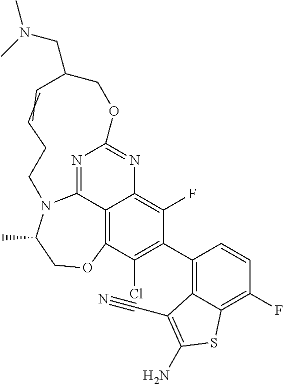 [SMILES: C[C@H]1COc2c(Cl)c(-c3ccc(F)c4sc(N)c(C#N)c34)c(F)c3nc4nc(c23)N1CCC=CC(CN(C)C)CO4] | 2-amino-4-((11S)-14-chloro-5- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 596.8 |
| 110 | 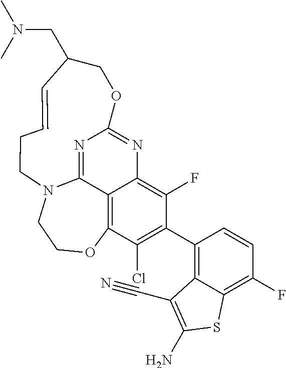 [SMILES: CN(C)CC1/C=C/CCN2CCOc3c(Cl)c(-c4ccc(F)c5sc(N)c(C#N)c45)c(F)c4nc(nc2c34)OC1] | (E)-2-amino-4-(14-chloro-5- ((dimethylamino)methyl)-16-fluoro-4,5,8,9,11,12- hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (E double bond geometry tentatively assigned) | 582.8 |
| 111 | 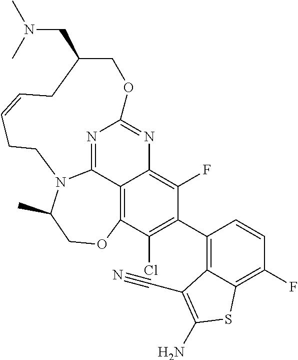 [SMILES: C[C@@H]1COc2c(Cl)c(-c3ccc(F)c4sc(N)c(C#N)c34)c(F)c3nc4nc(c23)N1CC/C=C\C[C@@H](CN(C)C)CO4] | 2-amino-4-((5S,12R,Z)-16-chloro-5- ((dimethylamino)methyl)-18-fluoro-12-methyl- 5,6,9,10,13,14-hexahydro-4H,12H-2,19- (azeno)benzo[2,3][1,5]oxazocino[4,5- d][1]oxa[3,5]diazacyclododecin-17-yl)-7- fluorobenzo[b]thiophene-3-carbonitrile (Z double bond geometry tentatively assigned) (diastereomer mixture 2) | 624.8 |
| 112 | 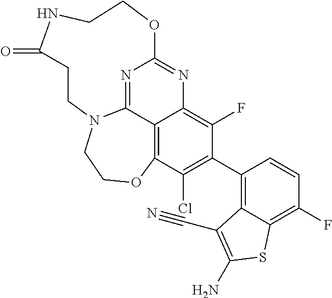 [SMILES: N#Cc1c(N)sc2c(F)ccc(-c3c(Cl)c4c5c6nc(nc5c3F)OCCNC(=O)CCN6CCO4)c12] | 2-amino-4-(14-chloro-16-fluoro-7-oxo- 4,5,6,7,8,9,11,12-octahydro-2,17- (azeno)benzo[6,7][1,4]oxazepino[5,4- d][1]oxa[3,5,9]triazacycloundecin-15-yl)-7- fluorobenzo[b]thiophene-3-carbonitrile | 642.9 |
| 113 | 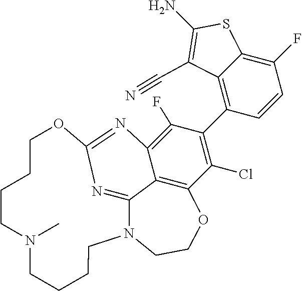 [SMILES: CN1CCCCOc2nc3c4c(c(Cl)c(-c5ccc(F)c6sc(N)c(C#N)c56)c(F)c4n2)OCCN3CCCC1] | 2-amino-4-(17-chloro-19-fluoro-8-methyl- 5,6,7,8,9,10,11,12,14,15-decahydro-4H-2,20- (azeno)benzo[6,7][1,4]oxazepino[5,4- d][1]oxa[3,5,10]triazacyclotetradecin-18-yl)-7- fluorobenzo[b]thiophene-3-carbonitrile | 585.4 |
| 114 | 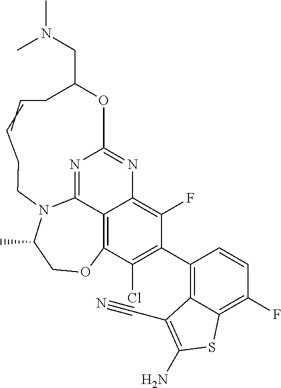 [SMILES: C[C@H]1COc2c(Cl)c(-c3ccc(F)c4sc(N)c(C#N)c34)c(F)c3nc4nc(c23)N1CCC=CCC(CN(C)C)O4] | 2-amino-4-((11S)-14-chloro-4- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 3) | 596.9 |
| 115 | 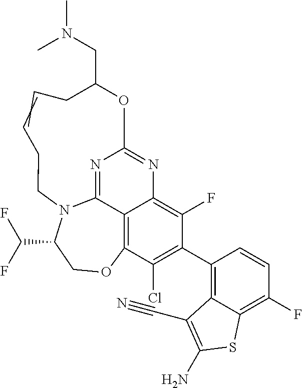 [SMILES: CN(C)CC1CC=CCCN2c3nc(nc4c(F)c(-c5ccc(F)c6sc(N)c(C#N)c56)c(Cl)c(c34)OC[C@@H]2C(F)F)O1] | 2-amino-4-((11R)-14-chloro-11-(difluoromethyl)- 4-((dimethylamino)methyl)-16-fluoro- 4,5,8,9,11,12-hexahydro-2,17- (azeno)benzo[6,7][1,4]oxazepino[5,4- d][1]oxa[3,5]diazacycloundecin-15-y1)-7- fluorobenzo[b]thiophene-3-carbonitrile (diastereomer mixture 1) | 632.9 |
| 116 | 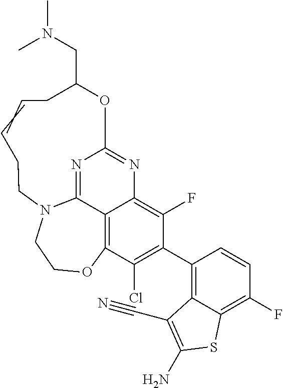 [SMILES: CN(C)CC1CC=CCCN2CCOc3c(Cl)c(-c4ccc(F)c5sc(N)c(C#N)c45)c(F)c4nc(nc2c34)O1] | 2-amino-4-(14-chloro-4-((dimethylamino)methyl)- 16-fluoro-4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 582.9 |
| 117 | 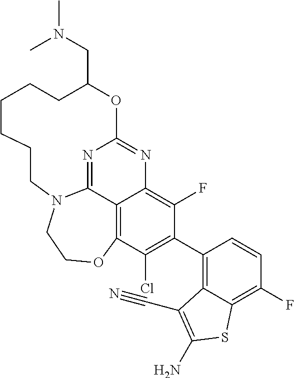 [SMILES: CN(C)CC1CCCCCCN2CCOc3c(Cl)c(-c4ccc(F)c5sc(N)c(C#N)c45)c(F)c4nc(nc2c34)O1] | 2-amino-4-(15-chloro-4-((dimethylamino)methyl)- 17-fluoro-5,6,7,8,9,10,12,13-octahydro-4H-2,18- (azeno)benzo[6,7][1,4]oxazepino[5,4- d][1]oxa[3,5]diazacyclododecin-16-yl)-7- fluorobenzo[b]thiophene-3-carbonitrile (diastereomer mixture 1) | 598.1 |
| 118 | 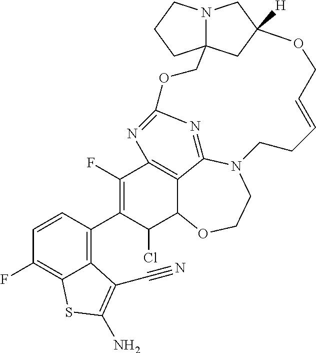 [SMILES: N#Cc1c(N)sc2c(F)ccc(C3=C(F)c4nc5nc6c4C(OCCN6CC/C=C/CO[C@H]4CN6CCCC6(CO5)C4)C3Cl)c12] | 2-amino-4-((15R,E)-4-chloro-2-fluoro- 6,7,10,13,15,16,19,20-octahydro-9H,18H,21H- 1,23-(azeno)-15,20a- methanobenzo[6,7][1,4]oxazepino[5,4- d]pyrrolo[1,2- n][1,11]dioxa[3,5,14]triazacyclohexadecin-3-yl)-7- fluorobenzo[b]thiophene-3-carbonitrile (diastereomer 3) | 651.2 |
| 119 | 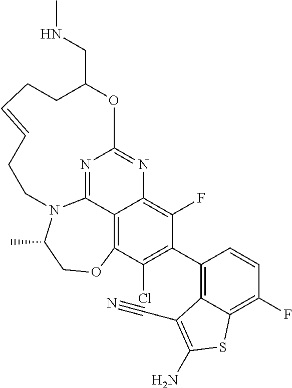 [SMILES: CNCC1CC/C=C/CCN2c3nc(nc4c(F)c(-c5ccc(F)c6sc(N)c(C#N)c56)c(Cl)c(c34)OC[C@@H]2C)O1] | (E)-2-amino-4-(15-chloro-17-fluoro-4- ((methylamino)methyl)-5,6,9,10,12,13-hexahydro- 4H-2,18-(azeno)benzo[6,7][1,4]oxazepino[5,4- d][1]oxa[3,5]diazacyclododecin-16-yl)-7- fluorobenzo[b]thiophene-3-carbonitrile (E double bond geometry tentatively assigned) | 582.8 |
| 120 | 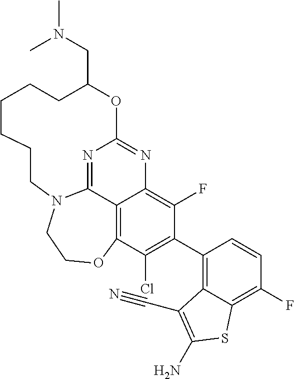 [SMILES: CN(C)CC1CCCCCCN2CCOc3c(Cl)c(-c4ccc(F)c5sc(N)c(C#N)c45)c(F)c4nc(nc2c34)O1] | 2-amino-4-(15-chloro-4-((dimethylamino)methyl)- 17-fluoro-5,6,7,8,9,10,12,13-octahydro-4H-2,18- (azeno)benzo[6,7][1,4]oxazepino[5,4- d][1]oxa[3,5]diazacyclododecin-16-yl)-7- fluorobenzo[b]thiophene-3-carbonitrile (diastereomer mixture 2) | 598.1 |
| 121 | 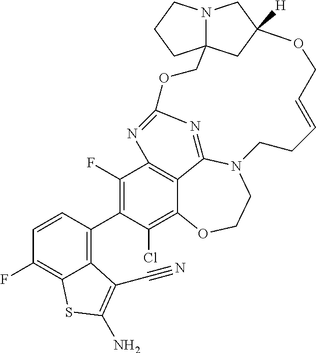 [SMILES: N#Cc1c(N)sc2c(F)ccc(-c3c(Cl)c4c5c6nc(nc5c3F)OCC35CCCN3C[C@@H](C5)OC/C=C/CCN6CCO4)c12] | 2-amino-4-((15R,E)-4-chloro-2-fluoro- 6,7,10,13,15,16,19,20-octahydro-9H,18H,21H- 1,23-(azeno)-15,20a- methanobenzo[6,7][1,4]oxazepino[5,4- d]pyrrolo[1,2-n][1,11]dioxa[3,5,14] triazacyclohexadecin-3-yl)-7- fluorobenzo[b]thiophene-3-carbonitrile (diastereomer 1) | 651.2 |
| 122 | 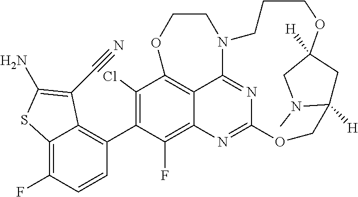 [SMILES: CN1C[C@@H]2C[C@H]1COc1nc3c4c(c(Cl)c(-c5ccc(F)c6sc(N)c(C#N)c56)c(F)c4n1)OCCN3CCCO2] | 2-amino-4-((5S,8S)-17-chloro-19-fluoro-6-methyl- 5,6,7,8,11,12,14,15-octahydro-4H,10H-2,20- (azeno)-5,8- methanobenzo[6,7][1,4]oxazepino[5,4- d][1,9]dioxa[3,5,12]triazacyclotetradecin-18-yl)-7- fluorobenzo[b]thiophene-3-carbonitrile (diastereomer mixture 2) | 598.8 |
| 123 | 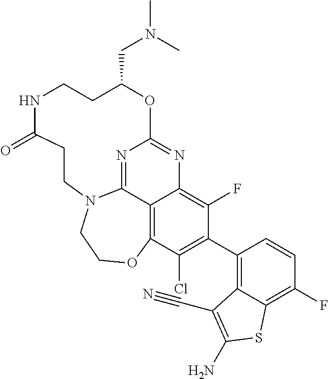 [SMILES: CN(C)C[C@H]1CCNC(=O)CCN2CCOc3c(Cl)c(-c4ccc(F)c5sc(N)c(C#N)c45)c(F)c4nc(nc2c34)O1] | 2-amino-4-((4R)-15-chloro-4- ((dimethylamino)methyl)-17-fluoro-8-oxo- 5,6,7,8,9,10,12,13-octahydro-4H-2,18- (azeno)benzo[6,7][1,4]oxazepino[5,4- d][1]oxa[3,5,9]triazacyclododecin-16-yl)-7- fluorobenzo[b]thiophene-3-carbonitrile (diastereomer mixture 2) | 613.7 |
| 124 | 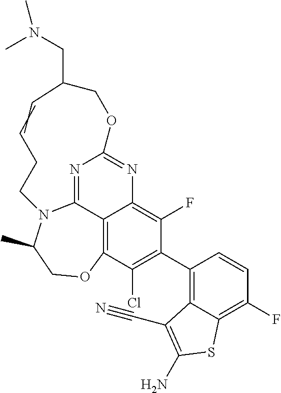 [SMILES: C[C@@H]1COc2c(Cl)c(-c3ccc(F)c4sc(N)c(C#N)c34)c(F)c3nc4nc(c23)N1CCC=CC(CN(C)C)CO4] | 2-amino-4-((11R,Z)-15-chloro-5- ((dimethylamino)methyl)-17-fluoro-11-methyl- 4,5,8,9,12,13-hexahydro-11H-2,18- (azeno)benzo[2,3][1,5]oxazocino[4,5- d][1]oxa[3,5]diazacycloundecin-16-yl)-7- fluorobenzo[b]thiophene-3-carbonitrile (diastereomer mixture 1) | 611.1 |
| 125 | 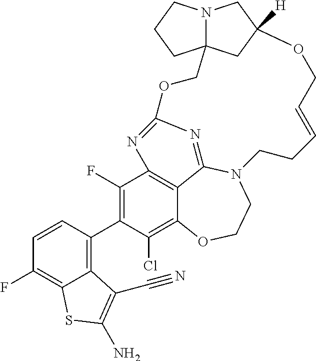 [SMILES: N#Cc1c(N)sc2c(F)ccc(-c3c(Cl)c4c5c6nc(nc5c3F)OCC35CCCN3C[C@@H](C5)OC/C=C/CCN6CCO4)c12] | 2-amino-4-((15R,E)-4-chloro-2-fluoro- 6,7,10,13,15,16,19,20-octahydro-9H,18H,21H- 1,23-(azeno)-15,20a- methanobenzo[6,7][1,4]oxazepino[5,4- d]pyrrolo[1,2- n][1,11]dioxa[3,5,14]triazacyclohexadecin-3-yl)-7- fluorobenzo[b]thiophene-3-carbonitrile (diastereomer 2) | 651.2 |
| 126 | 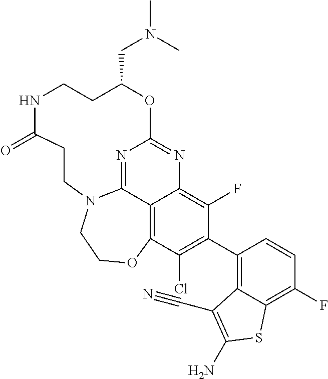 [SMILES: CN(C)C[C@H]1CCNC(=O)CCN2CCOc3c(Cl)c(-c4ccc(F)c5sc(N)c(C#N)c45)c(F)c4nc(nc2c34)O1] | 2-amino-4-((4R)-15-chloro-4- ((dimethylamino)methyl)-17-fluoro-8-oxo- 5,6,7,8,9,10,12,13-octahydro-4H-2,18- (azeno)benzo[6,7][1,4]oxazepino[5,4- d][1]oxa[3,5,9]triazacyclododecin-16-yl)-7- fluorobenzo[b]thiophene-3-carbonitrile (diastereomer mixture 1) | 613.7 |
| 127 | 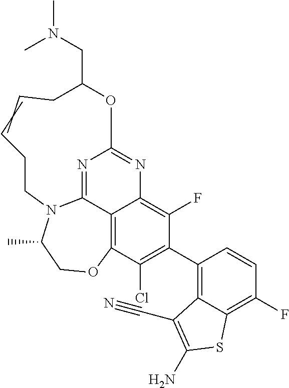 [SMILES: C[C@H]1COc2c(Cl)c(-c3ccc(F)c4sc(N)c(C#N)c34)c(F)c3nc4nc(c23)N1CCC=CCC(CN(C)C)O4] | 2-amino-4-((11S)-14-chloro-4- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 596.9 |
| 128 | 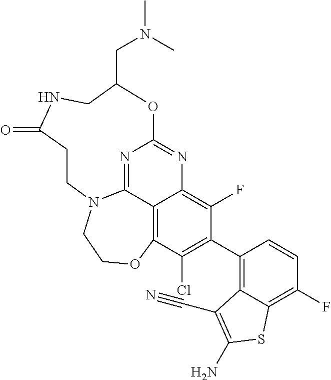 [SMILES: CN(C)CC1CNC(=O)CCN2CCOc3c(Cl)c(-c4ccc(F)c5sc(N)c(C#N)c45)c(F)c4nc(nc2c34)O1] | 2-amino-4-(14-chloro-4-((dimethylamino)methyl)- 16-fluoro-7-oxo-4,5,6,7,8,9,11,12-octahydro-2,17- (azeno)benzo[6,7][1,4]oxazepino[5,4- d][1]oxa[3,5,9]triazacycloundecin-15-yl)-7- fluorobenzo[b]thiophene-3-carbonitrile (diastereomer mixture 1) | 600.1 |
| 129 | 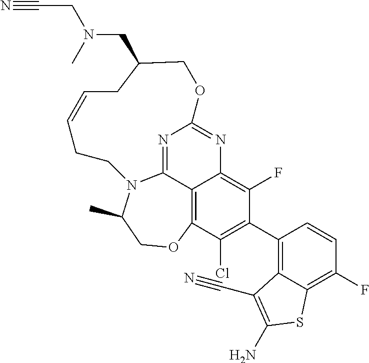 [SMILES: C[C@@H]1COc2c(Cl)c(-c3ccc(F)c4sc(N)c(C#N)c34)c(F)c3nc4nc(c23)N1CC/C=C\C[C@@H](CN(C)CC#N)CO4] | 2-amino-4-((5S,12R,Z)-16-chloro-5- (((cyanomethyl)(methyl)amino)methyl)-18-fluoro- 12-methyl-5,6,9,10,13,14-hexahydro-4H,12H- 2,19-(azeno)benzo[2,3][1,5]oxazocino[4,5- d][1]oxa[3,5]diazacyclododecin-17-yl)-7- fluorobenzo[b]thiophene-3-carbonitrile | 649.8 |
| 130 | 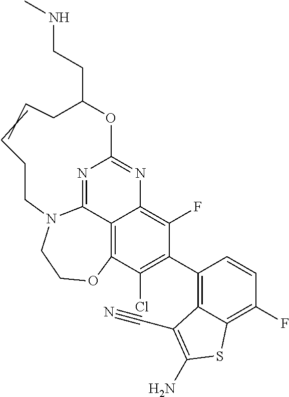 [SMILES: CNCCC1CC=CCCN2CCOc3c(Cl)c(-c4ccc(F)c5sc(N)c(C#N)c45)c(F)c4nc(nc2c34)O1] | 2-amino-4-(14-chloro-16-fluoro-4-(2- (methylamino)ethyl)-4,5,8,9,11,12-hexahydro- 2,17-(azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 583.1 |
| 131 | 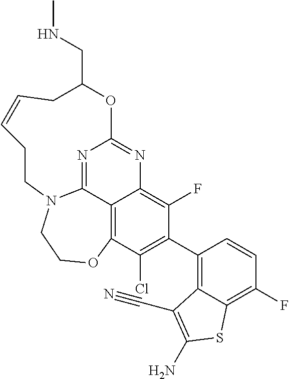 [SMILES: CNCC1C/C=C\CCN2CCOc3c(Cl)c(-c4ccc(F)c5sc(N)c(C#N)c45)c(F)c4nc(nc2c34)O1] | (Z)-2-amino-4-(14-chloro-16-fluoro-4- ((methylamino)methyl)-4,5,8,9,11,12-hexahydro- 2,17-(azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 568.9 |
| 132 | 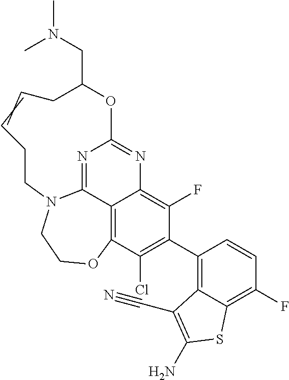 [SMILES: CN(C)CC1CC=CCCN2CCOc3c(Cl)c(-c4ccc(F)c5sc(N)c(C#N)c45)c(F)c4nc(nc2c34)O1] | 2-amino-4-(14-chloro-4-((dimethylamino)methyl)- 16-fluoro-4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 582.9 |
| 133 | 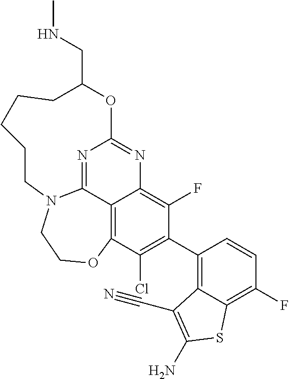 [SMILES: CNCC1CCCCCN2CCOc3c(Cl)c(-c4ccc(F)c5sc(N)c(C#N)c45)c(F)c4nc(nc2c34)O1] | 2-amino-4-(14-chloro-16-fluoro-4- ((methylamino)methyl)-4,5,6,7,8,9,11,12- octahydro-2,17- (azeno)benzo[6,7][1,4]oxazepino[5,4- d][1]oxa[3,5]diazacycloundecin-15-yl)-7- fluorobenzo[b]thiophene-3-carbonitrile | 570.9 |
| 134 | 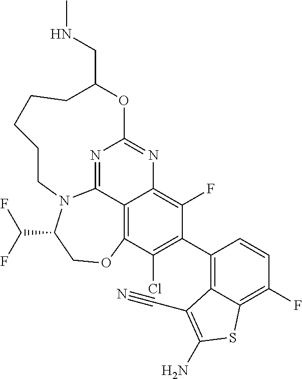 [SMILES: CNCC1CCCCCN2c3nc(nc4c(F)c(-c5ccc(F)c6sc(N)c(C#N)c56)c(Cl)c(c34)OC[C@@H]2C(F)F)O1] | 2-amino-4-((11R)-14-chloro-11-(difluoromethyl)- 4-((dimethylamino)methyl)-16-fluoro- 4,5,6,7,8,9,11,12-octahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 634.9 |
| 135 | 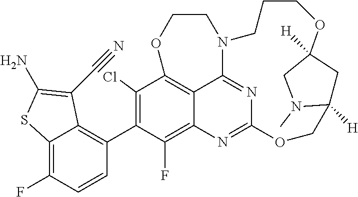 [SMILES: CN1C[C@@H]2C[C@H]1COc1nc3c4c(c(Cl)c(-c5ccc(F)c6sc(N)c(C#N)c56)c(F)c4n1)OCCN3CCCO2] | 2-amino-4-((5S,8S)-17-chloro-19-fluoro-6-methyl- 5,6,7,8,11,12,14,15-octahydro-4H,10H-2,20- (azeno)-5,8-methanobenzo[6,7] [1,4]oxazepino[5,4-d][1,9]dioxa [3,5,12]triazacyclotetradecin-18-y1)-7- fluorobenzo[b]thiophene-3-carbonitrile (diastereomer mixture 1) | 598.8 |
| 136 | 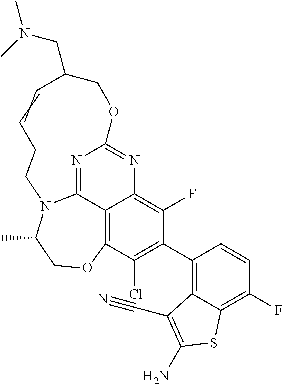 [SMILES: C[C@H]1COc2c(Cl)c(-c3ccc(F)c4sc(N)c(C#N)c34)c(F)c3nc4nc(c23)N1CCC=CC(CN(C)C)CO4] | 2-amino-4-((11S)-14-chloro-5- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 596.8 |
| 137 | 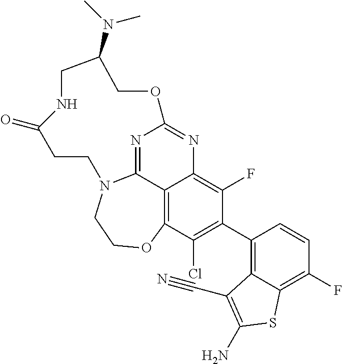 [SMILES: CN(C)[C@@H]1CNC(=O)CCN2CCOc3c(Cl)c(-c4ccc(F)c5sc(N)c(C#N)c45)c(F)c4nc(nc2c34)OC1] | 2-amino-4-((5R)-15-chloro-5-(dimethylamino)-17- fluoro-8-oxo-5,6,7,8,9,10,12,13-octahydro-4H- 2,18-(azeno)benzo[6,7][1,4]oxazepino[5,4- d][1]oxa[3,5,9]triazacyclododecin-16-yl)-7- fluorobenzo[b]thiophene-3-carbonitrile (diastereomer mixture 2) | 599.8 |
| 138 | 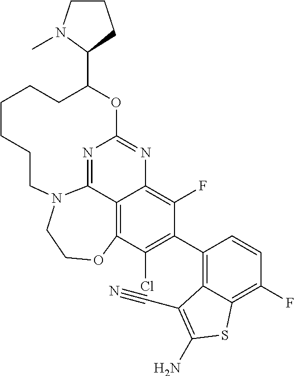 [SMILES: CN1CCC[C@H]1C1CCCCCCN2CCOc3c(Cl)c(-c4ccc(F)c5sc(N)c(C#N)c45)c(F)c4nc(nc2c34)O1] | 2-amino-4-(15-chloro-17-fluoro-4-((S)-1- methylpyrrolidin-2-yl)-5,6,7,8,9,10,12,13- octahydro-4H-2,18- (azeno)benzo[6,7][1,4]oxazepino[5,4- d][1]oxa[3,5]diazacyclododecin-16-yl)-7- fluorobenzo[b]thiophene-3-carbonitrile | 625.3 |
| 139 | 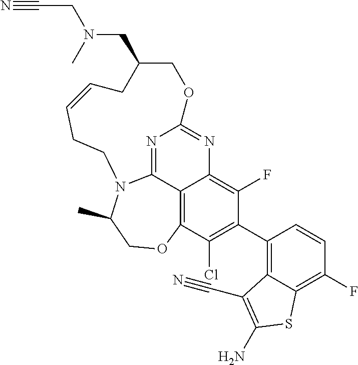 [SMILES: C[C@@H]1COc2c(Cl)c(-c3ccc(F)c4sc(N)c(C#N)c34)c(F)c3nc4nc(c23)N1CC/C=C\C[C@@H](CN(C)CC#N)CO4] | 2-amino-4-((5S,12R,Z)-16-chloro-5- (((cyanomethyl)(methyl)amino)methyl)-18-fluoro- 12-methyl-5,6,9,10,13,14-hexahydro-4H,12H- 2,19-(azeno)benzo[2,3][1,5]oxazocino[4,5- d][1]oxa[3,5]diazacyclododecin-17-yl)-7- fluorobenzo[b]thiophene-3-carbonitrile | 649.8 |
| 140 | 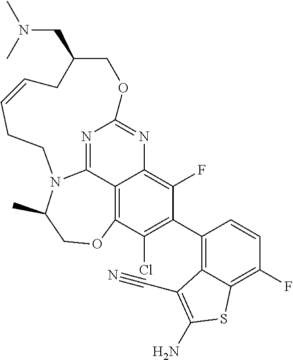 [SMILES: C[C@@H]1COc2c(Cl)c(-c3ccc(F)c4sc(N)c(C#N)c34)c(F)c3nc4nc(c23)N1CC/C=C\C[C@@H](CN(C)C)CO4] | 2-amino-4-((5S,12R,Z)-16-chloro-5- ((dimethylamino)methyl)-18-fluoro-12-methyl- 5,6,9,10,13,14-hexahydro-4H,12H-2,19- (azeno)benzo[2,3][1,5]oxazocino[4,5- d][1]oxa[3,5]diazacyclododecin-17-yl)-7- fluorobenzo[b]thiophene-3-carbonitrile (Z double bond geometry tentatively assigned) (diastereomer mixture 3) | 624.8 |
| 141 | 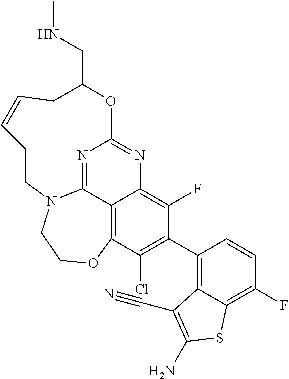 [SMILES: CNCC1C/C=C\CCN2CCOc3c(Cl)c(-c4ccc(F)c5sc(N)c(C#N)c45)c(F)c4nc(nc2c34)O1] | (Z)-2-amino-4-(14-chloro-16-fluoro-4- ((methylamino)methyl)-4,5,8,9,11,12-hexahydro- 2,17-(azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 568.9 |
| 142 | 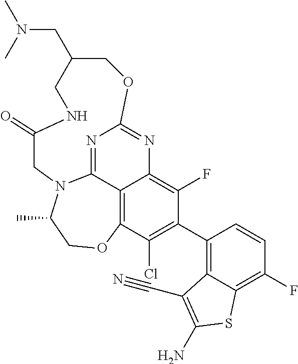 [SMILES: C[C@H]1COc2c(Cl)c(-c3ccc(F)c4sc(N)c(C#N)c34)c(F)c3nc4nc(c23)N1CC(=O)NCC(CN(C)C)CO4] | 2-amino-4-((11S)-14-chloro-5- ((dimethylamino)methyl)-16-fluoro-11-methyl-8- oxo-4,5,6,7,8,9,11,12-octahydro-2,17- (azeno)benzo[6,7][1,4]oxazepino[5,4- d][1]oxa[3,5,8]triazacycloundecin-15-yl)-7- fluorobenzo[b]thiophene-3-carbonitrile (diastereomer mixture 2) | 614.3 |
| 143 | 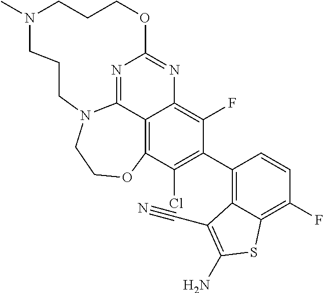 [SMILES: CN1CCCOc2nc3c4c(c(Cl)c(-c5ccc(F)c6sc(N)c(C#N)c56)c(F)c4n2)OCCN3CCC1] | 2-amino-4-(15-chloro-17-fluoro-7-methyl- 5,6,7,8,9,10,12,13-octahydro-4H-2,18- (azeno)benzo[6,7][1,4]oxazepino[5,4- d][1]oxa[3,5,9]triazacyclododecin-16-yl)-7- fluorobenzo[b]thiophene-3-carbonitrile | 556.8 |
| 144 | 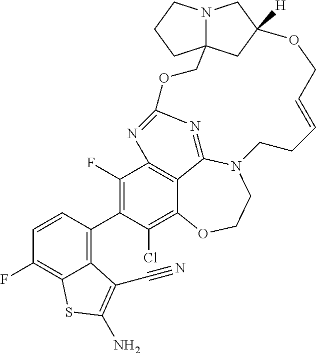 [SMILES: N#Cc1c(N)sc2c(F)ccc(-c3c(Cl)c4c5c6nc(nc5c3F)OCC35CCCN3C[C@@H](C5)OC/C=C/CCN6CCO4)c12] | 2-amino-4-((15R,E)-4-chloro-2-fluoro- 6,7,10,13,15,16,19,20-octahydro-9H,18H,21H- 1,23-(azeno)-15,20a- methanobenzo[6,7][1,4]oxazepino[5,4- d]pyrrolo[1,2- n][1,11]dioxa[3,5,14]triazacyclohexadecin-3-yl)-7- fluorobenzo[b]thiophene-3-carbonitrile (diastereomer 4) | 651.2 |
| 145^{†} | 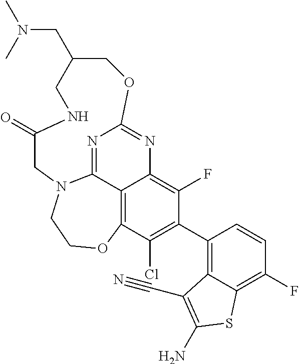 [SMILES: CN(C)CC1CNC(=O)CN2CCOc3c(Cl)c(-c4ccc(F)c5sc(N)c(C#N)c45)c(F)c4nc(nc2c34)OC1] | 2-amino-4-(14-chloro-5-((dimethylamino)methyl)- 16-fluoro-8-oxo-4,5,6,7,8,9,11,12-octahydro-2,17- (azeno)benzo[6,7][1,4]oxazepino[5,4- d][1]oxa[3,5,8]triazacycloundecin-15-yl)-7- fluorobenzo[b]thiophene-3-carbonitrile (diastereomer mixture 2) | 600.1 |
| 146 | 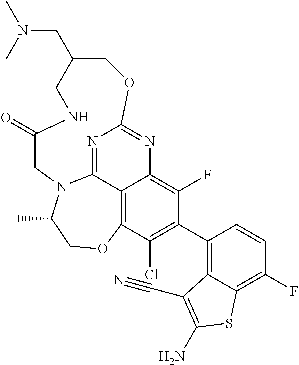 [SMILES: C[C@H]1COc2c(Cl)c(-c3ccc(F)c4sc(N)c(C#N)c34)c(F)c3nc4nc(c23)N1CC(=O)NCC(CN(C)C)CO4] | 2-amino-4-((11S)-14-chloro-5- ((dimethylamino)methyl)-16-fluoro-11-methyl-8- oxo-4,5,6,7,8,9,11,12-octahydro-2,17- (azeno)benzo[6,7][1,4]oxazepino[5,4- d][1]oxa[3,5,8]triazacycloundecin-15-yl)-7- fluorobenzo[b]thiophene-3-carbonitrile (diastereomer mixture 1) | 614.3 |
| 147^{†} | 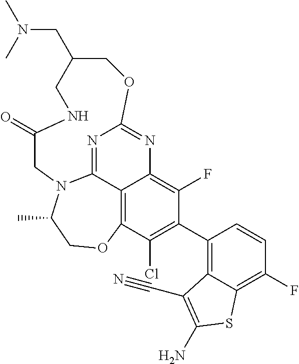 [SMILES: C[C@H]1COc2c(Cl)c(-c3ccc(F)c4sc(N)c(C#N)c34)c(F)c3nc4nc(c23)N1CC(=O)NCC(CN(C)C)CO4] | 2-amino-4-(14-chloro-5-((dimethylamino)methyl)- 16-fluoro-8-oxo-4,5,6,7,8,9,11,12-octahydro-2,17- (azeno)benzo[6,7][1,4]oxazepino[5,4- d][1]oxa[3,5,8]triazacycloundecin-15-yl)-7- fluorobenzo[b]thiophene-3-carbonitrile (diastereomer mixture 1) | 600.1 |
| 148 | 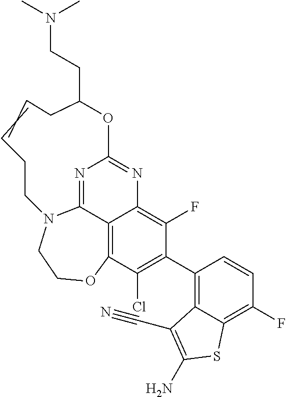 [SMILES: CN(C)CCC1CC=CCCN2CCOc3c(Cl)c(-c4ccc(F)c5sc(N)c(C#N)c45)c(F)c4nc(nc2c34)O1] | 2-amino-4-(14-chloro-4-(2-(dimethylamino)ethyl)- 16-fluoro-4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 597.1 |
| 149 | 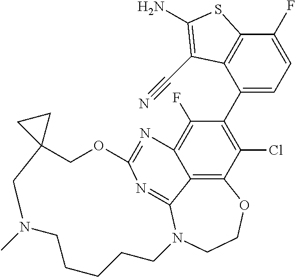 [SMILES: CN1CCCCCN2CCOc3c(Cl)c(-c4ccc(F)c5sc(N)c(C#N)c45)c(F)c4nc(nc2c34)OCC2(CC2)C1] | 2-amino-4-(17′-chloro-19′-fluoro-7′-methyl- 7′,8′,9′,10′,11′,12′,14′,15′-octahydro-4′H,6′H- spiro[cyclopropane-1,5′- [2,20](azeno)benzo[6,7][1,4]oxazepino[5,4- d][1]oxa[3,5,11]triazacyclotetradecin]-18′-yl)-7- fluorobenzo[b]thiophene-3-carbonitrile | 610.8 |
| 150 | 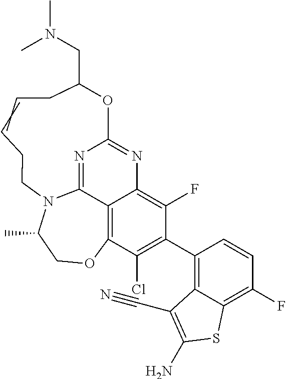 [SMILES: C[C@H]1COc2c(Cl)c(-c3ccc(F)c4sc(N)c(C#N)c34)c(F)c3nc4nc(c23)N1CCC=CCC(CN(C)C)O4] | 2-amino-4-((11S)-14-chloro-4- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 4) | 597.0 |
| 151 | 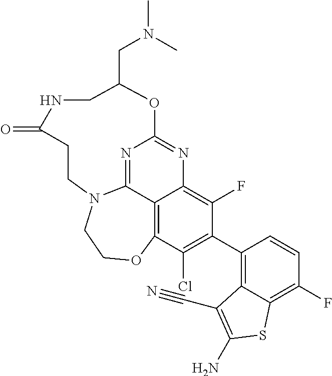 [SMILES: CN(C)CC1CNC(=O)CCN2CCOc3c(Cl)c(-c4ccc(F)c5sc(N)c(C#N)c45)c(F)c4nc(nc2c34)O1] | 2-amino-4-(14-chloro-4-((dimethylamino)methyl)- 16-fluoro-7-oxo-4,5,6,7,8,9,11,12-octahydro-2,17- (azeno)benzo[6,7][1,4]oxazepino[5,4- d][1]oxa[3,5,9]triazacycloundecin-15-yl)-7- fluorobenzo[b]thiophene-3-carbonitrile (diastereomer mixture 2) | 600.1 |
| 152 | 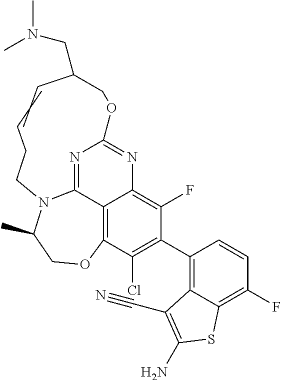 [SMILES: C[C@@H]1COc2c(Cl)c(-c3ccc(F)c4sc(N)c(C#N)c34)c(F)c3nc4nc(c23)N1CCC=CC(CN(C)C)CO4] | 2-amino-4-((11R)-15-chloro-5- ((dimethylamino)methyl)-17-fluoro-11-methyl- 4,5,8,9,12,13-hexahydro-11H-2,18- (azeno)benzo[2,3][1,5]oxazocino[4,5- d][1]oxa[3,5]diazacycloundecin-16-yl)-7- fluorobenzo[b]thiophene-3-carbonitrile (diastereomer mixture 2) | 611.1 |
| 153 | 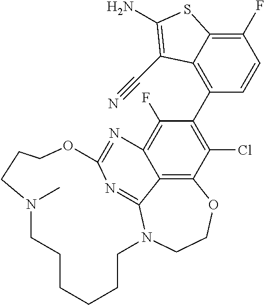 [SMILES: CN1CCCCCCN2CCOc3c(Cl)c(-c4ccc(F)c5sc(N)c(C#N)c45)c(F)c4nc(nc2c34)OCCC1] | 2-amino-4-(18-chloro-20-fluoro-7-methyl- 4,5,6,7,8,9,10,11,12,13,15,16-dodecahydro-2,21- (azeno)benzo[6,7][1,4]oxazepino[5,4- d][1]oxa[3,5,12]triazacyclopentadecin-19-yl)-7- fluorobenzo[b]thiophene-3-carbonitrile | 598.5 |
| 154 | 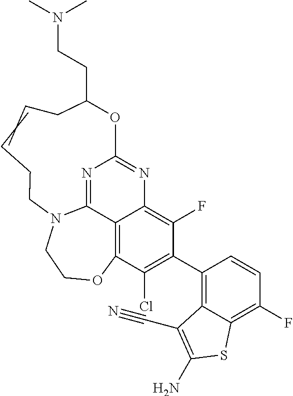 [SMILES: CN(C)CCC1CC=CCCN2CCOc3c(Cl)c(-c4ccc(F)c5sc(N)c(C#N)c45)c(F)c4nc(nc2c34)O1] | 2-amino-4-(14-chloro-4-(2-(dimethylamino)ethyl)- 16-fluoro-4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 597.1 |
| 155 | 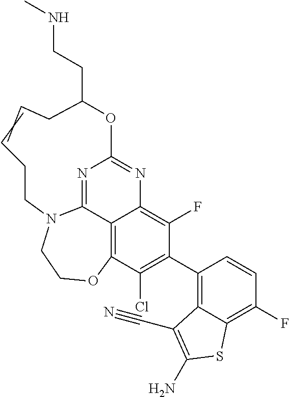 [SMILES: CNCCC1CC=CCCN2CCOc3c(Cl)c(-c4ccc(F)c5sc(N)c(C#N)c45)c(F)c4nc(nc2c34)O1] | 2-amino-4-(14-chloro-16-fluoro-4-(2- (methylamino)ethyl)-4,5,8,9,11,12-hexahydro- 2,17-(azeno)benzo[6,7][1,4]oxazepino[5,4- d][1]oxa[3,5]diazacycloundecin-15-yl)-7- fluorobenzo[b]thiophene-3-carbonitrile (diastereomer mixture 1) | 583.1 |
| 156 | 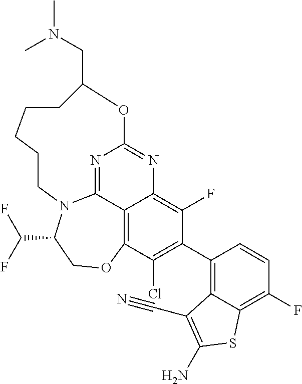 [SMILES: CN(C)CC1CCCCCN2c3nc(nc4c(F)c(-c5ccc(F)c6sc(N)c(C#N)c56)c(Cl)c(c34)OC[C@@H]2C(F)F)O1] | 2-amino-4-((11R)-14-chloro-11-(difluoromethyl)- 4-((dimethylamino)methyl)-16-fluoro- 4,5,6,7,8,9,11,12-octahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 2) | 634.9 |
| 157 | 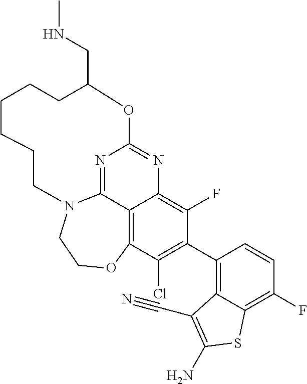 [SMILES: CNCC1CCCCCCN2CCOc3c(Cl)c(-c4ccc(F)c5sc(N)c(C#N)c45)c(F)c4nc(nc2c34)O1] | 2-amino-4-(15-chloro-17-fluoro-4- ((methylamino)methyl)-5,6,7,8,9,10,12,13- octahydro-4H-2,18- (azeno)benzo[6,7][1,4]oxazepino[5,4- d][1]oxa[3,5]diazacyclododecin-16-yl)-7- fluorobenzo[b]thiophene-3-carbonitrile | 584.9 |
| 158 | 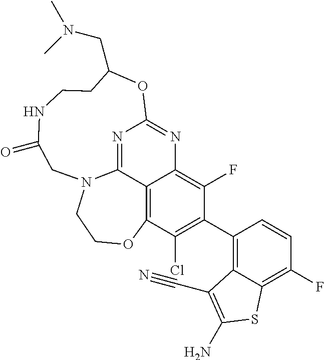 [SMILES: CN(C)CC1CCNC(=O)CN2CCOc3c(Cl)c(-c4ccc(F)c5sc(N)c(C#N)c45)c(F)c4nc(nc2c34)O1] | 2-amino-4-(14-chloro-4-((dimethylamino)methyl)- 16-fluoro-8-oxo-4,5,6,7,8,9,11,12-octahydro-2,17- (azeno)benzo[6,7][1,4]oxazepino[5,4- d][1]oxa[3,5,8]triazacycloundecin-15-yl)-7- fluorobenzo[b]thiophene-3-carbonitrile | 600.1 |
| 159 | 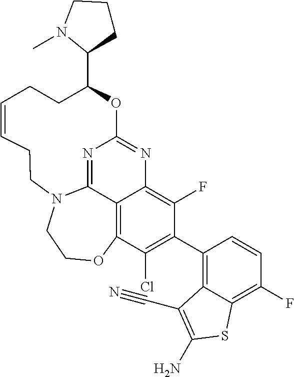 [SMILES: CN1CCC[C@H]1[C@@H]1CC/C=C\CCN2CCOc3c(Cl)c(-c4ccc(F)c5sc(N)c(C#N)c45)c(F)c4nc(nc2c34)O1] | 2-amino-4-((4S,Z)-15-chloro-17-fluoro-4-((S)-1- methylpyrrolidin-2-yl)-5,6,9,10,12,13-hexahydro- 4H-2,18-(azeno)benzo[6,7][1,4]oxazepino[5,4- d][1]oxa[3,5]diazacyclododecin-16-yl)-7- fluorobenzo[b]thiophene-3-carbonitrile | 623.3 |
| 160 | 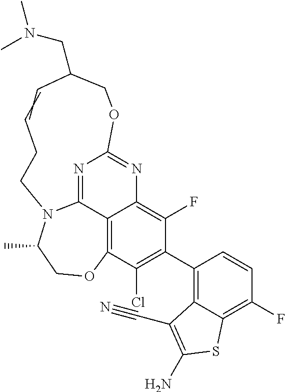 [SMILES: C[C@H]1COc2c(Cl)c(-c3ccc(F)c4sc(N)c(C#N)c34)c(F)c3nc4nc(c23)N1CCC=CC(CN(C)C)CO4] | 2-amino-4-((11S)-14-chloro-5- ((dimethylamino)methyl)-16-fluoro-11-methyl- 4,5,8,9,11,12-hexahydro-2,17- (azeno)benzo[6,7][1,4]oxazepino[5,4- d][1]oxa[3,5]diazacycloundecin-15-yl)-7- fluorobenzo[b]thiophene-3-carbonitrile (diastereomer mixture 3) | 596.8 |

Compounds of Table 1 are depicted with flat, wedged, and/or hashed wedged bonds. It is understood that compounds depicted in Table 1 encompass all possible stereoisomers, including atropisomers, of the compounds of Table 1. In some instances, the relative stereochemistry at one or more stereocenters of a compound has been determined; in some instances, the absolute stereochemistry has been determined. In some instances, a single compound number represents a mixture of stereoisomers, including atropisomers.
In some instances, a single compound number represents a single stereoisomer, such as a single atropisomer. As such, it is understood that if two or more compound numbers in Table 1 are provided with the same depicted structure, then different stereoisomers or mixtures of stereoisomers of the depicted structure are represented by each compound number.
^{†}Compound provided as a substantially pure single atropisomer.

In some embodiments, the compounds of the present disclosure exhibit one or more functional characteristics disclosed herein. For example, a subject compound binds to a Ras protein, Kras protein or a mutant form thereof. In some embodiments, a subject compound binds specifically and also inhibits a Ras protein, Kras protein or a mutant form thereof. In some embodiments, a subject compound selectively inhibits a Kras mutant relative to a wildtype Kras. In some embodiments, a subject compound selectively inhibits KrasG12D) and/or KrasG12V relative to wildtype Kras. In some embodiments, the IC50 of a subject compound for a Kras mutant (e.g., including G12D) is less than about 5 μM, less than about 1 μM, less than about 50 nM, less than about 10 nM, less than about 1 nM, less than about 0.5 nM, less than about 100 pM, or less than about 50 pM, as measured in an in vitro assay known in the art or exemplified herein. In some embodiments, a subject compound covalently binds to a Kras mutant (e.g., KrasG12D, KrasG12C, KrasG12S, and/or G13D).

In some embodiments, a compound of the present disclosure is capable of reducing Ras signaling output. Such reduction may be evidenced by one or more of the following: (i) an increase in steady state level of GDP-bound Ras protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, and (vi) reduction (e.g., inhibition) of cell growth of Ras-driven tumor cells (e.g., those derived from a tumor cell line disclosed herein). In some cases, the reduction in Ras signaling output can be evidenced by two, three, four, five, or all of (i)-(vi) above.

It shall be understood that different aspects of the disclosure can be appreciated individually, collectively, or in combination with each other. Various aspects described herein may be applied to any of the particular applications disclosed herein. The compositions of matter, including compounds of any formulae disclosed in the compound section, of the present disclosure may be utilized in the method section, including methods of use and production disclosed herein, or vice versa.

### Methods

The compounds described herein, or a pharmaceutically acceptable salt or solvate thereof, are Ras inhibitors capable of inhibiting a Ras protein. Ras proteins being inhibited can be Ras mutants (e.g., G12C, G12D, G12S, G12V, G13C, or G13D) from K-Ras, H-Ras or N-Ras. Compounds, including pharmaceutically acceptable salts or solvates thereof, disclosed herein have a wide range of applications in therapeutics, diagnostics, and other biomedical research.

In certain aspects, the present disclosure provides a method of treating cancer in a subject in need thereof, comprising administering to the subject a therapeutically effective amount of a compound described herein, or a pharmaceutically acceptable salt or solvate thereof.

In certain aspects, the present disclosure provides a method of treating cancer in a subject comprising a Ras mutant (e.g., G12C, G12D, G12S, G12V, G13C, or G13D) protein, comprising inhibiting the Ras mutant (e.g., G12C, G12D, G12S, G12V, G13C, or G13D) protein of said subject by administering to said subject a compound, wherein the compound is characterized in that upon contacting the Ras mutant (e.g., G12C, G12D, G12S, G12V, G13C, or G13D) protein, the Ras mutant (e.g., G12C, G12D, G12S, G12V, G13C, or G13D) protein activity or function is inhibited (e.g., partially inhibited or completely inhibited), such that said inhibited Ras mutant (e.g., G12C, G12D, G12S, G12V, G13C, or G13D) protein exhibits reduced Ras signaling output (e.g., compared to a corresponding Ras protein not contacted by the compound).

In certain aspects, the present disclosure provides a method of modulating activity of a Ras protein (e.g., K-Ras, mutant K-Ras, G12C, G12D, G12S, G12V, G13C, or G13D), comprising contacting a Ras protein with an effective amount of a compound described herein, or a pharmaceutically acceptable salt or solvate thereof, thereby modulating the activity of the Ras protein.

In certain aspects, the present disclosure provides a method of inhibiting cell growth, comprising administering an effective amount of a compound described herein, or a pharmaceutically acceptable salt or solvate thereof, to a cell expressing a Ras (e.g., K-Ras) protein, thereby inhibiting growth of said cells. In some embodiments, the subject method comprises administering an additional agent to said cell.

In certain aspects, the present disclosure provides a method of treating a disease mediated at least in part by a Ras protein, such as K-Ras or a mutant thereof, in a subject in need thereof, comprising administering to the subject an effective amount of a compound disclosed herein, or a pharmaceutically acceptable salt or solvate thereof. In some embodiments, the disease is cancer, such as a solid tumor or a hematological cancer. In some embodiments, the method further comprises administering an additional agent to the subject, such as a SHP2 inhibitor, a SOS inhibitor, an EGFR inhibitor, a MEK inhibitor, an ERK inhibitor, a CDK4/6 inhibitor, a BRAF inhibitor, or a combination thereof.

In certain aspects, the present disclosure provides a method of inhibiting activity of a Ras protein, such as K-Ras or a mutant thereof, comprising contacting the Ras protein with a compound disclosed herein, or a pharmaceutically acceptable salt or solvate thereof. In some embodiments, the compound exhibits an IC50 against the Ras protein of less than 10 μM, such as less than 5 μM, 1 μM, 500 nM, 100 nM, 50 nM, 10 nM, 5 nM, 1 nM, 500 pM, 50 pM, 10 pM or less.

In certain aspects, the present disclosure provides a method of treating a Ras-mediated cancer in a subject in need thereof, comprising administering to the subject a SHP2 inhibitor, a SOS inhibitor, an EGFR inhibitor, a MEK inhibitor, an ERK inhibitor, a CDK4/6 inhibitor, or a BRAF inhibitor and an effective amount of a compound disclosed herein, such as a compound of Formula (I), or a pharmaceutically acceptable salt or solvate thereof.

In some embodiments, the cancer is a solid tumor. In some embodiments, the cancer is a hematological cancer.

In practicing any of the methods disclosed herein, the Ras target to which a subject compound binds, either covalently or reversibly, can be a Ras mutant (e.g., G12C, G12D, G12S, G12V, G13C, or G13D), including a mutant of K-Ras, H-Ras, or N-Ras. In some embodiments, the methods of treating cancer can be applied to treat a solid tumor or a hematological cancer. In some embodiments, the cancer being treated can be, without limitation, prostate cancer, brain cancer, colon cancer, rectal cancer, renal-cell carcinoma, liver cancer, non-small cell carcinoma of the lung, cancer of the small intestine, cancer of the esophagus, melanoma, bone cancer, pancreatic cancer, skin cancer, cancer of the head or neck, cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), primary CNS lymphoma, tumor angiogenesis, spinal axis tumor, brain stem glioma, pituitary adenoma, Kaposi's sarcoma, epidermoid cancer, squamous cell cancer, T-cell lymphoma, environmentally induced cancers, combinations of said cancers, and metastatic lesions of said cancers. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer. In some embodiments is a method of treating cancer in a subject in need thereof, comprising administering to the subject a therapeutically effective amount of a compound described herein, or a pharmaceutically acceptable salt or solvate thereof, wherein the cancer is a hematological cancer selected from one or more of chronic lymphocytic leukemia (CLL), acute leukemias, acute lymphoid leukemia (ALL), B-cell acute lymphoid leukemia (B-ALL), T-cell acute lymphoid leukemia (T-ALL), chronic myelogenous leukemia (CML), B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, marginal zone lymphoma, multiple myeloma, myelodysplasia and myelodysplastic syndrome, non-Hodgkin's lymphoma, Hodgkin's lymphoma, plasmablastic lymphoma, plasmacytoid dendritic cell neoplasm, Waldenstrom macroglobulinemia, and pre-leukemia. In some embodiments is a method of treating cancer in a subject in need thereof, comprising administering to the subject a therapeutically effective amount of a compound described herein, or a pharmaceutically acceptable salt or solvate thereof, wherein the cancer is one or more cancers selected from the group consisting of chronic lymphocytic leukemia (CLL), acute myeloid leukemia (AML), T-cell acute lymphoblastic leukemia (T-ALL), B cell acute lymphoblastic leukemia (B-ALL), and/or acute lymphoblastic leukemia (ALL).

Any of the treatment methods disclosed herein can be administered alone or in combination or in conjunction with another therapy or another agent. By “combination” it is meant to include (a) formulating a subject composition containing a subject compound together with another agent, or (b) using the subject composition separate from the another agent as an overall treatment regimen. By “conjunction” it is meant that the another therapy or agent is administered either simultaneously, concurrently or sequentially with a subject composition comprising a compound disclosed herein, with no specific time limits, wherein such conjunctive administration provides a therapeutic effect.

In some embodiments, a subject treatment method is combined with surgery, cellular therapy, chemotherapy, radiation, and/or immunosuppressive agents. Additionally, compositions of the present disclosure can be combined with other therapeutic agents, such as other anti-cancer agents, anti-allergic agents, anti-nausea agents (or anti-emetics), pain relievers, cytoprotective agents, immunostimulants, and combinations thereof. In one embodiment, a subject treatment method is combined with a chemotherapeutic agent.

Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). Additional chemotherapeutic agents contemplated for use in combination include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®), anastrozole (Arimidex®), bicalutamide (Casodex®), bleomycin sulfate (Blenoxane®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), dacarbazine (DTIC-Dome®), dactinomycin (Actinomycin D, Cosmegan), dexamethasone, docetaxel (Taxotere®), 5-fluorouracil (Adrucil®, Efudex®), flutamide (Eulexin®), tezacitibine, Gemcitabine (difluorodeoxycitidine), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/M4X-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Anti-cancer agents of particular interest for combinations with a compound of the present disclosure include: anthracyclines; alkylating agents; antimetabolites; drugs that inhibit either the calcium dependent phosphatase calcineurin or the p70S6 kinase FK506 or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors: methotrexate (Rheumatrex®, Trexall®), 5-fluorouracil (Adrucil®, Efudex®, Fluoroplex®), floxuridine (FUDF®), cytarabine (Cytosar-U®, Tarabine PFS), 6-mercaptopurine (Puri-Nethol®)), 6-thioguanine (Thioguanine Tabloid®), fludarabine phosphate (Fludara®), pentostatin (Nipent®), pemetrexed (Alimta®), raltitrexed (Tomudex®), cladribine (Leustatin®), clofarabine (Clofarex®, Clolar®), azacitidine (Vidaza®), decitabine and gemcitabine (Gemzar®). Preferred antimetabolites include, cytarabine, clofarabine and fludarabine.

Exemplary alkylating agents include, without limitation, nitrogen mustards, ethylenimine derivatives, alkyl sulfonates, nitrosoureas and triazenes: uracil mustard (Aminouracil Mustard®, Chlorethaminacil®, Demethyldopan®, Desmethyldopan®, Haemanthamine®, Nordopan®, Uracil nitrogen Mustard®, Uracillost®, Uracilnostaza®, Uramustin®, Uramustine®), chlormethine (Mustargen®), cyclophosphamide (Cytoxan®, Neosar®, Clafen®, Endoxan®, Procytox®, Revimmune™), ifosfamide (Mitoxana®), melphalan (Alkeran®), Chlorambucil (Leukeran®), pipobroman (Amedel®, Vercyte®), triethylenemelamine (Hemel®, Hexalen®, Hexastat®), triethylenethiophosphoramine, Temozolomide (Temodar®), thiotepa (Thioplex®), busulfan (Busilvex®, Myleran®), carmustine (BiCNU®), lomustine (CeeNU®), streptozocin (Zanosar®), and Dacarbazine (DTIC-Dome®). Additional exemplary alkylating agents include, without limitation, Oxaliplatin (Eloxatin®); Temozolomide (Temodar® and Temodal®); Dactinomycin (also known as actinomycin-D, Cosmegen®); Melphalan (also known as L-PAM, L-sarcolysin, and phenylalanine mustard, Alkeran®); Altretamine (also known as hexamethylmelamine (HMM), Hexalen®); Carmustine (BiCNU®); Bendamustine (Treanda®); Busulfan (Busulfex® and Myleran®); Carboplatin (Paraplatin®); Lomustine (also known as CCNU, CeeNU®); Cisplatin (also known as CDDP, Platinol® and Platinol®-AQ); Chlorambucil (Leukeran®); Cyclophosphamide (Cytoxan® and Neosar®); Dacarbazine (also known as DTIC, DIC and imidazole carboxamide, DTIC-Dome®); Altretamine (also known as hexamethylmelamine (HMM), Hexalen®); Ifosfamide (Ifex®); Prednumustine; Procarbazine (Matulane®); Mechlorethamine (also known as nitrogen mustard, mustine and mechloroethamine hydrochloride, Mustargen®); Streptozocin (Zanosar®); Thiotepa (also known as thiophosphoamide, TESPA and TSPA, Thioplex®); Cyclophosphamide (Endoxan®, Cytoxan®, Neosar®, Procytox®, Revimmune®); and Bendamustine HCl (Treanda®).

In certain aspects, compositions provided herein can be administered in combination with radiotherapy, such as radiation. Whole body radiation may be administered at 12 Gy. A radiation dose may comprise a cumulative dose of 12 Gy to the whole body, including healthy tissues. A radiation dose may comprise from 5 Gy to 20 Gy. A radiation dose may be 5 Gy, 6 Gy, 7 Gy, 8 Gy, 9 Gy, 10 Gy, 11 Gy, 12, Gy, 13 Gy, 14 Gy, 15 Gy, 16 Gy, 17 Gy, 18 Gy, 19 Gy, or up to 20 Gy. Radiation may be whole body radiation or partial body radiation. In the case that radiation is whole body radiation it may be uniform or not uniform. For example, when radiation may not be uniform, narrower regions of a body such as the neck may receive a higher dose than broader regions such as the hips.

Where desirable, an immunosuppressive agent can be used in conjunction with a subject treatment method. Exemplary immunosuppressive agents include but are not limited to cyclosporin, azathioprine, methotrexate, mycophenolate, and FK506, antibodies, or other immunoablative agents such as CAMPATH, anti-CD3 antibodies (e.g., muromonab, otelixizumab) or other antibody therapies, cytoxin, fludarabine, cyclosporin, FK506, rapamycin, mycophenolic acid, steroids, FR901228, cytokines, and irradiation, peptide vaccine, and any combination thereof. In accordance with the presently disclosed subject matter, the above-described various methods can comprise administering at least one immunomodulatory agent. In certain embodiments, the at least one immunomodulatory agent is selected from the group consisting of immunostimulatory agents, checkpoint immune blockade agents (e.g., blockade agents or inhibitors of immune checkpoint genes, such as, for example, PD-1, PD-L1, CTLA-4, IDO, TIM3, LAG3, TIGIT, BTLA, VISTA, ICOS, KIRs and CD39), radiation therapy agents, chemotherapy agents, and combinations thereof. In some embodiments, the immunostimulatory agents are selected from the group consisting of IL-12, an agonist costimulatory monoclonal antibody, and combinations thereof. In one embodiment, the immunostimulatory agent is IL-12. In some embodiments, the agonist costimulatory monoclonal antibody is selected from the group consisting of an anti-4-11BB antibody (e.g., urelumab, PF-05082566), an anti-OX40 antibody (pogalizumab, tavolixizumab, PF-04518600), an anti-ICOS antibody (BMS986226, MEDI-570, GSK3359609, JTX-2011), and combinations thereof. In one embodiment, the agonist costimulatory monoclonal antibody is an anti-4-1 BB antibody. In some embodiments, the checkpoint immune blockade agents are selected from the group consisting of anti-PD-L1 antibodies (atezolizumab, avelumab, durvalumab, BMS-936559), anti-CTLA-4 antibodies (e.g., tremelimumab, ipilimumab), anti-PD-1 antibodies (e.g., pembrolizumab, nivolumab), anti-LAG3 antibodies (e.g., C9B7W, 410C9), anti-B7-113 antibodies (e.g., DS-5573a), anti-TIM3 antibodies (e.g., F38-2E2), and combinations thereof. In one embodiment, the checkpoint immune blockade agent is an anti-PD-L1 antibody. In some cases, a compound of the present disclosure can be administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In some cases, expanded cells can be administered before or following surgery. Alternatively, compositions comprising a compound described herein can be administered with immunostimulants. Immunostimulants can be vaccines, colony stimulating agents, interferons, interleukins, viruses, antigens, co-stimulatory agents, immunogenicity agents, immunomodulators, or immunotherapeutic agents. An immunostimulant can be a cytokine such as an interleukin. One or more cytokines can be introduced with modified cells provided herein. Cytokines can be utilized to boost function of modified T lymphocytes (including adoptively transferred tumor-specific cytotoxic T lymphocytes) to expand within a tumor microenvironment. In some cases, IL-2 can be used to facilitate expansion of the modified cells described herein. Cytokines such as IL-15 can also be employed. Other relevant cytokines in the field of immunotherapy can also be utilized, such as IL-2, IL-7, IL-12, IL-15, IL-21, or any combination thereof. An interleukin can be IL-2, or aldesleukin. Aldesleukin can be administered in low dose or high dose. A high dose aldesleukin regimen can involve administering aldesleukin intravenously every 8 hours, as tolerated, for up to about 14 doses at about 0.037 mg/kg (600,000 IU/kg). An immunostimulant (e.g., aldesleukin) can be administered within 24 hours after a cellular administration. An immunostimulant (e.g., aldesleukin) can be administered in as an infusion over about 15 minutes about every 8 hours for up to about 4 days after a cellular infusion. An immunostimulant (e.g., aldesleukin) can be administered at a dose from about 100,000 IU/kg, 200,000 IU/kg, 300,000 IU/kg, 400,000 IU/kg, 500,000 IU/kg, 600,000 IU/kg, 700,000 IU/kg, 800,000 IU/kg, 900,000 IU/kg, or up to about 1,000,000 IU/kg. In some cases, aldesleukin can be administered at a dose from about 100,000 IU/kg to 300,000 IU/kg, from 300,000 IU/kg to 500,000 IU/kg, from 500,000 IU/kg to 700,000 IU/kg, from 700,000 IU/kg to about 1,000,000 IU/kg.

In some embodiments, a compound described herein, or a pharmaceutically acceptable salt or solvate thereof, is administered in combination or in conjunction with one or more pharmacologically active agents selected from (1) an inhibitor of MEK (e.g., MEK1, MEK2) or of mutants thereof (e.g., trametinib, cobimetinib, binimetinib, selumetinib, refametinib); (2) an inhibitor of epidermal growth factor receptor (EGFR) and/or of mutants thereof (e.g., afatinib, erlotinib, gefitinib, lapatinib, cetuximab panitumumab, osimertinib, olmutinib, EGF-816); (3) an immunotherapeutic agent (e.g., checkpoint immune blockade agents, as disclosed herein); (4) a taxane (e.g., paclitaxel, docetaxel); (5) an anti-metabolite (e.g. antifolates such as methotrexate, raltitrexed, pyrimidine analogues such as 5-fluorouracil (5-FU), ribonucleoside and deoxyribonucleoside analogues, capecitabine and gemcitabine, purine and adenosine analogues such as mercaptopurine, thioguanine, cladribine and pentostatin, cytarabine (ara C), fludarabine); (6) an inhibitor of FGFR1 and/or FGFR2 and/or FGFR3 and/or of mutants thereof (e.g., nintedanib); (7) a mitotic kinase inhibitor (e.g., a CDK4/6 inhibitor, such as, for example, palbociclib, ribociclib, abemaciclib); (8) an anti-angiogenic drug (e.g., an anti-VEGF antibody, such as, for example, bevacizumab); (9) a topoisomerase inhibitor (e.g. epipodophyllotoxins such as for example etoposide and etopophos, teniposide, amsacrin, topotecan, irinotecan, mitoxantrone); (10) a platinum-containing compound (e.g. cisplatin, oxaliplatin, carboplatin); (11) an inhibitor of ALK and/or of mutants thereof (e.g. crizotinib, alectinib, entrectinib, brigatinib); (12) an inhibitor of c-MET and/or of mutants thereof (e.g., K252a, SU11274, PHA665752, PF2341066); (13) an inhibitor of BCR-ABL and/or of mutants thereof (e.g., imatinib, dasatinib, nilotinib); (14) an inhibitor of ErbB2 (Her2) and/or of mutants thereof (e.g., afatinib, lapatinib, trastuzumab, pertuzumab); (15) an inhibitor of AXL and/or of mutants thereof (e.g., R428, amuvatinib, XL-880); (16) an inhibitor of NTRK1 and/or of mutants thereof (e.g., Merestinib); (17) an inhibitor of RET and/or of mutants thereof (e.g., BLU-667, Lenvatinib); (18) an inhibitor of A-Raf and/or B-Raf and/or C-Raf and/or of mutants thereof (RAF-709, LY-3009120); (19) an inhibitor of ERK and/or of mutants thereof (e.g., ulixertinib); (20) an MDM2 inhibitor (e.g., HDM-201, NVP-CGM097, RG-71 12, MK-8242, RG-7388, SAR405838, AMG-232, DS-3032, RG-7775, APG-115); (21) an inhibitor of mTOR (e.g., rapamycin, temsirolimus, everolimus, ridaforolimus); (22) an inhibitor of BET (e.g., I-BET 151, I-BET 762, OTX-015, TEN-010, CPI-203, CPI-0610, olionon, RVX-208, ABBC-744, LY294002, AZD5153, MT-1, MS645); (23) an inhibitor of IGF1/2 and/or of IGF1-R (e.g., xentuzumab, MEDI-573); (24) an inhibitor of CDK9 (e.g., DRB, flavopiridol, CR8, AZD 5438, purvalanol B, AT7519, dinaciclib, SNS-032); (25) an inhibitor of farnesyl transferase (e.g., tipifarnib); (26) an inhibitor of SHIP pathway including SHIP2 inhibitor, as well as SHIP1 inhibitors; (27) an inhibitor of SRC (e.g., dasatinib); (28) an inhibitor of JAK (e.g. tofacitinib); (29) a PARP inhibitor (e.g. Olaparib, Rucaparib, Niraparib, Talazoparib), (30) a BTK inhibitor (e.g. Ibrutinib, Acalabrutinib, Zanubrutinib), (31) a ROS1 inhibitor (e.g., entrectinib), (32) an inhibitor of SHP pathway including SHP2 inhibitor (e.g., 6-(4-amino-4-methylpiperidin-1-yl)-3-(2,3-dichlorophenyl)pyrazin-2-amine, as well as SHP1 inhibitors, or (33) an inhibitor of Src, FLT3, HDAC, VEGFR, PDGFR, LCK, Bcr-Abl or AKT or (34) an inhibitor of KrasG12C mutant (e.g., including but not limited to AMG510, MRTX849, and any covalent inhibitors binding to the cysteine residue 12 of Kras, the structures of which are publicly known) (e.g., an inhibitor of Ras G12C as described in US20180334454, US20190144444, US20150239900, U.S. Ser. No. 10/246,424, US20180086753, WO2018143315, WO2018206539, WO20191107519, WO2019141250, WO2019150305, U.S. Pat. No. 9,862,701, US20170197945, US20180086753, U.S. Ser. No. 10/144,724, US20190055211, US20190092767, US20180127396, US20180273523, U.S. Ser. No. 10/280,172, US20180319775, US20180273515, US20180282307, US20180282308, WO2019051291, WO2019213526, WO2019213516, WO2019217691, WO2019241157, WO2019217307, WO2020047192, WO2017087528, WO2018218070, WO2018218069, WO2018218071, WO2020027083, WO2020027084, WO2019215203, WO2019155399, WO2020035031, WO2014160200, WO2018195349, WO2018112240, WO2019204442, WO2019204449, WO2019104505, WO2016179558, WO2016176338, or related patents and applications, each of which is incorporated by reference in its entirety), (35) an SHC inhibitor (e.g., PP2, AID371185), (36) a GAB inhibitor (e.g., GAB-0001), (37) a GRB inhibitor, (38) a PI-3 kinase inhibitor (e.g., Idelalisib, Copanlisib, Duvelisib, Alpelisib, Taselisib, Perifosine, Buparlisib, Umbralisib, NVP-BEZ235-AN), (39) a MARPK inhibitor, (40) CDK4/6 (e.g., palbociclib, ribociclib, abemaciclib), or (41) MAPK inhibitor (e.g., VX-745, VX-702, RO-4402257, SCIO-469, BIRB-796, SD-0006, PH-797804, AMG-548, LY2228820, SB-681323, GW-856553, RWJ67657, BCT-197), or (42) an inhibitor of SHP pathway including SHP2 inhibitor (e.g., 6-(4-amino-4-methylpiperidin-1-yl)-3-(2,3-dichlorophenyl)pyrazin-2-amine, RMC-4630, ERAS-601,

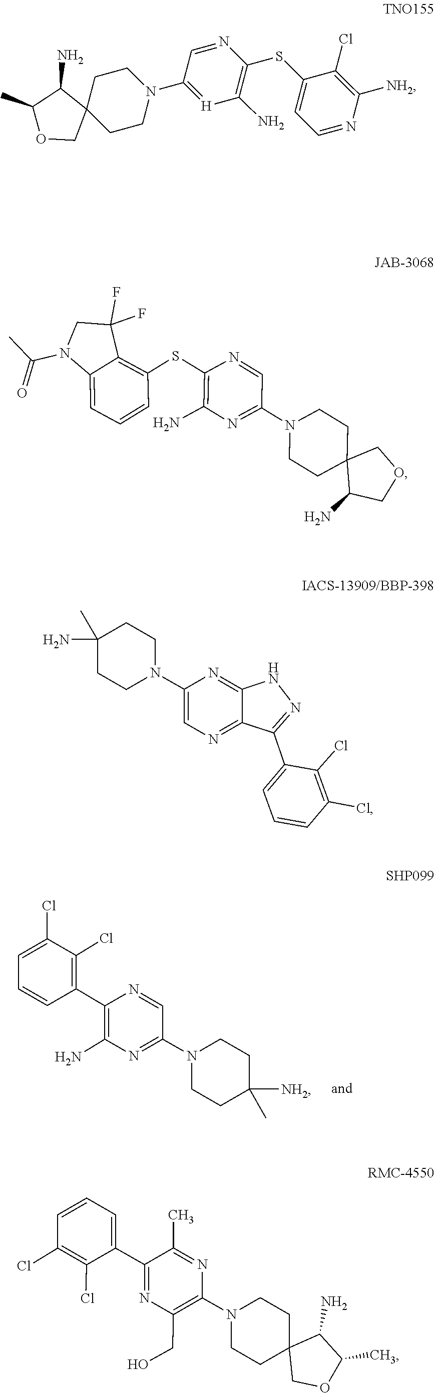

as well as SHP1 inhibitors. In some embodiments, a Ras inhibitor described herein, such as a compound, salt, or solvate of Formula (I), is administered in combination or in conjunction with one or more checkpoint immune blockade agents (e.g., anti-PD-1 and/or anti-PD-L1 antibody, anti-CLTA-4 antibody). In some embodiments, a Ras inhibitor described herein, such as a compound, salt, or solvate of Formula (I), is administered in combination or in conjunction with one or more pharmacologically active agents comprising an inhibitor against one or more targets selected from: MEK, epidermal growth factor receptor (EGFR), FGFR1, FGFR2, FGFR3, mitotic kinase, topoisomerase, ALK, ALK5, c-MET, ErbB2, AXL, NTRK1, RET, A-Raf, B-Raf, C-Raf, ERK, MDM2, mTOR, BET, IGF1/2, IGF1-R, CDK9, SHIP1, SHIP2, SHP2, SRC, JAK, PARP, BTK, FLT3, HDAC, VEGFR, PDGFR, LCK, Bcr-Abl, AKT, KrasG12C mutant, and ROS1. In some embodiments, any of the compounds herein that is capable of binding a Ras protein (e.g., KRAS, mutant Ras protein) to modulate activity of such Ras mutant (e.g., G12C, G12D, G12S, G12V, G13C, or G13D) may be administered in combination or in conjunction with one or more additional pharmacologically active agents comprising an inhibitor of SOS (e.g., SOS1, SOS2) or of mutants thereof. In some embodiments, the additional pharmacologically active agent administered in combination or in conjunction with a compound described herein (e.g., compound capable of binding a Ras protein) is an inhibitor of SOS (e.g., SOS1, SOS2). In some embodiments, the additional pharmacologically active agent administered in combination or in conjunction with a compound (e.g., compound capable of binding a Ras protein) described herein is an inhibitor of SOS (e.g., SOS1, SOS2). In some embodiments, the additional pharmacologically active agent administered in combination or in conjunction with a compound (e.g., compound capable of binding a Ras protein) described herein is an inhibitor of SOS (e.g., SOS1, SOS2) selected from RMC-5845, BI-1701963,

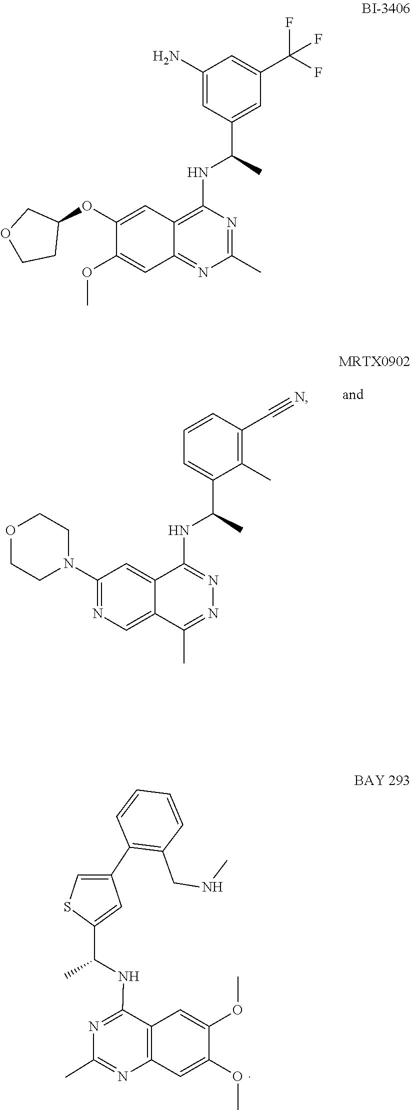 [SMILES: COc1cc2nc(C)nc(N[C@H](C)c3cc(N)cc(C(F)(F)F)c3)c2cc1O[C@H]1CCOC1 ; Cc1c(C#N)cccc1[C@@H](C)Nc1nnc(C)c2cnc(N3CCOCC3)cc12 ; CNCc1ccccc1-c1csc([C@@H](C)Nc2nc(C)nc3cc(OC)c(OC)cc23)c1]

In some embodiments, the additional pharmacologically active agent administered in combination or in conjunction with a compound described herein (e.g., compound capable of binding a Ras protein) is an inhibitor of SOS (e.g., SOS1, SOS2) described in WO2021092115, WO2018172250, WO2019201848, WO2019122129, WO2018115380, WO2021127429, WO2020180768, or WO2020180770, all of which are herein incorporated by reference in their entirety for all purposes.

In some embodiments, any of the compounds herein that is capable of binding a Ras protein (e.g., Kras) to modulate activity of such Ras protein may be administered in combination or in conjunction with one or more checkpoint immune blockade agents (e.g., anti-PD-1 and/or anti-PD-L1 antibody, anti-CLTA-4 antibody).

In some embodiments, a compound described herein, such as a compound, salt, or solvate of Formula (I), is administered in combination or in conjunction with one or more pharmacologically active agents comprising an inhibitor of: (1) SOS1 or a mutant thereof (e.g., RMC-5845, BI-3406, BAY-293, MRTX0902, BI-1701963); (2) SHP2 or a mutant thereof (e.g., 6-(4-amino-4-methylpiperidin-1-yl)-3-(2,3-dichlorophenyl)pyrazin-2-amine, TNO155, RMC-4630, ERAS-601, JAB-3068, IACS-13909/BBP-398, SHP099, RMC-4550); (3) SHC or a mutant thereof (e.g., PP2, AID371185); (4) GAB or a mutant thereof (e.g., GAB-0001); (5) GRB or a mutant thereof; (6) JAK or a mutant thereof (e.g., tofacitinib); (7) A-RAF, B-RAF, C-RAF, or a mutant thereof (e.g., RAF-709, LY-3009120); (8) BRAF or a mutant thereof (e.g., sorafenib, vemurafenib, dabrafenib, encorafenib, regorafenib, GDC-879); (9) MEK or a mutant thereof (e.g., trametinib, cobimetinib, binimetinib, selumetinib, refametinib, AZD6244); (10) ERK or a mutant thereof (e.g., ulixertinib, MK-8353, LTT462, AZD0364, SCH772984, BIX02189, LY3214996, ravoxertinib); (11) PI3K or a mutant thereof (e.g., idelalisib, copanlisib, duvelisib, alpelisib, taselisib, perifosine, buparlisib, umbralisib, NVP-BEZ235-AN); (12) MAPK or a mutant thereof (e.g., VX-745, VX-702, RO-4402257, SCIO-469, BIRB-796, SD-0006, PH-797804, AMG-548, LY2228820, SB-681323, GW-856553, RWJ67657, BCT-197); (13) EGFR or a mutant thereof (e.g., afatinib, erlotinib, gefitinib, lapatinib, cetuximab panitumumab, osimertinib, olmutinib, EGF-816); (14) c-MET or a mutant thereof (e.g., K252a, SU11274, PHA665752, PF2341066); (15) ALK or a mutant thereof (e.g. crizotinib, alectinib, entrectinib, brigatinib); (16) FGFR1, FGFR-2, FGFR-3, FGFR-4 or a mutant thereof (e.g., nintedanib); (17) BCR-ABL or a mutant thereof (e.g., imatinib, dasatinib, nilotinib); (18) ErbB2 (Her2) or a mutant thereof (e.g., afatinib, lapatinib, trastuzumab, pertuzumab); (19) AXL or a mutant thereof (e.g., R428, amuvatinib, XL-880); (20) NTRK1 or a mutant thereof (e.g., merestinib); (21) ROS1 or a mutant thereof (e.g., entrectinib); (22) RET or a mutant thereof (e.g., BLU-667, Lenvatinib); (23) MDM2 or a mutant thereof (e.g., HDM-201, NVP-CGM097, RG-71 12, MK-8242, RG-7388, SAR405838, AMG-232, DS-3032, RG-7775, APG-115); (24) mTOR or a mutant thereof (e.g., rapamycin, temsirolimus, everolimus, ridaforolimus); (25) BET or a mutant thereof (e.g., I-BET 151, I-BET 762, OTX-015, TEN-010, CPI-203, CPI-0610, olionon, RVX-208, ABBC-744, LY294002, AZD5153, MT-1, MS645); (26) IGF1, IGF2, IGF1R, or a mutant thereof (e.g., xentuzumab, MEDI-573); (27) CDK9 or a mutant thereof (e.g., DRB, flavopiridol, CR8, AZD 5438, purvalanol B, AT7519, dinaciclib, SNS-032); or (28) CDK4/6 (e.g., palbociclib, ribociclib, abemaciclib).

In combination therapy, a compound provided herein and other anti-cancer agent(s) may be administered either simultaneously, concurrently or sequentially with no specific time limits, wherein such administration provides therapeutically effective levels of the two compounds in the body of the patient.

In some embodiments, the compound of the present disclosure and the other anti-cancer agent(s) are generally administered sequentially in any order by a suitable route, such as infusion or orally. The dosing regimen may vary depending upon the stage of the disease, physical fitness of the patient, safety profiles of the individual drugs, and tolerance of the individual drugs, as well as other criteria well-known to the attending physician and medical practitioner(s) administering the combination. The compound of the present disclosure and other anti-cancer agent(s) may be administered within minutes of each other, hours, days, or even weeks apart depending upon the particular cycle being used for treatment. In addition, the cycle could include administration of one drug more often than the other during the treatment cycle and at different doses per administration of the drug.

In some cases, a treatment regime may be dosed according to a body weight of a subject. In subjects who are determined obese (BMI >35) a practical weight may need to be utilized. BMI is calculated by: BMI=weight (kg)/[height (m)]².

Body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In some cases, a body surface area may be utilized to calculate a dosage. A body surface area (BSA) may be calculated by: BSA (m2)=√Height (cm)*Weight (kg)/3600.

In an aspect is provided a method of modulating activity of a Ras (e.g., K-Ras) protein, comprising contacting a Ras protein with an effective amount of a compound described herein, or a pharmaceutically acceptable salt or solvate thereof, thereby modulating the activity of the Ras (e.g., K-Ras) protein. In some embodiments, the subject method comprises administering an additional agent or therapy.

In some embodiments is a method of modulating activity of a Ras protein, comprising contacting a Ras protein with an effective amount of a compound described, or a pharmaceutically acceptable salt or solvate thereof, wherein said modulating comprises inhibiting the Ras (e.g., K-Ras) protein activity. In some embodiments is a method of modulating activity of a Ras protein, including Ras mutant (e.g., G12C, G12D, G12S, G12V, G13C, or G13D) proteins of K-Ras, H-Ras, and N-Ras, comprising contacting the Ras protein with an effective amount of a compound described herein, or a pharmaceutically acceptable salt or solvate thereof.

In some embodiments, provided is a method of reducing Ras signaling output in a cell by contacting the cell with a compound described herein. A reduction in Ras signaling can be evidenced by one or more members of the following: (i) an increase in steady state level of GDP-bound modified protein; (ii) a reduction in steady state level of GTP-bound Ras protein; (iii) a reduction of phosphorylated AKTs473, (iv) a reduction of phosphorylated ERKT202/y204, (v) a reduction of phosphorylated S6S235/236, (vi) reduction of cell growth of a tumor cell expressing a Ras mutant (e.g., G12C, G12D, G12S, G12V, G13C, or G13D) protein, and (vii) reduction in Ras interaction with a Ras-pathway signaling protein. Non-limiting examples of Ras-pathway signaling proteins include SOS (including SOS1 and SOS2), RAF, SHC, SHP (including SHP1 and SHP2), MEK, MAPK, ERK, GRB, RASA1, and GNAQ. In some embodiments, the reduction in Ras signaling output can be evidenced by two, three, four, five, six, or all of (i)-(vii) above. In some embodiments, the reduction of any one or more of (i)-(vii) can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to a control not treated with a subject compound. A reduction in cell growth can be demonstrated with the use of tumor cells or cell lines. A tumor cell line can be derived from a tumor in one or more tissues, e.g., pancreas, lung, ovary, biliary tract, intestine (e.g., small intestine, large intestine, colon), endometrium, stomach, hematopoietic tissue (e.g., lymphoid tissue), etc. Examples of the tumor cell line with a K-Ras mutation may include, but are not limited to, A549 (e.g., K-Ras G12S), AGS (e.g., K-Ras G12D), ASPC1 (e.g., K-Ras G12D), Calu-6 (e.g., K-Ras Q61K), CFPAC-1 (e.g., K-Ras G12V), CL40 (e.g., K-Ras G12D), COL0678 (e.g., K-Ras G12D), COR-L23 (e.g., K-Ras G12V), DAN-G (e.g., K-Ras G12V), GP2D (e.g., K-Ras G12D), GSU (e.g., K-Ras G12F), HCT116 (e.g., K-Ras G13D), HEC1A (e.g., K-Ras G12D), HEC1B (e.g., K-Ras G12F), HEC50B (e.g., K-Ras G12F), HEYA8 (e.g., K-Ras G12D or G13D), HPAC (e.g., K-Ras G12D), HPAFII (e.g., K-Ras G12D), HUCCTI (e.g., K-Ras G12D), KARPAS620 (e.g., K-Ras G13D), KOPN8 (e.g., K-Ras G13D), KP-3 (e.g., K-Ras G12V), KP-4 (e.g., K-Ras G12D), L3.3 (e.g., K-Ras G12D), LoVo (e.g., K-Ras G13D), LS180 (e.g., K-Ras G12D), LS513 (e.g., K-Ras G12D), MCAS (e.g., K-Ras G12D), NB4 (e.g., K-Ras A18D), NCI-H1355 (e.g., K-Ras G13C), NCI-H1573 (e.g., K-Ras G12A), NCI-H1944 (e.g., K-Ras G13D), NCI-H2009 (e.g., K-Ras G12A), NCI-H441 (e.g., K-Ras G12V), NCI-H747 (e.g., K-Ras G13D), NOMO-1 (e.g., K-Ras G12D), OV7 (e.g., K-Ras G12D), PANC0203 (e.g., K-Ras G12D), PANC0403 (e.g., K-Ras G12D), PANC0504 (e.g., K-Ras G12D), PANC0813 (e.g., K-Ras G12D), PANC1 (e.g., K-Ras G12D), Panc-10.05 (e.g., K-Ras G12D), PaTu-8902 (e.g., K-Ras G12V), PK1 (e.g., K-Ras G12D), PK45H (e.g., K-Ras G12D), PK59 (e.g., K-Ras G12D), SK-CO-1 (e.g., K-Ras G12V), SKLU1 (e.g., K-Ras G12D), SKM-1 (e.g., K-Ras K117N), SNU1 (e.g., K-Ras G12D), SNU1033 (e.g., K-Ras G12D), SNU1197 (e.g., K-Ras G12D), SNU407 (e.g., K-Ras G12D), SNU410 (e.g., K-Ras G12D), SNU601 (e.g., K-Ras G12D), SNU61 (e.g., K-Ras G12D), SNU8 (e.g., K-Ras G12D), SNU869 (e.g., K-Ras G12D), SNU-C2A (e.g., K-Ras G12D), SU.86.86 (e.g., K-Ras G12D), SUIT2 (e.g., K-Ras G12D), SW1990 (e.g., K-Ras G12D), SW403 (e.g., K-Ras G12V), SW480 (e.g., K-Ras G12V), SW620 (e.g., K-Ras G12V), SW948 (e.g., K-Ras Q61L), T3M10 (e.g., K-Ras G12D), TCC-PAN2 (e.g., K-Ras G12R), TGBC11TKB (e.g., K-Ras G12D), and MIA Pa-Ca (e.g., MIA Pa-Ca 2 (e.g., K-Ras G12C)).

In an aspect is provided a modified Ras mutant protein comprising a compound described herein (or a remnant of a compound described herein wherein the remnant of said compound is modified from a stand alone compound described herein upon covalently bonding to the amino acid) covalently bonded to the amino acid corresponding to position 12 or 13 of SEQ ID No: 1. In some embodiments, such covalently bonded modified Ras mutant protein exhibits a reduced Ras signaling output (e.g., compared to a corresponding unmodified Ras mutant absent of the covalently bonded compound). In some embodiments, a modified Ras mutant protein is a K-Ras G12D mutant, an H-Ras G12D mutant, or an N-Ras G12D mutant. In some embodiments, a modified Ras mutant protein comprises an amino acid sequence selected from the group consisting of SEQ ID No. 2, SEQ ID No. 6, SEQ ID No. 8, and a respective fragment thereof comprising the aspartate residue corresponding to position 12 of SEQ ID No: 2. In some embodiments, the modified Ras mutant protein comprises a compound described herein covalently bonded to the amino acid residue corresponding to position 12 or 13 of SEQ ID No: 1, wherein the Ras mutant protein is a human protein selected from KRas G12D, KRas G12C, KRas G12S, KRas G13D, KRas G13C, and KRas G13S. In some embodiments, the modified Ras mutant protein comprises a compound described herein covalently bonded to the amino acid residue corresponding to position 12 or 13 of SEQ ID No: 1, wherein the Ras mutant protein is a mammalian Ras protein (including human protein) selected from NRas G12D, NRas G12C, NRas G12S, NRas G13D, NRas G13C, and NRas G13S. In some embodiments, the modified Ras mutant protein comprises a compound described herein covalently bonded to the amino acid residue corresponding to position 12 or 13 of SEQ ID No: 1, wherein the Ras mutant protein is a mammalian protein (including human protein) selected from HRas G12D, HRas G12C, HRas G12S, HRas G13D, HRas G13C, and HRas G13S. It will be understood that a compound described herein may be modified upon covalently binding an amino acid (e.g., mutant amino acid other than G) corresponding to position 12 or 13 of human KRas (e.g., SEQ ID. No: 1). A subject compound of the present disclosure encompasses a compound described herein immediately prior to covalently bonding the Ras mutant protein as well as the resulting compound covalently bonded to the modified Ras mutant protein. For example, a subject compound of the present disclosure can be covalently bonded to a mutant Ras protein to form a modified Ras mutant protein when a ring of the compound opened upon covalently bonding to the amino acid corresponding to position 12 or 13 of SEQ ID No: 1. The compound prior to and subsequent to such covalent binding are all considered a subject compound of the present disclosure.

In embodiments of a modified Ras mutant protein described herein, the reduced Ras signaling output is evidenced by one or more a reduced output selected from the group consisting of (i) an increase in steady state level of GDP-bound modified protein; (ii) a reduction in steady state level of GTP-bound Ras protein; (iii) a reduction of phosphorylated AKTs473, (iv) a reduction of phosphorylated ERK T202/Y204, (v) a reduction of phosphorylated S6 S235/236, (vi) reduction of cell growth of a tumor cell expressing a Ras mutant protein (e.g., G12D, G12C, G12S, G13D, G13C, or G13S), and (vii) reduction in Ras interaction with a Ras-pathway signaling protein.

In some embodiments, the modified Ras mutant protein described herein is formed by contacting a compound described herein with the aspartate residue of an unmodified Ras G12D mutant protein, wherein the compound comprises a moiety susceptible to reacting with a nucleophilic aspartate residue corresponding to position 12 of SEQ ID No: 2. In some embodiments, the compound comprises a staying group and a leaving group, and wherein said contacting results in release of the leaving group and formation of said modified protein. In some embodiments, the compound selectively labels the aspartate residue corresponding to position 12 of SEQ ID No. 2 (a G12D mutant) relative to a valine (G12V) residue at the same position. In some embodiments, the compound selectively labels the aspartate residue as compared to (i) a serine residue of a K-Ras G12S mutant protein, said serine corresponding to residue 12 of SEQ ID NO: 4, and/or (ii) a valine residue of a K-Ras G12V mutant protein, said valine corresponding to residue 12 of SEQ ID NO: 3. In some embodiments, the compound selectively labels the aspartate residue as compared to (i) an serine residue of a K-Ras G12S mutant protein, said serine corresponding to residue 12 of SEQ ID NO: 4, and/or (ii) a valine residue of a K-Ras G12V mutant protein, said valine corresponding to residue 12 of SEQ ID NO: 3, by at least 1, 2, 3, 4, 5, or 10 fold or more, when assayed under comparable conditions. In some embodiments, the compound selectively labels the aspartate residue corresponding to position 12 of SEQ ID No. 2 (a G12D KRas mutant) relative to a glycine residue at the same position in wildtype KRas.

In embodiments of the modified Ras mutant protein described herein, the compound interacts with the aspartate residue of an unmodified Ras G12D protein corresponding to position 12 of SEQ ID No: 2 in vitro. In embodiments of the modified Ras mutant protein described herein, the compound contacts the aspartate residue of an unmodified K-Ras G12D protein corresponding to position 12 of SEQ ID No: 2 in vivo.

In an aspect is provided a method of treating cancer in a subject comprising a Ras mutant protein (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S), the method comprising modifying the Ras mutant protein of said subject by administering to said subject a compound described herein, wherein the compound is characterized in that upon contacting a Ras mutant protein, said Ras mutant protein is modified covalently at a residue corresponding to residue 12 or 13 of SEQ ID No: 1, such that said modified Ras mutant protein exhibits reduced Ras signaling output (e.g., compared to a control, such as an unmodified Ras mutant protein not covalently bonded with any compound such as a compound disclosed herein).

In some aspects, a subject compound exhibits one or more of the following characteristics: it is capable of reacting with a mutant residue (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S) of a Ras mutant protein and covalently modifying such Ras mutant and/or it comprises a moiety susceptible to reacting with a nucleophilic amino acid residue corresponding to position 12 or 13 of SEQ ID No: 1 (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S). In some embodiments, a subject compound, when used to modify a Ras mutant protein, reduces the signaling output of the Ras protein. In some embodiments, a subject compound exhibits an IC50 (against a mutant Ras (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S), as ascertained by reduction of Ras::SOS1 interaction) of less than 10 μM, such as less than 5 μM, 1 μM, 500 nM, 100 nM, 50 nM, 10 nM, 5 nM, 1 nM, 500 pM, 50 pM, 10 pM or less.

In some embodiments, a modified Ras mutant protein disclosed herein exhibits a reduced Ras signaling output. A reduction of signaling output can be ascertained by a wide variety of methods known in the art. For example, phosphorylation of a substrate or a specific amino acid residue thereof can be detected and/or quantified using one or more techniques, such as kinase activity assays, phospho-specific antibodies, Western blot, enzyme-linked immunosorbent assays (ELISA), cell-based ELISA, intracellular flow cytometry, mass spectrometry, and multi-analyte profiling. A host of readout can evidence a reduction of Ras signaling output, including without limitation: (i) an increase in steady state level of GDP-bound modified protein; (ii) a reduction in steady state level of GTP-bound Ras protein; (iii) a reduction of phosphorylated AKTs473, (iv) a reduction of phosphorylated ERK T202/Y204, (v) a reduction of phosphorylated S6 S235/236, (vi) reduction of cell growth of a tumor cell expressing a Ras mutant protein (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S), and (vii) reduction in Ras interaction with a Ras-pathway signaling protein. In some embodiments, a reduction is evidenced by 2, 3, 4, 5, 6 or more of items (i)-(vii). In some embodiments, the reduction in Ras signaling output can be evidenced by any one of (i)-(vii) as compared to control unmodified corresponding Ras protein that is not covalently bonded to any compound disclosed herein. For example, a control Ras protein, as described herein, can be a Ras protein (e.g., wildtype or mutated) that is not complexed with any subject compound of the present disclosure. The increase in item (i) or reduction in items (ii) through (vii) can be at least about 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to the control Ras protein. In some embodiments, a reduction in Ras interaction with a Ras-pathway signaling protein is established by a reduced interaction with SOS (including SOS1 and SOS2), RAF, SHC, SHP (including SHP1 and SHP2), MEK, MAPK, ERK, GRB, RASA1, or GNAQ.

In some embodiments, the modified Ras mutant protein described herein is formed by contacting a compound with the aspartate residue of an unmodified Ras G12D mutant protein, wherein the compound comprises a moiety susceptible to reacting with a nucleophilic aspartate residue corresponding to position 12 of SEQ ID No: 2. Non-limiting examples of a moiety susceptible to reaction with a nucleophilic aspartate residue of a K-Ras G12D protein comprise an optionally substituted aziridinyl.

Signaling output measured in terms of IC50 values can be obtained and a ratio of IC50 against one mutant relative to another mutant can be calculated. For instance, a selective reduction of K-Ras G12D signaling output can be evidenced by a ratio greater than one. In particular, a selective reduction of K-Ras G12D signaling relative to K-Ras G12S signaling is evidenced if the ratio of IC50 (against K-Ras G12S) to IC50 (against K-Ras G12D) is greater than 1.

It will be understood that when a compound described herein selectively labels the aspartate residue of a K-Ras G12D protein compared to another K-Ras protein(s) (e.g., WT, G12S, or G12V), the compound labels the K-Ras G12D protein with greater speed or to a greater degree or by any other quantifiable measurement compared to the other K-Ras protein (e.g., WT, G12S, G12V), under similar or identical reaction conditions for the proteins being compared. In some embodiments, the greater labeling of K-Ras G12D can be 0.1-fold, 0.2-fold, 0.3-fold, 0.4-fold, 0.5-fold, 0.6-fold, 0.7-fold, 0.8-fold, 0.9-fold, 1-fold, 2-fold, 3-fold, 4-fold, 5-fold, 6- fold, 7-fold, 8-fold, 9-fold, 10-fold, 20-fold, 30-fold, 40-fold, 50-fold, 60-fold, 70-fold, 80-fold, 90-fold, 100-fold, 200-fold, 300-fold, 400-fold, 500-fold, 600-fold, 700-fold, 800-fold, 900-fold, 1000-fold, 2000-fold, 3000-fold, 4000-fold, 5000-fold, or more as compared to another K-Ras protein (e.g., WT, G12S, or G12V).

In some embodiments, the compounds described herein, or a pharmaceutically acceptable salt or solvate thereof, are Ras modulators (including Ras inhibitors) capable of covalently modifying a Ras protein. Ras proteins being modified can be Ras G12D mutants from K-Ras, H-Ras or N-Ras. The compounds disclosed herein, or pharmaceutically acceptable salts or solvates thereof, have a wide range of applications in therapeutics, diagnostics, and other biomedical research.

In an aspect is provided a method of treating cancer in a subject comprising a Ras G12D mutant protein, comprising modifying the Ras G12D mutant protein of said subject by administering to said subject a compound described herein, wherein said compound is characterized in that upon contacting the Ras G12D mutant protein, the Ras G12D mutant protein is modified covalently at an aspartate residue corresponding to residue 12 of SEQ ID No: 2, such that said modified K-Ras G12D protein exhibits reduced Ras signaling output (e.g., compared to a corresponding unmodified Ras protein unbound to the covalent compound).

In an aspect is provided a method of modulating activity of a Ras protein (e.g., K-Ras, mutant K-Ras, K-Ras G12D), comprising contacting a Ras protein with an effective amount of a compound described herein, or a pharmaceutically acceptable salt or solvate thereof, thereby modulating the activity of the Ras protein.

In practicing any of the methods disclosed herein, the Ras target to which a subject compound binds covalently can be a Ras mutant (e.g., KRas G12D, KRas G12C, KRas G12S, KRas G13D, KRas G13C, KRas G13S, NRas G12D, NRas G12C, NRas G12S, NRas G13D, NRas G13C, NRas G13S, HRas G12D, HRas G12C, HRas G12S, HRas G13D, HRas G13C, or HRas G13S).

### Pharmaceutical Compositions and Methods of Administration

In an aspect is provided a pharmaceutical composition comprising a compound described herein, or a pharmaceutically acceptable salt or solvate thereof, and a pharmaceutically acceptable excipient.

In some embodiments, a compound described herein, or a pharmaceutically acceptable salt or solvate thereof, is administered to a subject in a biologically compatible form suitable for administration to treat or prevent diseases, disorders, or conditions. Administration of a compound described herein can be in any pharmacological form including a therapeutically effective amount of a compound described herein, or a pharmaceutically acceptable salt or solvate thereof, alone or in combination with a pharmaceutically acceptable carrier.

In some embodiments, a compound described herein is administered as a pure chemical. In some embodiments, the compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, PA (2005)).

Accordingly, provided herein is a pharmaceutical composition comprising at least one compound described herein, or a pharmaceutically acceptable salt, together with one or more pharmaceutically acceptable excipients. The excipient(s) (or carrier(s)) is acceptable or suitable if the excipient is compatible with the other ingredients of the composition and not deleterious to the recipient (i.e., the subject) of the composition.

In some embodiments of the methods described herein, a compound described herein is administered either alone or in combination with pharmaceutically acceptable carriers, excipients or diluents, in a pharmaceutical composition. Administration of a compound or composition described herein can be affected by any method that enables delivery of the compound to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route may depend upon for example the condition and disorder of the recipient. By way of example only, a compound described herein can be administered locally to the area in need of treatment, by, for example, local infusion during surgery, topical application such as creams or ointments, injection, catheter, or implant. The administration can also be by direct injection at the site of a diseased tissue or organ. In some embodiments, a compound described herein, or a pharmaceutically acceptable salt or solvate thereof, is administered orally.

In some embodiments of the methods described herein, a pharmaceutical composition suitable for oral administration is presented as a discrete unit such as a capsule, cachet or tablet, each containing a predetermined amount of the active ingredient; as a powder or granules; as a solution or a suspension in an aqueous liquid or a non-aqueous liquid; or as an oil-in-water liquid emulsion or a water-in-oil liquid emulsion. In some embodiments, the active ingredient is presented as a bolus, electuary, or paste.

Pharmaceutical compositions which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound moistened with an inert liquid diluent. In some embodiments, the tablets are coated or scored and are formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In some embodiments, stabilizers are added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or Dragee coatings for identification or to characterize different combinations of active compound doses.

In some embodiments of the methods described herein, pharmaceutical compositions are formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. The compositions may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Pharmaceutical compositions for parenteral administration include aqueous and non-aqueous (oily) sterile injection solutions of the active compound which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical compositions may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, for example, as a sparingly soluble salt.

### EXAMPLES

The following examples are provided for illustrative purposes only and not to limit the scope of the claims provided herein. Unless noted otherwise, all materials, such as reagents, starting materials and solvents, were purchased from commercial suppliers, such as Sigma-Aldrich, VWR, and the like, and were used without further purification. Reactions were run under nitrogen atmosphere at room temperature, unless noted otherwise. The progress of reactions was monitored by thin layer chromatography (TLC), analytical high performance liquid chromatography (anal. HPLC), and mass spectrometry, the details of which may be provided in specific examples.

Reactions were worked up as described specifically in each preparation; commonly, reaction mixtures were purified by extraction and other purification methods such as temperature- and solvent-dependent crystallization, and precipitation. In addition, reaction mixtures were routinely purified by preparative HPLC, for example, using Microsorb C18 or Microsorb BDS column packings and conventional eluents. Progress of reactions was typically monitored by liquid chromatography mass spectrometry (LCMS). Characterization of isomers was typically done by Nuclear Overhauser effect spectroscopy (NOE). Characterization of reaction products was routinely carried out by mass spectrometry and/or ¹H-NMR spectroscopy. For NMR measurement, samples were dissolved in deuterated solvent (CD₃OD, CDCl₃, or DMSO-d).

Example 1a: Synthesis of 2-amino-4-(17′-chloro-19′-fluoro-7′-methyl-7′,8′,9′,10′,11′,12′,14′,15′-octahydro-4′H,6′H-spiro[cyclopropane-1,5′-[2,20](azeno)benzo[6,7][1,4]oxazepino[5,4-d][1]oxa[3,5,11]triazacyclotetradecin]-18′-yl)-7-fluorobenzo[b]thiophene-3-carbonitrile (149)

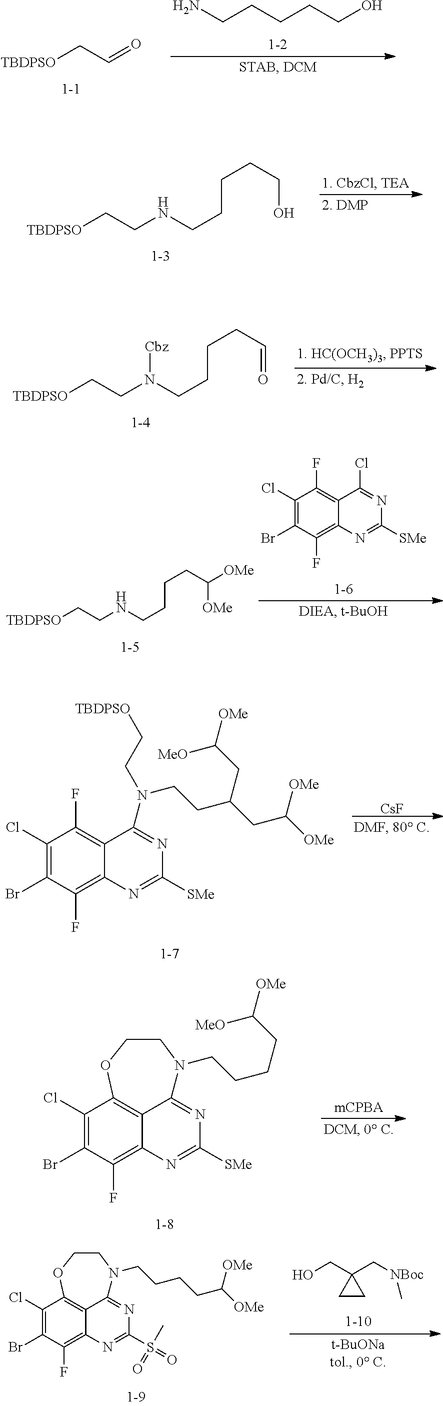

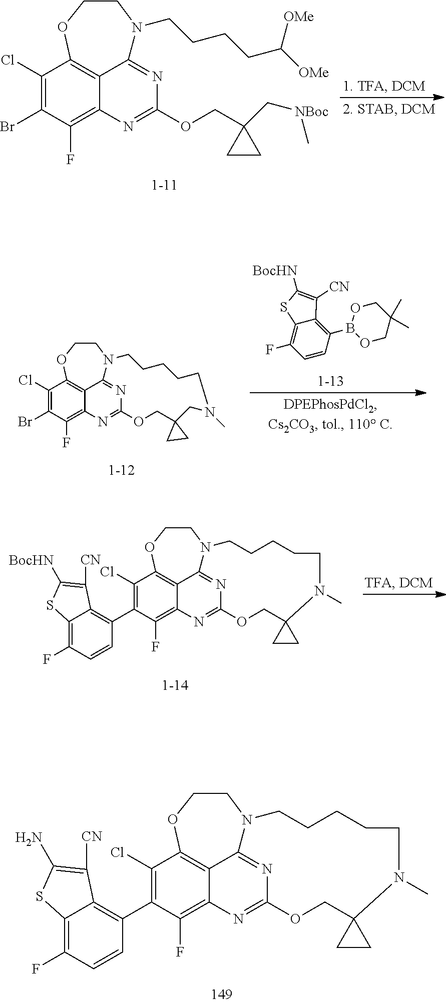 [SMILES: COC(CCCCN1CCOc2c(Cl)c(Br)c(F)c3nc(OCC4(CN(C)C(=O)OC(C)(C)C)CC4)nc1c23)OC ; CN1CCCCCN2CCOc3c(Cl)c(Br)c(F)c4nc(nc2c34)OCC2(CC2)C1 ; CC1(C)COB(c2ccc(F)c3sc(NC(=O)OC(C)(C)C)c(C#N)c23)OC1 ; CN1CCCCCN2CCOc3c(Cl)c(-c4ccc(F)c5sc(NC(=O)OC(C)(C)C)c(C#N)c45)c(F)c4nc(nc2c34)OCC12CC2 ; CN1CCCCCN2CCOc3c(Cl)c(-c4ccc(F)c5sc(N)c(C#N)c45)c(F)c4nc(nc2c34)OCC12CC2]

Step A: To a solution 5-aminopentanol (1-2, 4.15 g, 40.20 mmol, 4 equiv) in DCM (30 mL) was added 2-[(tert-butyldiphenylsilyl)oxy]acetaldehyde (1-1, 3 g, 10.052 mmol, 1 equiv). After stirring for 30 minutes, sodium triacetoxyborohydride (6.39 g, 30.15 mmol, 3 equiv) was added and the mixture was stirred for another 2 hours. The reaction mixture was extracted with DCM (3×10 mL) and the combined organic layers were washed with brine (2×10 mL), dried over anhydrous Na₂SO₄, filtered, and concentrated. The residue was purified by silica gel column chromatography, eluting with DCM/MeOH (10:1), to afford 1-3 as a yellow oil. (ESI, m/z): 386 [M+H]⁺.

Step B: To a solution of 1-3 (1.1 g, 2.85 mmol, 1 equiv) and triethylamine (1298 mg, 12.83 mmol, 4.5 equiv) in DCM (20 mL) was added benzyl chloroformate (729 mg, 4.28 mmol, 1.5 equiv) and the resulting mixture was stirred for 2 hours. The reaction was quenched with saturated NH₄Cl (30 mL) and the resulting mixture was extracted with DCM (3×10 mL). The combined organic layers were washed with brine (2×15 mL), dried over anhydrous Na₂SO₄, filtered, and concentrated under reduced pressure to give a residue. The residue was purified by silica gel column chromatography, eluting with DCM/MeOH (10/1) to afford the desired product as a dark yellow oil. (ESI, m/z): 520 [M+H]⁺.

Step C: A solution of benzyl N-(2-[(tert-butyldiphenylsilyl)oxy]ethyl-N-(5-hydroxypentyl)carbamate (800 mg, 1.53 mmol, 1 equiv) in DCM (15.0 mL) was treated with DMP (718 mg, 1.69 mmol, 1.1 equiv) for 2 hours. The reaction was quenched with sat. Na₂S₂O₃ and sat. NaHCO₃ and the resulting mixture was extracted with DCM (3×10 mL). The combined organic layers were washed with brine (2×10 mL), dried over anhydrous Na₂SO₄, filtered, and concentrated under reduced pressure to give 1-4 as a yellow oil, which was used in the next step without further purification. (ESI, m/z): 518 [M+H]⁺.

Step D: To a solution of 1-4 (710 mg, 1.371 mmol, 1 equiv) in MeOH (8 mL) were added trimethoxymethane (4365 mg, 41.13 mmol, 30 equiv) and PPTS (34 mg, 0.13 mmol, 0.1 equiv) in portions. After stirring for 16 hours, the reaction was quenched with water at 25° C. and extracted with EtOAc (3×10 mL). The combined organic layers were washed with brine (2×10 mL), dried over anhydrous Na₂SO₄, filtered, and concentrated to give a residue. The residue was purified by silica gel column chromatography, eluting with petroleum ether/ethyl acetate (10:1) to afford the desired product as a yellow oil. (ESI, m/z): 564 [M+H]⁺.

Step E: To a solution of benzyl N-(2-[(tert-butyldiphenylsilyl)oxy]ethyl-N-(5,5-dimethoxypentyl)carbamate (460 mg, 0.86 mmol, 1 equiv) in MeOH (4.6 mL) was added Pd/C (10%, 0.046 g). The mixture was stirred at room temperature under hydrogen atmosphere for 2 hours, then filtered through a Celite pad and the filtrate concentrated under reduced pressure to give 1-5 as a yellow oil, which was used directly in the next step without further purification. (ESI, m/z): 430 [M+H]⁺.

Step F: To a stirred solution of 1-5 (298 mg, 0.69 mmol, 1.5 equiv) in anhydrous t-BuOH (15 mL) were added 7-bromo-4,6-dichloro-5,8-difluoro-2-(methylsulfanyl)quinazoline (1-6, 167 mg, 0.46 mmol, 1.00 equiv) and DIEA (179 mg, 1.39 mmol, 3 equiv). The reaction mixture was stirred at 90° C. for 1 hour, then cooled to room temperature, quenched with saturated NH₄Cl (20 mL) and extracted with ethyl acetate (3×10 mL). The combined organic phase was washed with brine (2×10 mL), dried over anhydrous sodium sulfate, filtered, and concentrated under reduced pressure to give 1-7 as a yellow solid, which was used directly in the next step without further purification. (ESI, m/z): 752 [M+H]⁺.

Step G: To a stirred solution of 1-7 (420 mg, 0.55 mmol, 1 equiv) in anhydrous DMF (6 mL) was added CsF (254 mg, 1.67 mmol, 3 equiv). The reaction mixture was stirred at 80° C. for 2 hours, then cooled to room temperature, quenched with water (10 mL) and extracted with ethyl acetate (3×10 mL). The combined organic phase was washed with brine (2×10 mL), dried over anhydrous sodium sulfate, filtered and concentrated under reduced pressure to give a residue. The residue was purified by silica gel column chromatography, eluting with petroleum ether/ethyl acetate (5/1) to afford 1-8 as a white solid. (ESI, m/z): 494 [M+H]⁺.

Step H: To a stirred solution of 1-8 (150 mg, 0.30 mmol, 1 equiv) in anhydrous DCM (15 mL) was added m-CPBA (104 mg, 0.60 mmol, 2 equiv) at 0° C. The reaction mixture was stirred at 0° C. for 1 hour, then quenched with saturated Na₂S₂O₃ (20 mL) and NaHCO₃ (20 mL). The aqueous layer was extracted with DCM (3×10 mL), and the combined organic phase was washed with brine (2×10 mL), dried over anhydrous sodium sulfate, filtered and concentrated under reduced pressure to give 1-9, which was used directly in the next step without further purification. (ESI, m/z): 526 [M+H]⁺.

Step I: To a degassed solution of 1-9 (150 mg, 0.28 mmol, 1 equiv) in toluene (5 mL) was added tert-butyl N-([1-(hydroxymethyl)cyclopropyl]methyl-N-methylcarbamate (1-10, 122 mg, 0.570 mmol, 2 equiv) followed by t-BuONa (82 mg, 0.85 mmol, 3 equiv) at 0° C. The reaction mixture was stirred at 0° C. for 1 hour and quenched with saturated NH₄Cl (10 mL). The aqueous layer was extracted with ethyl acetate (2×10 mL). The combined organic phase was washed with brine (1×10 mL), dried over anhydrous Na₂SO₄, filtered, and concentrated to give a residue. The residue was purified by silica gel column chromatography, eluting with petroleum ether/ethyl acetate (5/1) to afford 1-11 as a white solid. (ESI, m/z): 661 [M+H]⁺.

Step J: A solution of 1-11 (130 mg, 0.27 mmol, 1 equiv) in DCM (0.6 mL) was treated with TFA (0.3 mL) overnight. The resulting mixture was extracted with DCM (3×10 mL). The combined organic layers were washed with brine (2×10 mL), dried over anhydrous Na₂SO₄, filtered, and concentrated to give the desired product. (ESI, m/z): 515 [M+H]⁺.

Step K: A solution of 5-(9-bromo-8-chloro-10-fluoro-2-((1-((methylamino)methyl)cyclopropyl)methoxy)-5,6-dihydro-4H-[1,4]oxazepino[5,6,7-de]quinazolin-4-yl)pentanal (120 mg, 0.233 mmol, 1 equiv) in DCM (120.0 mL) was treated with sodium triacetoxyborohydride (147 mg, 0.69 mmol, 3 equiv) for 2 hours. The reaction was then quenched with saturated NaHCO₃ and extracted with DCM (3×30 mL). The combined organic layers were washed with brine (2×50 mL), dried over anhydrous Na₂SO₄, filtered, and concentrated to give a residue. The residue was purified by silica gel column chromatography, eluting with DCM/MeOH (20/1) to afford 1-12 as a white solid. (ESI, m/z): 499 [M+H]⁺.

Step L: To a stirred solution of 1-12 (45 mg, 0.09 mmol, 1 equiv) in anhydrous toluene (2 mL) were added tert-butyl N-[3-cyano-4-(5,5-dimethyl-1,3,2-dioxaborinan-2-yl)-7-fluoro-1-benzothiophen-2-yl]carbamate (1-13, 90 mg, 0.22 mmol, 2.0 equiv), bis(diphenylphosphinophenyl)ether palladium (II) dichloride (12 mg, 0.018 mmol, 0.2 equiv) and Cs₂CO₃ (88 mg, 0.270 mmol, 3 equiv). The reaction mixture was stirred at 110° C. for 2 hours, then cooled to room temperature, quenched with water (10 mL) and extracted with ethyl acetate (3×10 mL). The combined organic phase was washed with brine (2×10 mL), dried over anhydrous sodium sulfate, filtered, and concentrated to give a residue. The residue was purified by prep-TLC (DCM/MeOH=10/1) to afford 1-14 as a yellow solid. (ESI, m/z): 711 [M+H]⁺.

Step M: A solution of 1-14 (18 mg, 0.025 mmol, 1 equiv) in DCM (3.6 mL) was treated with TFA (0.72 mL) for 2 hours at room temperature. The mixture was then concentrated to give a residue, which was purified by prep-HPLC (Water (10 mmol/L NH₄HCO₃) and ACN (35% ACN up to 75%) to afford 149 as a white solid. (ESI, m/z): 611 [M+H]⁺. ¹H NMR (400 MHz, DMSO-d₆, ppm) δ 8.06 (s, 2H), 7.22-7.08 (m, 2H), 4.82 (s, 1H), 4.69 (d, J=11.2 Hz, 1H), 4.54 (s, 2H), 3.96 (s, 2H), 3.76 (s, 1H), 3.65 (s, 1H), 2.67 (q, J=1.9 Hz, 1H), 2.40-2.30 (m, 2H), 2.23 (d, J=12.3 Hz, 1H), 2.11 (s, 3H), 1.79 (s, 2H), 1.55 (s, 2H), 1.46 (s, 2H), 0.55 (s, 2H), 0.28 (s, 2H).

Example 1b: Synthesis of 2-amino-4-(14-chloro-16-fluoro-7-oxo-4,5,6,7,8,9,11,12-octahydro-2,17-(azeno)benzo[6,7][1,4]oxazepino[5,4-d][1]oxa[3,5,9]triazacycloundecin-15-yl)-7-fluorobenzo[b]thiophene-3-carbonitrile (112)

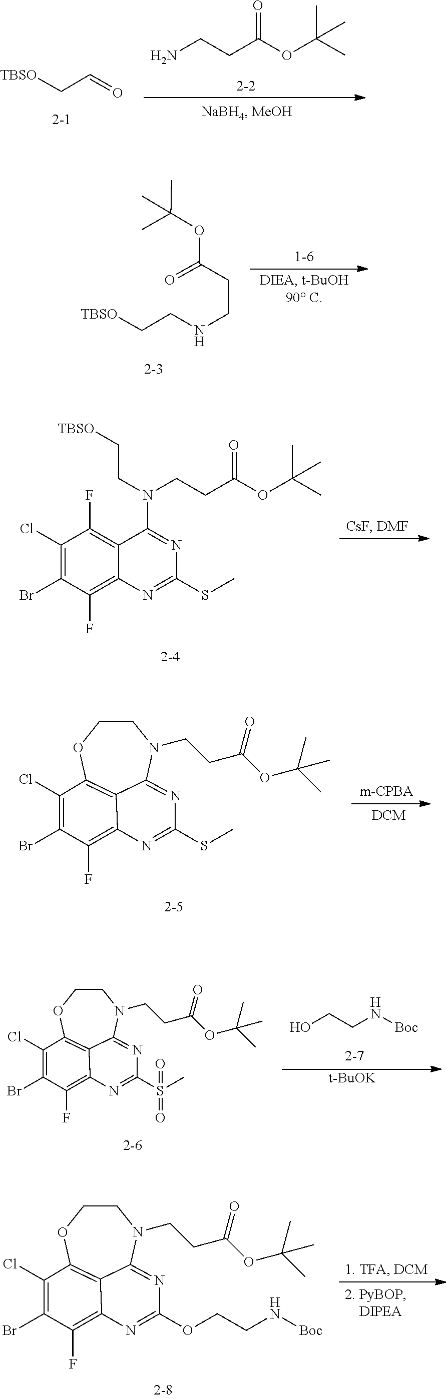

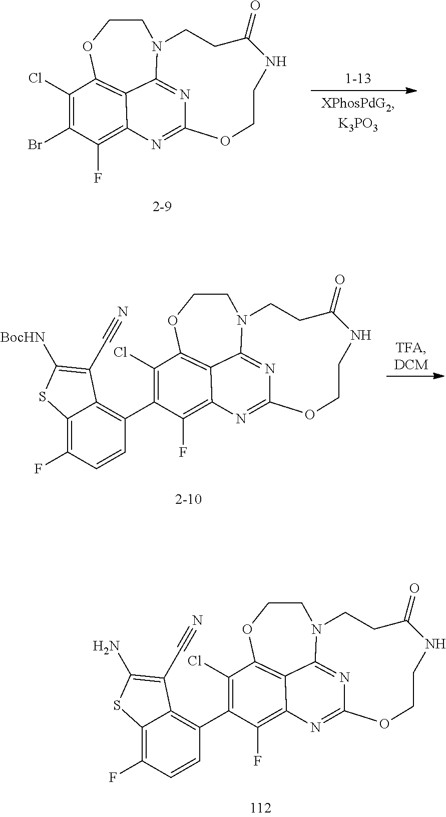 [SMILES: O=C1CCN2CCOc3c(Cl)c(Br)c(F)c4nc(nc2c34)OCCN1 ; CC(C)(C)OC(=O)Nc1sc2c(F)ccc(-c3c(Cl)c4c5c6nc(nc5c3F)OCCNC(=O)CCN6CCO4)c2c1C#N ; N#Cc1c(N)sc2c(F)ccc(-c3c(Cl)c4c5c6nc(nc5c3F)OCCNC(=O)CCN6CCO4)c12]

Step A: To a solution of tert-butyl 3-aminopropanoate (2-2, 2.0 g, 13.76 mmol, 1.20 equiv) and 2-[(tert-butyldimethylsilyl)oxy]acetaldehyde (2-1, 2.0 g, 11.47 mmol, 1 equiv) in methanol (10 mL) was added NaBH₄ (434 mg, 11.47 mmol, 1.00 equiv). The resulting mixture was stirred for 3 hours, then extracted with EtOAc (3×10 mL). The combined organic layers were washed with brine (1×10 mL), dried over anhydrous sodium sulfate, filtered, and concentrated to give a residue. The residue was purified by silica gel column chromatography, eluting with petroleum ether/ethyl acetate (5:1) to afford 2-3 as a light-yellow oil. (ESI, m/z): 304 [M+H]⁺.

Step B: A mixture of 2-3 (800 mg, 2.63 mmol, 1 equiv), 1-6 (948 mg, 2.63 mmol, 1 equiv) and DIEA (1.38 mL, 7.90 mmol, 3 equiv) in t-BuOH (2 mL) was stirred for 1 hour at 90° C. The resulting mixture was cooled to room temperature and extracted with EtOAc (2×5 mL). The combined organic layers were washed with brine (1×8 mL), dried over anhydrous Na₂SO₄, filtered, and concentrated to give a residue. The residue was purified by silica gel column chromatography, eluting with petroleum ether/ethyl acetate (10:1) to afford 2-4 as a light-yellow solid. ¹H NMR (400 MHz, CDCl₃) δ 3.92 (m, 2H), 3.80 (m, 2H), 3.73 (m, 2H), 2.72 (m, 2H), 2.58 (s, 3H), 1.42 (s, 9H), 0.77 (s, 9H), −0.06 (s, 6H).

Step C: A mixture of 2-4 (1.45 g, 2.31 mmol, 1 equiv) and CsF (1053 mg, 6.93 mmol, 3 equiv) in DMF (10 mL) was stirred for 16 hours at room temperature, then extracted with EtOAc (3×10 mL). The combined organic layers were washed with brine (4×10 mL), dried over anhydrous Na₂SO₄, filtered, and concentrated to give a residue. The residue was triturated with a mixture of EtOAc (20 mL) and petroleum ether (10 mL), then filtered. The filter cake was washed with petroleum ether (1×10 mL) to afford 2-5 as a dark yellow solid. (ES, m/z): 492 [M+H]⁺.

Step D: A mixture of 2-5 (550 mg, 1.11 mmol, 1 equiv) in DCM (5 mL) was treated with m-CPBA (577 mg, 3.34 mmol, 3 equiv) at 0° C., then stirred for 2 hours at room temperature. The reaction was quenched by the addition of sat. NaHSO₃ (5 mL) at room temperature. The mixture was basified to pH ˜8 with saturated NaHCO₃ (aq.) and extracted with CH₂Cl₂ (2×5 mL). The combined organic layers were washed with brine, dried over anhydrous Na₂SO₄, filtered, and concentrated to give a residue. The residue was purified by silica gel column chromatography, eluting with DCM/ethyl acetate (20:1) to afford 2-6 as a white solid. (ES, m/z): 524 [M+H]⁺.

Step E: A mixture of 2-6 (400 mg, 0.76 mmol, 1 equiv) and tert-butyl N-(2-hydroxyethyl)carbamate (2-7, 245 mg, 1.52 mmol, 2 equiv) in toluene (5 mL) was treated with KOt-Bu (94 mg, 0.88 mmol, 1.1 equiv) at 0° C., then stirred for 1 hour at room temperature. The resulting mixture was extracted with ethyl acetate (2×5 mL). The combined organic layers were washed with brine (1×10 mL), dried over anhydrous Na₂SO₄, filtered, and concentrated under reduced pressure to give a residue. The residue was purified by silica gel column chromatography, eluting with CH₂Cl₂/ethyl acetate (10:1) to afford 2-8 as a white solid. (ES, m/z): 606 [M+H]⁺.

Step F: To a stirred mixture of 2-8 (220 mg, 0.36 mmol, 1 equiv) in DCM (1.2 mL) was added TFA (0.40 mL) dropwise at room temperature. The resulting mixture was stirred for 2 hours, then concentrated under reduced pressure to give a residue. The residue was dissolved in MeCN (0.8 mL), then DIEA (0.63 mL, 3.63 mmol, 10 equiv) and PyBop (283 mg, 0.54 mmol, 1.5 equiv) were added. The resulting mixture was stirred for 3 hours, then the precipitated solids were collected by filtration and washed with acetonitrile (1×5 mL), MeOH (1×5 mL) and H₂O (1×5 mL) to afford 2-9 as a white solid. (ES, m/z): 431 [M+H]⁺.

Step G: A mixture of 2-9 (82 mg, 0.19 mmol, 1 equiv), 1-13 (230 mg, 0.57 mmol, 3 equiv), XPhos-PdCl-2nd G (57 mg, 0.076 mmol, 0.4 equiv), and K₃PO₄ (362 mg, 1.71 mmol, 9 equiv) in THF (4 mL, 49.37 mmol) was stirred for 5 hours at 65° C. under argon atmosphere. The resulting mixture was cooled to room temperature and extracted with ethyl acetate (2×5 mL). The combined organic layers were washed with brine (1×5 mL), dried over anhydrous Na₂SO₄, filtered, and concentrated to give a residue. The residue was purified by silica gel column chromatography, eluting with CH₂Cl₂/MeOH (10:1) to afford 2-10 as a grey solid. (ES, m/z): 643 [M+H]⁺.

Step H: A solution of 2-10 (35 mg, 0.054 mmol, 1 equiv) and TFA (0.5 mL, 6.732 mmol) in DCM (1.5 mL) was stirred for 7 hours at room temperature. The resulting mixture was concentrated under reduced pressure to give a residue. The residue was basified to pH-8 with NH₃·H₂O and concentrated. The crude product (40 mg) was purified by prep-HPLC (Water (10 mmol/L NH₄HCO₃) and 25-50% ACN), then further purified by Prep-Chiral HPLC (CHIRALPAK-IC; hexanes (0.1% DEA) and MeOH:DCM (1:1), isocratic 35 gradient) to afford 112 as a white solid. (ES, m/z): 543 [M+H]⁺. 1H NMR (400 MHz, DMSO-d6) δ 8.10 (s, 2H), 7.28-7.04 (m, 2H), 5.28-4.86 (m, 1H), 4.75-4.28 (m, 3H), 4.04-3.87 (m, 3H), 3.66-3.54 (m, 1H), 3.25-3.11 (m, 1H), 3.42-2.30 (m, 1H), 2.29-2.05 (m, 2H).

Example 1c: Synthesis of 2-amino-4-((5R,8S)-17-chloro-19-fluoro-6-methyl-5,6,7,8,11,12,14,15-octahydro-4H,10H-2,20-(azeno)-5,8-methanobenzo[6,7][1,4]oxazepino[5,4-d][1,9]dioxa[3,5,12]triaza-cyclotetradecin-18-yl)-7-fluorobenzo[b]thiophene-3-carbonitrile (122 and 135)

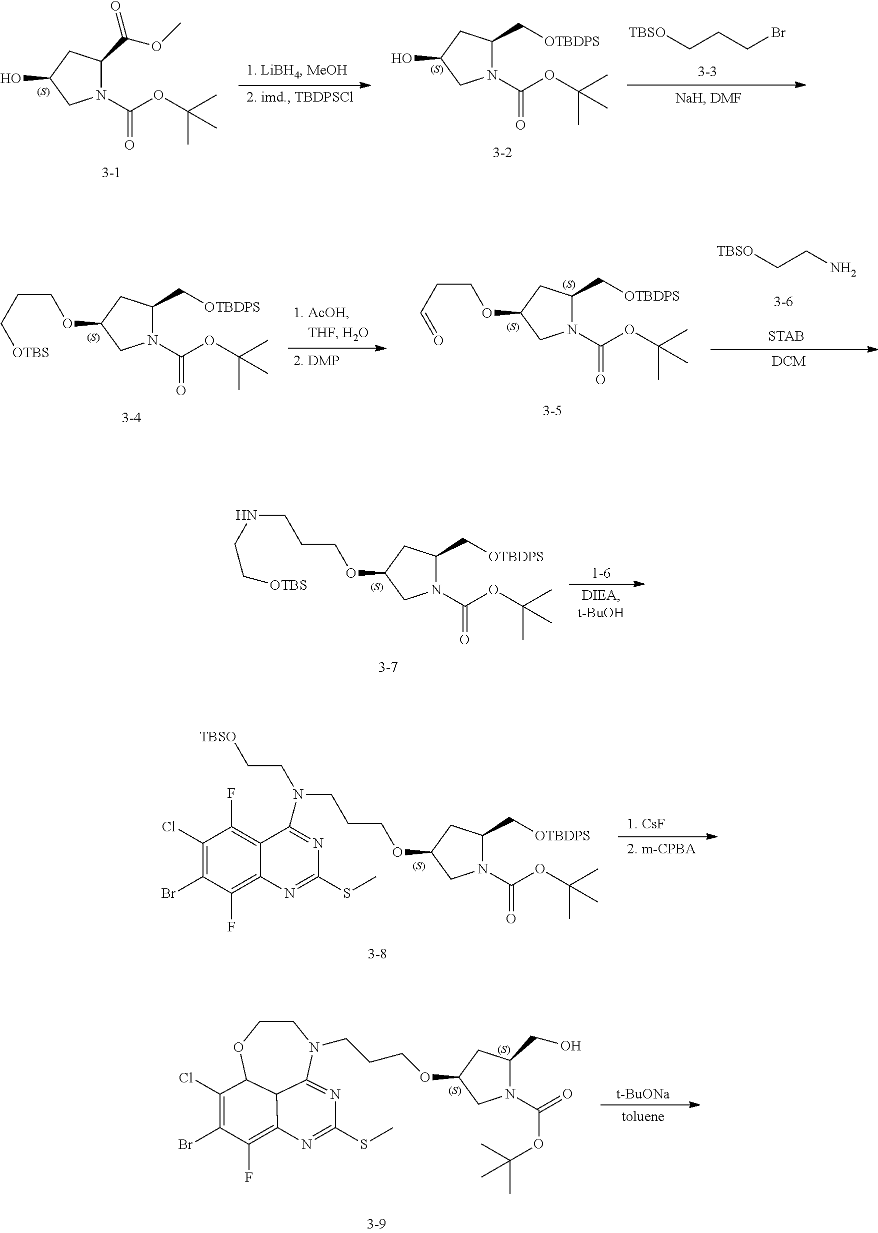

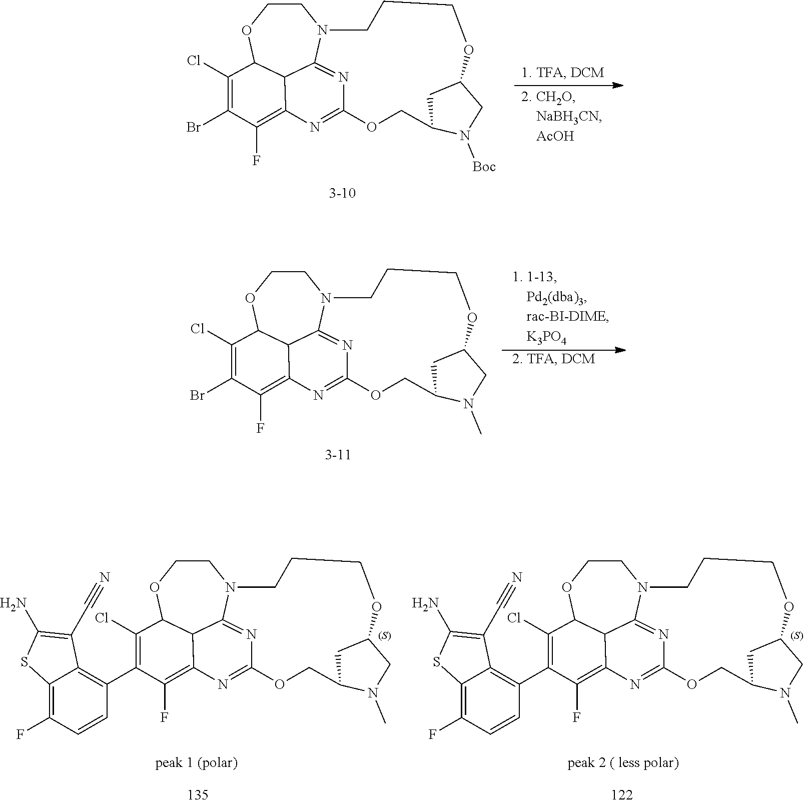

Step A: To a stirred solution of 1-tert-butyl 2-methyl (2S,4S)-4-hydroxypyrrolidine-1,2-dicarboxylate (3-1, 25 g, 101.92 mmol, 1 equiv) in anhydrous MeOH (500 mL) was added LiBH₄ (7.77 g, 356.74 mmol, 3.5 equiv). The reaction mixture was stirred at room temperature for 2 hours, quenched with water (300 mL), and extracted with DCM (3×200 mL). The combined organic phase was washed with brine (500 mL), dried over anhydrous sodium sulfate and concentrated under reduced pressure to give the desired product as a transparent oil which was used in the next step with further purification. (ESI, m/z): 218 [M+H]⁺.

Step B: To a stirred solution of tert-butyl (2S,4S)-4-hydroxy-2-(hydroxymethyl)pyrrolidine-1-carboxylate (14 g, 64.43 mmol, 1 equiv) in anhydrous DCM (200 mL) was added imidazole (8.77 g, 128.87 mmol, 2 equiv) and TBDPSCl (19.48 g, 70.88 mmol, 1.1 equiv). The reaction mixture was stirred at room temperature for 3 hours, quenched by addition of water (300 mL), and extracted with DCM (3×200 mL). The combined organic phase was washed with brine (300 mL), dried over anhydrous sodium sulfate, and concentrated under reduced pressure to give crude product which was purified by column chromatography using 0% to 15% ethyl acetate in petroleum ether to afford 3-2 as a white oil. (ESI, m/z): 456 [M+H]⁺.

Step C: To a stirred solution of 3-2 (9 g, 19.75 mmol, 1 equiv) in anhydrous DMF (80 mL) was added NaH (1.58 g, 39.50 mmol, 2 equiv, 60%) and (3-bromopropoxy)(tert-butyl)dimethylsilane (3-3, 7.50 g, 29.62 mmol, 1.5 equiv). The reaction mixture was stirred at 50° C. for a period of 1 hour, then cooled to room temperature, quenched with water (200 mL) and extracted with ethyl acetate (3×100 mL). The combined organic phase was washed with brine (3×100 mL), dried over anhydrous sodium sulfate and concentrated under reduced pressure to give crude product which was purified by column chromatography using 0% to 15% ethyl acetate in petroleum ether to afford 3-4 as an oil. (ESI, m/z): 628 [M+H]⁺.

Step D: A stirred solution of 3-4 (5.5 g, 8.758 mmol, 1 equiv) in AcOH (27.5 mL)/THF (55 mL)/H₂O (27.5 mL) was stirred at 40° C. for 16 hours, then extracted with ethyl acetate (3×30 mL). The combined organic phase was washed with brine (3×50 mL), dried over anhydrous sodium sulfate and concentrated under reduced pressure to give a residue, which was further purified by column chromatography using 0% to 5% MeOH in DCM to afford the desired product as an oil (ESI, m/z): 514 [M+H]⁺.

Step E: To a stirred solution of tert-butyl (2S,4S)-2-([(tert-butyldiphenylsilyl)oxy]methyl)-4-(3-hydroxypropoxy)pyrrolidine-1-carboxylate (2.8 g, 5.45 mmol, 1 equiv) in anhydrous DCM (50 mL) was added DMP (3.47 g, 8.17 mmol, 1.5 equiv). The reaction mixture was stirred at room temperature for 1 hour, then quenched by addition of saturated Na₂S₂O₃ and NaHCO₃ (200 mL, 1:1). The aqueous layer was extracted with DCM (3×30 mL). The combined organic phase was washed with brine (80 mL), dried over anhydrous sodium sulfate and concentrated under reduced pressure to give 3-5 as a yellow oil, which was used in the next step directly without further purification. (ESI, m/z): 512 [M+H]⁺.

Step F: To a stirred solution of 3-5 (2.6 g, 5.08 mmol, 1 equiv) in anhydrous DCM (30 mL) were added (2-aminoethoxy)(tert-butyl)dimethylsilane (3-6, 1.78 g, 10.16 mmol, 2 equiv) and sodium triacetoxyboronhydride (1.62 g, 7.62 mmol, 1.5 equiv). The reaction mixture was stirred at room temperature for 2 hours, then quenched by addition of water (100 mL). The aqueous layer was extracted with DCM (3×40 mL). The combined organic phase was washed with brine (100 mL), dried over anhydrous sodium sulfate, filtered and concentrated under reduced pressure to give a residue, which was purified by column chromatography using 0% to 5% MeOH in DCM to afford 3-7 as a white oil. (ESI, m/z): 671 [M+H]⁺.

Step G: To a stirred solution of 3-7 (1.8 g, 2.68 mmol, 1 equiv) in anhydrous t-BuOH (32 mL) were added DIEA (1040 mg, 8.046 mmol, 3 equiv) and 1-6 (965 mg, 2.68 mmol, 1 equiv). The reaction mixture was stirred at 80° C. for 1 hour and cooled to room temperature. The reaction was quenched by addition of water (100 mL) and the aqueous layer was extracted with ethyl acetate (3×30 mL). The combined organic phase was washed with brine (100 mL), dried over anhydrous sodium sulfate, filtered and concentrated to give 3-8 as a yellow oil, which was used directly in the next step without further purification. (ESI, m/z): 993 [M+H]⁺.

Step H: To a stirred solution of 3-8 (2.4 g, 2.41 mmol, 1 equiv) in anhydrous DMF (35 mL) was added CsF (1099 mg, 7.23 mmol, 3 equiv). The reaction mixture was stirred at 80° C. for 2 hours, then cooled to room temperature. The reaction was quenched by addition of water (60 mL) and the aqueous layer was extracted with ethyl acetate (3×30 mL). The combined organic phases were washed with brine (3×60 mL), dried over anhydrous sodium sulfate, filtered and concentrated to give a residue, which was purified by column chromatography using 0% to 5% MeOH in DCM to afford the desired product as a yellow solid. (ESI, m/z): 621 [M+H]⁺.

Step I: To a stirred solution of tert-butyl (2S,4S)-4-(3-(9-bromo-8-chloro-10-fluoro-2-(methylthio)-5,6-dihydro-4H-[1,4]oxazepino[5,6,7-de]quinazolin-4-yl)propoxy)-2-(hydroxymethyl)pyrrolidine-1-carboxylate (1.2 g, 1.92 mmol, 1 equiv) in anhydrous DCM (20 mL) was added m-CPBA (499 mg, 2.89 mmol, 1.5 equiv). The reaction mixture was stirred at room temperature for 1 hour, then quenched by addition of water (60 mL). The aqueous layer was extracted with DCM (3×20 mL). The combined organic phase was washed with brine (60 mL), dried over anhydrous sodium sulfate, filtered and concentrated to give 3-9 as a yellow solid, which used directly in the next step without further purification. (ESI, m/z): 653 [M+H]⁺.

Step J: To a stirred solution of 3-9 (1.2 g, 1.83 mmol, 1 equiv) in anhydrous toluene (20 mL) was added t-BuONa (529 mg, 5.50 mmol, 3 equiv). The reaction mixture was stirred at room temperature for 1 hour, quenched by addition of water (80 mL) and extracted with ethyl acetate (3×30 mL). The combined organic phase was washed with brine (100 mL), dried over anhydrous sodium sulfate, filtered and concentrated to give a residue, which was purified by column chromatography using 0% to 4% MeOH in DCM to afford 3-10 as a yellow solid. (ESI, m/z): 573 [M+H]⁺.

Step K: To a stirred solution of 3-10 (500 mg, 0.87 mmol, 1 equiv) in anhydrous DCM (10 mL) was added TFA (2 mL). The reaction mixture was stirred at room temperature for 1 hour, quenched by addition of saturated NaHCO₃ (50 mL) and extracted with DCM (3×30 mL). The combined organic phase was washed with brine (40 mL), dried over anhydrous sodium sulfate, filtered and concentrated to give the desired product as a solid, which was used in the next step without further purification. (ESI, m/z): 473 [M+H]⁺.

Step L: To a stirred solution of (5R,8S)-18-bromo-17-chloro-19-fluoro-5,6,7,8,11,12,14,15-octahydro-4H,10H-2,20-(azeno)-5,8-methanobenzo[6,7][1,4]oxazepino[5,4-d][1,9]dioxa[3,5,12]triazacyclotetradecine (540 mg, 1.14 mmol, 1 equiv) in anhydrous MeOH (10 mL) were added formaldehyde (171 mg, 5.70 mmol, 5 equiv) and AcOH (7 mg, 0.11 mmol, 0.1 equiv) followed by NaBH₃CN (143 mg, 2.28 mmol, 2 equiv). The reaction mixture was stirred at room temperature for 2 hours, then quenched by addition of water (30 mL) and extracted with DCM (3×20 mL). The combined organic phase was washed with brine (50 mL), dried over anhydrous sodium sulfate, filtered and concentrated to give a residue, which was purified by column chromatography using 0% to 7% MeOH in DCM to afford 3-11 as a white oil. (ESI, m/z): 487 [M+H]⁺.

Step M: To a stirred solution of 3-11 (200 mg, 0.41 mmol, 1 equiv) in anhydrous dioxane (8 mL) were added 1-13 (497 mg, 1.23 mmol, 3 equiv) and K₃PO₄ (783 mg, 3.69 mmol, 9 equiv) followed by Pd₂(dba)₃ (75 mg, 0.082 mmol, 0.2 equiv) and 3-tert-butyl-4-(2,6-dimethoxyphenyl)-2,3-dihydro-1,3-benzoxaphosphole (54 mg, 0.16 mmol, 0.4 equiv). The reaction mixture was stirred at 100° C. for 1.5 hours, then cooled to room temperature, quenched by addition of water (30 mL), and extracted with ethyl acetate (3×20 mL). The combined organic phase was washed with brine (30 mL), dried over anhydrous sodium sulfate, filtered and concentrated under reduced pressure to give a residue, which was further purified by column chromatography using 0% to 6% MeOH in DCM to afford the desired product as a yellow solid. (ESI, m/z): 699 [M+H]⁺.

Step N: To a stirred solution of tert-butyl (4-((5R,8S)-17-chloro-19-fluoro-6-methyl-5,6,7,8,11,12,14,15-octahydro-4H,10H-2,20-(azeno)-5,8-methanobenzo[6,7][1,4]oxazepino[5,4-d][1,9]dioxa[3,5,12]-triazacyclotetradecin-18-yl)-3-cyano-7-fluorobenzo[b]thiophen-2-yl)carbamate (200 mg, 0.28 mmol, 1 equiv) in anhydrous DCM (5 mL) was added TFA (1 mL). The reaction mixture was stirred at room temperature for 2 hours, then concentrated under reduced pressure and the residue purified by Prep-HPLC (water (10 mmol/L NH₄HCO₃) and 30% to 49% ACN) to afford 135 and 122 as white solids. Peak 1 (135): (ESI, m/z): 599 [M+H]⁺; ¹H NMR: (400 MHz, DMSO-d6, ppm) δ 8.07 (s, 2H), 7.19 (m, 1H), 7.12 (m, 1H), 5.23 (m, 2H), 4.63 (m, 1H), 4.47 (m, 1H), 3.89 (t, J=4.2 Hz, 4H), 3.28 (s, 1H), 3.24-3.16 (m, 1H), 3.13-2.81 (m, 3H), 2.59 (s, 1H), 2.43 (s, 3H), 1.98 (m, 1H), 1.89-1.74 (m, 2H), 1.66 (s, 1H). Peak 2 (122): (ESI, m/z): 599 [M+H]⁺; ¹H NMR: (400 MHz, DMSO-d6, ppm) δ 8.07 (s, 2H), 7.24-7.01 (m, 2H), 5.26 (m, 2H), 4.66-4.46 (m, 2H), 4.01-3.78 (m, 4H), 3.29 (s, 1H), 3.18 (d, J=8.9 Hz, 1H), 3.01 (m, 3H), 2.59 (d, J=9.8 Hz, 1H), 2.54-2.39 (m, 3H), 1.93 (d, J=12.5 Hz, 1H), 1.83 (d, J=13.6 Hz, 2H), 1.64 (d, J=9.7 Hz, 1H).

Example 1d: Synthesis of 2-amino-4-((11S,Z)-14-chloro-4-((dimethylamino)methyl)-16-fluoro-11-methyl-4,5,8,9,11,12-hexahydro-2,17-(azeno)benzo[6,7][1,4]oxazepino[5,4-d][1]oxa[3,5]diazacycloundecin-15-yl)-7-fluorobenzo[b]thiophene-3-carbonitrile (103, 114, and 127)

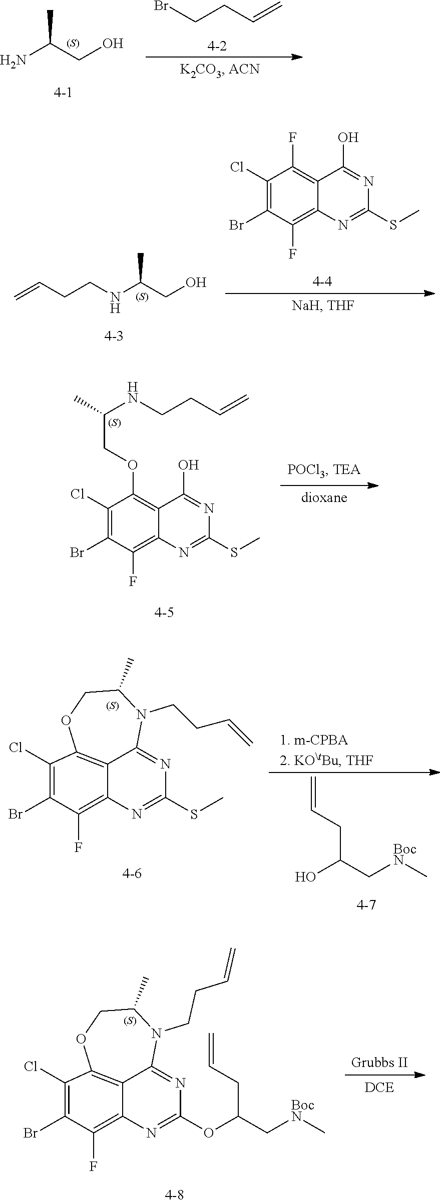

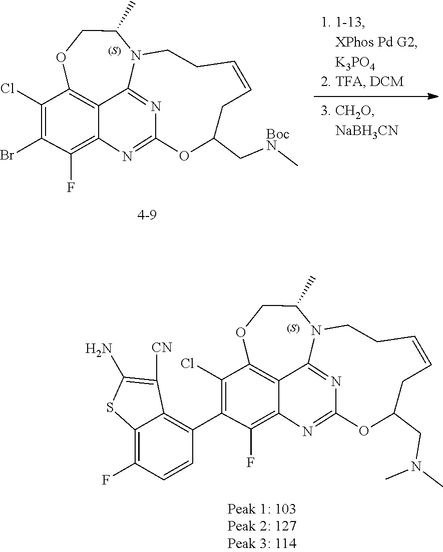

Step A: To a stirred mixture of (2S)-2-aminopropan-1-ol (4-1, 4 g, 53.255 mmol, 1 equiv) and 4-bromo-1-butene (4-2, 5.75 g, 42.60 mmol, 0.8 equiv) in MeCN (40 mL) was added K₂CO₃ (9.20 g, 66.56 mmol, 1.25 equiv) in portions. The resulting mixture was stirred for 6 hours at 80° C., then cooled to room temperature, filtered, and the filter cake washed with CH₂Cl₂ (3×10 mL). The filtrate was concentrated under reduced pressure to give a residue. The residue was purified by silica gel column chromatography, eluting with CH₂Cl₂/MeOH (10:1) to afford 4-3 as an off-white oil. (ESI, m/z): 130 [M+H]⁺.

Step B: To a stirred mixture of 4-3 (300 mg, 2.32 mmol, 1 equiv) and 7-bromo-6-chloro-5,8-difluoro-2-(methylsulfanyl)quinazolin-4-ol (4-4, 713 mg, 2.09 mmol, 0.9 equiv) in THF (6 mL) was added 60% NaH (743 mg, 18.57 mmol, 8 equiv) in portions at 0° C. The reaction mixture was stirred for 2 hours at room temperature, then quenched with water/ice at room temperature and extracted with EtOAc (3×30 mL). The combined organic layers were washed with brine, dried over anhydrous Na₂SO₄, filtered, and concentrated under reduced pressure to give 4-5, which was used in the next step directly without further purification. (ESI, m/z): 450 [M+H]⁺.

Step C: To a stirred mixture of 4-5 (1.1 g, 2.440 mmol, 1 equiv) and POCl₃ (2244 mg, 14.64 mmol, 6 equiv) in dioxane (22 mL) was added triethylamine (2963 mg, 29.28 mmol, 12 equiv) dropwise at room temperature. The resulting mixture was stirred for 30 minutes at room temperature, quenched with water/ice at room temperature, and extracted with CH₂Cl₂ (3×100 mL). The combined organic layers were washed with brine, dried over anhydrous Na₂SO₄, filtered, and concentrated to give a residue. The residue was purified by reverse-phase flash chromatography (10 to 50% MeCN in water (10 mmol/L NH₄HCO₃)) to afford 4-6 as a yellow solid. (ESI, m/z): 432 [M+H]⁺.

Step D: To a stirred solution of 4-6 (700 mg, 1.61 mmol, 1 equiv) in DCM (14 mL) was added m-CPBA (837 mg, 4.85 mmol, 3 equiv) in portions at 0° C. The resulting mixture was stirred for 1 hour at room temperature, then quenched with sat. NaHCO₃ (aq.) and extracted with CH₂Cl₂ (3×150 mL). The combined organic layers were washed with brine, dried over anhydrous Na₂SO₄, filtered, and concentrated to give the desired product, which was used in the next step directly without further purification. (ESI, m/z): 464 [M+H]⁺.

Step E: To a stirred mixture of (S)-9-bromo-4-(but-3-en-1-yl)-8-chloro-10-fluoro-5-methyl-2-(methylsulfonyl)-5,6-dihydro-4H-[1,4]oxazepino[5,6,7-de]quinazoline (900 mg, 1.93 mmol, 1 equiv) and tert-butyl N-(2-hydroxypent-4-en-1-yl)-N-methylcarbamate (4-7, 833 mg, 3.87 mmol, 2 equiv) in THF (18 mL) was added t-BuOK (325 mg, 2.90 mmol, 1.5 equiv) in portions at 0° C. The resulting mixture was stirred for 1 hour at room temperature, quenched with sat. NH₄Cl (aq.) and extracted with CH₂Cl₂ (3×200 mL). The combined organic layers were washed with brine, dried over anhydrous Na₂SO₄, filtered, and concentrated to give a residue. The residue was purified by reverse-phase flash chromatography (50% to 70% MeCN in water (10 mmol/L NH₄HCO₃)) to afford 4-8 as a light-yellow solid. (ESI, m/z): 599 [M+H]⁺.

Step F: To a stirred solution of 4-8 (330 mg, 0.55 mmol, 1 equiv) in dichloroethane (330 mL) was added Grubbs 2nd generation catalyst (186 mg, 0.22 mmol, 0.4 equiv). The resulting mixture was stirred for 2 hours at 80° C., then cooled to room temperature, concentrated, and extracted with CH₂Cl₂ (3×100 mL). The combined organic layers were washed with brine, dried over anhydrous Na₂SO₄, filtered, and concentrated to give a residue. The residue was purified by reverse-phase flash chromatography (50% to 70% MeCN in water (10 mmol/L NH₄HCO₃)) to afford 4-9 as a yellow green solid. (ESI, m/z): 571 [M+H]⁺.

Step G: To a stirred mixture of 4-9 (85 mg, 0.149 mmol, 1 equiv) and 1-13 (180 mg, 0.44 mmol, 3 equiv) in THF (8.5 mL) were added K₃PO₄ (283 mg, 1.34 mmol, 9 equiv) and 2nd generation XPhos Precatalyst/X-Phos aminobiphenyl palladium chloride precatalyst (23 mg, 0.030 mmol, 0.2 equiv). The resulting mixture was stirred for 2.5 hours at 65° C. under argon atmosphere, then cooled to room temperature, filtered, and the filter cake washed with CH₂Cl₂ (3×30 mL). The filtrate was concentrated under reduced pressure to give a residue. The residue was purified by reverse-phase flash chromatography (60% to 80% MeCN in water (10 mmol/L NH_{4H}CO₃)) to afford the desired product as a light-yellow solid. (ESI, m/z): 783 [M+H]⁺.

Step H: To a stirred solution tert-butyl (((11S,Z)-15-(2-((tert-butoxycarbonyl)amino)-3-cyano-7-fluorobenzo[b]thiophen-4-yl)-14-chloro-16-fluoro-11-methyl-4,5,8,9,11,12-hexahydro-2,17-(azeno)benzo[6,7][1,4]oxazepino[5,4-d][1]oxa[3,5]diazacycloundecin-4-yl)methyl)(methyl)carbamate (60 mg, 0.077 mmol, 1 equiv) in DCM (1.8 mL) was added TFA (0.6 mL) at room temperature. The resulting mixture was stirred for 30 minutes at room temperature, then concentrated under vacuum to afford the desired product, which was used in the next step directly without further purification. (ESI, m/z): 583 [M+H]⁺.

Step I: To a stirred mixture of 2-amino-4-((11S,Z)-14-chloro-16-fluoro-11-methyl-4-((methylamino)methyl)-4,5,8,9,11,12-hexahydro-2,17-(azeno)benzo[6,7][1,4]oxazepino[5,4-d][1]oxa[3,5]diazacycloundecin-15-yl)-7-fluorobenzo[b]thiophene-3-carbonitrile (40 mg, 0.069 mmol, 1 equiv) and formaldehyde (37 wt % in water) (55 mg, 0.69 mmol, 10 equiv) in MeOH (4 mL) was added NaBH₃CN (17 mg, 0.27 mmol, 4 equiv) at 0° C. The reaction mixture was stirred for 1 hour at room temperature, then concentrated and the residue purified by prep-HPLC (35% to 62% MeCN in water (10 mmol/L NH₄HCO₃)) to afford 103, 127, and 114 as white solids. Peak 1 (103): (ESI, m/z): 597 [M+H]⁺; ¹H NMR: (400 MHz, DMSO-d6, ppm) δ 8.06 (s, 2H), 7.24-7.21 (m, 1H), 7.15-7.11 (m, 1H), 5.77-5.66 (m, 2H), 4.76-4.70 (m, 1H), 4.64-4.60 (m, 1H), 4.44 (d, J=12.0 Hz, 1H), 4.19-4.05 (m, 2H), 3.66-3.61 (m, 1H), 2.79-2.71 (m, 1H), 2.69-2.66 (m, 1H), 2.59-2.54 (m, 2H), 2.33-2.27 (m, 1H), 2.23 (s, 6H), 2.20-1.93 (m, 1H), 1.33 (d, J=6.6 Hz, 3H). Peak 2 (127): (ESI, m/z): 597 [M+H]⁺; ¹H NMR: (400 MHz, DMSO-d6, ppm) δ 8.07 (d, J=13.2 Hz, 2H), 7.24-7.16 (m, 1H), 7.14-7.09 (m, 1H), 5.78-5.68 (m, 2H), 4.70-4.62 (m, 2H), 4.43-4.34 (m, 1H), 4.22-4.15 (m, 1H), 3.80-3.73 (m, 1H), 3.67-3.58 (m, 1H), 2.98-2.90 (m, 1H), 2.73-2.70 (m, 1H), 2.62-2.58 (m, 1H), 2.40-2.32 (m, 2H), 2.26-2.17 (m, 6H), 1.99-1.92 (m, 1H), 1.38-1.32 (s, 3H). Peak 3 (114): (ESI, m/z): 597 [M+H]⁺; ¹H NMR: (400 MHz, DMSO-d6, ppm) δ 8.07 (s, 2H), 7.22-7.20 (m, 11H), 7.15-7.09 (m, 11H), 5.77-5.66 (m, 2H), 4.76-4.71 (m, 11H), 4.66-4.62 (m, 11H), 4.44-4.08 (m, 3H), 3.66-3.54 (m, 1H), 2.79-2.66 (m, 2H), 2.57-2.52 (m, 1H), 2.34-2.29 (m, 2H), 2.23 (s, 6H), 1.38-1.33 (m, 3H).

Example 1e: Synthesis of (Z)-2-amino-4-(14-chloro-4-(2-(dimethylamino)ethyl)-16-fluoro-4,5,8,9,11,12-hexahydro-2,17-(azeno)benzo[6,7][1,4]oxazepino[5,4-d][1]oxa[3,5]diazacycloundecin-15-yl)-7-fluorobenzo[b]thiophene-3-carbonitrile (154 and 148)

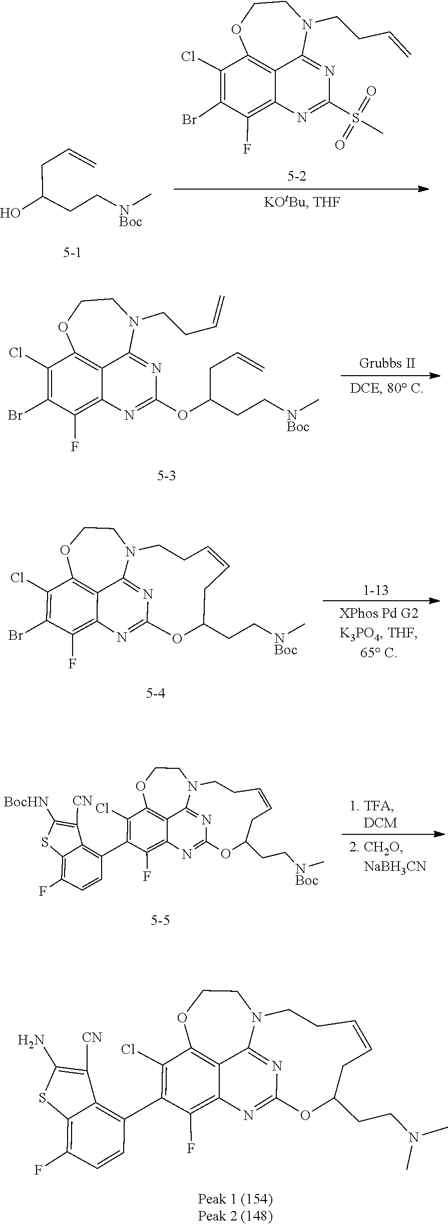 [SMILES: C=CCCN1CCOc2c(Cl)c(Br)c(F)c3nc(S(C)(=O)=O)nc1c23 ; C=CCC(O)CCN(C)C(=O)OC(C)(C)C ; C=CCCN1CCOc2c(Cl)c(Br)c(F)c3nc(OC(CC=C)CCN(C)C(=O)OC(C)(C)C)nc1c23 ; CN(CCC1C/C=C\CCN2CCOc3c(Cl)c(Br)c(F)c4nc(nc2c34)O1)C(=O)OC(C)(C)C ; CN(CCC1C/C=C\CCN2CCOc3c(Cl)c(-c4ccc(F)c5sc(NC(=O)OC(C)(C)C)c(C#N)c45)c(F)c4nc(nc2c34)O1)C(=O)OC(C)(C)C ; CN(C)CCC1C/C=C\CCN2CCOc3c(Cl)c(-c4ccc(F)c5sc(N)c(C#N)c45)c(F)c4nc(nc2c34)O1]

Step A: To a stirred mixture of tert-butyl N-(3-hydroxyhex-5-en-1-yl)-N-methylcarbamate (5-1, 1.15 g, 5.01 mmol, 1.5 equiv) and 9-bromo-4-(but-3-en-1-yl)-8-chloro-10-fluoro-2-(methylsulfonyl)-5,6-dihydro-4H-[1,4]oxazepino[5,6,7-de]quinazoline (5-2, 1.51 g, 3.34 mmol, 1 equiv) in THF (10 mL) was added potassium 2-methylpropan-2-olate (5 mL, 1 M in THF, 1.5 equiv) dropwise at 0° C. The resulting mixture was stirred for 1 hour, then concentrated under reduced pressure to give a residue. The residue was purified by silica gel column chromatography, eluting with petroleum ether/ethyl acetate (3:1) to afford 5-3 as a yellow oil. (ESI, m/z): 599 [M+H]⁺.

Step B: A solution of 5-3 (1.2 g, 2.00 mmol, 1 equiv) in dichloroethane (1 L) was treated with Grubbs 2nd generation catalyst (0.68 g, 0.80 mmol, 0.4 equiv) for 3 hours at 80° C. The resulting mixture was cooled to room temperature and concentrated under reduced pressure to give a residue. The residue was purified by silica gel column chromatography, eluting with petroleum ether/ethyl acetate (5:1) to afford 5-4 as a solid. (ESI, m/z): 571 [M+H]⁺.

Step C: To a stirred solution of 5-4 (170 mg, 0.29 mmol, 1 equiv) and 1-13 (180 mg, 0.446 mmol, 1.5 equiv) in THF (3 mL) were added XPhos-Pd G2 (45 mg, 0.059 mmol, 0.2 equiv) and K₃PO₄ (189 mg, 0.89 mmol, 3 equiv) in portions. The resulting mixture was stirred overnight at 65° C., then cooled to room temperature and concentrated to give a residue. The residue was purified by silica gel column chromatography, eluting with petroleum ether/ethyl acetate (2:1) to give 5-5 as a solid. (ESI, m/z): 783 [M+H]⁺.

Step D: To a solution of 5-5 (260 mg, 0.14 mmol, 1 equiv) in DCM (3 mL) was added TFA (1 mL) dropwise at 0° C. The resulting mixture was stirred for 2 hours at room temperature, then concentrated under reduced pressure to give a residue. The residue was purified by reverse-phase flash chromatography, eluting with 10% to 50% MeCN in water (10 mmol/L NH₄HCO₃) to afford the desired product. (ESI, m/z): 583 [M+H]⁺.

Step E: A mixture of (Z)-2-amino-4-(14-chloro-16-fluoro-4-(2-(methylamino)ethyl)-4,5,8,9,11,12-hexahydro-2,17-(azeno)benzo[6,7][1,4]oxazepino[5,4-d][1]oxa[3,5]diazacycloundecin-15-yl)-7-fluorobenzo[b]thiophene-3-carbonitrile (20 mg, 0.034 mmol, 1 equiv), NaBH₃CN (4 mg, 0.068 mmol, 2 equiv) and formaldehyde (10 mg, 0.34 mmol, 10 equiv) in MeOH (1 mL) was stirred overnight. The resulting mixture was concentrated under reduced pressure to give a residue, which was purified by reverse-phase flash chromatography, eluting with 10% to 50% MeCN in water (10 mmol/L NH₄HCO₃) to afford 154 and 148 as white solids. Peak 1 (154): (ESI, m/z): 597 [M+H]⁺; ¹H NMR: (400 MHz, Chloroform-d, ppm) δ 7.19 (dd, J=8.3, 5.1 Hz, 1H), 7.01 (t, J=8.7 Hz, 1H), 5.79-5.68 (m, 2H), 5.48 (s, 2H), 4.70-4.57 (m, 2H), 4.51-4.40 (m, 1H), 4.16-4.01 (m, 2H), 3.95 (dd, J=14.6, 4.9 Hz, 1H), 3.41-3.29 (m, 1H), 3.09 (t, J=11.3 Hz, 1H), 2.74 (s, 1H), 2.68-2.57 (m, 2H), 2.37 (s, 6H), 2.08-1.99 (m, 2H), 1.26 (s, 2H). Peak 2 (148): (ESI, m/z): 597 [M+H]⁺; ¹H NMR: (400 MHz, Chloroform-d, ppm) δ 7.21 (dd, J=8.3, 5.0 Hz, 1H), 7.04 (t, J=8.8 Hz, 1H), 5.76 (q, J=8.8, 6.9 Hz, 2H), 5.35 (s, 2H), 4.72-4.57 (m, 2H), 4.54-4.45 (m, 1H), 4.14-4.10 (m, 1H), 4.04 (d, J=4.1 Hz, 2H), 3.44-3.30 (m, 1H), 3.19-3.00 (m, 2H), 2.93 (s, 1H), 2.62 (s, 6H), 2.36 (s, 1H), 2.25 (s, 1H), 2.10-2.01 (m, 1H).

Example 1f: Synthesis of 2-amino-4-(14-chloro-5-((dimethylamino)methyl)-16-fluoro-8-oxo-4,5,6,7,8,9,11,12-octahydro-2,17-(azeno)benzo[6,7][1,4]oxazepino[5,4-d][1]oxa[3,5,8]triazacycloundecin-15-yl)-7-fluorobenzo[b]thiophene-3-carbonitrile (147)

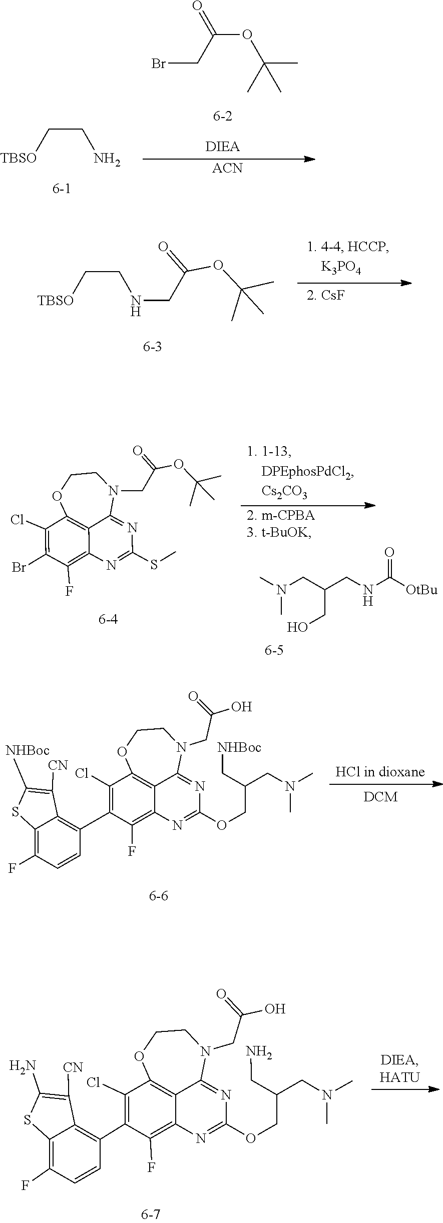

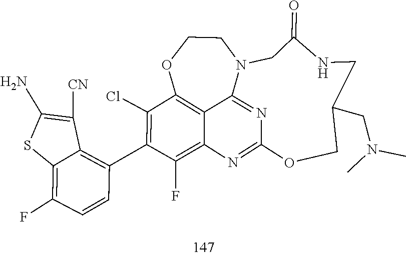 [SMILES: CN(C)CC1CNC(=O)CN2CCOc3c(Cl)c(-c4ccc(F)c5sc(N)c(C#N)c45)c(F)c4nc(nc2c34)OC1]

Step A: To a stirred solution of 2-((tert-butyldimethylsilyl)oxy)ethan-1-amine (6-1, 5 g, 28.55 mmol) and tert-butyl 2-bromoacetate (6-2, 1.11 g, 5.71 mmol) in ACN (100 mL) was added DIEA (7.38 g, 57.10 mmol). The resulting mixture was stirred for 2 hours, then concentrated to give a residue. The residue was purified by silica gel column chromatography, eluting with MeOH/DCM (1:10) to afford the desired product as a colorless oil. (ESI, m/z): 290.3 [M+H]⁺.

Step B: To a solution of 4-4 (1.7 g, 5.00 mmol) in acetonitrile (20 mL) were added HCCP (1.74 g, 5.00 mmol) and K₃PO₄ (3.18 g, 15.00 mmol). After the reaction mixture was stirred for 1 hour, 6-3 (1.45 g, 5.00 mmol) was added and the resulting mixture was stirred for an additional 1 hour. The mixture was diluted with ethyl acetate (300 mL), washed with brine, dried over sodium sulfate, filtered and concentrated to give a residue, which was purified by column chromatography eluting with 0-30% ethyl acetate in petroleum ether to afford the desired product as a yellow solid. (ESI, m/z): 612.1 [M+H]⁺.

Step C: A mixture of tert-butyl N-(7-bromo-6-chloro-5,8-difluoro-2-(methylthio)quinazolin-4-yl)-N-(2-((tert-butyldimethylsilyl)oxy)ethyl)glycinate (1.80 g, 2.93 mmol) and CsF (1.34 g, 8.80 mmol) in DMF (36 mL) was stirred for 30 hours at 80° C., then cooled to room temperature and extracted with EtOAc (3×50 mL). The combined organic layers were washed with brine (2×50 mL), dried over anhydrous Na₂SO₄, filtered, and concentrated under reduced pressure to give a residue. The residue was purified by reverse-phase flash chromatography, eluting with 60% to 90% MeCN in water (10 mmol/L NH₄HCO₃) to afford 6-4 as a yellow solid. (ESI, m/z): 478.0 [M+H]⁺.

Step D: To a stirred solution of 6-4 (600 mg, 1.26 mmol) and 1-13 (1.53 g, 3.77 mmol) in toluene (10 mL) were added DPEDHOS PdCl₂ (181 mg, 0.25 mmol) and Cs₂CO₃ (1.23 g, 3.77 mmol). The resulting mixture was stirred for 2.5 hours at 100° C., then cooled to room temperature, quenched with water/ice, and extracted with CH₂Cl₂ (3×20 mL). The combined organic layers were washed with brine, dried over anhydrous Na₂SO₄, filtered, and concentrated under reduced pressure to give a residue. The residue was purified by silica gel column chromatography, eluting with MeOH/DCM (1:15) to afford the desired product as a yellow solid. (ESI, m/z): 690.7 [M+H]⁺.

Step E: To a stirred solution of tert-butyl 2-(9-(2-((tert-butoxycarbonyl)amino)-3-cyano-7-fluorobenzo[b]thiophen-4-yl)-8-chloro-10-fluoro-2-(methylthio)-5,6-dihydro-4H-[1,4]oxazepino[5,6,7-de]quinazolin-4-yl)acetate (0.7 g, 1.01 mmol, 1 equiv) in DCM (20 mL) was added m-CPBA (0.43 g, 2.0 mmol, equiv) at −10° C. The resulting mixture was stirred for 1 hour at −10° C., then diluted with CH₂Cl₂ (50 mL) and washed with NaHCO₃ (aq) (3×50 mL). The combined organic layers were washed with brine, dried over anhydrous Na₂SO₄, filtered, and concentrated under reduced pressure to give a residue. The residue was purified by prep-TLC (petroleum ether:ethyl acetate=1:1) to afford the desired product as a yellow solid. (ESI, m/z): 706.1 [M+H]⁺.

Step F: To a stirred mixture of tert-butyl 2-(9-(2-((tert-butoxycarbonyl)amino)-3-cyano-7-fluorobenzo[b]thiophen-4-yl)-8-chloro-10-fluoro-2-(methylsulfinyl)-5,6-dihydro-4H-[1,4]oxazepino[5,6,7-de]quinazolin-4-yl)acetate (150 mg, 0.21 mmol, 1 equiv) and tert-butyl (3-(dimethylamino)-2-(hydroxymethyl)propyl)carbamate (6-5, 160 mg, 0.69 mmol, 3 equiv) in toluene (15 mL) was added t-BuOK (1 mol/L in THF) (1.2 mL, 1.2 mmol, 6 equiv) at 0° C. The resulting mixture was stirred for 1 hour, then quenched with water/ice and extracted with EtOAc (2×50 mL). The combined organic layers were washed with brine, dried over anhydrous Na₂SO₄, filtered, and concentrated to give a residue. The residue was purified by reverse-phase flash chromatography, eluting with 0% to 100% MeCN in water (10 mmol/L NH₄HCO₃) to afford 6-6 as a yellow solid. (ESI, m/z): 818 [M+H]⁺.

Step G: To a stirred solution of 6-6 (40 mg 0.049 mmol, 1 equiv) in DCM (3 mL) was added HCl in dioxane (1 mL) at room temperature. The resulting mixture was stirred for 1.5 hours at room temperature, then concentrated under reduced pressure to afford 6-7 as a brown solid. (ESI, m/z): 617.8 [M+H]⁺.

Step H: To a stirred mixture of 6-7 (40 mg, crude, 1 equiv) in MeOH (2 mL) were added DPPA (22 mg, 0.08 mmol, 1.2 equiv) and triethylamine (32 mg, 0.31 mmol, 5 equiv) at 0° C. The resulting mixture was stirred for 5 minutes at room temperature, then concentrated to provide a residue. The residue was purified by prep-HPLC, eluting with 24% to 34% MeCN in water (0.1% FA) to afford 147 as a yellow solid. (ESI, m/z): 600.1 [M+H]⁺; ¹H NMR: (400 MHz, DMSO-d6, ppm) 1H NMR (400 MHz, DMSO) δ 8.10-7.95 (m, 2H), 7.25-7.00 (m, 2H), 4.90-4.31 (m, 4H), 4.20-3.90 (m, 1H), 3.79-3.36 (m, 5H), 2.43-1.86 (m, 9H).

Example 1g: Synthesis of 2-amino-4-(15-chloro-17-fluoro-4-((S)-1-methylpyrrolidin-2-yl)-5,6,7,8,9,10,12,13-octahydro-4H-2,18-(azeno)benzo[6,7][1,4]oxazepino[5,4-d][1]oxa[3,5]diazacyclododecin-16-yl)-7-fluorobenzo[b]thiophene-3-carbonitrile (138)

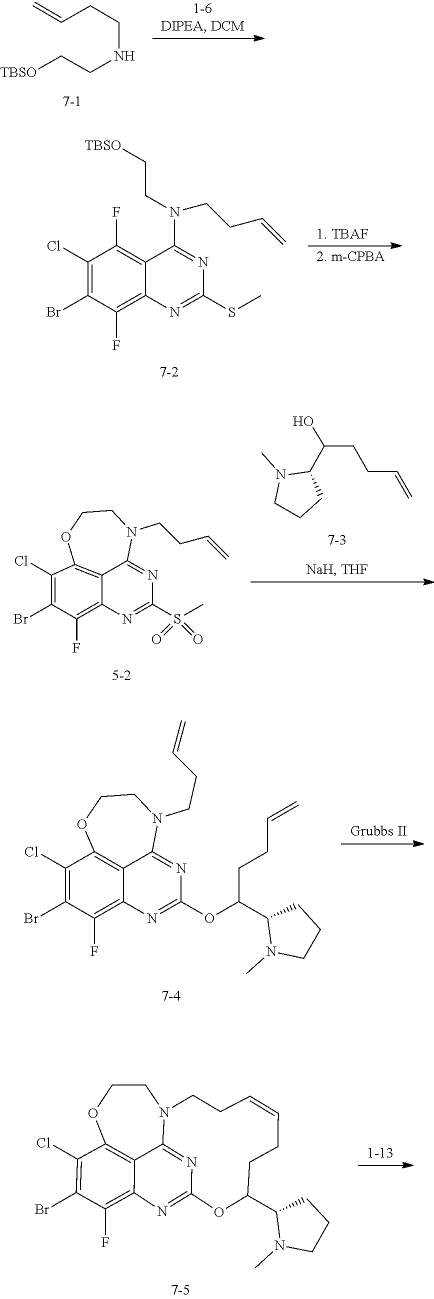 [SMILES: C=CCCNCCO[Si](C)(C)C(C)(C)C ; C=CCCN(CCO[Si](C)(C)C(C)(C)C)c1nc(SC)nc2c(F)c(Br)c(Cl)c(F)c12 ; C=CCCN1CCOc2c(Cl)c(Br)c(F)c3nc(S(C)(=O)=O)nc1c23 ; C=CCCC(O)[C@@H]1CCCN1C ; C=CCCC(Oc1nc2c3c(c(Cl)c(Br)c(F)c3n1)OCCN2CCC=C)[C@@H]1CCCN1C ; CN1CCC[C@H]1C1CC/C=C\CCN2CCOc3c(Cl)c(Br)c(F)c4nc(nc2c34)O1]

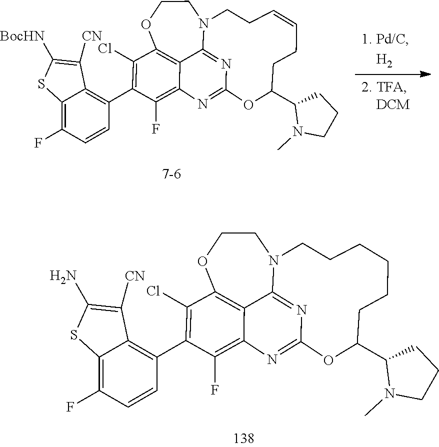 [SMILES: CN1CCC[C@H]1C1CC/C=C\CCN2CCOc3c(Cl)c(-c4ccc(F)c5sc(NC(=O)OC(C)(C)C)c(C#N)c45)c(F)c4nc(nc2c34)O1 ; CN1CCC[C@H]1C1CCCCCCN2CCOc3c(Cl)c(-c4ccc(F)c5sc(N)c(C#N)c45)c(F)c4nc(nc2c34)O1]

Step A: N,N-Diisopropylethylamine (194 μL, 1.12 mmol, 4.0 eq) was added to a mixture of 1-6 (100 mg, 0.27 mmol, 1.0 eq) and N-(2-((tert-butyldimethylsilyl)oxy)ethyl)but-3-en-1-amine (7-1, 96 mg, 0.41 mmol, 1.5 eq) in DCM (5 mL). The mixture was left stirring at room temperature for 2 hours. All the volatiles were removed under reduced pressure before purifying on a silica column eluting with 0 to 50% ethyl acetate/hexanes to afford 7-2 as a white solid. (ESI, m/z): 552.2 [M+H]⁺.

Step B: A TBAF solution (1 M in THF) (738 μL, 0.738 mmol, 3.0 eq) was added slowly to a solution of 7-2 (136 mg, 0.246 mmol, 1.0 eq) in THF (4 mL). The reaction mixture was stirred at ambient temperature for one hour, then sat. NH₄Cl was added. The resulting mixture was extracted with DCM and the combined organics were washed with brine, dried over sodium sulfate, filtered, and concentrated to give a residue. The residue was purified by silica gel column chromatography eluting with 0 to 40% ethyl acetate/hexanes to afford the desired product as a white solid. (ESI, m/z) 418.1 [M+H]⁺.

Step C: To a flask charged with 9-bromo-4-(but-3-en-1-yl)-8-chloro-10-fluoro-2-(methylthio)-5,6-dihydro-4H-[1,4]oxazepino[5,6,7-de]quinazoline (78 mg, 0.187 mmol, 1.0 eq) in DCM (4 ml) was added m-CPBA (96 mg, 0.39 mmol, 2.1 eq) at 0° C. The reaction was allowed to warm to room temperature and stirred for 30 minutes, then quenched by addition of sodium bicarbonate solution and extracted with DCM (2×10 mL). The organics were combined, washed with brine, dried over sodium sulfate, filtered and concentrated to give a residue. The residue was purified by silica gel column chromatography eluting with 0 to 100% ethyl acetate/hexanes to afford 5-2 as a white solid. (ESI, m/z): 450.1 [M+H]⁺.

Step D: NaH was added batchwise to a premixed solution of 5-2 (43 mg, 0.097 mmol, 1.0 eq) and 1-((S)-1-methylpyrrolidin-2-yl)pent-4-en-1-ol (7-3, 82 mg, 0.48 mmol, 5.0 eq) in anhydrous THF (4 mL). Around 50 equivalents of NaH were added in total until LCMS showed the complete consumption of the starting material. The reaction mixture was treated with sat. NH₄Cl solution at 0° C. and extracted with DCM (2×10 mL). The organics were combined, washed with brine, dried over Na₂SO₄, filtered, and concentrated to give a residue. The residue was purified by silica gel column chromatography to afford 7-4. (ESI, m/z): 539.3 [M+H]⁺.

Step E: To a reaction tube charged with a magnetic stir bar were added 7-4 (22 mg, 0.041 mmol, 1.0 eq) and Grubbs II catalyst (1.2 mg, 0.012 mmol, 0.20 eq). The reaction system was vacuumed and back filled with N₂ three times, then anhydrous dichloroethane (1 ml) was added via syringe. The mixture was vacuumed and back filled with N₂ three more times, then stirred at 40° C. for 2 hours. After cooling to room temperature, the reaction mixture was concentrated and the residue was purified by silica gel chromatography eluting with 0-20% MeOH/DCM to afford 7-5 as a solid. (ESI, m/z): 511.2 [M+H]⁺.

Step F: To a reaction tube charged with a magnetic stir bar were added 7-5 (5.5 mg, 0.010 mmol, 1.0 eq), 1-13 (8.7 mg, 0.021 mmol, 1.8 eq), DPEPhosPDCl₂ (1.22 mg, 0.002 mmol, 0.15 eq), and cesium carbonate (7.4 mg, 0.022 mmol, 2.0 eq). The reaction system was vacuumed and back filled with N₂ three times, then anhydrous toluene (2 mL) was added. The mixture was vacuumed and refilled with N₂ three more times, then stirred at 100° C. for 2 hours. After cooling to room temperature, the solvent was removed and the residue purified by silica gel chromatography eluting with 0-20% MeOH/DCM to afford 7-6 as a white solid (ESI, m/z): 723.4 [M+H]⁺.

Step G: To a round bottom flask charged with a stir bar were added 7-6 (4.3 mg, 0.006 mmol), ethyl acetate (0.6 mL), methanol (0.2 mL) and Pd/C (0.4 mg). The reaction mixture was left stirring under hydrogen atmosphere for 30 minutes, then filtered through a pad of celite and concentrated to give the desired product, which was used in the next step without further purification. (ESI, m/z): 725.4 [M+H]⁺.

Step H: The crude product from the previous step was dissolved in DCM (1 mL), then TFA (0.2 mL) was added slowly at 0° C. The reaction mixture was warmed to room temperature and left stirring for 1 hour. Solvent was removed to give a residue, which was purified by reverse phase chromatography eluting with 0-50% ACN/H₂O (+0.1% formic acid) to afford 138 as a solid. (ESI, m/z): 625.30 [M+H]⁺.

### Example 2: Ras Sequences

Human K-Ras Wildtype sequence (SEQ ID NO. 1)

| | |
|---|---|
| 1 | MTEYKLVVVG AGGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHHYREQI |
| 101 | KRVKDSEDVP MVLVGNKCDL PSRTVDTKQA QDLARSYGIP |
| | FIETSAKTRQ |
| 151 | GVDDAFYTLV REIRKHKEKM SKDGKKKKKK SKTKCVIM |

Human K-Ras G12D (SEQ ID NO. 2)

| | |
|---|---|
| 1 | MTEYKLVVVG ADGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHHYREQI |
| 101 | KRVKDSEDVP MVLVGNKCDL PSRTVDTKQA QDLARSYGIP |
| | FIETSAKTRQ |
| 151 | GVDDAFYTLV REIRKHKEKM SKDGKKKKKK SKTKCVIM |

Human K-Ras G12V (SEQ ID NO. 3)

| | |
|---|---|
| 1 | MTEYKLVVVG AVGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHHYREQI |
| 101 | KRVKDSEDVP MVLVGNKCDL PSRTVDTKQA QDLARSYGIP |
| | FIETSAKTRQ |
| 151 | GVDDAFYTLV REIRKHKEKM SKDGKKKKKK SKTKCVIM |

Human K-Ras G12S (SEQ ID NO. 4):

| | |
|---|---|
| 1 | MTEYKLVVVG ASGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHHYREQI |
| 101 | KRVKDSEDVP MVLVGNKCDL PSRTVDTKQA QDLARSYGIP |
| | FIETSAKTRQ |
| 151 | GVDDAFYTLV REIRKHKEKM SKDGKKKKKK SKTKCVIM |

Human N-Ras wildtype (SEQ ID NO. 5)

| | |
|---|---|
| 1 | MTEYKLVVVG AGGVGKSALT IQLIQNHFVD EYDPTIEDSY |
| | RKQVVIDGET |
| 51 | CLLDILDTAG QEEYSAMRDQ YMRTGEGFLC VFAINNSKSF |
| | ADINLYREQI |
| 101 | KRVKDSDDVP MVLVGNKCDL PTRTVDTKQA HELAKSYGIP |
| | FIETSAKTRQ |
| 151 | GVEDAFYTLV REIRQYRMKK LNSSDDGTQG CMGLPCVVM |

H-Ras G12D (SEQ ID NO. 6)

| | |
|---|---|
| 1 | MTEYKLVVVG ADGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHQYREQI |
| 101 | KRVKDSDDVP MVLVGNKCDL AARTVESRQA QDLARSYGIP |
| | YIETSAKTRQ |
| 151 | GVEDAFYTLV REIRQHKLRK LNPPDESGPG CMSCKCVLS |

H-Ras wildtype (SEQ ID NO. 7)

| | |
|---|---|
| 1 | MTEYKLVVVG AGGVGKSALT IQLIQNHFVD EYDPTIEDSY |
| | RKQVVIDGET |
| 51 | CLLDILDTAG QEEYSAMRDQ YMRTGEGFLC VFAINNTKSF |
| | EDIHQYREQI |
| 101 | KRVKDSDDVP MVLVGNKCDL AARTVESRQA QDLARSYGIP |
| | YIETSAKTRQ |
| 151 | GVEDAFYTLV REIRQHKLRK LNPPDESGPG CMSCKCVLS |

Human N-Ras G12D (SEQ ID NO. 8)

| | |
|---|---|
| 1 | MTEYKLVVVG ADGVGKSALT IQLIQNHFVD EYDPTIEDSY |
| | RKQVVIDGET |
| 51 | CLLDILDTAG QEEYSAMRDQ YMRTGEGFLC VFAINNSKSF |
| | ADINLYREQI |
| 101 | KRVKDSDDVP MVLVGNKCDL PTRTVDTKQA HELAKSYGIP |
| | FIETSAKTRQ |
| 151 | GVEDAFYTLV REIRQYRMKK LNSSDDGTQG CMGLPCVVM |

### Example 3: Protein Expression

DNA expression constructs encoding one or more protein sequences of interest (e.g., Kras fragments thereof, mutant variants thereof, etc.) and its corresponding DNA sequences are optimized for expression in E. coli and synthesized by, for example, the GeneArt Technology at Life Technologies. In some cases, the protein sequences of interest are fused with a tag (e.g., glutathione S-transferase (GST), histidine (His), or any other affinity tags) to facilitate recombinant expression and purification of the protein of interest. Such tag can be cleaved subsequent to purification. Alternatively, such tag may remain intact to the protein of interest and may not interfere with activities (e.g., target binding and/or phosphorylation) of the protein of interest

A resulting expression construct is additionally encoded with (i) att-site sequences at the 5′ and 3′ ends for subcloning into various destination vectors using, for example, the Gateway Technology, as well as (ii) a Tobacco Etch Virus (TEV) protease site for proteolytic cleavage of one or more tag sequences. The applied destination vectors can be a pET vector series from Novagen (e.g., with ampicillin resistance gene), which provides an N-terminal fusion of a GST-tag to the integrated gene of interest and/or a pET vector series (e.g., with ampicillin resistance gene), which provides an N-terminal fusion of a HIS-tag to the integrated gene. To generate the final expression vectors, the expression construct of the protein of interest is cloned into any of the applied destination vectors. The expression vectors are transformed into an E. coli strain, e.g., BL21 (DE3). Cultivation of the transformed strains for expression is performed in a 10 L or 1 L fermenter. The cultures are grown, for example, in Terrific Broth media (MP Biomedicals, Kat. #1 13045032) with 200 μg/mL ampicillin at a temperature of 37° C. to a density of 0.6 (OD600), shifted to a temperature of ˜27° C. (for K-Ras expression vectors) induced for expression with 100 mM IPTG, and further cultivated for 24 hours. After cultivation, the transformed E. coli cells are harvested by centrifugation and the resulting pellet is suspended in a lysis buffer, as provided below, and lysed by passing three-times through a high pressure device. The lysate is centrifuged (49000 g, 45 min, 4° C.) and the supernatant is used for further purification.

### Example 4: Ras Protein Purification

A Ras (e.g., K-Ras wildtype or a mutant such as K-Ras G12S, K-Ras G12D, K-Ras G12V or K-Ras G12C) construct or a variant thereof is tagged with GST. E. coli culture from a 10 L fermenter is lysed in lysis buffer (50 mM Tris HCl 7.5, 500 mM NaCl, 1 mM DTT, 0.5% CHAPS, Complete Protease Inhibitor Cocktail-(Roche)). As a first chromatography step, the centrifuged lysate is incubated with 50 mL Glutathione Agarose 4B (Macherey-Nagel; 745500.100) in a spinner flask (16 h, 10° C.). The Glutathione Agarose 4B loaded with protein is transferred to a chromatography column connected to a chromatography system, e.g., an Akta chromatography system. The column is washed with wash buffer (50 mM Tris HCl 7.5, 500 mM NaCl, 1 mM DTT) and the bound protein is eluted with elution buffer (50 mM Tris HCl 7.5, 500 mM NaCl, 1 mM DTT, 15 mM Glutathione). The main fractions of the elution peak (monitored by OD280) are pooled. For further purification by size-exclusion chromatography, the above eluate volume is applied to a column Superdex 200 HR prep grade (GE Healthcare) and the resulting peak fractions of the eluted fusion protein is collected. Native mass spectrometry analyses of the final purified protein construct can be performed to assess its homogeneous load with GDP.

### Example 5: HTRF (Homogenous Time-Resolved Fluorescence) Resonance Energy Transfer Assay

The ability of a compound of the present disclosure to reduce a Ras signaling output can be demonstrated by an HTRF assay. This assay can be also used to assess a selective inhibition or reduction of signaling output of a mutant Ras protein relative to a wildtype, or relative to a different mutant Ras protein. For example, the equilibrium interaction of wildtype Kras or K-Ras mutant (e.g., wildtype or a mutant thereof) with SOS1 (e.g., hSOS1) can be assessed as a proxy or an indication for a subject compound's ability to bind and inhibit Ras protein. The HTRF assay detects from (i) a fluorescence resonance energy transfer (FRET) donor (e.g., antiGST-Europium) that is bound to GST-tagged K-Ras mutant to (ii) a FRET acceptor (e.g., anti-6His-XL665) bound to a His-tagged hSOS1.

The assay buffer can contain ˜5 mM HEPES pH 7.4, ˜150 mM NaCl, ˜ 1 mM DTT, 0.05% BSA and 0.0025% (v/v) Igepal. A Ras working solution is prepared in an assay buffer containing typically a suitable amount of the protein construct (e.g., GST-tagged K-Ras mutant) and the FRET donor (e.g., antiGST-Eu(K) from Cisbio, France). A SOS1 working solution is prepared in an assay buffer containing suitable amount of the protein construct (e.g., His-hSOS1) and the FRET acceptor (e.g., anti-6His-XL665 from Cisbio, France). A suitable amount of the protein construct will depend on the range of activity or range of IC50 values being detected or under investigation. For detecting an IC50 within a range of 500 nM, the protein constructs of the same range of molarity can be utilized. An inhibitor control solution is prepared in an assay buffer containing comparable amount of the FRET acceptor without the SOS1 protein.

A fixed volume of DMSO with or without test compound is transferred into a 384-well plate. Ras working solution is added to all wells of the test plate. SOS1 working solution is added to all wells except for those that are subsequently filled with inhibitor control solution. Upon incubation for about 10 minutes or longer, the fluorescence is measured with a M1000Pro plate reader (Tecan) using HTRF detection (excitation 337 nm, emission 1: 620 nm, emission 2: 665 nm). Compounds are tested in duplicate at different concentrations (for example, 10 μM, 2.5 μM, 0.63 μM, 0.16 μM, 0.04 μM, 0.01 μM test compound). The ratiometric data (i.e., emission 2 divided by emission 1) is used to calculate IC50 values against Ras using GraphPad Prism (GraphPad software). Signaling output measured in terms of IC50 values can be obtained and a ratio of IC50 against one mutant relative to another mutant can be calculated. For instance, a selective reduction of K-Ras G12D signaling output can be evidenced by a ratio greater than one. In particular, a selective reduction of K-Ras G12D signaling relative to K-Ras WT signaling is evidenced if the ratio of IC50 (against K-Ras WT) to IC50 (against K-Ras G12D) is greater than 1. In some embodiments, one or more subject compounds disclosed herein exhibits selective inhibition of a Ras mutant (e.g., G12C, G12D, G12S, G12V, G13C, or G13D) over WT by at least 1-fold, and in some instances greater than 2-, 3-, 4- or 5-fold. In some embodiments, one or more subject compounds disclosed herein exhibits an IC50 against KRas mutants (e.g., G12C, G12D, G12S, G12V, G13C, or G13D) less than 500 nM, such as less than 100 nM, 50 nM, 10 nM or even less.

The ability of one or more compounds exemplified in Table 1 to inhibit wildtype KRAS or a KRAS mutant is demonstrated utilizing the procedures described above. Table 2 shows the resulting IC50 values of exemplary compounds against KRAS G12D, KRAS G12V, and wildtype KRAS using the HTRF assay described herein. Compound numbers correspond to the numbers and structures provided in Table 1 and Example 1.

TABLE 2

| | ≤5 μM |
|---|---|
| Inhibition of | 101, 103, 104, 107, 108, 110, 112, 114, 115, 116, 117, 119, 120, 123, |
| KRAS G12D | 126, 127, 128, 130, 131, 132, 133, 134, 135, 137, 138, 141, 143, 145, |
| (IC₅₀) | 147, 148, 149, 150, 151, 153, 154, 155, 156, 157, 158, 159 |
| Inhibition of | 101, 103, 104, 107, 108, 110, 112, 114, 115, 116, 117, 119, 120, 123, |
| KRAS WT | 126, 127, 128, 130, 131, 132, 133, 134, 135, 137, 138, 141, 143, 145, |
| (IC₅₀) | 147, 148, 149, 150, 151, 153, 154, 155, 156, 157, 158, 159 |
| Inhibition of | 101, 103, 104, 107, 108, 110, 112, 113, 114, 115, 116, 117, 119, 120, |
| KRAS G12V | 123, 126, 127, 128, 130, 131, 132, 133, 134, 135, 137, 138, 141, 143, |
| (IC₅₀) | 145, 147, 148, 149, 150, 151, 153, 154, 155, 156, 157, 158, 159 |

### Example 6: GTPase Activity Assay

The ability of a compound of the present disclosure to inhibit Ras protein signaling can be demonstrated by a reduced GTPase activity. This assay can also be used to assess selective inhibition of a mutant Ras protein relative to a wildtype, or relative to a different mutant Ras protein. For instance, the assay can be used to establish a subject compound's ability to selectively inhibit Kras G12D relative to wildtype, Kras G12S relative to wildtype, Kras G12V relative to wildtype, Kras G12S relative Kras G12V, Kras G12S relative Kras G12D, Kras G12D relative Kras G12S, or Kras G12D relative Kras G12V. In particular, intrinsic and GTPase-activating protein (GAP)-stimulated GTPase activity for a K-Ras construct or a mutant thereof can be measured using EnzCheck phosphate assay system (Life Technologies). For example, K-Ras WT, K-Ras D154Q mutant, K-Ras G12D mutant, K-Ras G12S mutant, and K-Ras G12D/D154Q mutant proteins (2.5 mg/mL) in buffer (20 mmol/L Tris, pH 8.0, 50 mM NaCl) are loaded with GTP at room temperature for 2 hours by exposing to exchange buffer containing EDTA. Proteins are buffer exchanged to assay buffer (30 mM Tris, pH 7.5, 1 mM DTT) and the concentration is adjusted to 2 mg/mL. GTP loading is verified by back extraction of nucleotide using 6M urea and evaluation of nucleotide peaks by HPLC using an ion-exchange column. The assay is performed in a clear 384-well plate (Costar) by combining GTP-loaded K-Ras proteins (50 mM final) with 2-amino-6-mercapto-7-methylpurine ribonucleoside (MESG) (200 mM final), and purine nucleotide phosphorylase (5 U/mL final). GTP hydrolysis is initiated by the addition of MgCl₂ at a working concentration of 40 mM. For GAP stimulation, Ras p21 protein activator 1 (P120GAP) can be included at 50 mM. Absorbance at 360 nm can be measured every 8 to 15 s for 1,000 s at 20° C. Samples are tested with or without a subject compound disclosed herein to assess each compound's ability to inhibit signaling of a given Ras protein (e.g., a given mutant Kras) of interest.

### Example 7: Nucleotide Exchange Assay

The ability of a compound of the present disclosure to inhibit Ras protein signaling can be demonstrated by reduced nucleotide exchange activity. This assay can be also used to assess selective inhibition of a mutant Ras protein relative to a wildtype, or relative to a different mutant Ras protein. For example, 250 nM or 500 nM GDP-loaded K-Ras protein (e.g., wildtype or a mutant thereof including those mentioned in Example 6) is incubated with different concentrations of compounds (for example ˜60 μM, ˜20 μM, ˜6.7 μM, ˜2.2 μM, ˜0.7 μM, or ˜0.2 μM subject compound). A control reaction without subject compound is also included. SOS1 (catalytic domain) protein is added to the K-Ras protein solution. The nucleotide exchange reaction is initiated by adding fluorescent labelled GDP (Guanosine 5′-Diphosphate, BODIPY™ FL 2′-(or-3′)-O—(N-(2-Aminoethyl) Urethane) to a final concentration of 0.36 μM. Fluorescence is measured every 30 s for 70 minutes at 490 nm/515 nm (excitation/emission) in a M1000Pro plate reader (Tecan). Data is exported and analyzed to calculate an IC50 using GraphPad Prism (GraphPad Software). Sample(s) can be tested with or without a subject compound disclosed herein to assess the compound's ability to inhibit K-Ras signaling or its IC50 against a given Ras protein (e.g., a given mutant K-Ras) of interest.

### Example 8: Testing for Modification of Ras Protein

Test compounds are prepared as 10 mM stock solutions in DMSO (Fisher cat #BP231-100). KRAS protein (e.g., His-tagged GDP-loaded wildtype 1-169, His-tagged GDP-loaded G12C 1-169, His-tagged GDP-loaded G12D 1-169, or His-tagged GDP-loaded G12S 1-169) is diluted to ˜2 μM in appropriate buffer (e.g., a Hepes buffer at physiological conditions). For testing KRAS modification, compounds are diluted to 50× final test concentration in DMSO in 96-well storage plates. 1 μL of the diluted 50× compounds are added to appropriate wells in the PCR plate (Fisher cat #AB-0800). ˜49 μL of the stock protein solution is added to each well of the 96-well PCR plate. Reactions are mixed carefully. The plate is sealed well with aluminum plate seal and stored in a drawer at room temperature for 24 hrs. 5 μL of 2% formic acid (Fisher cat #A117-50) in MilliQ H₂O is then added to each well followed by mixing with a pipette. The plate is then resealed with aluminum seal and stored until mass spectrometry analysis. The extent of covalent modification of KRAS proteins can be determined by liquid chromatography electrospray mass spectrometry analysis of the intact proteins on a Thermo Q-Exactive Plus mass spectrometer. 20 μL of sample is injected onto a bioZen 3.6 μm Intact C4 column (Phenomenex cat #00B-4767-AN) placed in a column oven set to 40° C. and separated using a suitable LC gradient from −20% to ˜60% solvent B. Solvent A is 0.1% formic acid and solvent B is 0.1% formic acid in acetonitrile. HESI source settings are set to 40, 5 and 1 for the sheath, auxiliary and sweep gas flow, respectively. The spray voltage is 4 kV, and the capillary temperature is 320° C. S-lens RF level is 50 and auxiliary gas heater temperature is set to 200° C. The mass spectrometry is acquired using a scan range from 650 to 1750 m/z using positive polarity at a mass resolution of 70,000, AGC target of 1e6 ions and maximum injection time of 250 ms. The recorded protein mass spectrum is deconvoluted from the raw data file using Protein Deconvolution v4.0 (Thermo). The protein mass and adduct masses are exported with their peak intensities. The peak intensities for the unmodified and modified protein are used to calculate the percent covalent modification of the KRAS protein based on the following equation:

$$\% KRAS protein modification = ( ( KRAS - compound ) / ( KRAS ) + ( KRAS - Compound ) ) * 100.$$

### Example 9: Ras Cellular Assay

The ability of a compound of the present disclosure to inhibit Ras protein signaling can be demonstrated by inhibiting growth of a given Kras mutant cell line. For example, this assay can be also used to assess selective growth inhibition of a mutant Ras protein relative to a wildtype, or relative to a different mutant Ras protein.

a. Growth of Cells with K-Ras G12C Mutation

MIA PaCa-2 (ATCC CRL-1420) and NCI-H1792 (ATCC CRL-5895) cell lines comprise a G12C mutation and can be used to assess Ras cellular signaling in vitro, e.g., in response to an inhibitor compound of the present disclosure. This cellular assay can also be used to discern selective inhibition of a subject compound against certain types of Kras mutants, e.g., more potent inhibition against Kras G12D relative to Kras G12C mutant, by comparing inhibition of MIA PaCa-2 (G12C driven tumor cell line) to inhibition of GP2d (G12D driven tumor cell line). MIA PaCa-2 culture medium is prepared with DMEM/Ham's F12 (e.g., with stable Glutamine, 10% FCS, and 2.5% Horse Serum. NCI-H1792 culture medium is prepared with RPMI 1640 (e.g., with stable Glutamine) and 10% FCS.

On a first day (e.g., Day 1), Softagar (Select Agar, Invitrogen, 3% in ddH₂O autoclaved) is boiled and tempered at 48° C. Appropriate culture medium (i.e., medium) is tempered to 37° C. Agar (3%) is diluted 1:5 in medium (=0.6%) and plated into 96 well plates (Corning, #3904), then incubated at room temperature for agar solidification. A 3% agar is diluted to 0.25% in medium (1:12 dilution) and tempered at 42° C. Cells are trypsinized, counted, and tempered at 37° C. The cells (e.g., MIA PaCa-2 at about 125-150 cells, NCI-H1792 at about 1000 cells) are resuspended in 100 mL 0.25% Agar and plated, followed by incubation at room temperature for agar solidification. The wells are overlaid with 50 mL of the medium. Sister wells in a separate plate are plated for time zero determination. All plates are incubated overnight at 37° C. and 5% CO₂.

On a second day (e.g., Day 2), time zero values are measured. A 40 mL volume of Cell Titer 96 Aqueous Solution (Promega) is added to each well and incubated in the dark at 37° C. and 5% CO₂. Absorption can be measured at 490 nm and reference wavelength 660 nm. DMSO-prediluted test compounds are added to wells of interest, e.g., with HP Dispenser, to one or more desired concentrations (e.g., a final DMSO concentration of 0.3%).

On a tenth day (e.g., Day 10), absorption by wells treated with the test compounds and control wells are measured with, for example, Cell Titer 96 AQueous and analyzed in comparison to the time zero measurements. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRas G12C cell line (including MIA PaCa-2 and NCI-H1792).

b. Growth of Cells with K-Ras G12D Mutation

ASPC-1 (ATCC CRL-1682), Panc-10.05 (ATCC CRL-2547), A427, GP2d cell lines or any other cell lines comprising a G12D mutation can be used to assess Ras cellular signaling in vitro, e.g., in response to a compound described herein. For example, ASPC-1 culture medium is prepared with RPMI-1640 and 10% heat-inactivated FBS. Panc-10.05 culture medium is prepared with RPMI-1640, 10 Units/mL human recombinant insulin, and 10% FBS. A427 cell culture is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384UZ). The day after plating, cells are treated with a dilution series (e.g., a 9 point 3-fold dilution series) of the compounds herein (e.g., approximately 40 μL final volume per well). Cell viability can be monitored (e.g., approximately 5 days later) according to the manufacturer's recommended instructions, where the CellTiter-Glo reagent is added (e.g., approximately 10 μL), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRas G12D cell line (including GP2D, HPAC, AsPC-1, Panc04.03).

c. Growth of Cells with K-Ras G12S Mutation

A549 (ATCC CRL-185) and LS123 (ATCC CRL-255) cell lines comprise a G12S mutation and can be used to assess Ras cellular signaling in vitro, e.g., in response to treatment with a compound described herein. A549 culture medium is prepared with RPMI-1640 and 10% heat-inactivated FBS. LS123 culture medium is prepared with RPMI-1640 and 10% heat-inactivated FBS. A CellTiter-Glo (CTG) luminescent based assay (Promega) is used to assess growth of the cells, as a measurement of the ability of the compounds herein to inhibit Ras signaling in the cells. The cells (e.g., 800 per well) are seeded in their respective culture medium in standard tissue culture-treated 384-well format plates (Falcon #08-772-116) or ultra-low attachment surface 384-well format plates (S-Bio #MS-9384WZ). The day after plating, cells are treated with a dilution series (e.g., a 10 point, 3-fold dilution series) of the compounds herein (e.g., approximately 40 μL final volume per well). Cell viability can be monitored (e.g., approximately 6 days later) according to the manufacturer's recommended instructions, where CellTiter-Glo reagent is added (e.g., approximately 10 μL), vigorously mixed, covered, and placed on a plate shaker (e.g., approximately for 20 min) to ensure sufficient cell lysis prior to assessment of luminescent signal. The IC50 values are determined using the four parameter fit. The resulting IC50 value is a measurement of the ability of the test compound to reduce cell growth of Ras-driven cells (e.g., tumor cell lines) in vitro and/or in vivo. One or more compounds disclosed herein is expected to exhibit an IC50 value less than 5 μM, 1 μM, 100 nM, or even less, against one or more KRAS G12S cell line (including A549 and LS123).

### Example 10: In Vivo Ras Inhibition

The in vivo reduction in Ras signaling output by a compound of the present disclosure is determined in a mouse tumor xenograft model, particularly by using a mutant K-Ras model including without limitation a K-Ras G12S model, a K-Ras G12C model, a K-Ras G12D model, a K-Ras G13D model, and a K-Ras G13C model. These models can be generated by the methods and procedures described below. In particular, the methods disclosed below involving the use of a K-Ras G12D mutant cell line for generating a K-Ras G12D xenograft model can be applied to other K-Ras mutant animal models using the respective K-Ras mutant cell lines described above.

Xenograft with K-Ras G12D, G12C, or G12S Mutation

Tumor xenografts are established by administration of tumor cells with a K-Ras G12D mutation (e.g., ASPC-1 cells), a K-Ras G12C mutation (e.g., MIA PaCa-2 cells), or a K-Ras G12S mutation (e.g., A549 or LS123 cells) into mice. Female 6- to 8-week-old athymic BALB/c nude (NCr) nu/nu mice are used for xenografts. The tumor cells (e.g., approximately 5×10⁶) are harvested on the day of use and injected in growth-factor-reduced Matrigel/PBS (e.g., 50% final concentration in 100 μL). One flank is inoculated subcutaneously per mouse. Mice are monitored daily, weighed twice weekly, and caliper measurements begin when tumors become visible. For efficacy studies, animals are randomly assigned to treatment groups by an algorithm that assigns animals to groups to achieve best case distributions of mean tumor size with lowest possible standard deviation. Tumor volume can be calculated by measuring two perpendicular diameters using the following formula: (L×w²)/2, in which L and w refer to the length and width of the tumor, respectively. Percent tumor volume change can be calculated using the following formula: (V_{final}−Vᵢₙᵢₜᵢₐₗ)/Vᵢₙᵢₜᵢₐₗ×100. Percent of tumor growth inhibition (% TGI) can be calculated using the following formula: % TGI=100×(1−(average V_{final}−Vᵢₙᵢₜᵢₐₗ of treatment group)/(average V_{final}−Vᵢₙᵢₜᵢₐₗ of control group). When tumors reach a threshold average size (e.g., approximately 200-400 mm³), mice are randomized into 3-10 mice per group and are treated with vehicle (e.g., 100% Labrasol®) or a compound disclosed herein, using, for example, a daily schedule by oral gavage. Results can be expressed as mean and standard deviation of the mean.

### Example 11: Metabolic (Microsomal) Stability Assay

The metabolic stability of a test compound is assayed at 37° C. using pooled liver microsomes (mouse or human liver microsomes). An aliquot of 10 μL of 50 μM test compound is mixed with 490 μL of 0.611 mg/mL liver microsomes, then 50 μL of the mixtures are dispensed to the 96 well tubes and warmed at 37° C. for 10 minutes. The reactions are initiated by adding 50 μL of the pre-warmed NADPH regeneration system solution (add 1.2 μL solution, 240 μL solution B, mix with 10.56 mL KPBS) and then incubated at 37° C. The final incubation solution contains 100 mM potassium phosphate (pH 7.4), 1.3 mM NADP⁺, 3.3 mM glucose 6-phosphate, 0.4 unit/mL of glucose 6-phosphate dehydrogenase, 3.3 mM magnesium chloride, 0.3 mg/mL liver microsomes and 0.5 μM test article. After 0, 15, 30 and 60 minutes in a shaking incubator, the reactions are terminated by adding 100 μL of acetonitrile containing 200 nM buspirone as an internal standard. All incubations are conducted in duplicate. Plates are vortexed vigorously by using Fisher Scientific microplate vortex mixer (Henry Troemner, US). Samples are then centrifuged at 3500 rpm for 10 minutes (4° C.) using Sorvall Legend XRT Centrifuge (Thermo Scientific, GE). Supernatants (40 μL) are transferred into clean 96-deep well plates. To each well is added with 160 μL of ultrapure water (Milli-Q, Millipore Corporation) with 0.1% (v/v) formic acid (Fisher Chemical) and the resulting solutions mixed thoroughly and subjected to LC/MS/MS analysis in MRM positive ionization mode.

All samples are measured using a mass spectrometer (QTrap 5500 quadrupole/ion trap) coupled with a Shimadzu HPLC system. The HPLC system consists of a Shimadzu series degasser, binary quaternary gradient pumps, column heater coupled to an autosampler, and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+).

The half-life for the metabolic degradation of the test compound is calculated by plotting the time-course disappearance of the test compound during the incubation with liver microsomes. Each plot is fitted to a first-order equation for the elimination of the test compound (% remaining compound) versus time using non-linear regression (Equation 1).

$$\frac{{C}_{t}}{{C}_{0}} = {e}^{- kt} \qquad Equation 1$$

where Cₜ is the mean relative substrate concentration at time t and Co is the initial concentration (0.5 μM) at time 0. Note that the area ratio of the substrate peak to an internal standard peak is proportional to the analyte concentration and is used for regression analysis to derive a value of k.

The half-life t_{1/2} for metabolic (microsome) stability is derived from the test compound elimination constant k using Equation 2 below.

$${t}_{1 / 2} = \frac{0.693}{k} \qquad Equation 2$$

### Example 12: CYP2C19 Inhibition Assay

Some xenobiotics can inhibit cytochrome P450 (CYP) enzyme function, which alters their ability to metabolize drugs. Administration of a CYP inhibitor with a drug whose clearance is dependent on CYP metabolism can result in increased plasma concentrations of this concomitant drug, leading to potential toxicity. The inhibition of CYP2C19 by a test compound is assayed in human liver microsomes using S-mephenytoin as a CYP2C19 substrate. The stock solution of the test compound or known CYP2C19 inhibitor as a positive control (10 mM) is diluted with KPBS to 40 μM. In a similar way, the stock solutions of the human liver microsomes and S-mephenytoin are diluted with KPBS buffer. The pre-incubations are started by incubating a plate containing 25 μL human liver microsomes (final concentration of 0.2 mg/mL), 25 μL NADPH-generating system, and a 25 μL test compound (final concentration 10 μM) or the positive control for 30 min at 37±1° C. After the pre-incubation, 25 μL S-mephenytoin (final concentration 200 μM) is added and incubated another 12 minutes at 37±1° C. for substrate metabolism. The reactions are terminated by addition of 100 μL of ice-cold acetonitrile containing an internal standard (buspirone). Precipitated proteins are removed by centrifugation at 3500 rpm for 10 minutes at 4° C. (Allegra 25R, Beckman Co. Fullerton, CA), then an aliquot of the supernatant is transferred to an assay plate.

All the samples are assessed using a mass spectrometer (QTrap 5500 quadrupole/ion trap) coupled with a Shimadzu HPLC system following the manufacturer's instructions. The metabolism of S-mephenytoin in human liver microsomes is monitored by LC/MS/MS as representative of CYP2C19 inhibitory activity. The amount of metabolite formed is assessed by the peak area ratio (metabolite/IS) and % inhibition at 10 μM is expressed as a percentage of the metabolite signal reduced compared to the control (i.e. an incubation that contained no inhibitor and represented 100% enzyme activity): % inhibition=(1−A/B)×100%, where A is the metabolite peak area ratio formed in the presence of test compound or inhibitor at 10 μM and B is the metabolite peak area ratio formed without test compound or inhibitor in the incubation.

### Example 13: Mouse and Human Protein Binding Assay to Assess Free Drug Concentration

This assay can be used to determine the plasma protein binding of the test compound in the plasma of human and animal species using a Rapid Equilibrium Dialysis (RED) device for equilibrium dialysis and LC-MS/MS for sample analysis. Test compound is spiked in. The stock solution of the test compound is prepared at 5 mM concentration. One μL of 5 mM working solution is added into 1000 μL plasma to achieve a final concentration of 5 μM. The spiked plasma is placed on a rocker and gently agitated for approximately 20 minutes. A volume of 300 μL of the plasma sample containing 5 μM test compound from each species is added to designated RED device donor chambers followed by addition of 500 μL of potassium phosphate buffer to the corresponding receiver chambers in duplicate. The RED device is then sealed with sealing tape and shaken at 150 RPM for 4 hours at 37° C. Post-dialysis donor and receiver compartment samples are prepared for LC-MS/MS analysis, including spiking samples with an internal standard for the bioanalytical analysis. Warfarin and propranolol are purchased from Sigma-Aldrich (St. Louis, MO), and used as positive controls for low and high plasma protein binding, respectively.

All the samples are analyzed using an Agilent Technologies 6430 Triple Quad LC/MS system. The HPLC system consists of an Agilent 1290 Infinity Liquid Chromatograph coupled to an autosampler (Agilent 1290 Infinity LC Injector HTC), and a Phenomenex Gemini-NX, C18, 3.0 μm or Phenomenex Lunar, C8, 5.0 μM HPLC column (Phenomenex, Torrance, CA), eluting with a mobile phase gradient consisting of Solution A (0.1% formic acid water) and Solution B (0.1% formic acid acetonitrile). The column temperature is maintained at 40° C. All the analytes are detected with positive-mode electrospray ionization (ES+). The percentage of the test compound bound to plasma is calculated following Equations 3 and 4.

$$\% Free test compound = \frac{\begin{matrix}Peak ratio ( \frac{test compound}{Internal standard} ) , \\ receiver compartment\end{matrix}}{\begin{matrix}Peak ratio ( \frac{test compound}{Internal standard} ) , \\ donor compartment\end{matrix}} * 100 \qquad Equation 3$$

$$\% Plasma protein bound test compound = 100 - \% Free test compound \qquad Equation 4$$

### Example 14: hERG (Automated Patch-Clamp) Assay

The human ether-a-go-go related gene (hERG) encodes the voltage gated potassium channel in the heart (IKr) which is involved in cardiac repolarization. Inhibition of the hERG causes QT interval prolongation and can lead to potential fatal events in humans. It is thus important to assess hERG inhibition early in drug discovery. A hERG automated patch-clamp assay is done using a hERG CHO-K1 cell line using an incubation time of 5 min. The degree of hERG inhibition (%) is obtained by measuring the tail current amplitude, which is induced by a one second test pulse to −40 mV after a two second pulse to +20 mV, before and after drug incubation (the current difference is normalized to control and multiplied by 100 to obtain the percent of inhibition). The percent hERG inhibition is measured in the presence of 10 μM test compound.

### Example 15: Rat Oral Exposure (% F)

A pharmacokinetic profile for a test compound is measured by single dosing in jugular vein cannulated male Sprague-Dawley rats. Animal weights are typically over 200 grams, and animals are allowed to acclimate to their new environment for at least 3 days prior to the initiation of any studies. One set of animals is dosed intravenously (IV) with test compound (2 mg/kg in 20% HP-beta-CD or 20% Captisol, pH adjusted to ˜4 by citric acid). The IV dosing solution concentration is 0.4 mg/mL test compound. Blood is sampled at 5 minutes, 15 minutes, 30 minutes, 90 minutes, 360 minutes, and 24 hours following IV dosing. Another set of animals is dosed oral (po) with test compound (10 mg/kg in 20% HP-beta-CD or 20% Captisol, pH adjusted to ˜4 by citric acid). The oral dosing solution concentration is 1 mg/mL test compound. Blood is sampled at 15 minutes, 30 minutes, 90 minutes, 180 minutes, 360 minutes and 24 hours following oral (po) dosing. Blood samples (˜0.2 mL/sample) is collected via the jugular vein, placed in tubes containing EDTA-K2 and stored on ice until centrifuged. The blood samples are centrifuged at approximately 6800 g for 6 minutes at 2-8° C. and the resulting plasma is separated and stored frozen at approximately −80° C.

The plasma samples are analyzed using an Agilent Technologies 6430 Triple Quad LC/MS system, following the manufacturer's instructions. The analytes are detected with positive-mode electrospray ionization (ES+). A standard curve for each test compound is generated and used to measure test compound concentrations in the rat plasma samples. Based on the time course sampling, an area under the curve is calculated for the oral dose group and the intravenous dose group. Percentage rat bioavailability is calculated based on equation 5.

$$\% F ( rat ) = \frac{{AUC}_{po} * {Dose}_{IV}}{{AUC}_{IV} * {Dose}_{po}} . \qquad Equation 5$$

where F is bioavailability, AUCₚₒ is area under curve of oral drug, AUC_{IV} is area under curve of intravenous drug, Dose_{IV} is the intravenous dose and Doseₚₒ is the oral dose.

What is claimed is:

1. A compound of Formula (I): 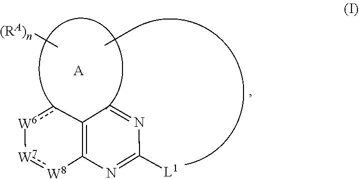
or a pharmaceutically acceptable salt or solvate thereof, wherein:
A is selected from C₅₋₁₈ carbocycle and 5- to 18-membered heterocycle;
W⁶ is C(R⁶), W⁷ is C(R⁷), W⁸ is C(R⁸), and each [symbol] indicates a double bond; W⁶ is N, W⁷ is C(R⁷), W⁸ is C(R⁸), and each [symbol] indicates a double bond; or W⁶ is C(R⁶)(R^{6a}) or C(O), W⁷ is N(R^{7b}), W⁸ is C(R⁸)(R^{8a}) or C(O), and each [symbol] indicates a single bond;
R⁶, R^{6a}, R⁸, and R^{8a} are each independently selected from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹⁵, —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R⁷ is -L⁷-R¹⁷;
R^{7b} is -L^{7b}-R¹⁷;
L⁷ is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
L^{7b} is selected from a bond, —O—, —C(O)—, —S—, —S(O)₂—, —S(O)—, —P(O)R^{7d}—, —C(R^{7c})₂—, —OC(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₁₋₄ alkylene, and 2- to 4-membered heteroalkylene, wherein C₁₋₄ alkylene and 2- to 4-membered heteroalkylene are optionally substituted with one, two, or three R²⁰;
R¹⁷ is selected from C₃₋₁₂ carbocycle and 3- to 12-membered heterocycle, each of which is optionally substituted with one or more R²⁰;
L¹ is -L²-L³-L⁴-L⁵-L⁶-;
L² is selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle, wherein —(C₁₋₆ alkylene)-(C₃₋₁₀ carbocycle)-, —(C₁₋₆ alkylene)-(3- to 10-membered heterocycle)-, —O—(C₁₋₆ alkylene)-(C₃₋₁₀ carbocycle)-, —O—(C₁₋₆ alkylene)-(3- to 10-membered heterocycle)-, —N(R^{7d})—(C₁₋₆ alkylene)-(C₃₋₁₀ carbocycle)-, —N(R^{7d})—(C₁₋₆ alkylene)-(3- to 10-membered heterocycle)-, —S—(C₁₋₆ alkylene)-(C₃₋₁₀ carbocycle)-, —S—(C₁₋₆ alkylene)-(3- to 10-membered heterocycle)-, C₃₋₁₀ carbocycle, and 3- to 10-membered heterocycle are optionally substituted with one, two, or three R^{L};
L³ is selected from C₁₋₆ alkyl, C₂₋₆ alkenyl, C₂₋₆ alkynyl, and 2- to 6-membered heteroalkyl, each of which is optionally substituted with one, two, or three R^{L};
L⁴ and L⁶ are each independently selected from a bond, —O—, —N(R^{7d})—, —C(O)—, —S—, —S(O)₂—, —S(O)—, —P(O)R^{7d}—, —C(R^{7c})₂—, —OC(R^{7c})₂—, —N(R^{7d})C(R^{7c})₂—, —C(O)C(R^{7c})₂—, —SC(R^{7c})₂—, —S(O)₂C(R^{7c})₂—, —S(O)C(R^{7c})₂—, —P(O)R^{7d}C(R^{7c})₂—, —C(R^{7c})₂C(R^{7c})₂, —C(R^{7c})₂O—, —C(R^{7c})₂N(R^{7d})—, —C(R^{7c})₂C(O)—, —C(R^{7c})₂S—, —C(R^{7c})₂S(O)₂—, —C(R^{7c})₂S(O)—, —C(R^{7c})₂P(O)R^{7d}—, —N(R^{7d})C(O)—, —N(R^{7d})S(O)₂—, —N(R^{7d})S(O)—, —N(R^{7d})P(O)R^{7d}—, —C(O)N(R^{7d})—, —S(O)₂N(R^{7d})—, —S(O)N(R^{7d})—, —P(O)R^{7d}N(R^{7d})—, —OC(O)—, —OS(O)₂—, —OS(O)—, —OP(O)R^{7d}—, —C(O)O—, —S(O)₂O—, —S(O)O—, —P(O)R^{7d}O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₂₋₆ alkenyl, C₂₋₆ alkynyl, 2- to 6-membered heteroalkyl, C₃₋₁₀ carbocycle, and 3- to 10-membered heterocycle, wherein C₁₋₆ alkyl, C₂₋₆ alkenyl, C₂₋₆ alkynyl, 2- to 6-membered heteroalkyl, C₃₋₁₀ carbocycle, and 3- to 10-membered heterocycle are optionally substituted with one, two, or three R^{L};
R^{A} and R^{L} are each independently selected at each occurrence from halogen, oxo, —CN, C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, —CH₂-(3- to 12-membered heterocycle), —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₁₂ carbocycle, —CH₂—(C₃₋₁₂ carbocycle), 3- to 12-membered heterocycle, and —CH₂-(3- to 12-membered heterocycle) are optionally substituted with one, two, or three R²⁰; or two R^{A} or two R^{L} are taken together with the atom to which they are attached to form a C₃₋₈ carbocycle or 3- to 8-membered heterocycle, each of which is optionally substituted with one, two, or three R²⁰;
n is an integer from 0 to 6;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —N(R¹²)(R¹³), —C(O)OR¹², —OC(O)N(R¹²)(R¹³), —N(R¹⁴)C(O)N(R¹²)(R¹³), —N(R¹⁴)C(O)OR¹⁵, —N(R¹⁴)S(O)₂R¹⁵, —C(O)R¹², —S(O)R¹⁵, —OC(O)R¹⁵, —C(O)N(R¹²)(R¹³), —C(O)C(O)N(R¹²)(R¹³), —N(R¹⁴)C(O)R¹⁵, —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰;
R^{7d} is independently selected at each occurrence from hydrogen, —CN, C₁₋₆ alkyl, C₂₋₆ alkenyl, C₂₋₆ alkynyl, C₃₋₁₀ carbocycle, 3- to 10-membered heterocycle, —OR¹², —SR¹², —C(O)OR¹², —OC(O)N(R¹²)(R¹³), —C(O)R¹², —S(O)R¹⁵, —OC(O)R¹⁵, —C(O)N(R¹²)(R¹³), —C(O)C(O)N(R¹²)(R¹³), —S(O)₂R¹⁵, —S(O)₂N(R¹²)(R¹³), —S(═O)(═NH)N(R¹²)(R¹³), —CH₂C(O)N(R¹²)(R¹³), —CH₂N(R¹⁴)C(O)R¹⁵, —CH₂S(O)₂R¹⁵, and —CH₂S(O)₂N(R¹²)(R¹³), wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰;
R¹² is independently selected at each occurrence from hydrogen, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three R²⁰;
R¹³ is independently selected at each occurrence from hydrogen, C₁₋₆ alkyl, and C₁₋₆ haloalkyl; or R¹² and R¹³ are taken together with the nitrogen atom to which they are attached to form a 3- to 10-membered heterocycle, optionally substituted with one, two, or three R²⁰;
R¹⁴ is independently selected at each occurrence from hydrogen, C₁₋₆ alkyl, and C₁₋₆ haloalkyl;
R¹⁵ is independently selected at each occurrence from C₁₋₆ alkyl, C₂₋₆ alkenyl, C₂₋₆ alkynyl, C₃₋₁₀ carbocycle, and 3- to 10-membered heterocycle, each of which is optionally substituted with one, two, or three R²⁰;
R²⁰ is independently selected at each occurrence from halogen, oxo, —CN, C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, —CH₂-(3- to 10-membered heterocycle), —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³)—, —S(═O)(═NH)N(R²²)(R²³), —OCH₂C(O)OR²², —CH₂C(O)N(R²²)(R²³), —CH₂N(R²⁴)C(O)R²⁵, —CH₂S(O)₂R²⁵, and —CH₂S(O)₂N(R²²)(R²³), wherein C₁₋₆ alkyl, C₂₋₆ alkenyl, C₂₋₆ alkynyl, C₃₋₁₀ carbocycle, —CH₂—(C₃₋₁₀ carbocycle), 3- to 10-membered heterocycle, and —CH₂-(3- to 10-membered heterocycle) are optionally substituted with one, two, or three substituents independently selected from halogen, oxo, —CN, C₁₋₆ alkyl, C₁₋₆ haloalkyl, C₁₋₆ alkoxy, C₁₋₆ haloalkoxy, —OR²², —SR²², —N(R²²)(R²³), —C(O)OR²², —OC(O)N(R²²)(R²³), —N(R²⁴)C(O)N(R²²)(R²³), —N(R²⁴)C(O)OR²⁵, —N(R²⁴)S(O)₂R²⁵, —C(O)R²⁵, —S(O)R²⁵, —OC(O)R²⁵, —C(O)N(R²²)(R²³), —C(O)C(O)N(R²²)(R²³), —N(R²⁴)C(O)R²⁵, —S(O)₂R²⁵, —S(O)₂N(R²²)(R²³), and —S(═O)(═NH)N(R²²)(R²³);
R²² is independently selected at each occurrence from hydrogen, C₁₋₆ alkyl, C₁₋₆ haloalkyl, C₂₋₆ alkenyl, C₂₋₆ alkynyl, C₃₋₁₀ carbocycle, and 3- to 10-membered heterocycle;
R²³ and R²⁴ are each independently selected at each occurrence from hydrogen and C₁₋₆ alkyl; and
R²⁵ is independently selected at each occurrence from C₁₋₆ alkyl, C₂₋₆ alkenyl, C₂₋₆ alkynyl, C₃₋₁₀ carbocycle, and 3- to 10-membered heterocycle.

2. The compound of claim 1, having the structure of Formula (I′): 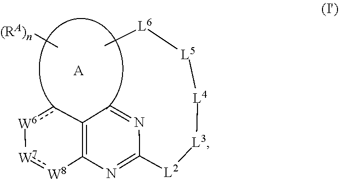
or a pharmaceutically acceptable salt or solvate thereof.

3. The compound of claim 1 or 2, or a pharmaceutically acceptable salt or solvate thereof, wherein A is 5- to 18-membered heterocycle.

4. The compound of claim 3, or a pharmaceutically acceptable salt or solvate thereof, wherein A is 5- to 8-membered monocyclic heterocycloalkenyl.

5. The compound of claim 3, or a pharmaceutically acceptable salt or solvate thereof, wherein A is 8- to 12-membered fused bicyclic heterocycloalkenyl.

6. The compound of any one of claims 1 to 4, having the structure of Formula (I-A): 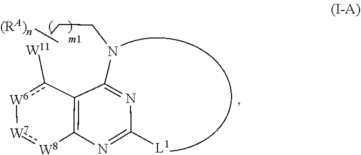
or a pharmaceutically acceptable salt or solvate thereof, wherein:
W¹¹ is selected from —O—, —N(R^{7d})—, —S—, and —C(R^{7c})₂—; and
m1 is 1 or 2.

7. The compound of any one of claims 1 to 3 or 5, having the structure of Formula (I-B): 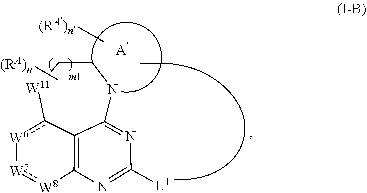
or a pharmaceutically acceptable salt or solvate thereof, wherein:
W¹¹ is selected from —O—, —N(R^{7d})—, —S—, and —C(R^{7c})₂—;
A′ is 5- to 7-membered heterocycle;
R^{A′} is independently selected at each occurrence from R^{A};
n′ is an integer from 0 to (6-n); and
m1 is 1 or 2.

8. The compound of claim 6 or 7, or a pharmaceutically acceptable salt or solvate thereof, wherein W¹¹ is —O— and m1 is 1.

9. The compound of claim 6, having the structure of Formula (I-A1), (I-A2), or (I-A3): 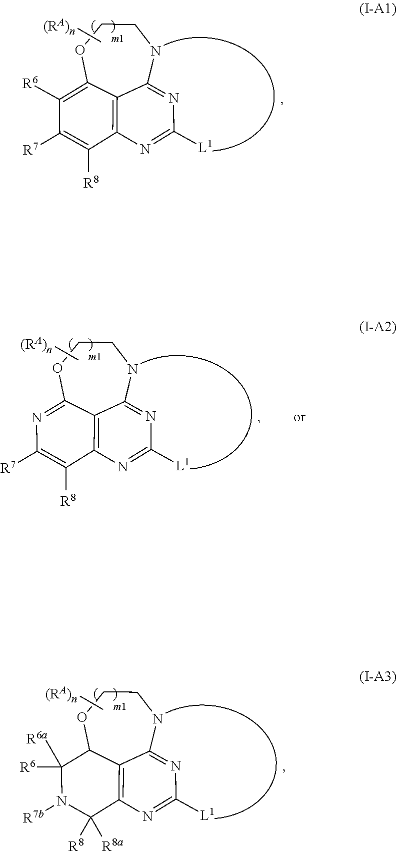
or a pharmaceutically acceptable salt or solvate thereof.

10. The compound of claim 7, having the structure of Formula (I-B1), (I-B2), or (I-B3): 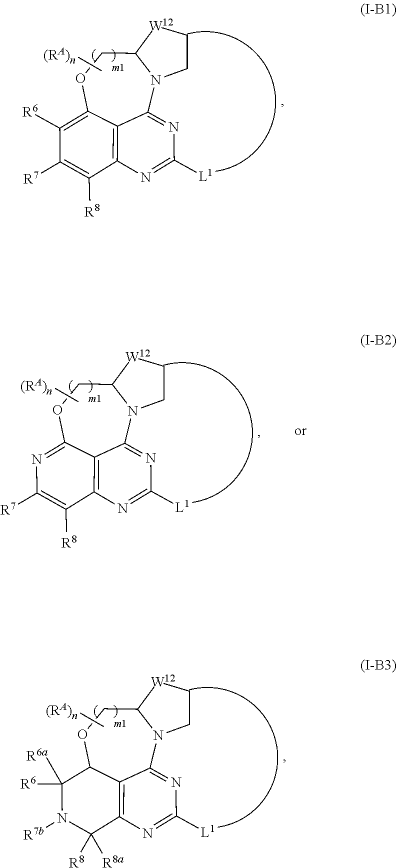
or a pharmaceutically acceptable salt or solvate thereof, wherein W¹² is selected from —O—, —N(R^{7d})—, —C(R^{7c})₂—, —OC(R^{7c})₂—, —N(R^{7d})C(R^{7c})₂—, —C(R^{7c})₂C(R^{7c})₂, —C(R^{7c})₂O—, and —C(R^{7c})₂N(R^{7d})—.

11. The compound of claim 10, or a pharmaceutically acceptable salt or solvate thereof, wherein W¹² is selected from —O—, —N(R^{7d})—, —CH₂—, —OCH₂—, —N(R^{7d})CH₂—, —CH₂CH₂, —CH₂O—, and —CH₂N(R^{7d})—.

12. The compound of any one of claims 1 to 11, or a pharmaceutically acceptable salt or solvate thereof, wherein W⁶ is C(R⁶), W⁷ is C(R⁷), W⁸ is C(R⁸), and each [symbol] indicates a double bond.

13. The compound of any one of claims 1 to 11, or a pharmaceutically acceptable salt or solvate thereof, wherein W⁶ is N, W⁷ is C(R⁷), W⁸ is C(R⁸), and each [symbol] indicates a double bond.

14. The compound of any one of claims 1 to 11, or a pharmaceutically acceptable salt or solvate thereof, wherein W⁶ is C(R⁶)(R^{6a}) or C(O), W⁷ is N(R^{7b}), W⁸ is C(R⁸)(R^{8a}) or C(O), and each [symbol] indicates a single bond.

15. The compound of any one of claims 1 to 12 or 14, or a pharmaceutically acceptable salt or solvate thereof, wherein R⁶ is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰.

16. The compound of claim 15, or a pharmaceutically acceptable salt or solvate thereof, wherein R⁶ is selected from hydrogen, halogen, —CN, C₁₋₆ alkyl, and —OR¹², wherein C₁₋₆ alkyl is optionally substituted with one, two, or three R²⁰.

17. The compound of claim 15, or a pharmaceutically acceptable salt or solvate thereof, wherein R⁶ is halogen.

18. The compound of any one of claims 1 to 13 or 15 to 17, or a pharmaceutically acceptable salt or solvate thereof, wherein L⁷ is selected from a bond, —O—, —N(R^{7d})—, —C(O)—, and —C(R^{7c})₂—.

19. The compound of claim 18, or a pharmaceutically acceptable salt or solvate thereof, wherein L⁷ is a bond.

20. The compound of any one of claims 1 to 19, or a pharmaceutically acceptable salt or solvate thereof, wherein R⁸ is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰.

21. The compound of claim 20, or a pharmaceutically acceptable salt or solvate thereof, wherein R⁸ is selected from hydrogen, halogen, —CN, C₁₋₆ alkyl, and —OR¹², wherein C₁₋₆ alkyl is optionally substituted with one, two, or three R²⁰.

22. The compound of claim 20, or a pharmaceutically acceptable salt or solvate thereof, wherein R⁸ is halogen.

23. The compound of any one of claims 1 to 11 or 14 to 22, or a pharmaceutically acceptable salt or solvate thereof, wherein R⁶ is selected from hydrogen, halogen, —CN, C₁₋₆ alkyl, C₃₋₆ cycloalkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl and C₃₋₆ cycloalkyl are optionally substituted with one, two, or three R²⁰.

24. The compound of claim 23, or a pharmaceutically acceptable salt or solvate thereof, wherein R^{6a} is selected from hydrogen, halogen, —CN, C₁₋₆ alkyl, and —OR¹², wherein C₁₋₆ alkyl is optionally substituted with one, two, or three R²⁰.

25. The compound of claim 23, or a pharmaceutically acceptable salt or solvate thereof, wherein R⁶ and R^{6a} are each hydrogen.

26. The compound of any one of claims 1 to 11 or 14 to 25, or a pharmaceutically acceptable salt or solvate thereof, wherein L^{7b} is selected from a bond, —O—, —C(O)—, and —C(R^{7c})₂—.

27. The compound of claim 26, or a pharmaceutically acceptable salt or solvate thereof, wherein L^{7b} is a bond.

28. The compound of any one of claims 1 to 11 or 14 to 27, or a pharmaceutically acceptable salt or solvate thereof, wherein R^{8a} is selected from hydrogen, halogen, —CN, C₁₋₆ alkyl, —OR¹², —SR¹², —N(R¹²)(R¹³), —C(O)OR¹², —C(O)R¹⁵, —C(O)N(R¹²)(R¹³), —S(O)₂R¹⁵, and —S(O)₂N(R¹²)(R¹³), wherein C₁₋₆ alkyl is optionally substituted with one, two, or three R²⁰.

29. The compound of claim 28, or a pharmaceutically acceptable salt or solvate thereof, wherein R^{8a} is selected from hydrogen, halogen, —CN, C₁₋₆ alkyl, and —OR¹², wherein C₁₋₆ alkyl is optionally substituted with one, two, or three R²⁰.

30. The compound of claim 28, or a pharmaceutically acceptable salt or solvate thereof, wherein R⁸ and R^{8a} are each hydrogen.

31. The compound of any one of claims 1 to 30, or a pharmaceutically acceptable salt or solvate thereof, wherein R¹⁷ is selected from C₆₋₁₂ aryl and 5- to 12-membered heteroaryl, each of which is optionally substituted with one, two, or three R²⁰.

32. The compound of claim 31, or a pharmaceutically acceptable salt or solvate thereof, wherein R¹⁷ is selected from C₁₀ aryl and 9-membered heteroaryl, each of which is optionally substituted with one, two, or three R²⁰.

33. The compound of claim 31, or a pharmaceutically acceptable salt or solvate thereof, wherein R¹⁷ is selected from naphthalenyl and benzothiophenyl, each of which is optionally substituted with one, two, or three R²⁰.

34. The compound of any one of claims 31 to 33, or a pharmaceutically acceptable salt or solvate thereof, wherein R¹⁷ is substituted with one, two, or three substituents independently selected from halogen, —CN, C₁₋₃ alkyl, C₂₋₃ alkenyl, C₂₋₃ alkynyl, —OR²², and —N(R²²)(R²³).

35. The compound of claim 34, or a pharmaceutically acceptable salt or solvate thereof, wherein R¹⁷ is substituted with one, two, or three substituents independently selected from halogen, —CN, —CH₃, —C═CH, —OH, and —NH₂.

36. The compound of claim 34, or a pharmaceutically acceptable salt or solvate thereof, wherein R¹⁷ is substituted with —F, —CN, and —NH₂.

37. The compound of any one of claims 1 to 30, or a pharmaceutically acceptable salt or solvate thereof, wherein R¹⁷ is selected from

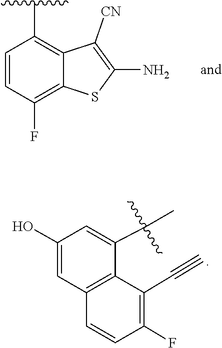 [SMILES: *c1ccc(F)c2sc(N)c(C#N)c12 ; *c1cc(O)cc2ccc(F)c(C#C)c12]

38. The compound of any one of claims 1 to 30, or a pharmaceutically acceptable salt or solvate thereof, wherein R¹⁷ is selected from

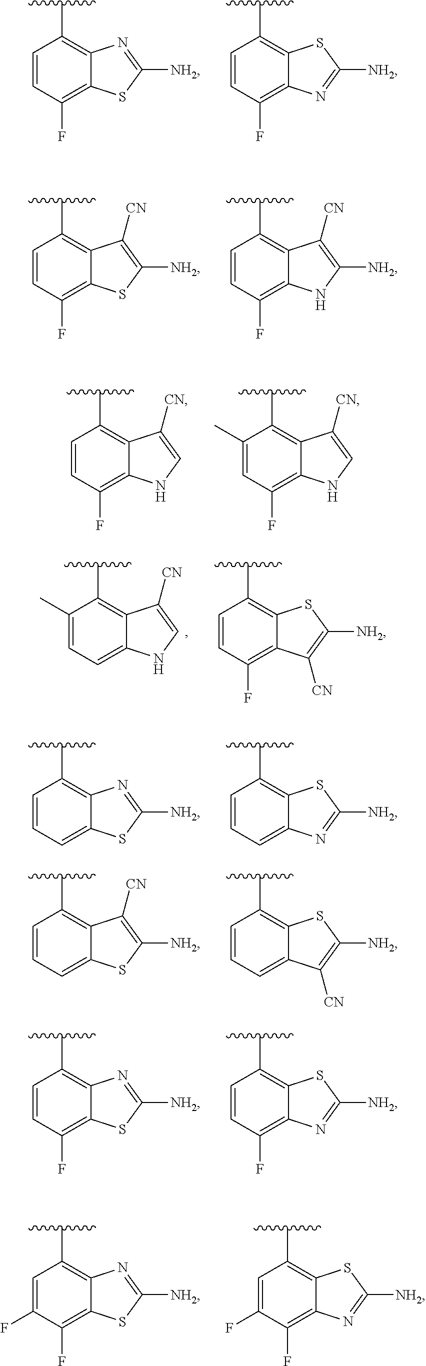

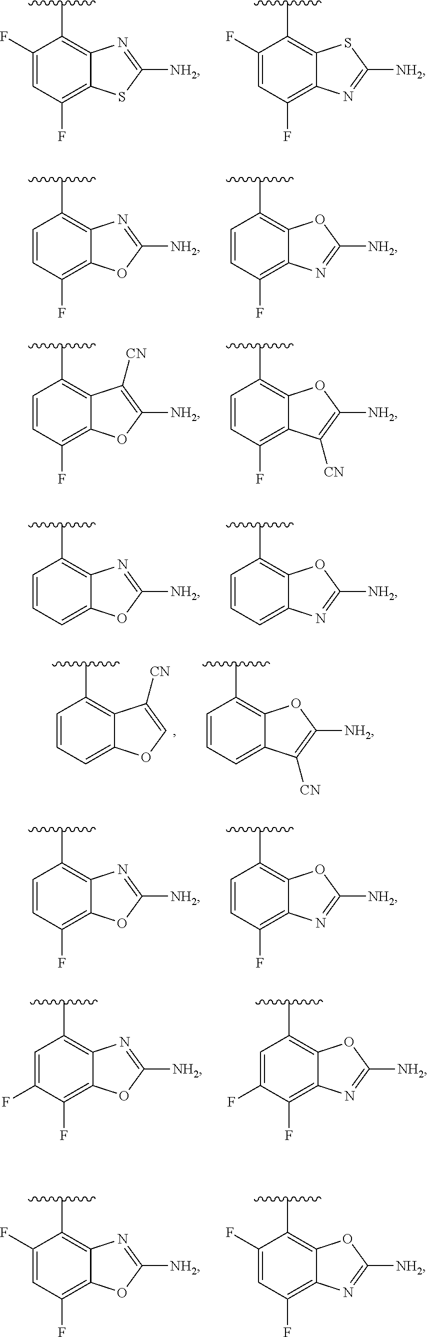

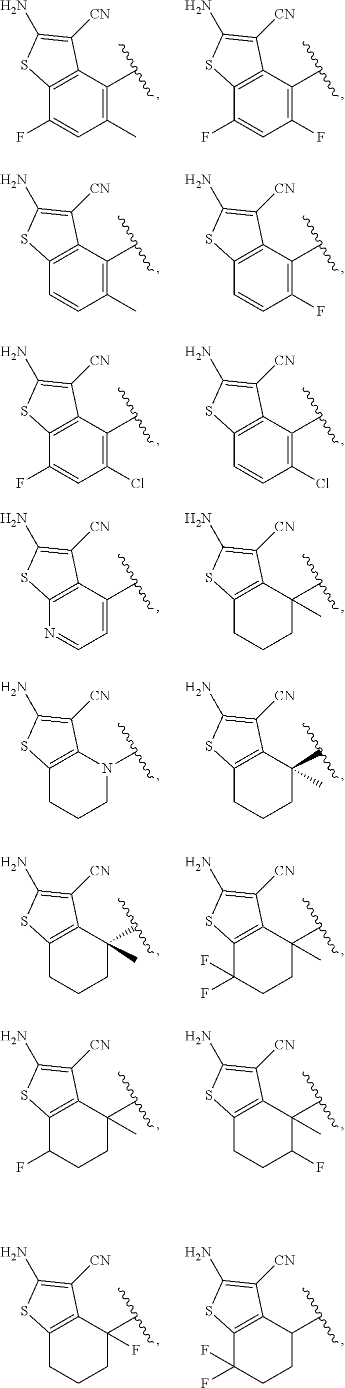

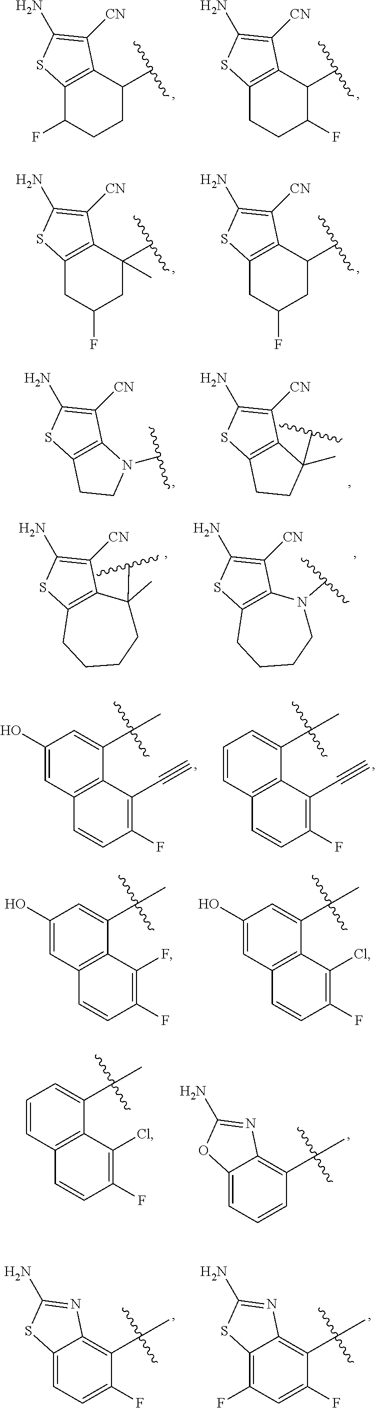

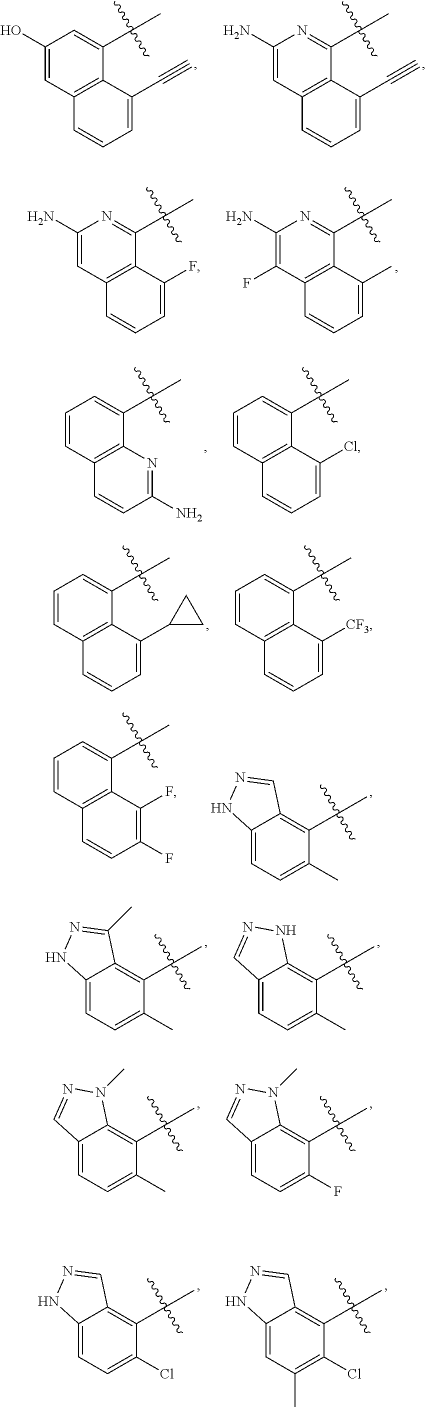

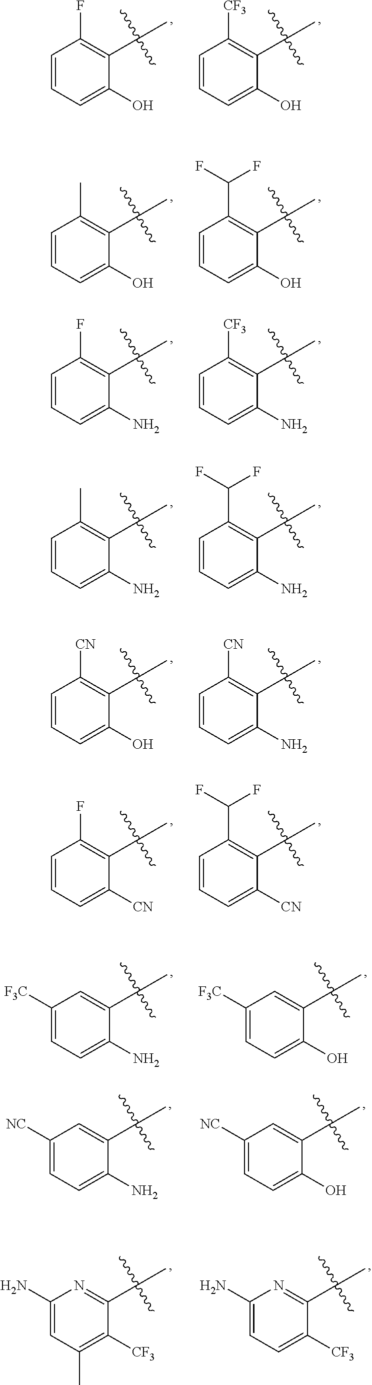

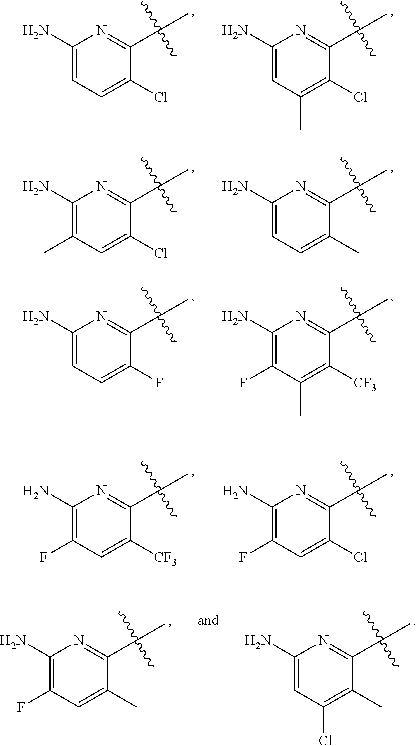

39. The compound of any one of claims 1 to 38, or a pharmaceutically acceptable salt or solvate thereof, wherein R^{A} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 8-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 8-membered heterocycle are optionally substituted with one, two, or three R²⁰.

40. The compound of claim 39, or a pharmaceutically acceptable salt or solvate thereof, wherein R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³).

41. The compound of any one of claims 1 to 40, or a pharmaceutically acceptable salt or solvate thereof, wherein n is an integer from 0 to 3.

42. The compound of claim 41, or a pharmaceutically acceptable salt or solvate thereof, wherein n is 0.

43. The compound of any one of claims 1 to 42, or a pharmaceutically acceptable salt or solvate thereof, wherein L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—.

44. The compound of claim 43, or a pharmaceutically acceptable salt or solvate thereof, wherein L² is selected from —O— and —OC(R^{7c})₂—.

45. The compound of claim 43, or a pharmaceutically acceptable salt or solvate thereof, wherein L² is —OCH(3- to 10-membered heterocycle)-, wherein the 3- to 10-membered heterocycle is optionally substituted with one, two, or three substituents selected from halogen and C₁₋₃ alkyl.

46. The compound of any one of claims 1 to 42, or a pharmaceutically acceptable salt or solvate thereof, wherein L² is selected from

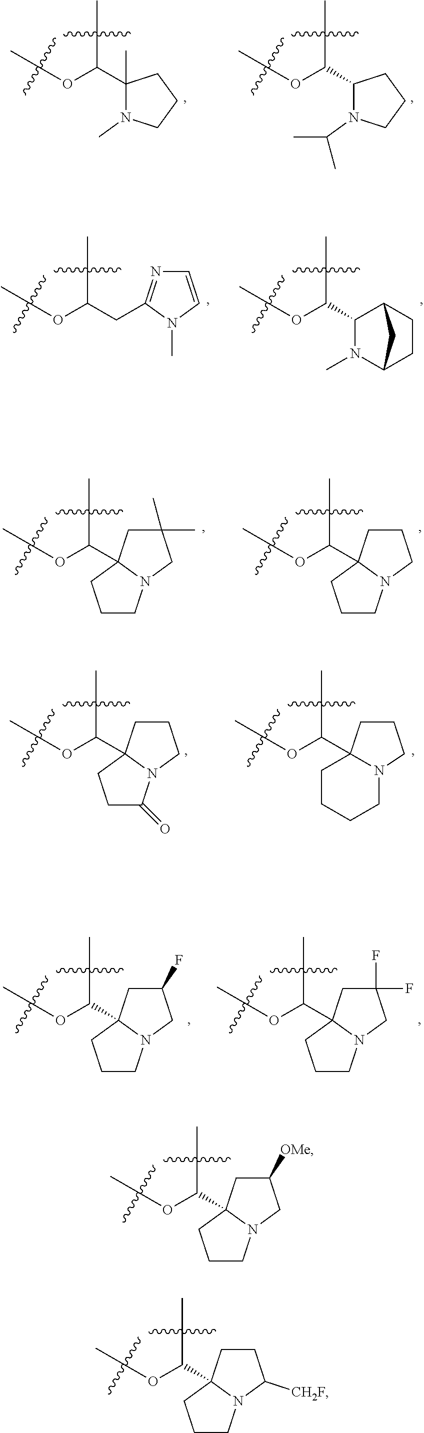

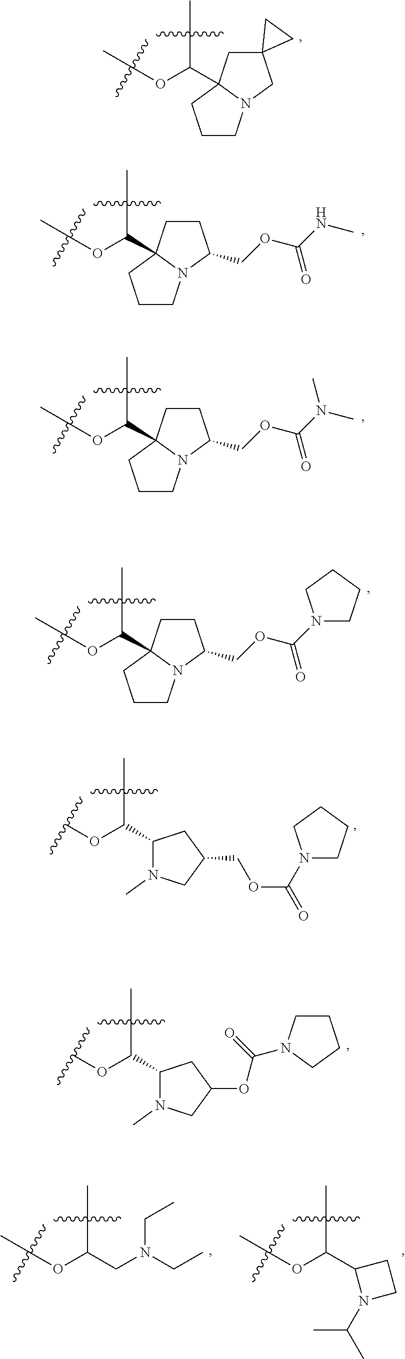

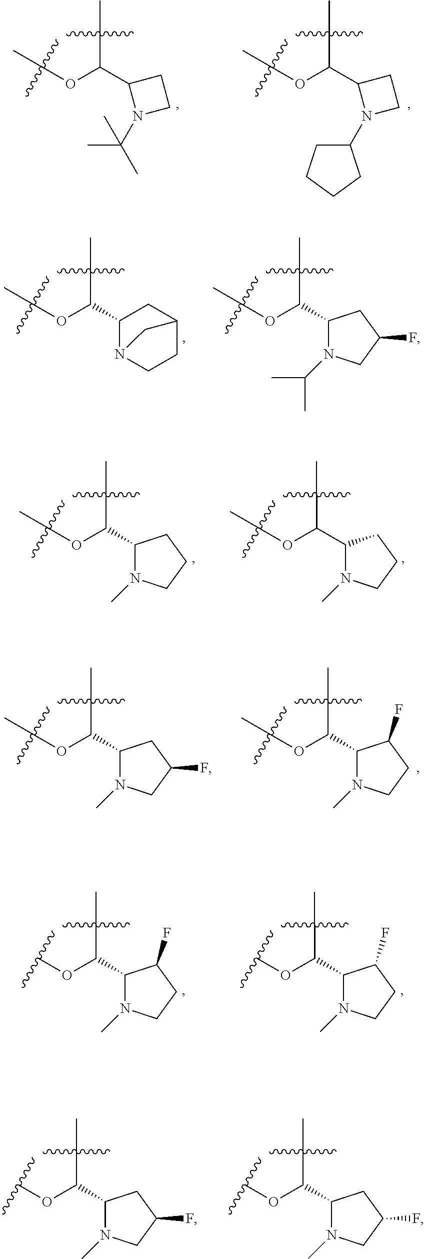

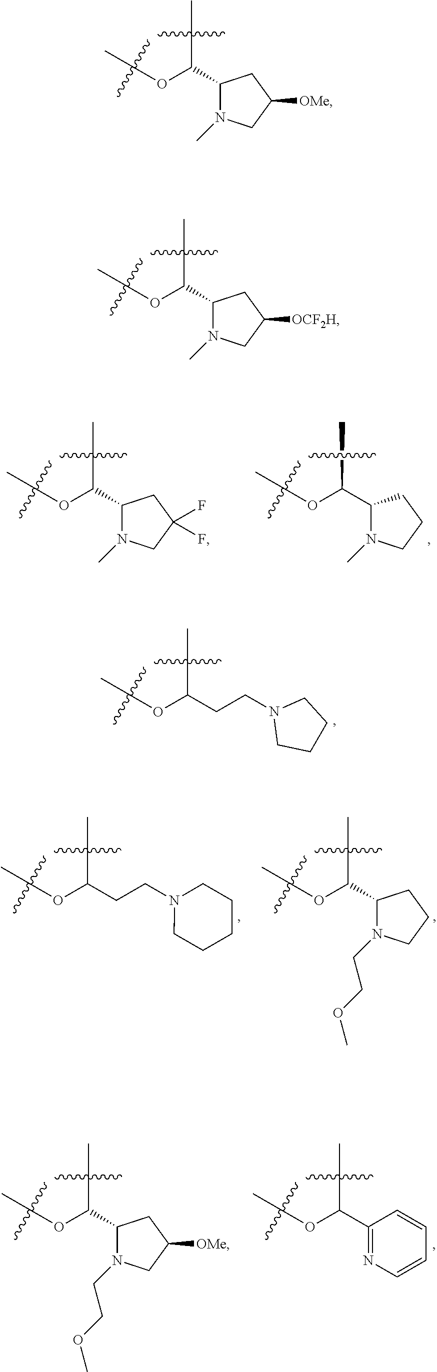

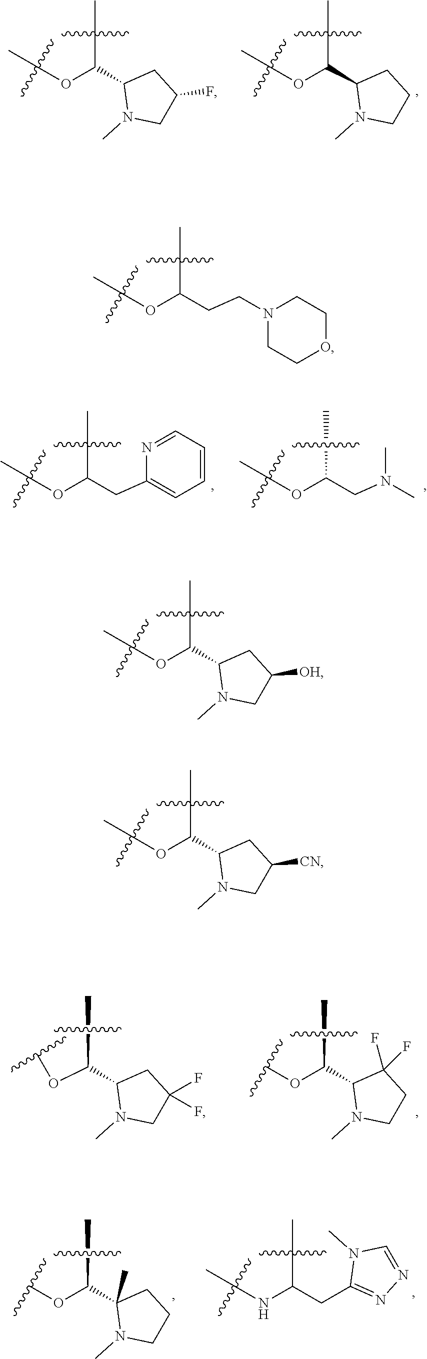

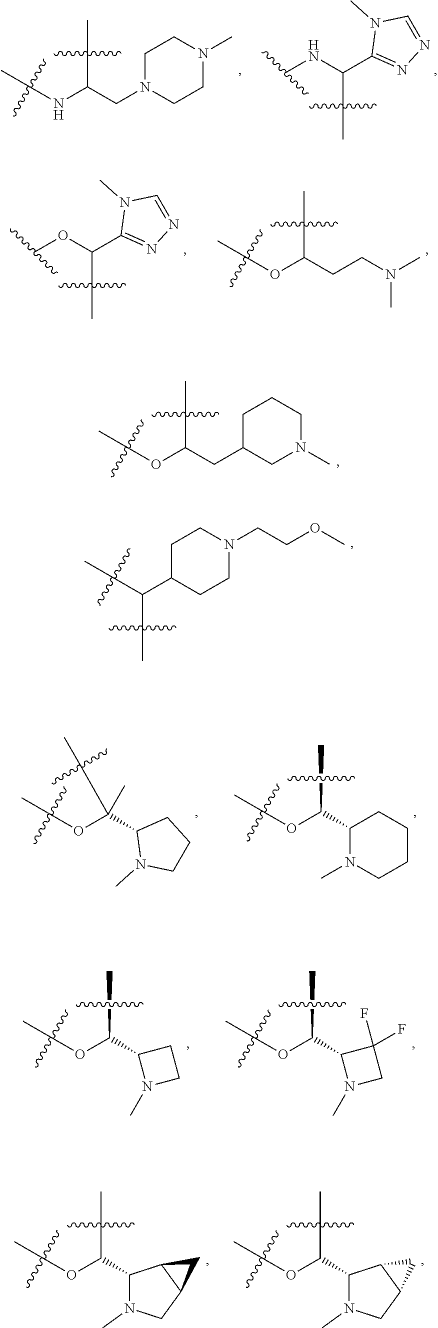

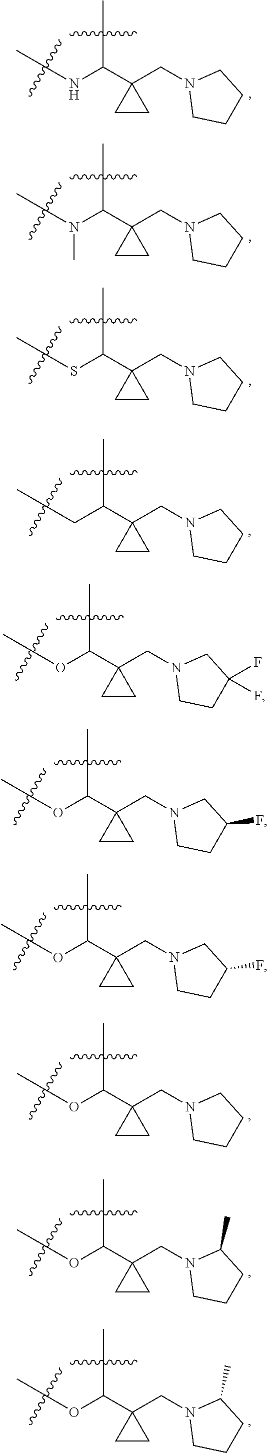

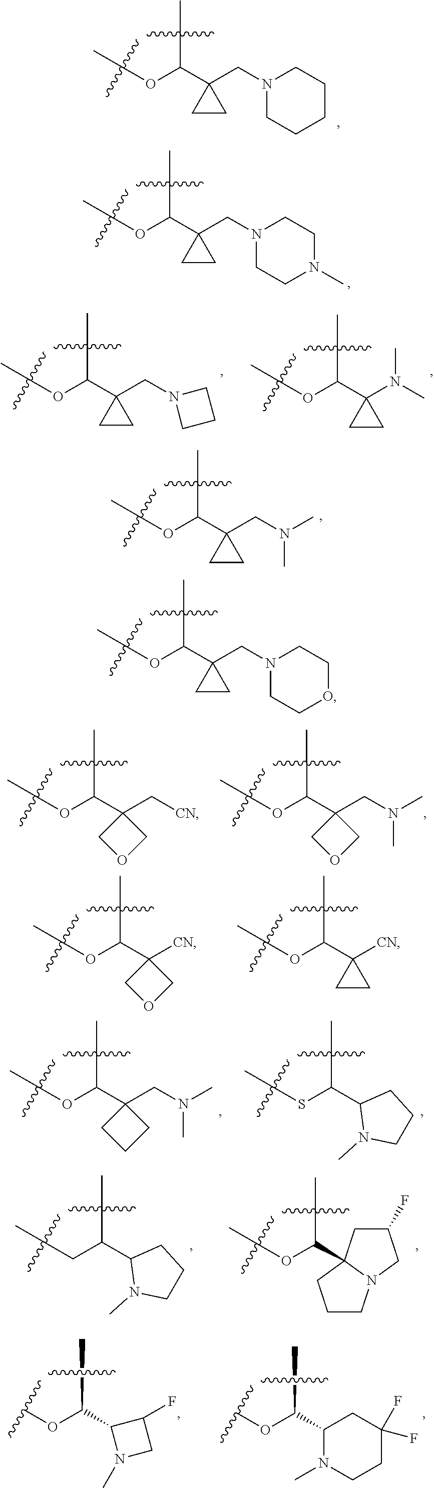

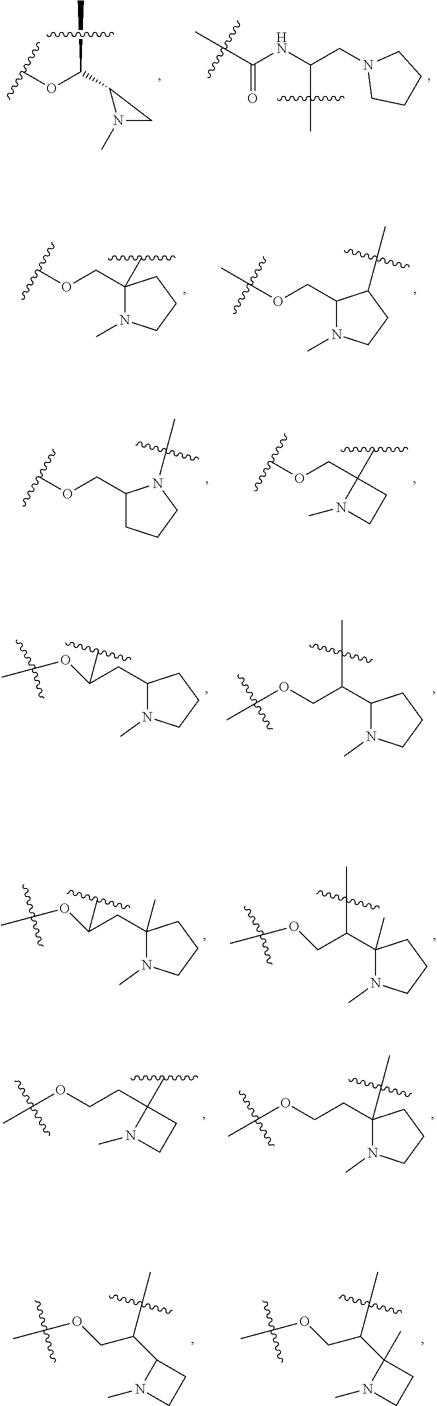

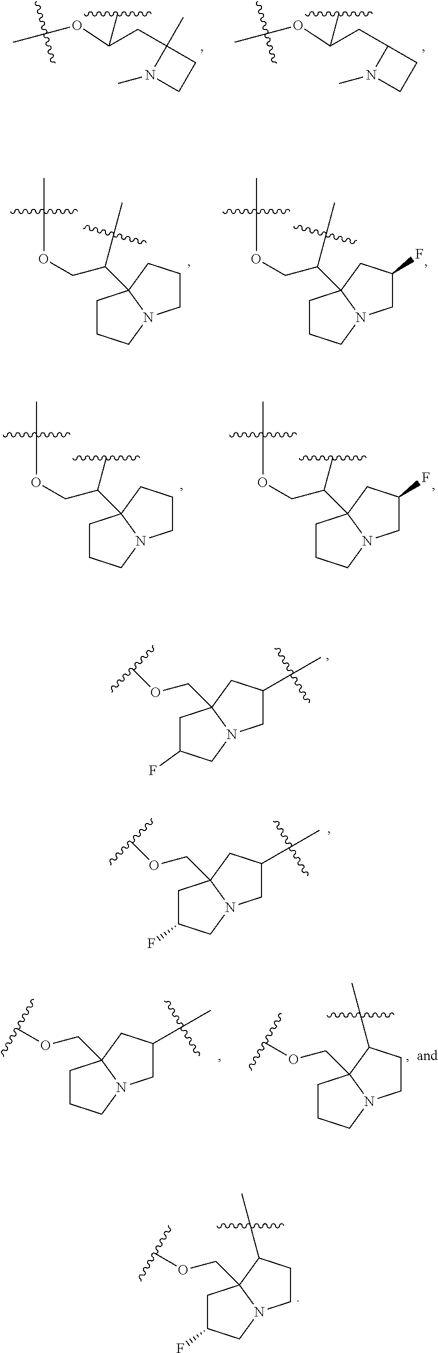

47. The compound of any one of claims 1 to 46, or a pharmaceutically acceptable salt or solvate thereof, wherein L³ is selected from C₁₋₆ alkyl, C₂₋₆ alkenyl, and 2- to 6-membered heteroalkyl, each of which is optionally substituted with one, two, or three R^{L}.

48. The compound of claim 47, or a pharmaceutically acceptable salt or solvate thereof, wherein L³ is selected from C₁₋₆ alkyl and C₂₋₆ alkenyl.

49. The compound of any one of claims 1 to 48, or a pharmaceutically acceptable salt or solvate thereof, wherein L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

50. The compound of claim 49, or a pharmaceutically acceptable salt or solvate thereof, wherein L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L}.

51. The compound of any one of claims 1 to 50, or a pharmaceutically acceptable salt or solvate thereof, wherein L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}.

52. The compound of any one of claims 1 to 50, or a pharmaceutically acceptable salt or solvate thereof, wherein L⁵ is selected from a bond and C₁₋₆ alkyl, wherein C₁₋₆ alkyl is optionally substituted with one, two, or three R^{L}.

53. The compound of any one of claims 1 to 52, or a pharmaceutically acceptable salt or solvate thereof, wherein L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

54. The compound of claim 53, or a pharmaceutically acceptable salt or solvate thereof, wherein L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

55. The compound of any one of claims 1 to 42, or a pharmaceutically acceptable salt or solvate thereof, wherein:
L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—;
L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and
L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

56. The compound of any one of claims 1 to 42, or a pharmaceutically acceptable salt or solvate thereof, wherein:
L² is selected from —O— and —OC(R^{7c})₂—;
L³ is selected from C₁₋₆ alkyl and C₂₋₆ alkenyl;
L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L};
L⁵ is selected from a bond and C₁₋₆ alkyl, wherein C₁₋₆ alkyl is optionally substituted with one, two, or three R^{L}; and
L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

57. The compound of any one of claims 1 to 56, or a pharmaceutically acceptable salt or solvate thereof, wherein R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰.

58. The compound of claim 57, or a pharmaceutically acceptable salt or solvate thereof, wherein R^{L} is independently selected at each occurrence from halogen, oxo, C₁₋₆ alkyl, C₃₋₈ carbocycle, 3- to 10-membered heterocycle, and —C(O)R¹², wherein C₁₋₆ alkyl, C₃₋₈ carbocycle, and 3- to 10-membered heterocycle are optionally substituted with one, two, or three R²⁰.

59. The compound of any one of claims 1 to 58, or a pharmaceutically acceptable salt or solvate thereof, wherein R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰.

60. The compound of claim 59, or a pharmaceutically acceptable salt or solvate thereof, wherein R^{7c} is independently selected at each occurrence from hydrogen, halogen, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰.

61. The compound of any one of claims 1 to 60, or a pharmaceutically acceptable salt or solvate thereof, wherein R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰.

62. The compound of any one of claims 1, 2, 6, 7, 9, and 10, or a pharmaceutically acceptable salt or solvate thereof, wherein:
R⁶ is selected from hydrogen and halogen;
R^{6a} is hydrogen;
L⁷ is selected from a bond, —O—, —N(R^{7d})—, —C(O)—, and —C(R^{7c})₂—;
L^{7b} is selected from a bond, —O—, —C(O)—, and —C(R^{7c})₂—;
R¹⁷ is selected from C₆₋₁₂ aryl and 6- to 12-membered heteroaryl, each of which is optionally substituted with one, two, or three R²⁰;
R⁸ is selected from hydrogen, halogen, —CN, C₁₋₆ alkyl, and —OR¹², wherein C₁₋₆ alkyl is optionally substituted with one, two, or three R²⁰;
R^{8a} is hydrogen;
R^{A} is independently selected at each occurrence from halogen, oxo, —CN, C₁₋₃ alkyl, C₁₋₃ haloalkyl, —OH, —OCH₃, —N(R¹²)(R¹³), —C(O)R¹², and —C(O)N(R¹²)(R¹³);
n is an integer from 0 to 3;
L² is selected from —O—, —N(R^{7d})—, —S—, —S(O)₂—, —C(R^{7c})₂—, —OC(R^{7c})₂—, —N(R^{7d})C(R^{7c})₂—, —SC(R^{7c})₂—, —S(O)₂C(R^{7c})₂—, —N(R^{7d})C(O)—, and —C(O)N(R^{7d})—;
L³ is selected from C₁₋₆ alkyl, C₂₋₆ alkenyl, and 2- to 6-membered heteroalkyl, each of which is optionally substituted with one, two, or three R^{L};
L⁴ is selected from a bond, —O—, —N(R^{7d})—, —C(O)—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L};
L⁵ is selected from a bond, C₁₋₆ alkyl, C₃₋₁₀ carbocycle, and 3- to 10-membered heterocycle, wherein C₁₋₆ alkyl, C₃₋₁₀ carbocycle, and 3- to 10-membered heterocycle are optionally substituted with one, two, or three R^{L}; and
L⁶ is selected from a bond, —O—, —N(R^{7d})—, —C(O)—, —C(R^{7c})₂—, —OC(R^{7c})₂—, —N(R^{7d})C(R^{7c})₂—, —C(O)C(R^{7c})₂—, —N(R^{7d})C(O)—, —C(O)N(R^{7d})—, —OC(O)—, —C(O)O—, C₃₋₁₀ carbocycle, and 3- to 10-membered heterocycle, wherein C₃₋₁₀ carbocycle and 3- to 10-membered heterocycle are optionally substituted with one, two, or three R^{L}.

63. The compound of any one of claims 1, 2, 6, 7, 9, and 10, or a pharmaceutically acceptable salt or solvate thereof, wherein:
R⁶ is selected from hydrogen and halogen;
R⁶ is hydrogen;
L⁷ is a bond;
L^{7b} is a bond;
R¹⁷ is selected from naphthalenyl and benzothiophenyl, each of which is optionally substituted with one, two, or three R²⁰;
R⁸ is halogen;
R^{8a} is hydrogen;
n is 0;
L² is selected from —O— and —OC(R^{7c})₂—;
L³ is selected from C₁₋₆ alkyl and C₂₋₆ alkenyl;
L⁴ is selected from a bond, —O—, —N(R^{7d})—, —C(O)—, —N(R^{7d})C(O)—, —C(O)N(R^{7d})—, and 3- to 10-membered heterocycle, wherein 3- to 10-membered heterocycle is optionally substituted with one, two, or three R^{L};
L⁵ is selected from a bond and C₁₋₆ alkyl, wherein C₁₋₆ alkyl is optionally substituted with one, two, or three R^{L}; and
L⁶ is selected from a bond, —O—, —N(R^{7d})—, —C(O)—, —C(R^{7c})₂—, —N(R^{7d})C(O)—, —C(O)N(R^{7d})—, and 3- to 10-membered heterocycle, wherein 3- to 10-membered heterocycle is optionally substituted with one, two, or three R^{L}.

64. The compound of claim 62 or 63, or a pharmaceutically acceptable salt or solvate thereof, wherein R¹⁷ is selected from

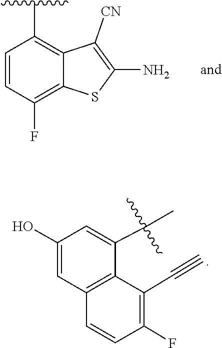 [SMILES: *c1ccc(F)c2sc(N)c(C#N)c12 ; *c1cc(O)cc2ccc(F)c(C#C)c12]

65. The compound of any one of claims 62 to 64, or a pharmaceutically acceptable salt or solvate thereof, wherein:
R^{L} is independently selected at each occurrence from halogen, oxo, —CN, C₁₋₆ alkyl, C₂₋₆ alkenyl, C₂₋₆ alkynyl, C₁₋₆ haloalkyl, C₃₋₈ carbocycle, 3- to 10-membered heterocycle, —OR¹², —N(R¹²)(R¹³), —C(O)R¹², —C(O)N(R¹²)(R¹³), —S(O)₂N(R¹²)(R¹³), —CH₂C(O)N(R¹²)(R¹³), and —CH₂S(O)₂N(R¹²)(R¹³), wherein C₁₋₆ alkyl, C₂₋₆ alkenyl, C₂₋₆ alkynyl, C₁₋₆ haloalkyl, C₃₋₈ carbocycle, and 3- to 10-membered heterocycle are optionally substituted with one, two, or three R²⁰; or two R^{L} are taken together with the atom to which they are attached to form a C₃₋₈ carbocycle or 3- to 8-membered heterocycle, each of which is optionally substituted with one, two, or three R²⁰;
R^{7c} is independently selected at each occurrence from hydrogen, halogen, —CN, C₁₋₆ alkyl, C₂₋₆ alkenyl, C₂₋₆ alkynyl, C₃₋₁₀ carbocycle, 3- to 10-membered heterocycle, —OR¹², —N(R¹²)(R¹³), —N(R¹⁴)C(O)N(R¹²)(R¹³), —C(O)R¹², —C(O)N(R¹²)(R¹³), and —N(R¹⁴)C(O)R¹⁵, wherein C₁₋₆ alkyl, C₂₋₆ alkenyl, C₂₋₆ alkynyl, C₃₋₁₀ carbocycle, and 3- to 10-membered heterocycle are optionally substituted with one, two, or three R²⁰; or two R^{7c} are taken together with the carbon atom to which they are attached to form a C₃₋₈ carbocycle or 3- to 8-membered heterocycle, each of which is optionally substituted with one, two, or three R²⁰; and
R^{7d} is independently selected at each occurrence from hydrogen, C₁₋₆ alkyl, C₃₋₁₀ carbocycle, 3- to 10-membered heterocycle, —C(O)R¹², and —C(O)N(R¹²)(R¹³), wherein C₁₋₆ alkyl, C₃₋₁₀ carbocycle, and 3- to 10-membered heterocycle are optionally substituted with one, two, or three R²⁰.

66. A compound selected from Table 1, or a pharmaceutically acceptable salt or solvate thereof.

67. A compound having the formula B-L^{BE}-E wherein:
B is a monovalent form of a compound of one of claims 1 to 66;
L^{BE} is a covalent linker bonded to B and E; and
E is a monovalent form of a degradation enhancer.

68. The compound of claim 67, wherein the degradation enhancer is capable of binding a protein selected from E3A, mdm2, APC, EDD1, SOCS/BC-box/eloBC/CUL5/RING, LNXp80, CBX4, CBLL1, HACE1, HECTD1, HECTD2, HECTD3, HECTD4, HECW1, HECW2, HERC1, HERC2, HERC3, HERC4, HER5, HERC6, HUWE1, ITCH, NEDD4, NEDD4L, PPIL2, PRPF19, PIAS1, PIAS2, PIAS3, PIAS4, RANBP2, RNF4, RBX1, SMURF1, SMURF2, STUB1, TOPORS, TRIP12, UBE3A, UBE3B, UBE3C, UBE3D, UBE4A, UBE4B, UBOX5, UBR5, VHL (von-Hippel-Lindau ubiquitin ligase), WWP1, WWP2, Parkin, MKRN1, CMA (chaperon-mediated autophage), SCFb-TRCP (Skip-Cullin-F box (Beta-TRCP) ubiquitin complex), b-TRCP (b-transducing repeat-containing protein), cIAP1 (cellular inhibitor of apoptosis protein 1), APC/C (anaphase-promoting complex/cyclosome), CRBN (cereblon), CUL4-RBX1-DDB1-CRBN (CRL4^{CRBN}) ubiquitin ligase, XIAP, IAP, KEAP1, DCAF15, RNF114, DCAF16, AhR, SOCS2, KLHL12, UBR2, SPOP, KLHL3, KLHL20, KLHDC2, SPSB1, SPSB2, SPSB4, SOCS6, FBXO4, FBXO31, BTRC, FBW7, CDC20, PML, TRIM21, TRIM24, TRIM33, GID4, avadomide, iberdomide, and CC-885.

69. The compound of claim 67, wherein the degradation enhancer is capable of binding a protein selected from UBE2A, UBE2B, UBE2C, UBE2D1, UBE2D2, UBE2D3, UBE2DR, UBE2E1, UBE2E2, UBE2E3, UBE2F, UBE2G1, UBE2G2, UBE2H, UBE2I, UBE2J1, UBE2J2, UBE2K, UBE2L3, UBE2L6, UBE2L1, UBE2L2, UBE2L4, UBE2M, UBE2N, UBE20, UBE2Q1, UBE2Q2, UBE2R1, UBE2R2, UBE2S, UBE2T, UBE2U, UBE2V1, UBE2V2, UBE2W, UBE2Z, ATG3, BIRC6, and UFC1.

70. The compound of any one of claims 67 to 69, wherein L^{BE} is -L^{BE1}-L^{BE2}-L^{BE3}-L^{BE4}-L^{BE5}-;
L^{BE1}, L^{BE2}, L^{BE3}, L^{BE4}, and L^{BE5} are independently a bond, —O—, —N(R¹⁴)—, —C(O)—, —N(R¹⁴)C(O)—, —C(O)N(R¹⁴)—, —S—, —S(O)₂—, —S(O)—, —S(O)₂N(R¹⁴)—, —S(O)N(R¹⁴)—, —N(R¹⁴)S(O)—, —N(R¹⁴)S(O)₂—, C₁₋₆ alkylene, (—O—C₁₋₆ alkyl)₂-, (—C₁₋₆ alkyl-O)₂—, C₂₋₆ alkenylene, C₂₋₆ alkynylene, C₁₋₆ haloalkylene, C₃₋₁₂ cycloalkylene, C₁₋₁₁ heterocycloalkylene, C₆₋₁₂ arylene, or C₁₋₁₁ heteroarylene, wherein C₁₋₆ alkylene, C₂₋₆ alkenylene, C₂₋₆ alkynylene, C₁₋₆ haloalkylene, C₃₋₁₂ cycloalkylene, C₁-r heterocycloalkylene, C₆₋₁₂ arylene, or C₁-r heteroarylene are optionally substituted with one, two, or three R²⁰; and wherein each C₁₋₆ alkyl of (—O—C₁₋₆ alkyl)₂- and (—C₁₋₆ alkyl-O)₂— is optionally substituted with one, two, or three R²⁰; and
z is independently an integer from 0 to 10.

71. The compound of any one of claims 67 to 70, wherein L^{BE} is —(O-C₂alkyl)_{z}- and z is an integer from 1 to 10.

72. The compound of any one of claims 67 to 70, wherein L^{BE} is —(C₂alkyl-O—)_{z}— and z is an integer from 1 to 10.

73. The compound of any one of claims 67 to 70, wherein L^{BE} is —(CH₂)_{zz1}L^{BE2}(CH₂O)_{zz2}—, wherein L^{BE2} is a bond, a 5 or 6 membered heterocycloalkylene or heteroarylene, phenylene, —(C₂-C₄)alkynylene, —SO₂— or —NH—; and zz1 and zz2 are independently an integer from 0 to 10.

74. The compound of any one of claims 67 to 70, wherein L^{BE} is —(CH₂)_{zz1}(CH₂O)_{zz2}—, wherein zz1 and zz2 are each independently an integer from 0 to 10.

75. The compound of any one of claims 67 to 70, wherein L^{BE} is a PEG linker.

76. The compound of any one of claims 67 to 75, wherein E is a monovalent form of a compound selected from

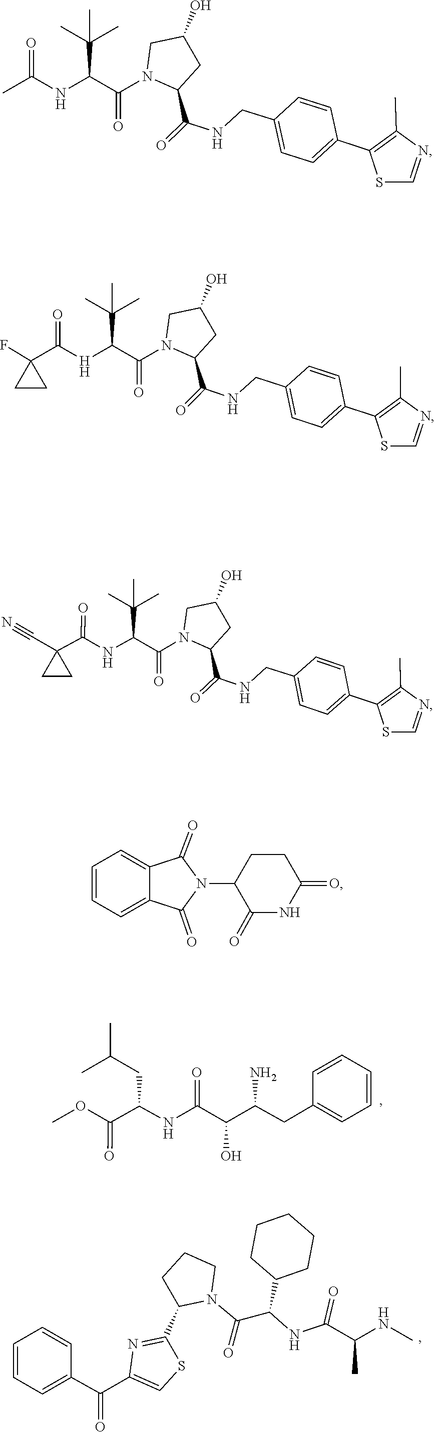

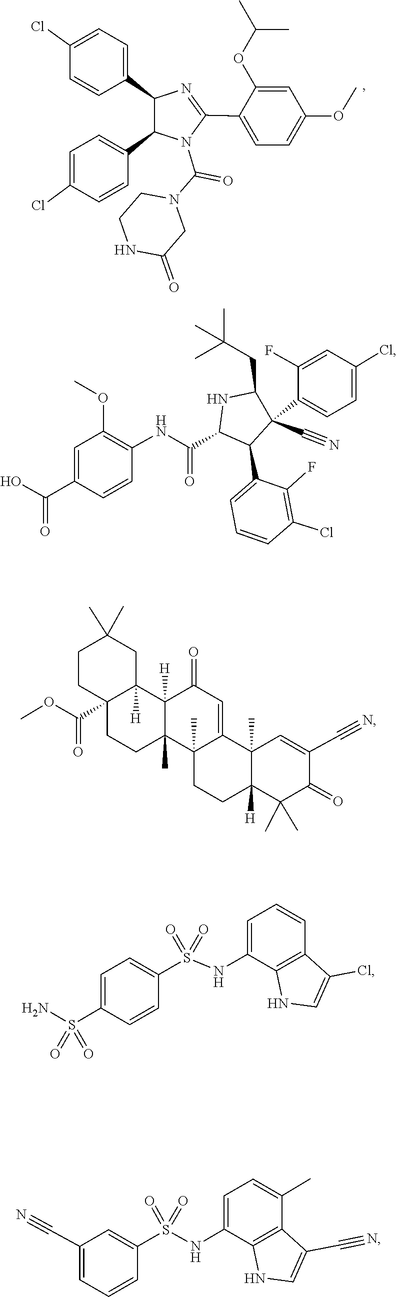

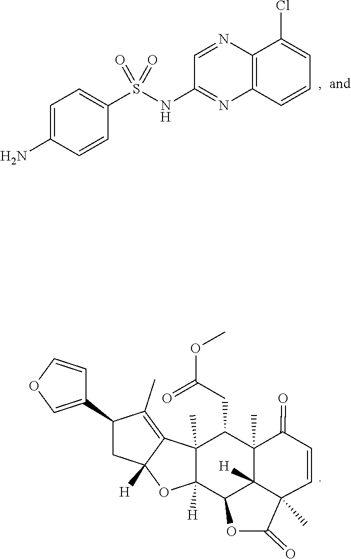

77. A pharmaceutical composition comprising a compound of any one of claims 1 to 76, or a pharmaceutically acceptable salt or solvate thereof, and a pharmaceutically acceptable excipient.

78. A method of treating cancer in a subject in need thereof, comprising administering to the subject a therapeutically effective amount of a compound of any one of claims 1 to 76, or a pharmaceutically acceptable salt or solvate thereof.

79. A method of treating cancer in a subject comprising a Ras mutant protein, the method comprising: inhibiting the Ras mutant protein of said subject by administering to said subject a compound, wherein the compound is characterized in that upon contacting the Ras mutant protein, said Ras mutant protein exhibits reduced Ras signaling output.

80. The method of claim 78 or 79, wherein the cancer is a solid tumor.

81. The method of claim 78 or 79, wherein the cancer is a hematological cancer.

82. The method of any one of claims 79 to 81, wherein the compound is a compound of any one of claims 1 to 76, or a pharmaceutically acceptable salt or solvate thereof.

83. A method of modulating signaling output of a Ras protein, comprising contacting a Ras protein with an effective amount of a compound of any one of claims 1 to 76, or a pharmaceutically acceptable salt or solvate thereof, thereby modulating the signaling output of the Ras protein.

84. A method of inhibiting cell growth, comprising administering an effective amount of a compound of any one of claims 1 to 76, or a pharmaceutically acceptable salt or solvate thereof, to a cell expressing a Ras protein, thereby inhibiting growth of said cells.

85. The method of any one of claims 78 to 84, comprising administering an additional agent.

86. The method of claim 85, wherein the additional agent comprises (1) an inhibitor of MEK; (2) an inhibitor of epidermal growth factor receptor (EGFR) and/or of mutants thereof; (3) an immunotherapeutic agent; (4) a taxane; (5) an anti-metabolite; (6) an inhibitor of FGFR1 and/or FGFR2 and/or FGFR3 and/or of mutants thereof; (7) a mitotic kinase inhibitor; (8) an anti-angiogenic drug; (9) a topoisomerase inhibitor; (10) a platinum-containing compound; (12) an inhibitor of c-MET and/or of mutants thereof; (13) an inhibitor of BCR-ABL and/or of mutants thereof; (14) an inhibitor of ErbB2 (Her2) and/or of mutants thereof; (15) an inhibitor of AXL and/or of mutants thereof; (16) an inhibitor of NTRK1 and/or of mutants thereof; (17) an inhibitor of RET and/or of mutants thereof; (18) an inhibitor of A-Raf and/or B-Raf and/or C-Raf and/or of mutants thereof; (19) an inhibitor of ERK and/or of mutants thereof; (20) an MDM2 inhibitor; (21) an inhibitor of mTOR; (23) an inhibitor of IGF1/2 and/or of IGF1-R; (24) an inhibitor of CDK9; (25) an inhibitor of farnesyl transferase; (26) an inhibitor of SHIP pathway; (27) an inhibitor of SRC; (28) an inhibitor of JAK; (29) a PARP inhibitor, (31) a ROS1 inhibitor; (32) an inhibitor of SHP pathway, or (33) an inhibitor of Src, FLT3, HDAC, VEGFR, PDGFR, LCK, Bcr-Abl or AKT; (34) an inhibitor of KrasG12C mutant; (35) a SHC inhibitor (e.g., PP2, AID371185); (36) a GAB inhibitor; (38) a PI-3 kinase inhibitor; (39) a MARPK inhibitor; (40) a CDK4/6 inhibitor; (41) a MAPK inhibitor; (42) a SHP2 inhibitor; (43) a checkpoint immune blockade agent; (44) a SOS1 inhibitor; or (45) a SOS2 inhibitor.

87. The method of claim 85, wherein the additional agent comprises an inhibitor of SHP2 selected from RMC-4630, ERAS-601,

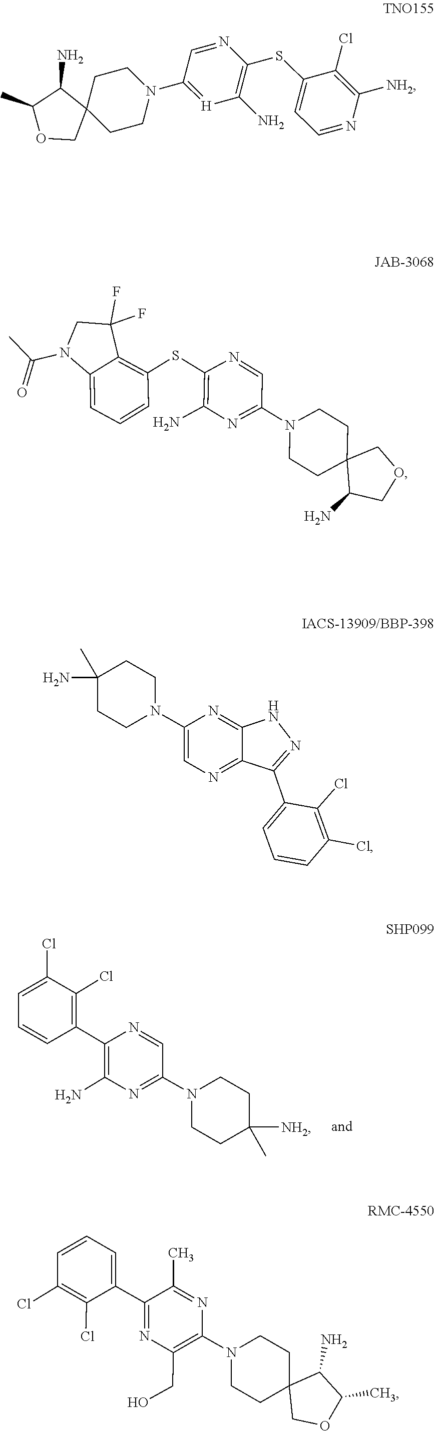

88. The method of claim 85, wherein the additional agent comprises an inhibitor of SOS selected from RMC-5845, BI-1701963

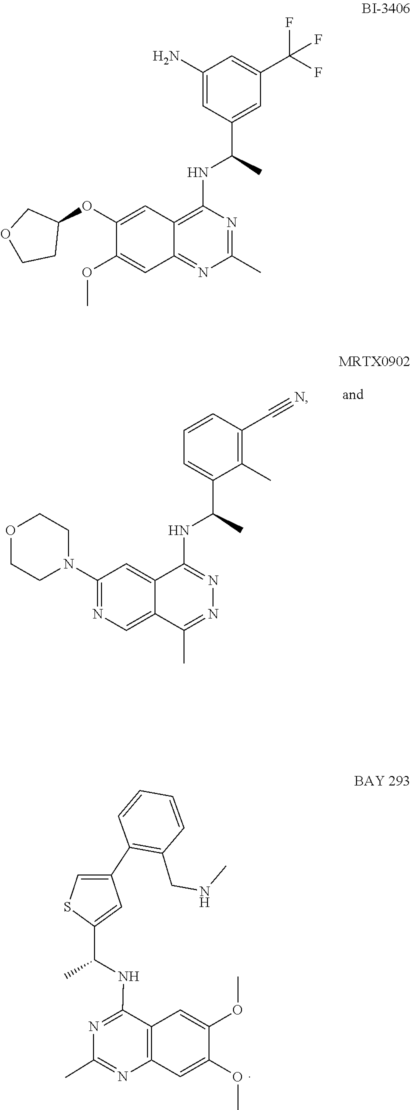 [SMILES: COc1cc2nc(C)nc(N[C@H](C)c3cc(N)cc(C(F)(F)F)c3)c2cc1O[C@H]1CCOC1 ; Cc1c(C#N)cccc1[C@@H](C)Nc1nnc(C)c2cnc(N3CCOCC3)cc12 ; CNCc1ccccc1-c1csc([C@@H](C)Nc2nc(C)nc3cc(OC)c(OC)cc23)c1]

89. The method of claim 85, wherein the additional agent comprises an inhibitor of EGFR selected from afatinib, erlotinib, gefitinib, lapatinib, cetuximab panitumumab, osimertinib, olnutinib, and EGF-816.

90. The method of claim 85, wherein the additional agent comprises an inhibitor of MEK selected from trametinib, cobimetinib, binimetinib, selumetinib, refametinib, and AZD6244.

91. The method of claim 85, wherein the additional agent comprises an inhibitor of ERK selected from ulixertinib, MK-8353, LTT462, AZD0364, SCH772984, BIX02189, LY3214996, and ravoxertinib.

92. The method of claim 85, wherein the additional agent comprises an inhibitor of CDK4/6 selected from palbociclib, ribociclib, and abemaciclib.

93. The method of claim 85, wherein the additional agent comprises an inhibitor of BRAF selected from sorafenib, vemurafenib, dabrafenib, encorafenib, regorafenib, and GDC-879.
